(19) United States
(12) Patent Application Publication
(10) Pub. No.: US 2024/0117011 A1
(43) Pub. Date: Apr. 11, 2024

(54) ANTIBODIES TARGETING THE SPIKE PROTEIN OF CORONAVIRUSES

(71) Applicant: The U.S.A., as Represented by the Secretary, Department of Health and Human Services, Bethesda, MD (US)

(72) Inventors: John Misasi, Kensington, MD (US); Lingshu Wang, North Potomac, MD (US); Chaim Aryeh Schramm, New York, NY (US); John R. Mascola, Rockville, MD (US); Daniel Cesar Douek, Bethesda, MD (US); Nancy J. Sullivan, Brookline, MA (US); Amy Ransier Henry, Monrovia, MD (US); Tongqing Zhou, Boyds, MD (US); Peter D. Kwong, Washington, DC (US); Wei Shi, Rockville, MD (US); Yi Zhang, North Potomac, MD (US); Eun Sung Yang, Bethesda, MD (US); Mario Roederer, Bethesda, MD (US); Rosemarie Diana Mason, Washington, DC (US); Amarendra Pegu, Rockville, MD (US); Julie E. Ledgerwood, Bethesda, MD (US)

(73) Assignee: The U.S.A., as Represented by the Secretary, Department of Health and Human Services, Bethesda, MD (US)

(21) Appl. No.: 18/263,995

(22) PCT Filed: Feb. 4, 2022
(86) PCT No.: PCT/US2022/015341

§ 371 Date: Aug. 2, 2023

Related U.S. Application Data
(60) Provisional application No. 63/147,419, filed on Feb. 9, 2021.

(51) Int. Cl. C07K 16/10; A61P 31/14; C12N 15/63; G01N 33/569

(52) U.S. Cl. CPC C07K 16/1003; A61P 31/14; C12N 15/63; G01N 33/56983; C07K 2317/31; C07K 2317/565; C07K 2317/567

(57) ABSTRACT

Disclosed are monoclonal antibodies, antigen binding fragments, and bi-specific antibodies that specifically bind a coronavirus spike protein, such as SARS-CoV-2. Also disclosed is the use of these antibodies for inhibiting a coronavirus infection, such as a SARS-CoV-2 infection. In addition, disclosed are methods for detecting a coronavirus, such as SARS-CoV-2, in a biological sample, using the disclosed antibodies.

### CROSS REFERENCE TO RELATED APPLICATIONS

This claims the benefit of U.S. Application No. 63/147,419, filed Feb. 9, 2021, incorporated herein by reference.

### FIELD OF THE DISCLOSURE

This relates to monoclonal antibodies and antigen binding fragments that specifically bind a coronavirus spike protein, and their use for inhibiting a beta coronavirus infection, such as a severe acute respiratory syndrome coronavirus 2 (SARS-CoV-2) infection in a subject, and are of use for detecting a coronavirus, such as SARS-CoV-2.

### BACKGROUND

In 2019, the International Committee on the Taxonomy of Viruses (ICTV) describes the Coronaviridae subfamily Orthocoronavirinae which included several viruses that are pathogenic to humans.

The most common human coronaviruses cause the common cold and include the alpha-coronaviruses 229E and NL63, and the beta-coronaviruses OC43 and HKU1. In addition to the coronaviruses that cause common cold symptoms, three beta-coronaviruses have been shown to be highly pathogenic in humans.

These viruses, Middle East Respiratory Syndrome Coronavirus (MERS), Severe Acute Respiratory Syndrome Coronavirus 1 (SARS-CoV-1) and SARS-CoV-2, can produce severe symptoms that can lead to death in human patients.

The genome of coronavirus is a large, enveloped, positive-sense, single-stranded RNA whose genome length varies by species and encodes multiple structural and non-structural proteins, encoded in several reading frames. The Spike protein (S) is expressed on the surface of the viral particle and is responsible for virus entry and infection of target cells. Transmission of coronaviruses can occur through multiple methods, including respiratory droplets, aerosols, fecal-oral and fomite routes.

At the end of 2019, a novel coronavirus was identified as the cause of a serve respiratory distress syndrome outbreak in Wuhan, China. This virus was later sequenced and identified to be highly similar to SARS-CoV-1 and based on this result, the novel Coronavirus was renamed SARS-CoV-2. The incubation period is typically between 4 to 14 days but can be as short as 1 day. Infection is characterized by fever, fatigue, cough, difficulty breathing and diarrhea. A subset of patients has significant respiratory distress, requiring hospitalization and oxygen supplementation. These patients can rapidly deteriorate and require intensive care unit admission and intubation. Severe disease is also characterized by abnormalities in multi-organ failure, blood clots and an apparent systemic inflammatory response syndrome.

In survivors of COVID-19, both humoral and cellular immunity are detected, however, their relative contribution to protection is unknown. The humoral immunity includes memory immunoglobulin responses that can be measures using assays for binding to viral antigens and neutralization of virus particles by recovered patient's serum. A need remains for antibodies that are highly potent for binding a coronavirus, and can be used as therapeutics and diagnostics.

### SUMMARY OF THE DISCLOSURE

Isolated monoclonal antibody or antigen binding fragments are disclosed that specifically bind to a coronavirus spike protein and neutralize SARS-CoV-2. In some embodiments, the antibody or antigen binding fragment includes one of:
a) a heavy chain variable region (V_{H}) and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as SEQ ID NOs: 1 and 5, respectively;
b) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 9 and 13, respectively;
c) a V_{H} and a V_{L} comprising a HCDR1, a HCDR2, and a HCDR3, and a 1LCDR1, a LCDR2, and a LCDR3 of the V_{H} and V_{L} set forth as SEQ ID NOs: 17 and 21, respectively;
d) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 25 and 29, respectively;
e) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 33 and 37, respectively;
f) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 41 and 45, respectively;
g) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 49 and 53, respectively;
h) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 57 and 61, respectively;
i) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 65 and 69, respectively;
j) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 73 and 77, respectively,
k) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 81 and 85;
1) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 89 and 93;
m) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 97 and 101;
n) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 105 and 109. The monoclonal antibody specifically binds to a coronavirus spike protein and neutralizes SARS-CoV-2; or
o) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 143 and 5, respectively.

In further embodiments, disclosed are multi-specific antibodies that including combinations of 2 or more these antibodies and/or antigen binding fragments.

In more embodiments, methods are disclosed for inhibiting a SARS-CoV-2 infection in a subject.

In further embodiments, methods are disclosed for detecting SARS-CoV-2 in a biological sample.

The foregoing and other features and advantages of the invention will become more apparent from the following detailed description of several embodiments which proceeds with reference to the accompanying figures.

### BRIEF DESCRIPTION OF THE FIGURES

FIGS. 1A-1E. Binding and pseudotyped virus neutralization of antibodies to coronavirus spike protein. (A) SARS CoV2 variants tested for binding and neutralization. (B) Cell surface binding of indicated antibodies to SARS-CoV2 spike variants. White indicates no change in binding relative to D614G variant, increasing red indicates increased binding of the antibody to the variant and increasing blue indicated decreasing binding of the antibody to the variant. (C) Neutralization of pseudotyped lentiviruses with Wuhan 1 (wt) spike (S) or the indicated SARS CoV2 variant with the mutations as shown in panel A. Neutralization was determined by incubating virus and antibodies at various dilutions prior to the addition to cells. Infection % was used to generate values for the inhibitory concentration 50 (IC₅₀) and 80 (IC₈₀). These values indicate the amount of antibody required in μg/mL to reduce infection by 50% and 80%, respectively. (D) Protein domains from the spike protein of SARS CoV2 stabilized 2-Proline (S2P), N-terminal domain (NTD), receptor binding domain (RBD) and S1 domains or SARS CoV S2P were coated onto ELISA plate and the indicated antibodies tested for reactivity. Positive reactivity for SARS-CoV-2 ELISA to the S2P, NTD, RBD or S1 domain is indicated by t, no reactivity indicated by - and low reactivity indicated by æ-. Binding domain classification based on the results is indicated in the column labeled “Target”. Binding to SARS CoV-1 (a.k.a., SARS1) or SARS-CoV2 S2P in ELISA is shown. Reactivity level is shown a −, +, ++, +++, ++++ or +++++ for each antibody. (E) Initial variant neutralization data, see also FIG. 28 for additional neutralization data.”

FIG. 2. Competition group determination by BLI for A23-58.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 3. Cryo-EM structure of A23-58.1 Fab in complex with SARS-CoV-2 spike. 3 Fabs are shown binding to RBD on the spike protein. The epitope is located at the tip of RBD with residues 417, 453, 455, 456, 473, 475-480, 483-488, 489 and 493 contributing to antibody binding. Residues 417 and E484 are located at the edge of the epitope and contributed ˜8% the antibody binding surface.

FIG. 4A. Competition group determination by BLI for A19-61.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition.

FIG. 4B. Mapping of Epitopes by Negative Stain EM.

FIG. 5. Competition group determination by BLI for A19-46.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 6. Competition group determination by BLI for A23-105.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 7. Competition group determination by BLI for A789-1.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 8. Competition group determination by BLI for A20-29.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 9. Competition group determination by BLI for A19-30.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 10. Competition group determination by BLI for A20-36.1 and A20-9.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 11. Competition group determination by BLI for A23-97.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 12. Competition group determination by BLI for A23-113.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 13. Competition group determination by BLI for A23-80.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 14. Competition group determination by BLI for A19-82.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIG. 15. Competition group determination by BLI for B1-182.1. The order of addition to the biosensors is SARS CoV2 S2P (antigen), competitor mAb, analyte mAb, and mAb114 is an isotype control mAb that does not bind S2P. Red indicates >80% competition, Yellow 60-79.9% competition and white <60% or no competition. Black boxes are not relevant to the competition group assignment and were removed for clarity.

FIGS. 16A-16H. Identification and classification of highly potent antibodies from convalescent SARS-CoV-2 subjects. (A) Sera from twenty-two convalescent subjects were tested neutralizing (y-axis, ID₅₀) and binding antibodies (x-axis, S-2P ELISA AUC) and four subjects, A19, A20, A23 and B1 (colored) with both high neutralizing and binding activity against the WA-1 were selected for antibody isolation. (B) Final flow cytometry sorting gate of CD19+/CD20+/IgG+ or IgA+ PBMCs for four convalescent subjects (A19, A20, A23 and B1). Shown is the staining for RBD-SD1 BV421, S1 BV786 and S-2P APC or Ax647. Cells were sorted using indicated sorting gate (pink) and percent positive cells that were either RBD-SD1, S1 or S-2P positive is shown for each subject. (C) Gross binding epitope distribution was determined using an MSD-based ELISA testing against RBD, NTD, S1, S-2P or HexaPro. S2 binding was inferred by S-2P or HexaPro binding without binding to other antigens. Indeterminant epitopes showed a mixed binding profile. Total number of antibodies (i.e., 200) and absolute number of antibodies within each group is shown. (D) Neutralization curves using WA-1 spike pseudotyped lentivirus and live virus neutralization assays to test the neutralization capacity of the indicated antibodies (n=2-3). (E) Table showing antibody binding target, IC₅₀ for pseudovirus and live virus neutralization and Fab:S-2P binding kinetics (n=2) for the indicated antibodies. (F) SPR-based epitope binning experiment. Competitor antibody (y-axis) is bound to S-2P prior to incubation with the analyte antibody (x-axis) as indicated and percent competition range bins are shown as red (>=75%), orange (60-75%) or white <60%) (n=2). Negative control antibody is anti-Ebola glycoprotein antibody mAb114 (37). (G) Competition of ACE2 binding. The indicated antibodies (y-axis) compete binding of S-2P to soluble ACE2 protein using biolayer interferometry (left column, percent competition (>=75% shown as red, <60% as white) or to cell surface expressed ACE2 using cell surface staining (right column, EC₅₀ at ng/ml shown). (H) Negative stain 3D reconstructions of SARS-CoV-2 spike and Fab complexes. A19-46.1 and A19-61.1 bind to RBD in the down position while A23-58.1 and B1-182.1 bind to RBD in the up position. Representative classes were shown with 2 Fabs bound, though stoichiometry at 1 to 3 were observed.

FIG. 17A-17D. Antibody binding and neutralization of variants of concern or interest
(A) Table showing domain and mutations relative to WA-1 for each of the 10 variants tested in panels B-C.
(B) Spike protein variants were expressed on the surface of HEK293T cells and binding to the indicated antibody was measured using flow cytometry. Data is shown as Mean Fluorescence intensity (MFI) normalized to the MFI for the same antibody against the D614G parental variant. Percent change is indicated by a color gradient from red (increased binding, Max 500%) to white (no change, 100%) to blue (no binding, 0%). (C) IC₅₀ and IC₈₀ values for the indicated antibodies against 10 variants shown in (A). Ranges are indicated by colors white (>10000 ng/mL), light blue (1000-10000 ng/mL), yellow (100-1000 ng/mL), orange (50-100 ng/mL), red (10-50 ng/mL), maroon (1-10 ng/mL) and purple (<1 ng/mL). (D) Location of spike protein variant mutations on the spike glycoprotein for B.1.1.7, B.1.351, B.1.429, P.1 v2. P681 and V1176 are not resolved in the structure and therefore their locations are not noted in B.1.1.7 and P.1 v2.

FIG. 18A-18E. Structural basis of binding and neutralization for antibodies A23-58.1 and B1-182.1. (A) Cryo-EM structure of A23-58.1 Fab in complex with SARS-CoV-2 HexaPro spike. Overall density map is shown to the left with protomers in shades of grey. One of the A23-58.1 Fab bound to the RBD is shown. Structure of the RBD and A23-58.1 after local focused refinement was shown to the right. The heavy chain CDRs are identified for CDR H1, CDR H2 and CDR H3, respectively. The light chain CDRs are also identified for CDR L1, CDR L2 and CDR L3, respectively. The contour level of Cryo-EM map is 5.7 s. (B) Cryo-EM structure of B1-182.1 Fab in complex with SARS-CoV-2 HexaPro spike. Overall density map is shown to the left with protomers indicated. One of the B1-182.1 Fab bound to the RBD is shown. Structure of the RBD and B1-182.1 after local focused refinement was shown to the right. The heavy chain CDRs are shown for CDR H1, CDR H2 and CDR H3, respectively. The light chain CDRs are shown for CDR L1, CDR L2 and CDR L3, respectively. The contour level of Cryo-EM map is 4.0 s. (C) Interaction between A23-58.1 and RBD. All CDRs were involved in binding of RBD. Epitope of A23-58.1 is shown in bright green surface. RBD mutations in current circulating SARS-CoV-2 variants are colored red. K417 and E484 are located at the edge of the epitope. (D) Interaction details at the antibody-RBD interface. The tip of the RBD binds to a cavity formed by the CDRs (shown viewing down to the cavity). Interactions between aromatic/hydrophobic residues are prominent at the lower part of the cavity. Hydrogen bonds at the rim of the cavity are marked with dashed lines. RBD residues were labeled with italicized font. (E) Paratopes of A23-58.1, B1-182.1, S2E12 (PDB ID: 7K45) and COVOX253 (PDB ID: 7BEN) from the same germline. Sequences of B1-182.1 (SEQ ID NO: 1 V_{H}, SEQ ID NO: 5, V_{L}), A23-58.1 (SEQ ID NO: 25, V_{H} and SEQ ID NO: 29, V_{L}) S2E12 and COVOX253 were aligned with variant residues underlined. Paratope residues for A23-58.1, B1-182.1, S2E12 and COVOC253 were highlighted. IGHV1-58*01 V_{H} is SEQ ID NO: 147, A23-58.1 V_{H} is SEQ ID NO: 25; B1-182.1 V_{H} is SEQ ID NO: 1; S2E1V_{H} is SEQ ID NO: 148, COVOX253 V_{H} is SEQ ID NO: 149; IGHV1-58*01 V_{L} is SEQ ID NO: 150, A23-58.1 V_{H} is SEQ ID NO: 29; B1-182.1 V_{H} is SEQ ID NO: 5; S2E1V_{L} is SEQ ID NO: 151, COVOX253 V_{L} is SEQ ID NO: 152.

FIGS. 19A-19E. Unique binding modes of A23-58.1 and B1-182.1 enable neutralization to VOCs. (A) Mapping of epitopes of A23-58.1, B1-182.1 and other antibodies on RBD (SEQ ID NO: 141). Epitope residues for different RBD-targeting antibodies are marked with * under the RBD sequence. (B) Comparison of binding modes of A23-58.1 and B1-182.1. Analysis indicated that axis of Fab B1-182.1 is rotated 6 degrees from that of A23-58.1 (left). This rotation resulted in a slight shift of the epitope of B1-182.1 on RBD which reduced its contact to E484 (right). RBD mutations of concern are highlighted, epitope surface of B1-182.1, the borders of ACE2-binding site and A23-58.1 epitope are shown. (C) Comparison of binding modes of A23-58.1, CB6 and REGN10933. For clarity, one Fab is shown to bind to the RBD on the spike. The shift of the binding site to the saddle of RBD encircled K417, E484 and Y453 inside the CB6 (black line) and REGN10933 epitopes (surface), explaining their sensitivity to the K417N, Y453F and E484K mutations. (D) Comparison of binding modes of A23-58.1 and LY-CoV555. One Fab is shown to bind to the RBD on the spike (left). E484 is located inside the LY-CoV555 epitope (Right, top), E484K/Q mutation abolishes critical contacts between RBD and CDR H2 and CDR L3, moreover, E484K/Q and L452R cause potential clashes with heavy chain of LY-CoV555, explaining its sensitivity to the E484K/Q and L452R mutations (Right, bottom). (E) IGHV1-58-derived antibodies target a supersite with minimal contacts to mutational hotspots. Supersite defined by common atoms contacted by the IGHV1-58-derived antibodies (A23-58.1, B1-182.1, S2E12 and COVOX253) on RBD is shown. Boundaries of the ACE2-binding site, epitopes of class I, II and III antibodies represented by C102 (PDB ID 7K8M), C144 (PDB ID 7K90) and C135 (PDB ID 7K8Z) are shown.

FIGS. 20A-20B. Critical binding residues for antibodies A23-58.1 and B1-182.1. (A) The indicated Spike protein mutations predicted by structural analysis were expressed on the surface of HEK293T cells and binding to the indicated antibody was measured using flow cytometry. Data is shown as Mean Fluorescence intensity (MFI) normalized to the MFI for the same antibody against the WA-1 parental binding. Percent change is indicated by a color gradient from red (increased binding, Max 200%) to white (no change, 100%) to blue (no binding, 0%). (B) IC₅₀ and IC₈₀ values for the indicated antibodies against WA-1 and the 9 spike mutations. Ranges are indicated by colors white (>10000 ng/mL), light blue (1000-10000 ng/mL), yellow (100-1000 ng/mL), orange (50-100 ng/mL), red (10-50 ng/mL), maroon (1-10 ng/mL) and purple (<1 ng/mL).

FIGS. 21A-21E. Mitigation of escape risk using dual antibody combinations. (A) Replication competent vesicular stomatitis virus (rcVSV) whose genome expressed SARS-CoV-2 WA-1 was incubated with serial dilutions of the indicated antibodies and wells with cytopathic effect (CPE) were passaged forward into subsequent rounds (Figure S8) after 48-72 hours. Total supernatant RNA was harvested and viral genomes shotgun sequenced to determine the frequency of amino acid changes. Shown are the spike protein amino acid/position change and frequency as a logo plot. Amino acid changes observed in two independent experiments are indicated in blue and green letters. (B) The indicated Spike protein mutations predicted by structural analysis (FIGS. 18A-18E) or observed by escape analysis (FIG. 21A) were expressed on the surface of HEK293T cells and binding to the indicated antibody was measured using flow cytometry. Data is shown as Mean Fluorescence intensity (MFI) normalized to the MFI for the same antibody against the WA-1 parental binding. Percent change is indicated by a gradient from grey (increased binding, Max 200%) to white (no change, 100%) to grey (no binding, 0%). (C) IC₅₀ and IC₈₀ values for the indicated antibodies against WA-1 and the mutations predicted by structural analysis (FIGS. 18A-18E) or observed by escape analysis (FIG. 21A). (D) Negative stain 3D reconstruction of the ternary complex of spike with Fab B1-182.1 and A19-46.1 (left) or A19-61.1 (right). (E) rcVSV SARS-CoV-2 was incubated with increasing concentrations (1.3e-4 to 50 mg/mL) of either single antibodies (A19-46.1, A19-61.1 and B1-182.1) and combinations of antibodies (B1-182.1/A19-46.1 and B1-182.1/A19-61.1). Every 3 days, wells were assessed for CPE and the highest concentration well with the >20% CPE was passaged forward onto fresh cells and antibody containing media. Shown is the maximum concentration with >20% CPE for each of the test conditions in each round of selection. Once 50 mg/mL has been reached, virus was no longer passaged forward and a dashed line is used to indicate maximum antibody concentration was reached in subsequent rounds.

FIGS. 22A-22E. Cryo-EM structure of the SARS-CoV-2 B.1.1.529 (Omicron) spike. (A) Cryo-EM map of the SARS-CoV-2 B.1.1.529 spike. Reconstruction density map at 3.29 Å resolution is shown with side and top views. The contour level of cryo-EM map is 4.0s. (B) B.1.1.529 amino acid substitutions introduced inter-protomer interactions. Substitutions in one protomer are shown as spheres. Examples of inter-protomer interactions introduced by B.1.1.529 substitutions were highlighted in box with zoom-in view to the side. Mutations are described as a percentage of the domain surface (surface) or as a percentage of the sequence (seq). (C) The NTD supersite of vulnerability is shown in semi-transparent surface along with a backbone ribbon. Amino acid substitutions, deletions, and insertions are shown. (D) The 15 amino acid substitutions clustered on the rim of RBD, changed 16% of the RBD surface areas (left) and increased electro-positivity of the ACE2-binding site (right). Mutated residues were shown as sticks. The ACE2-binding site on the electrostatic potential surface were also marked. (E) Mapping B.1.1.529 RBD substitutions on the epitopes of Barnes Class I-IV antibodies. The locations of the substitutions were shown on the surface. Those may potentially affect the activity of antibodies in each class were labeled with their residue numbers. Class I footprint were defined by epitopes of CB6 and B1-182.1, Class II footprint were defined by epitopes of A19-46.1 and LY-CoV555, Class III footprint were defined by epitopes of A19-61.1, COV2-2130, LY-CoV1404 and 5309, Class IV footprint were defined by epitopes of DH1407 and 5304. Class I and II epitope have overlap with the ACE2 binding site, while class III and IV do not. Class II and III epitopes allow binding to WA-1 when RBD is in the up or down conformation.

FIGS. 23A-23C. SARS-CoV-2 monoclonal antibody binding and neutralization. (A) Models of SARS-CoV-2 WA-1 spike protein (PDB: 6XM3) with the locations of substitutions present of variants indicate as red dots. Also noted is the total number of mutation and the number and locations of receptor binding domain (RBD) mutants in variant of concern spike proteins. (B) Full length spike proteins from the indicated SARS-CoV-2 variants were expressed on the surface of transiently transfected 293T cells and binding to indicated monoclonal antibodies was assessed by flow cytometry. Shown is the mean fluorescence intensity (MFI) of bound antibody on the indicated cell relative to the MFI of the same antibody bound to D614G expressing cells. The data is expressed as a percentage. Shown is a representative experiment (n=2-3 for each antibody). (C) Lentiviruses pseudotyped with SARS-CoV-2 spike proteins from D614G, B.1.1.7, B.1.351, P.1, B.1.617.2 or B.1.1.529 were incubated with serial dilutions of the indicated antibodies and IC₅₀ and IC₈₀ values determined. S309 was tested on 293 flpin-TMPRSS2-ACE2 cells while all the other antibodies were tested on 293T-ACE2 cells. *n.d.=not determined due to incomplete neutralization that plateaued at <80%.

FIGS. 24A-24D. Functional and structural basis of Class I antibody neutralization and mechanistic basis of retained potency against B.1.1.529 VOC. (A) Lentiviruses pseudotyped with SARS-CoV-2 spike proteins from D614G or D614G plus the indicated point mutations found within the B.1.1.529 spike were incubated with serial dilutions of the indicated antibodies, transduced 293T-ACE2 cells and IC₅₀ and IC₈₀ values determined. S375F and G496S viruses were not available and are shown as “not tested” (n.t). G496R was available and substituted for G496S. (B) Mapping of B.1.1.529 amino acid substitutions at the epitope of Class I antibody CB6. RBD-bound CB6 was docked onto the B.1.1.529 spike structure. B.1.1.529 amino acid substitutions incompatible with CB6 binding were identified and labeled. K417N mutation caused clash in the center of the paratope. B.1.1.529 RBD is shown in cartoon with amino acid substitutions in sticks. CB6 is shown in surface representation with heavy and light chains shown, 153-156 respectively. (C) Docking of RBD-bound VH1-58-derived Class I antibody B1-182.1 onto the B.1.1.529 spike structure identified 4 substitutions with potential steric hindrance. B1-182 is shown in surface representation with heavy and light chains. B.1.1.529 amino acid substitutions that may affect binding of VH1-58 antibodies were labeled. (D) Structural basis for effective neutralization of the B.1.1.529 VOC by VH1-58-derived antibodies. Even though VH1-58 antibodies, such as the S2E12, COV2-2196, A23-58.1 and B1-182.1, share high sequence homology (right, top), their neutralization potency against B.1.1.529 vary. Structural analysis indicated that CDR H3 residue 100C, located at the interfacial cavity formed by RBD, heavy and light chains, may determine their potency against B.1.1.529 (left). Size of this residue correlated with potency with two-tailed p=0.046 (right, bottom). In FIG. 24D, SEQ ID NO: 153-156 are shown.

FIGS. 25A-25E. Functional and structural basis of Class II antibody binding, neutralization, and escape. (A) Lentiviruses pseudotyped with SARS-CoV-2 spike proteins from D614G or D614G plus the indicated point mutations found within the B.1.1.529 spike were incubated with serial dilutions of the indicated antibodies, transduced 293T-ACE2 cells and IC₅₀ and IC₈₀ values determined. S375F and G496S viruses were not available and are shown as “not tested” (n.t). G496R was available and substituted for G496S. (B) Cryo-EM structure of class II antibody A19-46.1 Fab in complex with the B.1.1.529 spike. Overall density map is shown to the left with protomers. Two A19-46.1 Fabs bound to the RBD in the up-conformation are shown. Structure of the RBD and A19-46.1 after local focused refinement was shown to the right in cartoon representation. The heavy chain CDRs (CDR H1, CDR H2 and CDR H3) are shown. The light chain CDRs (CDR L1, CDR L2 and CDR L3, respectively) are also shown. The contour level of Cryo-EM map is 4.0 s. (C) Interaction between A19-46.1 and RBD. CDR H3 and all light chain CDRs were involved in binding of RBD (left). Epitope of A19-46.1 is shown on the B.1.1.529 RBD surface with amino acid substitutions. Ser446, A484 and R493 are located at the edge of the epitope of Fab A19-46.1 (right). RBD residues are labeled with italicized font. (D) Binding of A19-46.1 to RDB prevents binding of the ACE2 receptor. ACE2 and A19-46.1 are shown in cartoon representation. (E) Comparison of binding modes to RBD for antibody A19-46.1 and LY-CoV555. Even though both antibodies target similar regions on RBD, different approaching angle caused clash between LY-CoV555 CDR H3 and B.1.1.529 mutation Arg493 (left and inset). B.1.1.529 mutations involved in binding of A19-46.1 are only at the edge of its epitope while both Arg493 and A484 locate in the middle of LY-CoV555 epitope (right). Leu452 to Arg mutation that knockouts A19-46.1 and LY-CoV555 binding in other SARS-CoV-2 variants is colored in blue.

FIGS. 26A-26G. Functional and structural basis of Class III antibody binding, neutralization, and retained potency against the B.1.1.529 VOC. (A) Lentiviruses pseudotyped with SARS-CoV-2 spike proteins from D614G or D614G plus the indicated point mutations found within the B.1.1.529 spike were incubated with serial dilutions of the indicated antibodies and IC₅₀ and IC₈₀ values determined. A19-61.1 and LY-COV1404 were assayed on 293T-ACE2 cells while S309 and CoV2-2130 were tested on 293 flpin-TMPRSS2-ACE2 cells. S375F and G496S viruses were not available and are shown as “not tested” (n.t). G496R was available and substituted for G496S. (B) Cryo-EM structure of SARS-CoV-2 WA-1 spike in complex with class I antibody B1-182.1 and class III antibody A19-61.1 at 2.83 Å resolution. Overall density map is shown with protomers. Two RBDs were in the up conformation with each binding both Fabs, and one RBD was in the down position with A19-61.1 bound. RBD, B1-182.1 and A19-61.1 are shown (left). Structure of the RBD with both Fabs bound after local focused refinement was shown to the right in cartoon representation. RBD is shown green cartoon and antibody light chains are shown (middle). Epitope of A19-61.1 is shown as a surface on RBD with interacting CDRs labeled (right). The contour level of cryo-EM map is 5.2s. (C) Structural basis of B.1.1.529 resistance to A19-61.1. Mapping of the A19-61.1 epitope onto the B.1.1.529 RBD indicated G446S clashed with CDR H3 of A19-61.1. RBD is shown in cartoon with mutation residues in sticks, epitope of A19-61.1 is shown in on the surface. (D) Structural basis of CoV2-2130 neutralization of the B.1.1.529 VOC. Docking of the CoV2-2130 onto the B.1.1.529 RBD showed Y50 in CDR L2 posed a minor clash with 5446. RBD is shown in cartoon with mutation residues in sticks, epitope of CoV2-2130 is shown on the surface. (E) Structural basis of 5309 neutralization of the B.1.1.529 VOC. Docked complex of 5309 and B.1.1.529 RBD showed the S371L/S373P/S375F. Loop. Changed conformation, and the S371L mutation is adjacent to 5309 epitope while G339D mutation located inside the epitope. D339 sidechain clash with CDR H3 Y100. B.1.1.529 RBD is shown in cartoon with mutation residues in sticks, WA-1 RBD is shown in gray cartoon. (F) Structural basis of LY-CoV1404 neutralization of the B.1.1.529 VOC. Docking of the LY-CoV1404 onto the B.1.1.529 RBD identified 4 amino acid substitutions in the epitope with G446S causing potential clash with CDR H2 R60. However, comparison of both LY-CoV1404-bound and non-bound B.1.1.529 RBD indicated the S446 loop has the flexibility to allow LY-CoV1404 binding. B.1.1.529 residues at LY-CoV1404 epitope are shown with corresponding WA-1 residues. CDR H3 is shown in cartoon representation. (G) Overlay of epitope footprints of class III antibodies onto the B.1.1.529 RBD. B.1.1.529 RBD amino acid substitution locations are shown

FIG. 27A-27C. Potent neutralization of SARS-CoV-2 B.1.1.529 using combinations of antibodies. (A) Lentiviruses pseudotyped with SARS-CoV-2 spike proteins from D614G or D614G plus the indicated point mutations found within the B.1.1.529 spike were incubated with serial dilutions of the indicated combination of antibodies and IC₅₀ and IC₈₀ values determined. S375F and G496S viruses were not available and are shown as “not tested” (n.t). G496R was available and substituted for G496S. (B) Neutralization IC₅₀ (ng/mL) values for each of the indicated cocktail (x-axis) or its component antibodies. The IC₅₀ for first antibody is listed as mAb1, the second antibody as mAb2 or cocktail. (C) Cryo-EM structure of B.1.1.529 spike in complex with antibodies A19-46.1 and B-182.1 at 3.86 Å resolution. Overall density map is shown to the left with protomers (left). All RBD are in up-conformation with both Fabs bound (middle). Binding of one Fab (such as B1-182.1) induces RBD into the up-conformation and potentially facilitates binding of the other Fab (such as A19-46.1) which only recognizes the up-conformation of RBD (right). A19-46.1 and B-182.1 are shown, respectively. The contour level of cryo-EM map is 6.5 s.

FIGS. 28A-28B. Neutralization of pseudotyped lentiviruses with Wuhan 1 (wt) spike (S) or the indicated SARS-CoV-2 variants. Neutralization was determined by incubating virus and antibodies at various dilutions prior to the addition to cells. Infection % was used to generate values for the inhibitory concentration 50 (IC₅₀) and 80 (IC₈₀). These values indicate the amount of antibody required in μg/mL to reduce infection by 50% and 80%, respectively. (A) Neutralization IC50s and IC80s to variants containing single or combined mutations. (B) Neutralization IC50s and IC80s to variants (VOCs/VOIs).

FIGS. 29A-29C. Tables for Example 30. (A) Yield and precipitation for B1-182.1 and A23.58.1. (B) Yield, concentration and precipitation properties for B1-182.1_58CDRH3 heavy/B1-182.1 light and B1-182 heavy/B1-182.1 light_5Mut. (C) Neutralization by B1-182.1_58CDRH3 heavy/B1-182.1 of lentiviruses pseudotyped with the indicated spike proteins from SARS-CoV-2 (IC50, IC80 at μg/ml).

FIG. 30. Sera levels of SARS-CoV-2 antibodies in human FcRn transgenic mice administered at 5 mg/kg via the IV route.

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Feb. 2, 2022, 67.6 KB], which is incorporated by reference herein.
SEQ ID NO: 1 is the amino acid sequence of the B1-182.1 V_{H}. SEQ ID NOs: 2, 3, and 4 are the amino acid sequences of the HCDR1, HCDR2, and HCDR3, respectively.
SEQ ID NO: 5 is the amino acid sequence of the B1-182.1 V_{L}. SEQ ID NOs: 6, 7, and 8 are the amino acid sequences of the LCDR1, LCDR2, and LCDR3, respectively.
SEQ ID NO: 9 is the amino acid sequence of the A19-61.1 V_{H}. SEQ ID NOs: 10, 11 and 12 are the amino acid sequences of the HCDR1, HCDR2, and HCDR3, respectively.
SEQ ID NO: 13 is the amino acid sequence of the A19-61.1 V_{L}. SEQ ID NOs: 14, 15, and 16 are the amino acid sequences of the LCDR1, LCDR2, and LCDR3, respectively.
SEQ ID NO: 17 is the amino acid sequence of the A19-46.1 V_{H}. SEQ ID NOs: 18, 19, and 20 are the amino acid sequences of the HCDR1, HCDR2, and HCDR3, respectively.
SEQ ID NO: 21 is the amino acid sequence of the A19-46.1 V_{L}. SEQ ID NOs: 22, 23, and 24 are the amino acid sequences of the LCDR1, LCDR2, and LCDR3, respectively.
SEQ ID NO: 25 is the amino acid sequence of the A23-58.1 V_{H}. SEQ ID NOs: 26, 27, and 28 are the amino acid sequences of the HCDR1, HCDR2, and HCDR3, respectively.
SEQ ID NO: 29 is the amino acid sequence of the A23-58.1 V_{L}. SEQ ID NOs: 30, 31, and 32 are the amino acid sequences of the LCDR1, LCDR2, and LCDR3, respectively.
SEQ ID NO: 33 is the amino acid sequence of the A20-29.1 V_{H}. SEQ ID NOs: 34, 35, and 36 are the amino acid sequences of the HCDR1, HCDR2, and HCDR3, respectively.
SEQ ID NO: 37 is the amino acid sequence of the A20-29.1 V_{L}. SEQ ID NOs: 38, 39, and 40 are the amino acid sequences of the LCDR1, LCDR2, and LCDR3, respectively.
SEQ ID NO: 41 is the amino acid sequence of the A23-105.1 V_{H}. SEQ ID NOs: 42, 43, and 44 are the amino acid sequences of the HCDR1, HCDR2, and HCDR3, respectively.
SEQ ID NO: 45 is the amino acid sequence of the A23-105.1 V_{L}. SEQ ID NOs: 46, 47, and 48 are the amino acid sequences of the LCDR1, LCDR2, and LCDR3, respectively.
SEQ ID NO: 49 is the amino acid sequence of the A19-1.1 V_{H}. SEQ ID NOs: 50, 51, and 52 are the amino acid sequences of the HCDR1, HCDR2, and HCDR3, respectively.
SEQ ID NO: 53 is the amino acid sequence of the A19-1.1 V_{L}. SEQ ID NOs: 54, 55, and 56 are the amino acid sequences of the LCDR1, LCDR2, and LCDR3, respectively.
SEQ ID NO: 57 is the amino acid sequence of the A19-30.1 V_{H}. SEQ ID NOs: 58, 59, and 60 are the amino acid sequences of the HCDR1, HCDR2, and HCDR3, respectively.
SEQ ID NO: 61 is the amino acid sequence of the A19-30.1 V_{L}. SEQ ID NOs: 62, 63, and 64 are the amino acid sequences of the LCDR1, LCDR2, and LCDR3, respectively.
SEQ ID NO: 65 is the amino acid sequence of the A20-36.1 V_{H}. SEQ ID NOs: 66, 67, and 68 are the amino acid sequences of the HCDR1, HCDR2, and HCDR3, respectively.
SEQ ID NO: 69 is the amino acid sequence of the A20-36.1 V_{L}. SEQ ID NOs: 70, 71, and 72 are the amino acid sequences of the LCDR1, LCDR2, and LCDR3, respectively.
SEQ ID NO: 73 is the amino acid sequence of the A23-97.1 V_{H}. SEQ ID NOs: 74, 75, and 76 are the amino acid sequences of the HCDR1, HCDR2, and HCDR3, respectively.
SEQ ID NO: 77 is the amino acid sequence of the A23-97.1 V_{L}. SEQ ID NOs: 78, 79, and 80 are the amino acid sequences of the LCDR1, LCDR2, and LCDR3, respectively.
SEQ ID NO: 81 is the amino acid sequence of the A23-113.1 V_{H}. SEQ ID NOs: 82, 83, and 84 are the amino acid sequences of the HCDR1, HCDR2, and HCDR3, respectively.
SEQ ID NO: 85 is the amino acid sequence of the A23-113.1 V_{L}. SEQ ID NOs: 86, 87, and 88 are the amino acid sequences of the LCDR1, LCDR2, and LCDR3, respectively.
SEQ ID NO: 89 is the amino acid sequence of the A23-80.1 V_{H}. SEQ ID NOs: 90, 91, and 92 are the amino acid sequences of the HCDR1, HCDR2, and HCDR3, respectively.
SEQ ID NO: 93 is the amino acid sequence of the A23-80.1 V_{L}. SEQ ID NOs: 94, 95, and 96 are the amino acid sequences of the LCDR1, LCDR2, and LCDR3, respectively.
SEQ ID NO: 97 is the amino acid sequence of the A19-82.1 V_{H}. SEQ ID NOs: 98, 99, and 100 are the amino acid sequences of the HCDR1, HCDR2, and HCDR3, respectively.
SEQ ID NO: 101 is the amino acid sequence of the A19-82.1 V_{L}. SEQ ID NOs: 102, 103, and 104 are the amino acid sequences of the LCDR1, LCDR2, and LCDR3, respectively.
SEQ ID NO: 105 is the amino acid sequence of the A20-9.1 V_{H}. SEQ ID NOs: 106, 107, and 108 are the amino acid sequences of the HCDR1, HCDR2, and HCDR3, respectively.
SEQ ID NO: 109 is the amino acid sequence of the A20-9.1 V_{L}. SEQ ID NOs: 110, 111, and 112 are the amino acid sequences of the LCDR1, LCDR2, and LCDR3, respectively.
SEQ ID NOs: 113-140 are nucleic acid sequences encoding a V_{H} or a V_{L}.
SEQ ID NO: 141 is the RBD sequence in FIG. 19A.
SEQ ID NO: 142 is a nucleic acid sequence of a portion of a nucleic acid molecule encoding IgA.
SEQ ID NO: 143 is the amino acid sequence of the B1-182.1_58.1CDRH3 heavy/B1-182.1 light.
SEQ ID NOs: 2, 3, and 28 are the amino acid sequences of the HCDR1, HCDR2, and HCDR3, respectively.
SEQ ID NO: 144 is the amino acid sequence of the B1-182.1 heavy/B1-182.1 light_5Mut chain.
SEQ ID NOs: 6, 145 and 146 are the CDR sequences.
SEQ ID NO: 147 is the amino acid sequence of the IGHV1-58*01 V_{H}, SEQ ID NO: 148 is the amino acid sequence of the S2E12V_{H}.
SEQ ID NO: 149 is the amino acid sequence of the COVOX253 V_{H}.
SEQ ID NO: 150 is the amino acid sequence of the IGHV1-58*01 V_{L}.
SEQ ID NO: 151 is the amino acid sequence of the S2E12V_{L}.
SEQ ID NO: 152 is the amino acid sequence of the COVOX253 V_{L}.
SEQ ID NO: 153 is the amino acid sequence of a portion of A23-58.1.
SEQ ID NO: 154 is the amino acid sequence of a portion of B1-182.1.
SEQ ID NO: 155 is the amino acid sequence of a portion of CoV2-2196.
SEQ ID NO: 156 is the amino acid sequence of a portion of S2E12.

### DETAILED DESCRIPTION OF SEVERAL EMBODIMENTS

Monoclonal antibodies that specifically bind the spike protein of a coronavirus, such as SARS-CoV-2, are disclosed herein. In some embodiments, these antibodies are of use for inhibiting a coronavirus infection, and for detecting a coronavirus in a biological sample. The antibodies are potent neutralizing antibodies and target unique epitopes in the spike glycoprotein of SARS-CoV-2.

Worldwide genomic sequencing has revealed the occurrence of SARS-CoV-2 variants that increase transmissibility and reduce potency of vaccine-induced and therapeutic antibodies (see, for example, Wibmer et al., Nat. Med. 27, 622-625 (2021); Wang et al., Nature. 593, 130-135 (2021); Muik et al., Science. 371, 1152-1153 (2021); Wang et al., Nature. 592, 616-622 (2021)). Recently, there has been a significant concern that antibody responses to natural infection and vaccination using ancestral spike sequences may result in focused responses that lack potency against mutations present in more recent variants (e.g., K417N, L452R, T478K, E484K/Q, N501Y in B.1.351, B.1.617.1 and B.1.617.2) (see, for example, Wibmer et al., Nat. Med. 27, 622-625 (2021); Wang et al., Nature. 593, 130-135 (2021); Muik et al., Science. 371, 1152-1153 (2021); Wang et al., Nature. 592, 616-622 (2021)). Additionally, neutralization of P.1 viruses can be achieved using sera obtained from subjects infected by B.1.351 (Moyo-Gwete et al., N. Engl. J. Med. 2 (2021), doi:10.1056/NEJMc2104192), suggesting that shared epitopes in RBD (i.e., K417N, E484K, N501Y) are mediating the cross-reactivity. While the mechanism of B.1.351 and P.1 cross reactivity is likely focused on the 3 RBD mutations, the mechanism of broadly neutralizing antibody responses between WA-1 and later variants is not as well established. It is disclosed herein that antibodies were isolated and defined with neutralization breadth covering newly emerging SARS-CoV-2 variants, including, but not limited to, the highly transmissible variants B.1.1.7, B.1.351, B.1.617.2 and B.1.1.529. Increased potency and breadth were mediated by binding to regions of the RBD tip that are offset from E484K/Q, L452R and other mutational hot spots that are major determinant of resistance in VOCs.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Unless context indicated otherwise, “about” refers to plus or minus 5% of a reference value. For example, “about” 100 refers to 95 to 105.

Administration: The introduction of an agent, such as a disclosed antibody, into a subject by a chosen route. Administration can be local or systemic. For example, if the chosen route is intravascular, the agent (such as antibody) is administered by introducing the composition into a blood vessel of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody and Antigen Binding Fragment: An immunoglobulin, antigen-binding fragment, or derivative thereof, that specifically binds and recognizes an analyte (antigen) such as a coronavirus spike protein, such as a spike protein from SARS-CoV-2. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (a) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “V_{H}” refer to the variable region of an antibody heavy chain, including that of an antigen binding fragment, such as Fv, scFv, dsFv or Fab. References to “V_{L}” or “V_{L}” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some embodiments, a disclosed antibody includes a heterologous constant domain. For example, the antibody includes a constant domain that is different from a native constant domain, such as a constant domain including one or more modifications (such as the “LS” mutation) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} a ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print; Lonberg, Nat. Biotech., 23: 1117-1125, 2005; Lonenberg, Curr. Opin. Immunol., 20:450-459, 2008).

Antibody or antigen binding fragment that neutralizes SARS-CoV-2: An antibody or antigen binding fragment that specifically binds to a SARS-CoV-2 antigen (such as the spike protein) in such a way as to inhibit a biological function associated with SARS-CoV-2 that inhibits infection. The antibody can neutralize the activity of SARS-CoV-2. For example, an antibody or antigen binding fragment that neutralizes SARS-CoV-2 may interfere with the virus by binding it directly and limiting entry into cells. Alternately, an antibody may interfere with one or more post-attachment interactions of the pathogen with a receptor, for example, by interfering with viral entry using the receptor. In some examples, an antibody that is specific for a coronavirus spike protein neutralizes the infectious titer of SARS-CoV-2.

In some embodiments, an antibody or antigen binding fragment that specifically binds to SARS-CoV-2 and neutralizes SARS-CoV-2 inhibits infection of cells, for example, by at least 50% compared to a control antibody or antigen binding fragment.

A “broadly neutralizing antibody” is an antibody that binds to and inhibits the function of related antigens, such as antigens that share at least 85%, 90%, 95%, 96%, 97%, 98% or 99% identity antigenic surface of antigen. With regard to an antigen from a pathogen, such as a virus, the antibody can bind to and inhibit the function of an antigen from more than one class and/or subclass of the pathogen. For example, with regard to a coronavirus, the antibody can bind to and inhibit the function of an antigen, such as the spike protein from coronaviruses including SARS-CoV-2.

Biological sample: A sample obtained from a subject. Biological samples include all clinical samples useful for detection of disease or infection in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a SARS-CoV-2 infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one embodiment, an antibody is linked to an effector molecule; for example, an antibody that specifically binds to SARS-CoV-2 covalently linked to an effector molecule, such as a detectable label. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a SARS-CoV-2-specific antibody can include up to 1, 2, 3, 4, 5, 6, 7, 8, 9, or up to 10 conservative substitutions compared to a reference antibody sequence and retain specific binding activity for spike protein binding, and/or SARS-CoV-2 neutralization activity. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the antibody, such as the ability to specifically bind to a coronavirus spike protein. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control, such as sample obtained from a healthy patient not infected a coronavirus. In other embodiments, the control is a positive control, such as a tissue sample obtained from a patient diagnosed with a coronavirus infection. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Coronavirus: A family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future.

Non-limiting examples of betacoronaviruses include SARS-CoV-2, Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

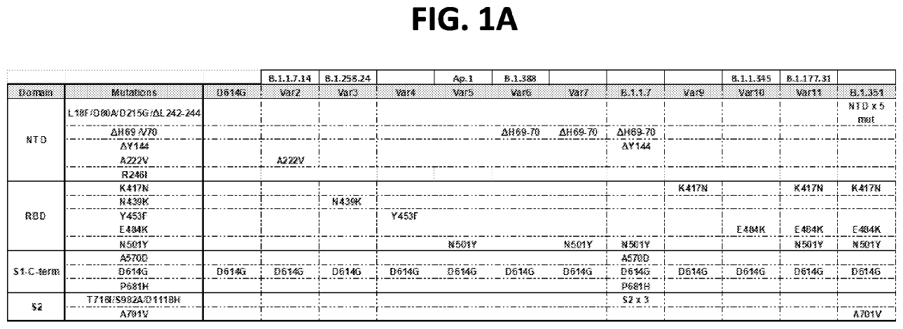

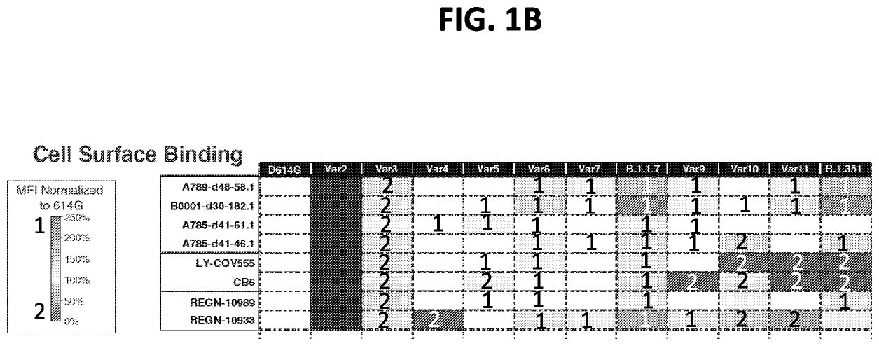

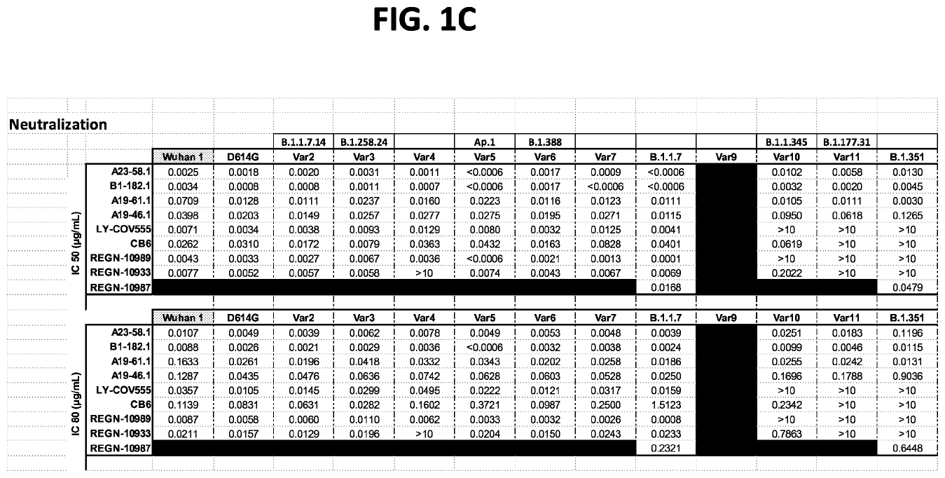

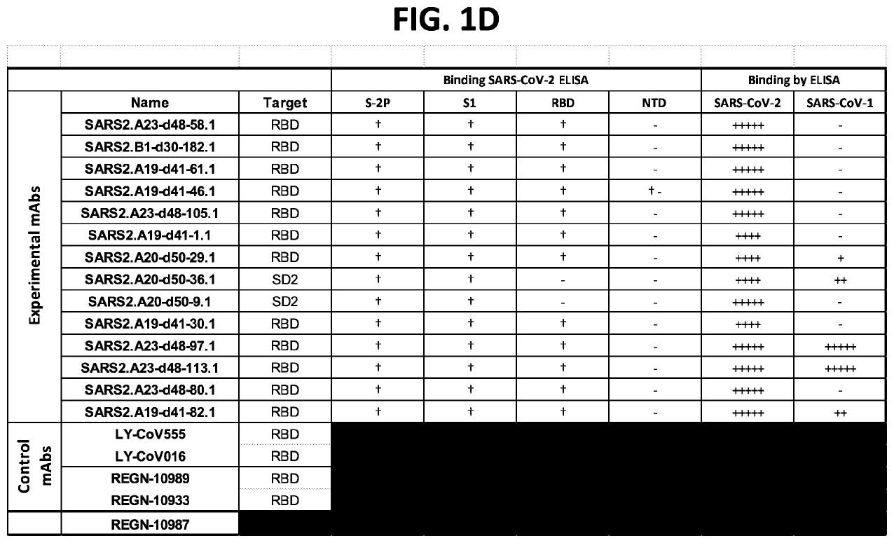

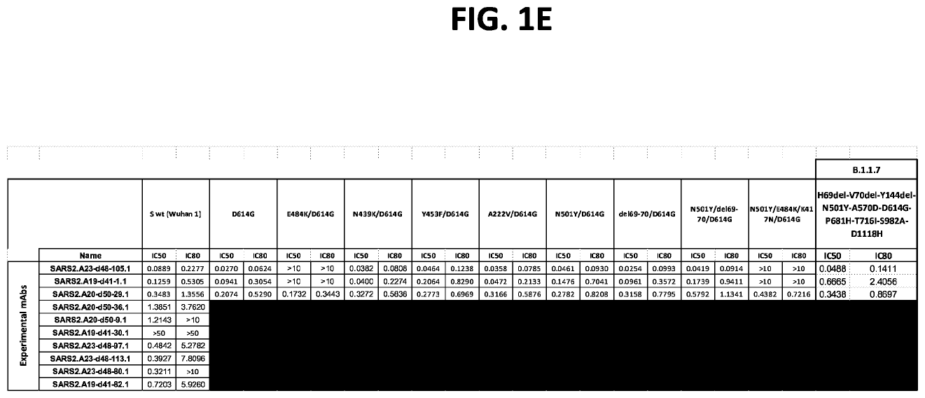

Dual variable domain immunoglobulin: A bi-specific antibody that includes two heavy chain variable domains and two light chain variable domains. Unlike IgG, however, both heavy and light chains of a DVD-immunoglobulin molecule contain an additional variable domain (VD) connected via a linker sequence at the N-termini of the V_{H} and V_{L} of an existing monoclonal antibody (mAb). Thus, when the heavy and the light chains combine, the resulting DVD-immunoglobulin molecule contains four antigen recognition sites, see Jakob et al., Mabs 5: 358-363, 2013, incorporated herein by reference, see FIG. 1 of Jaakob et al. for schematic and space-filling diagrams. A DVD-Ig™ molecule functions to bind two different antigens on each DFab simultaneously.

Effective amount: A quantity of a specific substance sufficient to achieve a desired effect in a subject to whom the substance is administered. For instance, this can be the amount necessary to inhibit a coronavirus infection, such as a SARS-CoV-2 infection, or to measurably alter outward symptoms of such an infection.

In one example, a desired response is to inhibit or reduce or prevent SARS-CoV-2 infection. The SARS-CoV-2 infection does not need to be completely eliminated or reduced or prevented for the method to be effective.

In some embodiments, administration of an effective amount of a disclosed antibody or antigen binding fragment that binds to a coronavirus spike protein can reduce or inhibit a SAR-CoV-2 infection (for example, as measured by infection of cells, or by number or percentage of subjects infected by the coronavirus or by an increase in the survival time of infected subjects, or reduction in symptoms associated with the infection) by a desired amount, for example by at least 10%, at least 20%, at least 50%, at least 60%, at least 70%, at least 80%, at least 90%, at least 95%, at least 98%, or even at least 100% (elimination or prevention of detectable infection), as compared to a suitable control.

The effective amount of an antibody or antigen binding fragment that specifically binds the coronavirus spike protein that is administered to a subject to inhibit infection will vary depending upon a number of factors associated with that subject, for example the overall health and/or weight of the subject. An effective amount can be determined by varying the dosage and measuring the resulting response, such as, for example, a reduction in pathogen titer. Effective amounts also can be determined through various in vitro, in vivo or in situ immunoassays.

An effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted, or a detectable marker. Effector molecules can include, for example, polypeptides and small molecules. Some effector molecules may have or produce more than one desired effect.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on a coronavirus spike protein.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cyl and Cγ2.

Although the boundaries of the Fc region may vary, the human IgG heavy chain Fc region is usually defined to include residues following C226 or P230 to the Fc carboxyl-terminus, wherein the numbering is according to the EU numbering system. The residues can also be identified by Kabat position. For IgA, the Fc region includes immunoglobulin domains Cα2 and Cα3 and optionally the lower part of the hinge between Cal and Cα2.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgA: A polypeptide belonging to the class of antibodies that are substantially encoded by a recognized immunoglobulin alpha gene. In humans, this class or isotype comprises IgA₁ and IgA₂. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG₁, IgG₂, IgG3, and IgG₄.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting or treating a disease: Inhibiting the full development of a disease or condition, for example, in a subject who is at risk for a disease such as a SARS-CoV-2 infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a disease or condition if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, a composition that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} Edition, Department of Health and Human Services, Public Health Service, National Institutes of Health, Bethesda, NIH Publication No. 91-3242, 1991).

Linker: A bi-functional molecule that can be used to link two molecules into one contiguous molecule, for example, to link a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some embodiments, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

SARS-CoV-2: Also known as Wuhan coronavirus or 2019 novel coronavirus, SARS-CoV-2 is a positive-sense, single stranded RNA virus of the genus betacoronavirus that has emerged as a highly fatal cause of severe acute respiratory infection. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

B.1.1.529, also known as the omicron variant, is a variant of the original SARS-CoV-2 first reported to the World Health Organization on Nov. 21, 2021. This variant has a total of 60 mutations compared to the original strain of SARS-CoV-2, specifically 50 nonsynonymous mutations, 8 synonymous mutations, and 2 non-coding mutations. Thirty-two mutations affect the spike protein (Δ67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F), or which approximately half are located in the receptor binding domain (319-541).

SARS Spike (S) protein: A class I fusion glycoprotein initially synthesized as a precursor protein of approximately 1256 amino acids in size for SARS-CoV, and 1273 for SARS-CoV-2. Individual precursor S polypeptides form a homotrimer and undergo glycosylation within the Golgi apparatus as well as processing to remove the signal peptide, and cleavage by a cellular protease between approximately position 679/680 for SARS-CoV, and 685/686 for SARS-CoV-2, to generate separate S1 and S2 polypeptide chains, which remain associated as S1/S2 protomers within the homotrimer and is therefore a trimer of heterodimers. The S1 subunit is distal to the virus membrane and contains the N-terminal domain (NTD) and the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain.

The numbering used in the disclosed SARS-CoV-2 S proteins and fragments thereof is relative to the S protein of SARS-CoV-2, the sequence of which was deposited as NCBI Ref. No. YP_009724390.1, which is incorporated by reference herein in its entirety.

Sequence identity: The identity between two or more nucleic acid sequences, or two or more amino acid sequences, is expressed in terms of the percentage identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994, Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example a coronavirus spike protein and does not bind in a significant amount to other proteins present in the sample or subject. With regard to a spike protein, the epitope may be present on SARS-CoV-2 spike protein, such that the antibody binds to the spike protein on both types of virus, but does not bind to other proteins. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 108 Molar, 10⁻⁹, or even less than about 10⁻¹⁰ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on a coronavirus spike protein an antibody that binds substantially to the coronavirus spike protein, such as the NTD or RBD of a spike protein from SARS-CoV-2, including viruses, substrate to which the spike protein is attached, or the protein in a biological specimen. It is, of course, recognized that a certain degree of non-specific interaction may occur between an antibody and a non-target. Typically, specific binding results in a much stronger association between the antibody and a spike protein than between the antibody other different coronavirus proteins (such as MERS), or from non-coronavirus proteins. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Living multi-cellular vertebrate organisms, a category that includes human and non-human mammals, such as non-human primates, pigs, camels, bats, sheep, cows, dogs, cats, rodents, and the like. In an example, a subject is a human. In a particular example, the subject is a human. In an additional example, a subject is selected that is in need of inhibiting a SARS-CoV-2 infection. For example, the subject is either uninfected and at risk of the SARS-CoV-2 infection or is infected and in need of treatment.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some embodiments, a viral vector comprises a nucleic acid molecule encoding a disclosed antibody or antigen binding fragment that specifically binds to a coronavirus spike protein and neutralizes the coronavirus. In some embodiments, the viral vector can be an adeno-associated virus (AAV) vector.

Under conditions sufficient for: A phrase that is used to describe any environment that permits a desired activity.

### II. Description of Several Embodiments

Isolated monoclonal antibodies and antigen binding fragments that specifically bind a coronavirus spike protein are provided. The antibodies and antigen binding fragments can be fully human. The antibodies and antigen binding fragments can neutralize a coronavirus, such as SARS-CoV-2. In some embodiments the disclosed antibodies can inhibit a coronavirus infection in vivo, and can be administered prior to, or after, an infection with a coronavirus, such as SARS-CoV-2. Bispecific antibodies including the variable domains of these antibodies are also provided. In addition, disclosed herein are compositions comprising the antibodies and antigen binding fragments and a pharmaceutically acceptable carrier. Nucleic acids encoding the antibodies, antigen binding fragments, variable domains, and expression vectors (such as adeno-associated virus (AAV) viral vectors) comprising these nucleic acids are also provided. The antibodies, antigen binding fragments, nucleic acid molecules, host cells, and compositions can be used for research, diagnostic, treatment and prophylactic purposes. For example, the disclosed antibodies and antigen binding fragments can be used to diagnose a subject with a coronavirus infection or can be administered to inhibit a coronavirus infection in a subject. Binding characteristics of each of the antibodies listed below are also provided in the Examples section.

### A. Monoclonal Antibodies that Specifically Bind a Coronavirus Spike Protein and Antigen Binding Fragments Thereof

The discussion of monoclonal antibodies below refers to isolated monoclonal antibodies that include heavy and/or light chain variable domains (or antigen binding fragments thereof) comprising a CDR1, CDR2, and/or CDR3 with reference to the IMGT numbering scheme (unless the context indicates otherwise). Various CDR numbering schemes (such as the Kabat, Chothia or IMGT numbering schemes) can be used to determine CDR positions. The amino acid sequence and the CDRs of the heavy and light chain of the disclosed monoclonal antibody according to the IMGT numbering scheme are provided in the listing of sequences, but these are exemplary only.

In some embodiments, a monoclonal antibody is provided that comprises the heavy and light chain CDRs of any one of the antibodies described herein. In some embodiment, a monoclonal antibody is provided that comprises the heavy and light chain variable regions of any one of the antibodies described herein.

TABLE A
IMGT CDRs of Antibodies and SEQ ID NOs
B1-182.1 V_{H}

| | SEQ ID NO: 1 | | CDR |
|---|---|---|---|
| V_{H} | positions | CDR protein sequence | SEQ ID NO |
| HCDR1 | 26-33 | GFTFTSSA | 2 |
| HCDR2 | 51-58 | IVVGSGNT | 3 |
| HCDR3 | 96-113 | CAAPYCSGGSCFDGFDIW | 4 |

B1-182.1 V_{L}

| | SEQ ID NO: 5 | | CDR |
|---|---|---|---|
| V_{L} | positions | CDR protein sequence | SEQ ID NO |
| LCDR1 | 27-33 | QSVSSSY | 6 |
| LCDR2 | 51-53 | GAS | 7 |
| LCDR3 | 89-99 | CQQYGNSPWTF | 8 |

A19-61.1 V_{H}

| | SEQ ID NO: 9 | | CDR |
|---|---|---|---|
| V_{H} | positions | CDR protein sequence | SEQ ID NO |
| HCDR1 | 26-33 | GFTFSSYA | 10 |
| HCDR2 | 51-58 | ISYDGSNQ | 11 |
| HCDR3 | 96-117 | CARDLAIAVAGTWHYYNGM | 12 |
| | | DVW | |

A19-61.1 V_{L}

| | SEQ ID NO: 13 | | CDR |
|---|---|---|---|
| V_{L} | positions | CDR protein sequence | SEQ ID NO |
| LCDR1 | 27-32 | QGISSW | 14 |
| LCDR2 | 50-52 | DAS | 15 |
| LCDR3 | 88-98 | CQQAKSFPITF | 16 |

A19-46.1 V_{H}

| | SEQ ID NO: 17 | | CDR |
|---|---|---|---|
| V_{H} | positions | CDR protein sequence | SEQ ID NO |
| HCDR1 | 26-33 | GFTLSSYG | 18 |
| HCDR2 | 51-58 | ISYDGSNK | 19 |
| HCDR3 | 96-116 | CARGWAYWELLPDYYYGM | 20 |
| | | DVW | |

A19-46.1 V_{L}

| | SEQ ID NO: 21 | | CDR |
|---|---|---|---|
| V_{L} | positions | CDR protein sequence | SEQ ID NO |
| LCDR1 | 26-34 | SGSVSTAYF | 22 |
| LCDR2 | 53-54 | GTN | 23 |
| LCDR3 | 90-101 | CVLYMGRGIVVF | 24 |

A23-58.1 V_{H}

| | SEQ ID NO: 25 | | CDR |
|---|---|---|---|
| V_{H} | positions | CDR protein sequence | SEQ ID NO |
| HCDR1 | 26-33 | GFTFTSSA | 26 |
| HCDR2 | 51-58 | IVVGSGNT | 27 |
| HCDR3 | 96-113 | CAAPNCSNVVCYDGFDIW | 28 |

A23-58.1 VL

| | SEQ ID NO: 29 | | CDR |
|---|---|---|---|
| V_{L} | positions | CDR protein sequence | SEQ ID NO |
| LCDR1 | 27-33 | QSVSSSY | 30 |
| LCDR2 | 51-53 | SAS | 31 |
| LCDR3 | 89-99 | CQQYGTSPWTF | 32 |

A20-29.1 V_{H}

| | SEQ ID NO: 33 | | CDR |
|---|---|---|---|
| V_{H} | positions | CDR protein sequence | SEQ ID NO |
| HCDR1 | 26-33 | GFTFDDYA | 34 |
| HCDR2 | 51-58 | ISWNSGDI | 35 |
| HCDR3 | 96-114 | CTKGWFGEFFGAGSICDYW | 36 |

A20-29.1 V_{L}

| | SEQ ID NO: 37 | | CDR |
|---|---|---|---|
| V_{L} | positions | CDR protein sequence | SEQ ID NO |
| LCDR1 | 27-32 | QSVSNN | 38 |
| LCDR2 | 50-52 | GAS | 39 |
| LCDR3 | 88-97 | CQQYNNWPLF | 40 |

A23-105.1 V_{H}

| | SEQ ID NO: 41 | | CDR |
|---|---|---|---|
| V_{H} | positions | CDR protein sequence | SEQ ID NO |
| HCDR1 | 26-33 | GFTFSNYA | 42 |
| HCDR2 | 51-58 | ISYDGSNK | 43 |
| HCDR3 | 96-113 | CARVGPYQYDSSAAFDIW | 44 |

A23-105.1 V_{L}

| | SEQ ID NO: 45 | | CDR |
|---|---|---|---|
| V_{L} | positions | CDR protein sequence | SEQ ID NO |
| LCDR1 | 27-32 | QSISSW | 46 |
| LCDR2 | 50-52 | DAS | 47 |
| LCDR3 | 88-98 | CQQYNSYSRTF | 48 |

A19-1.1 V_{H}

| | SEQ ID NO: 49 | | CDR |
|---|---|---|---|
| V_{H} | positions | CDR protein sequence | SEQ ID NO |
| HCDR1 | 26-33 | GFTFTNYA | 50 |
| HCDR2 | 51-58 | ISNDGSDK | 51 |
| HCDR3 | 96-110 | CARDPPQVHWSLDYW | 52 |

A19-1.1 V_{L}

| | SEQ ID NO: 53 | | CDR |
|---|---|---|---|
| V_{H} | positions | CDR protein sequence | SEQ ID NO |
| LCDR1 | 26-34 | SSDVGDYNY | 54 |
| LCDR2 | 52-54 | DVS | 55 |
| LCDR3 | 90-101 | CSSYAGNNNAVF | 56 |

A19-30.1 V_{H}

| | SEQ ID NO: 57 | | CDR |
|---|---|---|---|
| V_{H} | positions | CDR protein sequence | SEQ ID NO |
| HCDR1 | 26-33 | GFTFSNYG | 58 |
| HCDR2 | 51-58 | ISYDGSNK | 59 |
| HCDR3 | 96-112 | CAKESQFGELFEALDYW | 60 |

A19-30.1 V_{L}

| | SEQ ID NO: 61 | | CDR |
|---|---|---|---|
| V_{L} | positions | CDR protein sequence | SEQ ID NO |
| LCDR1 | 26-31 | ALPRKY | 62 |
| LCDR2 | 49-51 | EDS | 63 |
| LCDR3 | 87-99 | CYSTDSSGNHRVF | 64 |

A20-36.1 V_{H}

| | SEQ ID NO: 65 | | CDR |
|---|---|---|---|
| V_{H} | positions | CDR protein sequence | SEQ ID NO |
| HCDR1 | 26-33 | GFIFSSYG | 66 |
| HCDR2 | 51-58 | IWHDESNK | 67 |
| HCDR3 | 96-113 | CARDGYDFLTGAYELDYW | 68 |

A20-36.1 V_{L}

| | SEQ ID NO: 69 | | CDR |
|---|---|---|---|
| V_{L} | positions | CDR protein sequence | SEQ ID NO |
| LCDR1 | 26-31 | ALPKQY | 70 |
| LCDR2 | 49-51 | KDS | 71 |
| LCDR3 | 87-98 | CQSADSSGTWVF | 72 |

A23-97.1 V_{H}

| | SEQ ID NO: 73 | | CDR |
|---|---|---|---|
| V_{H} | positions | CDR protein sequence | SEQ ID NO |
| HCDR1 | 26-32 | GFTFSSFG | 74 |
| HCDR2 | 51-57 | IRYDGSNK | 75 |
| HCDR3 | 95-110 | CAKTELYYYDSSGPLGW | 76 |

A23-97.1 V_{L}

| | SEQ ID NO: 77 | | CDR |
|---|---|---|---|
| V_{L} | positions | CDR protein sequence | SEQ ID NO |
| LCDR1 | 26-31 | QSITSW | 78 |
| LCDR2 | 49-51 | DAS | 79 |
| LCDR3 | 87-98 | CQQYNSYPWTF | 80 |

A23-113.1 V_{H}

| | SEQ ID NO: 81 | | CDR |
|---|---|---|---|
| V_{H} | positions | CDR protein sequence | SEQ ID NO |
| HCDR1 | 26-33 | GFTFSSYG | 82 |
| HCDR2 | 51-58 | ISHDGSYK | 83 |
| HCDR3 | 96-110 | CAKSYGYWMAYFDYW | 84 |

A23-113.1 V_{L}

| | SEQ ID NO: 85 | | CDR |
|---|---|---|---|
| V_{L} | positions | CDR protein sequence | SEQ ID NO |
| LCDR1 | 27-32 | QDISNY | 86 |
| LCDR2 | 50-52 | AAS | 87 |
| LCDR3 | 88-98 | CQKYNSPWHTF | 88 |

A23-80.1 V_{H}

| | SEQ ID NO: 89 | | CDR |
|---|---|---|---|
| V_{H} | positions | CDR protein sequence | SEQ ID NO |
| HCDR1 | 26-33 | GYTFTSNG | 90 |
| HCDR2 | 51-58 | ISTYNGDT | 91 |
| HCDR3 | 96-115 | CARVGDAYCSGGSCYHFDY | 92 |
| | | W | |

A23-80.1 V_{L}

| | SEQ ID NO: 93 | | CDR |
|---|---|---|---|
| V_{L} | positions | CDR protein sequence | SEQ ID NO |
| LCDR1 | 27-32 | QSVSTN | 94 |
| LCDR2 | 50-52 | GAS | 95 |
| LCDR3 | 88-100 | CQQYDNWPPEFTF | 96 |

A19-82.1 V_{H}

| | SEQ ID NO: 97 | | CDR |
|---|---|---|---|
| V_{H} | positions | CDR protein sequence | SEQ ID NO |
| HCDR1 | 26-33 | GLIFSTYD | 98 |
| HCDR2 | 51-58 | ISYDGSYK | 99 |
| HCDR3 | 96-112 | CAKGEGVVAGTGKFDYW | 100 |

A19-82.1 V_{L}

| | SEQ ID NO: 101 | | CDR |
|---|---|---|---|
| V_{L} | positions | CDR protein sequence | SEQ ID NO |
| LCDR1 | 26-31 | NIGSKS | 102 |
| LCDR2 | 49-51 | DDS | 103 |
| LCDR3 | 87-99 | CQVWDGSGDPWVF | 104 |

A20-9.1 V_{H}

| | SEQ ID NO: 105 | | CDR |
|---|---|---|---|
| V_{H} | positions | CDR protein sequence | SEQ ID NO |
| HCDR1 | 26-33 | GFTFSSYG | 106 |
| HCDR2 | 51-58 | ISYDGSNK | 107 |
| HCDR3 | 96-113 | CAKDYWSVAAGTSWFDPW | 108 |

A20-9.1 V_{L}

| | SEQ ID NO: 109 | | CDR |
|---|---|---|---|
| V_{L} | positions | CDR protein sequence | SEQ ID NO |
| LCDR1 | 26-31 | ALPKQY | 110 |
| LCDR2 | 49-51 | KDS | 111 |
| LCDR3 | 87-98 | CQSADSSGTWVF | 112 |

B1-182.1_58.1CDR3 V_{H}

| | SEQ ID NO: 143 | | CDR |
|---|---|---|---|
| V_{H} | positions | CDR protein sequence | SEQ ID NO |
| HCDR1 | 26-33 | GFTFTSSA | 2 |
| HCDR2 | 51-58 | IVVGSGNT | 3 |
| HCDR3 | 96-113 | CAAPNCSNVVCYDGFDIW | 58 |

B1-182.1 VL (paired with B1-182.1_58.1CDR3 V_{H})

| | SEQ ID NO: 5 | | CDR |
|---|---|---|---|
| V_{L} | positions | CDR protein sequence | SEQ ID NO |
| LCDR1 | 27-33 | QSVSSSY | 6 |
| LCDR2 | 51-53 | GAS | 7 |
| LCDR3 | 89-99 | CQQYGNSPWTF | 8 |

B1-182.1 light_5 mut V_{H}

| | SEQ ID NO: 1 | | CDR |
|---|---|---|---|
| V_{H} | positions | CDR protein sequence | SEQ ID NO |
| HCDR1 | 26-33 | GFTFTSSA | 2 |
| HCDR2 | 51-58 | IVVGSGNT | 3 |
| HCDR3 | 96-113 | CAAPYCSGGSCFDGFDIW | 4 |

B1-182.1 light_5 mut V_{L}

| | SEQ ID NO: 144 | | CDR |
|---|---|---|---|
| V_{L} | positions | CDR protein sequence | SEQ ID NO |
| LCDR1 | 27-33 | QSVSSSY | 6 |
| LCDR2 | 51-53 | SAS | 145 |
| LCDR3 | 89-99 | CQQYGTSPWTF | 146 |

Binding to SARS-CoV-1 and/or SARS-CoV-2 is shown in FIG. 1D.

### a. Monoclonal Antibody B1-182.1

In some embodiments, the antibody or antigen binding fragment is based on or derived from the B1-182.1 antibody, and specifically binds to a coronavirus spike protein, and neutralizes a coronavirus.

Monoclonal antibody B1-182.1 binds an epitope in the receptor binding domain (RBD) of the SARS COV-2 spike protein. As disclosed below, the B1-182.1 antibody prevents infection by directly blocking the binding of the virus to the ACE2 viral receptor and it has a unique competition profile compared to other antibodies. The antibody neutralizes SARS-CoV-2 pseudotyped lentivirus particles and Nanoluc live virus particles. This is 3 to 4-fold more potent than the leading clinical candidate, LY-COV555 and is amongst the most potent reported for antibodies targeting SARS COV-2. Information on the LY-COV55 antibody is provided in Jones det al., bioRxiv.2020 Oct. 1; 2020.09.30.318972 (revised Oct. 9, 2020), preprint, PMID 33024963, available electronically through pubmed.ncbi.nlm.nih.gov/33024963/, incorporated herein by reference, and clinical data for this antibody is available in Chen et al., New Engl. J. Med. 384(3): 229-237, 2021, incorporated herein by reference, see also pubmed.ncbi.nlm.nih.gov/33113295/.

In some embodiments, B1-182.1 maintains high potency against the following variants: D614G, N439K/D614G, Y543F/D614G, A222V/D614G, de169-70/D614G and N501/E484K/K417N/D614G and B.1.1.7 (VOC 202012/01) that contains amino acid changes at H69del-V70del-Y144del-N501Y-A570D-D614G-P681H-T716I-S982A-D1118H; and has increased potency against N501Y/D614G. The D614G variant is a dominant variant in circulation. The E484K variants are not neutralized by leading antibodies (e.g. LY-COV555, REGN-10989) or show significant loss in potency (REGN-10933). Y453F variants are not neutralized by REGN-10933. In some embodiments, B1-182.1 binds the B.1.1.529 variant.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 5, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, 7, and 8, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, 7, and 8, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 5, such as 95%, 96%, 97%, 98% or 99% identical to SEQ ID NO: 5, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 1, and specifically binds to a coronavirus spike protein, and neutralizes a coronavirus. In more embodiments, the antibody or antigen binding fragment comprises a V_{L} comprising the amino acid sequence set forth as SEQ ID NO: 5, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 1 and 5, respectively, and specifically binds to a coronavirus spike protein, and neutralizes a coronavirus. The coronavirus can be SARS-CoV-2.

In some embodiments, the disclosed antibodies inhibit viral entry and/or replication.

### b. Monoclonal Antibody A19-61.1

In some embodiments, the antibody or antigen binding fragment is based on or derived from the A19-61.1 antibody, and specifically binds to a coronavirus spike protein, and neutralizes a coronavirus.

Monoclonal antibody A19-61.1 binds an epitope in the RBD of the SARS COV-2 spike protein. This antibody prevents infection by directly blocking the binding of the virus to the ACE2 viral receptor and it has a unique competition profile compared to other antibodies. The antibody neutralizes pseudotyped lentivirus and Nanoluc live virus particle particles. Nanoluc live virus neutralization is amongst the most potent reported for antibodies targeting SARS COV-2.

In some embodiments, monoclonal antibody A19-61.1 has increased potency against the D614G variant and maintains that potency against the variants: N439K/D614G, Y543F/D614G, A222V/D614G, N501Y/D614G, de169-70/D614G and N501/E484K/K417N/D614G and B.1.1.7 (VOC 202012/01) that contains amino acid changes at H69del-V70del-Y144del-N501Y-A570D-D614G-P681H-T716I-S982A-D1118H.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A19-61.1 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 9, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 13, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 9 and 13, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 10, 11, and 12, respectively, and/or a V_{L} comprising a LCDR1, a LCDR2, and a LCDR3 as set forth as SEQ ID NOs: 14, 15, and 16, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 10, 11, and 12, respectively, a V_{L} comprising a LCDR1, a LCDR2, and a LCDR3 as set forth as SEQ ID NOs: 14, 15, and 16, respectively, wherein the V_{H} comprises an amino acid sequence at least 90% identical to SEQ ID NO: 9, such as 95%, 96%, 97%, 98% or 99% identical to SEQ ID NO: 9, and wherein the V_{L} comprises an amino acid sequence at least 90% identical to SEQ ID NO: 13, such as 95%, 96%, 97%, 98% or 99% identical to SEQ ID NO: 13, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 9, and specifically binds to a coronavirus spike protein, and neutralizes a coronavirus. In more embodiments, the antibody or antigen binding fragment comprises a V_{L} comprising the amino acid sequence set forth as SEQ ID NO: 13, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 9 and 13, respectively, and specifically binds to a coronavirus spike protein, and neutralizes a coronavirus. The coronavirus can be SARS-CoV-2.

In some embodiments, the disclosed antibodies inhibit viral entry and/or replication.

### c. Monoclonal Antibody A19-46.1

In some embodiments, the antibody or antigen binding fragment is based on or derived from the A19-46.1 antibody, and specifically binds to a coronavirus spike protein, and neutralizes a coronavirus.

Monoclonal antibody A19-46.1 binds an epitope in the S1 domain of the SARS COV-2 spike protein. The antibody prevents infection by directly blocking the binding of the virus to the ACE2 viral receptor and it has a unique competition profile compared to other antibodies. The antibody neutralizes SARS-CoV-2 pseudotyped lentivirus and Nanoluc live virus particles. The SARS CoV-2 Nanoluc live virus neutralization is amongst the most potent reported for antibodies targeting SARS CoV-2.

In some embodiments, monoclonal antibody A19-46.1 has increased potency against the D614G variant and maintains that potency against the variants: N439K/D614G, Y543F/D614G, A222V/D614G, N501Y/D614G, de169-70/D614G and N501/E484K/K417N/D614G and B.1.1.7 (VOC 202012/01) that contains amino acid changes at H69del-V70del-Y144del-N501Y-A570D-D614G-P681H-T716I-S982A-D1118H.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A19-46.1 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 17, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 21, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 17 and 21, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 18, 19, and 20 respectively, and/or a V_{L} comprising a LCDR1, a LCDR2, and a LCDR3 as set forth as SEQ ID NOs: 22, 23, and 24, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 18, 19, and 20, respectively, a V_{L} comprising a LCDR1, a LCDR2, and a LCDR3 as set forth as SEQ ID NOs: 22, 23, and 24, respectively, wherein the V_{H} comprises an amino acid sequence at least 90% identical to SEQ ID NO: 17, such as 95%, 96%, 97%, 98% or 99% identical to SEQ ID NO: 17, and wherein the V_{L} comprises an amino acid sequence at least 90% identical to SEQ ID NO: 21, such as 95%, 96%, 97%, 98% or 99% identical to SEQ ID NO: 21, and the antibody or antigens binding fragment specifically binds to a coronavirus spike protein, and neutralizes a coronavirus. In this embodiment, variations due to sequence identify fall outside the CDRs. The coronavirus can be SARS-CoV-2 and/or SARS-CoV-1.

In some embodiments, the antibody or antigen binding fragment comprises a V_{H} comprising the amino acid sequence set forth as SEQ ID NO: 17, and specifically binds to a coronavirus spike protein, and neutralizes a coronavirus. In more embodiments, the antibody or antigen binding fragment comprises a V_{L} comprising the amino acid sequence set forth as SEQ ID NO: 21, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 17 and 21, respectively, and specifically binds to a coronavirus spike protein, and neutralizes a coronavirus. The coronavirus can be SARS-CoV-2.

In some embodiments, the disclosed antibodies inhibit viral entry and/or replication.

### d. Monoclonal Antibody A23-58.1

In some embodiments, the antibody or antigen binding fragment is based on or derived from the A784-58.1 antibody, and specifically binds to a coronavirus spike protein, and neutralizes a coronavirus.

Monoclonal antibody 789-58.1 has an epitope in the receptor binding domain (RBD) of the SARS COV-2 spike protein. The antibody prevents infection by directly blocking the binding of the virus to the ACE2 viral receptor and it has a unique competition profile compared to other antibodies. The antibody has a strong IC₅₀ and IC₈₀ for SARS-CoV-2 pseudotyped lentivirus particles. The antibody is 3 to 4-fold more potent than monoclonal antibody LY-COV555. SARS COV-2 Nanoluc live virus neutralization is amongst the most potent reported for antibodies targeting SARS COV-2.

In some embodiments, monoclonal antibody A23-58.1 has slightly increased by highly potency against E484K/D614G and maintains high potency against the D614G, N439K/D614G, Y543F/D614G, A222V/D614G, N501Y/D614G, de169-70/D614G and N501/E484K/K417N/D614G and B.1.1.7 (VOC 202012/01) that contains amino acid changes at H69del-V70del-Y144del-N501Y-A570D-D614G-P681H-T716I-S982A-D1118H. In some embodiments, A23-58.1 binds the B.1.1.529 variant.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 25, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 29, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 25 and 29, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 26, 27, and 28 respectively, and/or a V_{L} comprising a LCDR1, a LCDR2, and a LCDR3 as set forth as SEQ ID NOs: 30, 31, and 32, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 26, 27, and 28, respectively, a V_{L} comprising a LCDR1, a LCDR2, and a LCDR3 as set forth as SEQ ID NOs: 30, 31, and 32, respectively, wherein the V_{H} comprises an amino acid sequence at least 90% identical to SEQ ID NO: 25, such as 95%, 96%, 97%, 98% or 99% identical to SEQ ID NO: 25, and wherein the V_{L} comprises an amino acid sequence at least 90% identical to SEQ ID NO: 29, such as 95%, 96%, 97%, 98% or 99% identical to SEQ ID NO: 29, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 25, and specifically binds to a coronavirus spike protein, and neutralizes a coronavirus. In more embodiments, the antibody or antigen binding fragment comprises a V_{L} comprising the amino acid sequence set forth as SEQ ID NO: 29, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 25 and 29, respectively, and specifically binds to a coronavirus spike protein, and neutralizes a coronavirus. The coronavirus can be SARS-CoV-2.

In some embodiments, the disclosed antibodies inhibit viral entry and/or replication.

### e. Monoclonal Antibody A20-29.1

In some embodiments, the antibody or antigen binding fragment is based on or derived from the A20-29.1 antibody, and specifically binds to a coronavirus spike protein, and neutralizes a coronavirus.

Monoclonal antibody A20-29.1 binds an epitope in the RBD domain of the SARS COV-2 spike protein. A20-29.1 is also able to bind to the original SARS CoV-1 spike protein. The antibody prevents infection by directly blocking the binding of the virus to the ACE2 viral receptor and has a unique competition profile compared to other antibodies. Since it does not compete with antibodies in the LY-COV555 competition group, it is of potential use in therapeutic cocktails with antibodies in that class. As disclosed herein, this antibody neutralizes SARS-CoV-2 pseudotyped lentivirus particles.

In some embodiments, monoclonal antibody A20-29.1 maintains similar potency against D614G, N439K/D614G, E484K/D61G, Y543F/D614G, A222V/D614G, N501Y/D614G, de169-70/D614G and N501/E484K/K417N/D614G variants.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A20-29.1 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 33, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 37, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 33 and 37, respectively, and specifically binds to a coronavirus spike protein and neutralizes a coronavirus. The coronavirus can be SARS-CoV-2 and/or SARS-CoV-1.

In some embodiments, the antibody or antigen binding fragment comprises a V_{H} comprising a HCDR1, a HCDR2, and a HCDR3 as set forth as SEQ ID NOs: 34, 35, and 36, respectively, and/or a V_{L} comprising a LCDR1, a LCDR2, and a LCDR3 as set forth as SEQ ID NOs: 38, 39, and 40, respectively, and specifically binds to a coronavirus spike protein, and neutralizes a coronavirus. The coronavirus can be SARS-CoV-2 and/or SARS-CoV-1.

In some embodiments, the antibody or antigen binding fragment comprises a V_{H} comprising a HCDR1, a HCDR2, and a HCDR3 as set forth as SEQ ID NOs: 34, 35, and 36, respectively, a V_{L} comprising a LCDR1, a LCDR2, and a LCDR3 as set forth as SEQ ID NOs: 38, 39, and 40, respectively, wherein the V_{H} comprises an amino acid sequence at least 90% identical to SEQ ID NO: 33, such as 95%, 96%, 97%, 98% or 99% identical to SEQ ID NO: 33, and wherein the V_{L} comprises an amino acid sequence at least 90% identical to SEQ ID NO: 37, such as 95%, 96%, 97%, 98% or 99% identical to SEQ ID NO: 37, and the antibody or antigens binding fragment specifically binds to a coronavirus spike protein, and neutralizes a coronavirus. In this embodiment, variations due to sequence identify fall outside the CDRs. The coronavirus can be SARS-CoV-2 and/or SARS-CoV-1.

In some embodiments, the antibody or antigen binding fragment comprises a V_{H} comprising the amino acid sequence set forth as SEQ ID NO: 33, and specifically binds to a coronavirus spike protein, and neutralizes a coronavirus. In more embodiments, the antibody or antigen binding fragment comprises a V_{L} comprising the amino acid sequence set forth as SEQ ID NO: 37, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 33 and 37, respectively, and specifically binds to a coronavirus spike protein, and neutralizes a coronavirus. The coronavirus can be SARS-CoV-2 and/or SARS-CoV-1.

In some embodiments, the disclosed antibodies inhibit viral entry and/or replication.

### f. Monoclonal Antibody A23-105.1

In some embodiments, the antibody or antigen binding fragment is based on or derived from the A23-105.1 antibody, and specifically binds to a coronavirus spike protein, and neutralizes a coronavirus. In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-105.1 antibody, and specifically binds to a coronavirus spike protein, and neutralizes a coronavirus. The coronavirus can be SARS-CoV-2.

Monoclonal antibody A23-105.1 binds an epitope in the RBD of the SARS COV-2 spike protein. This antibody prevents infection by directly blocking the binding of the virus to the ACE2 viral receptor and shares a similar competition profile as LY-COV555. Monoclonal antibody A23-105.1 is neutralizing. SARS COV-2 Nanoluc live virus neutralization by A23-105.1 documents that it is highly potent.

In some embodiments, A23-105.1 has increased potency against the D614G variant maintains similar potency against D614G, N439K/D614G, Y543F/D614G, A222V/D614G, N501Y/D614G and de169-70/D614G variants. Like LY-CoV555, it loses activity against variants E484K/D61G and N501/E484K/K417N/D614G.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 41, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 45, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 41 and 45, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 42, 43, and 44 respectively, and/or a V_{L} comprising a LCDR1, a LCDR2, and a LCDR3 as set forth as SEQ ID NOs: 46, 47, and 48, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 42, 43, and 44, respectively, a V_{L} comprising a LCDR1, a LCDR2, and a LCDR3 as set forth as SEQ ID NOs: 46, 47, and 48, respectively, wherein the V_{H} comprises an amino acid sequence at least 90% identical to SEQ ID NO: 41, such as 95%, 96%, 97%, 98% or 99% identical to SEQ ID NO: 41, and wherein the V_{L} comprises an amino acid sequence at least 90% identical to SEQ ID NO: 45, such as 95%, 96%, 97%, 98% or 99% identical to SEQ ID NO: 45, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 41, and specifically binds to a coronavirus spike protein, and neutralizes a coronavirus. In more embodiments, the antibody or antigen binding fragment comprises a V_{L} comprising the amino acid sequence set forth as SEQ ID NO: 45, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 41 and 45, respectively, and specifically binds to a coronavirus spike protein, and neutralizes a coronavirus. The coronavirus can be SARS-CoV-2.

In some embodiments, the disclosed antibodies inhibit viral entry and/or replication.

### g. Monoclonal Antibody A19-1.1

In some embodiments, the antibody or antigen binding fragment is based on or derived from the A19-1.1 antibody, and specifically binds to a coronavirus spike protein, and neutralizes a coronavirus.

Monoclonal antibody A19-1.1 binds to the original SARS CoV-1 spike protein. This antibody prevents infection by directly blocking the binding of the virus to the ACE2 viral receptor and shares a similar competition profile as LY-COV555.

In some embodiments, monoclonal antibody A19-1.1 has increased potency against the D614G variant maintains similar potency against D614G, N439K/D614G, Y543F/D614G, A222V/D614G, N501Y/D614G and de169-70/D614G variants. Like LY-CoV555, it loses activity against variants E484K/D61G and N501/E484K/K417N/D614G.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A19-1.1 antibody, and specifically binds to a coronavirus spike protein, and neutralizes a coronavirus. The coronavirus can be SARS-CoV-1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 49, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 53, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 49 and 53, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 50, 51, and 52 respectively, and/or a V_{L} comprising a LCDR1, a LCDR2, and a LCDR3 as set forth as SEQ ID NOs: 54, 55 and 56, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 50, 51, and 52, respectively, a V_{L} comprising a LCDR1, a LCDR2, and a LCDR3 as set forth as SEQ ID NOs: 54, 55, and 56, respectively, wherein the V_{H} comprises an amino acid sequence at least 90% identical to SEQ ID NO: 49, such as 95%, 96%, 97%, 98% or 99% identical to SEQ ID NO: 49, and wherein the V_{L} comprises an amino acid sequence at least 90% identical to SEQ ID NO: 53, such as 95%, 96%, 97%, 98% or 99% identical to SEQ ID NO: 53, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 49, and specifically binds to a coronavirus spike protein, and neutralizes a coronavirus. In more embodiments, the antibody or antigen binding fragment comprises a V_{L} comprising the amino acid sequence set forth as SEQ ID NO: 53, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 49 and 53, respectively, and specifically binds to a coronavirus spike protein, and neutralizes a coronavirus. The coronavirus can be SARS-CoV-2.

In some embodiments, the disclosed antibodies inhibit viral entry and/or replication.

### h. Monoclonal Antibody A19-30.1

In some embodiments, the antibody or antigen binding fragment is based on or derived from the A19-30.1 antibody, and specifically binds to a coronavirus spike protein.

Monoclonal antibody A19-30.1 binds an epitope in the RBD domain of the SARS COV-2 spike protein. This antibody does not prevent infection by directly blocking the binding of the virus to the ACE2 viral receptor. It has a unique competition profile compared to other antibodies. As this antibody does not compete with antibodies in the LY-COV555 competition group, it is of use in therapeutic cocktails with antibodies in the LY-COV555 class. It does not act by neutralizing of SARS-CoV-2 pseudotyped lentivirus particles.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A19-30.1 antibody, and specifically binds to a coronavirus spike protein, and acts using non-neutralizing mechanisms against coronavirus infection such as antibody-dependent cellular cytotoxicity, antibody-dependent phagocytosis or antibody-dependent complement killing of cells or virus particles. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 57, and specifically binds to a coronavirus spike, and inactivates coronavirus or kills infected cells. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 61, and specifically binds to a coronavirus spike protein, and inactivates coronavirus or kills infected cells. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 57 and 61, respectively, and specifically binds to a coronavirus spike protein and inactivates coronavirus or kills infected cells. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 58, 59, and 60 respectively, and/or a V_{L} comprising a LCDR1, a LCDR2, and a LCDR3 as set forth as SEQ ID NOs: 62, 63, and 64, respectively, and specifically binds to a coronavirus spike protein, and inactivates coronavirus or kills infected cells. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 58, 59, and 60, respectively, a V_{L} comprising a LCDR1, a LCDR2, and a LCDR3 as set forth as SEQ ID NOs: 62, 63, and 64, respectively, wherein the V_{H} comprises an amino acid sequence at least 90% identical to SEQ ID NO: 57, such as 95%, 96%, 97%, 98% or 99% identical to SEQ ID NO: 57, and wherein the V_{L} comprises an amino acid sequence at least 90% identical to SEQ ID NO: 61, such as 95%, 96%, 97%, 98% or 99% identical to SEQ ID NO: 61, and the antibody or antigens binding fragment specifically binds to a coronavirus spike protein, and inactivates coronavirus or kills infected cells. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 57, and specifically binds to a coronavirus spike protein, and neutralizes a coronavirus. In more embodiments, the antibody or antigen binding fragment comprises a V_{L} comprising the amino acid sequence set forth as SEQ ID NO: 61, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 57 and 61, respectively, and specifically binds to a coronavirus spike protein, and inactivates coronavirus or kills infected cells. The coronavirus can be SARS-CoV-2.

In some embodiments, the disclosed antibodies inhibit viral entry and/or replication.

### i. Monoclonal Antibody A20-36.1

In some embodiments, the antibody or antigen binding fragment is based on or derived from the A20-36.1 antibody, and specifically binds to a coronavirus spike protein, and neutralizes a coronavirus.

Monoclonal antibody A20-36.1 binds an epitope in the SD2 region of S1 domain of the SARS COV-2 spike protein. Monoclonal antibody A20-36.1 is also able to bind to the original SARS CoV-1 spike protein. This antibody does not prevent infection by directly blocking the binding of the virus to the ACE2 viral receptor. It has a unique competition profile. As this antibody does not compete with antibodies in the LY-COV555 competition group, it is of use in therapeutic cocktails with antibodies in that class. The monoclonal antibody neutralizes SARS-CoV-2 pseudotyped lentivirus particles.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A20-36.1 antibody, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 65, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 69, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 65 and 69, respectively, and specifically binds to a coronavirus spike protein and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 66, 67, and 68 respectively, and/or a V_{L} comprising a LCDR1, a LCDR2, and a LCDR3 as set forth as SEQ ID NOs: 70, 71, and 72, respectively, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 66, 67, and 68, respectively, a V_{L} comprising a LCDR1, a LCDR2, and a LCDR3 as set forth as SEQ ID NOs: 70, 71, and 72, respectively, wherein the V_{H} comprises an amino acid sequence at least 90% identical to SEQ ID NO: 65, such as 95%, 96%, 97%, 98% or 99% identical to SEQ ID NO: 65, and wherein the V_{L} comprises an amino acid sequence at least 90% identical to SEQ ID NO: 69, such as 95%, 96%, 97%, 98% or 99% identical to SEQ ID NO: 69, and the antibody or antigens binding fragment specifically binds to a coronavirus spike protein, and neutralizes a coronavirus. In this embodiment, variations due to sequence identify fall outside the CDRs. The coronavirus can be SARS-CoV-2 or SARS-CoV-1.

In some embodiments, the antibody or antigen binding fragment comprises a V_{H} comprising the amino acid sequence set forth as SEQ ID NO: 65, and specifically binds to a coronavirus spike protein, and neutralizes a coronavirus. In more embodiments, the antibody or antigen binding fragment comprises a V_{L} comprising the amino acid sequence set forth as SEQ ID NO: 69, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 65 and 69, respectively, and specifically binds to a coronavirus spike protein, and neutralizes a coronavirus. The coronavirus can be SARS-CoV-2 or SARS-CoV-1.

In some embodiments, the disclosed antibodies inhibit viral entry and/or replication.

### j. Monoclonal Antibody A23-97.1

In some embodiments, the antibody or antigen binding fragment is based on or derived from the A23-97.1 antibody, and specifically binds to a coronavirus spike protein, and neutralizes a coronavirus.

Monoclonal antibody A23-97.1 binds an epitope in the RBD of the SARS CoV-2 spike protein. Monoclonal antibody A23-97.1 is also able to bind to the original SARS CoV-1 spike protein. This antibody does not prevent infection by directly blocking the binding of the virus to the ACE2 viral receptor.

It has a unique competition profile as compared to other antibodies. This antibody does not compete with antibodies in the LY-COV555 competition group and is of use in therapeutic cocktails with antibodies in that class.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-97.1 antibody, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 73, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 77, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 73 and 77, respectively, and specifically binds to a coronavirus spike protein and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 74, 75, and 76 respectively, and/or a V_{L} comprising a LCDR1, a LCDR2, and a LCDR3 as set forth as SEQ ID NOs: 78, 79, and 80, respectively, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 74, 75, and 76, respectively, a V_{L} comprising a LCDR1, a LCDR2, and a LCDR3 as set forth as SEQ ID NOs: 78, 79, and 80, respectively, wherein the V_{H} comprises an amino acid sequence at least 90% identical to SEQ ID NO: 73, such as 95%, 96%, 97%, 98% or 99% identical to SEQ ID NO: 73, and wherein the V_{L} comprises an amino acid sequence at least 90% identical to SEQ ID NO: 77, such as 95%, 96%, 97%, 98% or 99% identical to SEQ ID NO: 77, and the antibody or antigens binding fragment specifically binds to a coronavirus spike protein, and neutralizes a coronavirus. In this embodiment, variations due to sequence identify fall outside the CDRs. The coronavirus can be SARS-CoV-2 or SARS-CoV-1.

In some embodiments, the antibody or antigen binding fragment comprises a V_{H} comprising the amino acid sequence set forth as SEQ ID NO: 73, and specifically binds to a coronavirus spike protein, and neutralizes a coronavirus. In more embodiments, the antibody or antigen binding fragment comprises a V_{L} comprising the amino acid sequence set forth as SEQ ID NO: 77, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 73 and 77, respectively, and specifically binds to a coronavirus spike protein, and neutralizes a coronavirus. The coronavirus can be SARS-CoV-2 or SARS-CoV-1.

In some embodiments, the disclosed antibodies inhibit viral entry and/or replication.

### k. Monoclonal Antibody A23-113.1

In some embodiments, the antibody or antigen binding fragment is based on or derived from the A23-113.1 antibody, and specifically binds to a coronavirus spike protein, and neutralizes a coronavirus.

Monoclonal antibody A23-113.1 binds an epitope in the RBD of the SARS COV-2 spike protein. Monoclonal antibody A23-113.1 is also able to bind to the original SARS CoV-1 spike protein. This monoclonal antibody prevents infection by directly blocking the binding of the virus to the ACE2 viral receptor and shares a similar competition profile to A23-97.1.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-113.1 antibody, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 81, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 85, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 81 and 85, respectively, and specifically binds to a coronavirus spike protein and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 82, 83 and 84 respectively, and/or a V_{L} comprising a LCDR1, a LCDR2, and a LCDR3 as set forth as SEQ ID NOs: 86, 87 and 88, respectively, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 82, 83, and 84, respectively, a V_{L} comprising a LCDR1, a LCDR2, and a LCDR3 as set forth as SEQ ID NOs:86, 87 and 88, respectively, wherein the V_{H} comprises an amino acid sequence at least 90% identical to SEQ ID NO: 81, such as 95%, 96%, 97%, 98% or 99% identical to SEQ ID NO: 81, and wherein the V_{L} comprises an amino acid sequence at least 90% identical to SEQ ID NO: 85, such as 95%, 96%, 97%, 98% or 99% identical to SEQ ID NO: 85, and the antibody or antigens binding fragment specifically binds to a coronavirus spike protein, and neutralizes a coronavirus. In this embodiment, variations due to sequence identify fall outside the CDRs. The coronavirus can be SARS-CoV-2 or SARS-CoV-1.

In some embodiments, the antibody or antigen binding fragment comprises a V_{H} comprising the amino acid sequence set forth as SEQ ID NO: 81, and specifically binds to a coronavirus spike protein, and neutralizes a coronavirus. In more embodiments, the antibody or antigen binding fragment comprises a V_{L} comprising the amino acid sequence set forth as SEQ ID NO: 85, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 81 and 85, respectively, and specifically binds to a coronavirus spike protein, and neutralizes a coronavirus. The coronavirus can be SARS-CoV-2 or SARS-CoV-1.

In some embodiments, the disclosed antibodies inhibit viral entry and/or replication.

### l. Monoclonal Antibody A23-80.1

In some embodiments, the antibody or antigen binding fragment is based on or derived from the A23-80.1 antibody, and specifically binds to a coronavirus spike protein, and neutralizes a coronavirus.

Monoclonal antibody A23-80.1 binds has an epitope in the RBD of the SARS COV-2 spike protein. This antibody does not prevent infection by directly blocking the binding of the virus to the ACE2 viral receptor. The antibody has a unique competition profile compared to other antibodies. It neutralizes SARS-CoV-2 pseudotyped lentivirus particles.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-80.1 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 89, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 93, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 89 and 93, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 90, 91 and 92 respectively, and/or a V_{L} comprising a LCDR1, a LCDR2, and a LCDR3 as set forth as SEQ ID NOs: 94, 95 and 96, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 90, 91, and 92, respectively, a V_{L} comprising a LCDR1, a LCDR2, and a LCDR3 as set forth as SEQ ID NOs:94, 95 and 96, respectively, wherein the V_{H} comprises an amino acid sequence at least 90% identical to SEQ ID NO: 89, such as 95%, 96%, 97%, 98% or 99% identical to SEQ ID NO: 89, and wherein the V_{L} comprises an amino acid sequence at least 90% identical to SEQ ID NO: 93, such as 95%, 96%, 97%, 98% or 99% identical to SEQ ID NO: 93, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 89, and specifically binds to a coronavirus spike protein, and neutralizes a coronavirus. In more embodiments, the antibody or antigen binding fragment comprises a V_{L} comprising the amino acid sequence set forth as SEQ ID NO: 93, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 89 and 93, respectively, and specifically binds to a coronavirus spike protein, and neutralizes a coronavirus. The coronavirus can be SARS-CoV-2.

In some embodiments, the disclosed antibodies inhibit viral entry and/or replication.

### m. Monoclonal Antibody A19-82.1

In some embodiments, the antibody or antigen binding fragment is based on or derived from the A19-82.1 antibody, and specifically binds to a coronavirus spike protein, and neutralizes a coronavirus.

Monoclonal antibody A19-82.1 binds an epitope in the RBD of the SARS-CoV-2 spike protein. This antibody is also able to bind to the original SARS CoV-1 spike protein. This antibody does not prevent infection by directly blocking the binding of the virus to the ACE2 viral receptor. It has a unique competition profile compared to other antibodies. As this antibody does not compete with antibodies in the LY-COV555 competition group, it is of use in therapeutic cocktails with antibodies in that class.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A19-82.1 antibody, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 97, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 101, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 97 and 101, respectively, and specifically binds to a coronavirus spike protein and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 98, 99, 100 respectively, and/or a V_{L} comprising a LCDR1, a LCDR2, and a LCDR3 as set forth as SEQ ID NOs: 102, 103, 104, respectively, and specifically binds to a coronavirus spike protein, and neutralizes a coronavirus. The coronavirus can be SARS-CoV-2 or SARS-CoV-1.

In some embodiments, the antibody or antigen binding fragment comprises a V_{H} comprising a HCDR1, a HCDR2, and a HCDR3 as set forth as SEQ ID NOs: 98, 99, and 100, respectively, a V_{L} comprising a LCDR1, a LCDR2, and a LCDR3 as set forth as SEQ ID NOs:102, 103 and 104, respectively, wherein the V_{H} comprises an amino acid sequence at least 90% identical to SEQ ID NO: 97, such as 95%, 96%, 97%, 98% or 99% identical to SEQ ID NO: 97, and wherein the V_{L} comprises an amino acid sequence at least 90% identical to SEQ ID NO: 101, such as 95%, 96%, 97%, 98% or 99% identical to SEQ ID NO: 101, and the antibody or antigens binding fragment specifically binds to a coronavirus spike protein, and neutralizes a coronavirus. In this embodiment, variations due to sequence identify fall outside the CDRs. The coronavirus can be SARS-CoV-2 or SARS-CoV-1.

In some embodiments, the antibody or antigen binding fragment comprises a V_{H} comprising the amino acid sequence set forth as SEQ ID NO: 97, and specifically binds to a coronavirus spike protein, and neutralizes a coronavirus. In more embodiments, the antibody or antigen binding fragment comprises a V_{L} comprising the amino acid sequence set forth as SEQ ID NO: 101, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 97 and 101, respectively, and specifically binds to a coronavirus spike protein, and neutralizes a coronavirus. The coronavirus can be SARS-CoV-2 or SARS-CoV-1.

In some embodiments, the disclosed antibodies inhibit viral entry and/or replication.

### n. Monoclonal Antibody A20-9.1

In some embodiments, the antibody or antigen binding fragment is based on or derived from the A20-9.1 antibody, and specifically binds to a coronavirus spike protein, and neutralizes a coronavirus.

Monoclonal antibody A20-9.1 binds an epitope in the SD2 region of S1 domain of the SARS COV-2 spike protein. This antibody does not prevent infection by directly blocking the binding of the virus to the ACE2 viral receptor. It has a unique competition profile compared to other antibodies. This antibody does not compete with antibodies in the LY-COV555 competition group, and thus is of use in therapeutic cocktails with antibodies in that class.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A20-9.1 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 105, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 109, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 105 and 109, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 106, 107 and 108, respectively, and/or a V_{L} comprising a LCDR1, a LCDR2, and a LCDR3 as set forth as SEQ ID NOs: 110, 111, and 112, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 106, 107, and 108, respectively, a V_{L} comprising a LCDR1, a LCDR2, and a LCDR3 as set forth as SEQ ID NOs: 110, 111, and 112, respectively, wherein the V_{H} comprises an amino acid sequence at least 90% identical to SEQ ID NO: 105, such as 95%, 96%, 97%, 98% or 99% identical to SEQ ID NO: 105, and wherein the V_{L} comprises an amino acid sequence at least 90% identical to SEQ ID NO: 109, such as 95%, 96%, 97%, 98% or 99% identical to SEQ ID NO: 109, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 105, and specifically binds to a coronavirus spike protein, and neutralizes a coronavirus. In more embodiments, the antibody or antigen binding fragment comprises a V_{L} comprising the amino acid sequence set forth as SEQ ID NO: 109, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 105 and 109, respectively, and specifically binds to a coronavirus spike protein, and neutralizes a coronavirus. The coronavirus can be SARS-CoV-2.

In some embodiments, the disclosed antibodies inhibit viral entry and/or replication.

### o. Monoclonal Antibody B1-182.1_58CDRH3

In some embodiments, the antibody or antigen binding fragment is based on or derived from the B1-182.1_58CDRH3 antibody, and specifically binds to a coronavirus spike protein, and neutralizes a coronavirus.

Monoclonal antibody B1-182.1_58CDRH3 binds an epitope in the receptor binding domain (RBD) of the SARS COV-2 spike protein, the same epitope as B1-182.1. The B1-182.1_58CDRH3 antibody prevents infection by directly blocking the binding of the virus to the ACE2 viral receptor and it has a competition profile similar to B1-182.1 and A23-58.1. The antibody neutralizes SARS-CoV-2 pseudotyped lentivirus particles. This is 3 to 4-fold more potent than the leading clinical candidate, LY-COV555 and is amongst the most potent reported for antibodies targeting SARS COV-2. Neutralization data is shown in FIG. 29C.

In some embodiments, B1-182.1_58CDRH3 maintains high potency against the following variants: D614G, N439K/D614G, Y543F/D614G, A222V/D614G, de169-70/D614G and N501/E484K/K417N/D614G and B.1.1.7 (VOC 202012/01) that contains amino acid changes at H69del-V70del-Y144del-N501Y-A570D-D614G-P681H-T716I-S982A-D1118H; and has increased potency against N501Y/D614G. The D614G variant is a dominant variant in circulation. The E484K variants are not neutralized by leading antibodies (e.g. LY-COV555, REGN-10989) or show significant loss in potency (REGN-10933). Y453F variants are not neutralized by REGN-10933. In some embodiments, B1-182.1_58CDRH3 binds the B.1.1.529 variant.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1_58CDRH3 antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 143, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 143 and 5, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 58, respectively, and/or a V_{L} comprising a LCDR1, a LCDR2, and a LCDR3 as set forth as SEQ ID NOs: 6, 7, and 8, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 58, respectively, a V_{L} comprising a LCDR1, a LCDR2, and a LCDR3 as set forth as SEQ ID NOs: 6, 7, and 8, respectively, wherein the V_{H} comprises an amino acid sequence at least 90% identical to SEQ ID NO: 143, such as 95%, 96%, 97%, 98% or 99% identical to SEQ ID NO: 143, and wherein the V_{L} comprises an amino acid sequence at least 90% identical to SEQ ID NO: 5, such as 95%, 96%, 97%, 98% or 99% identical to SEQ ID NO: 5, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 143, and specifically binds to a coronavirus spike protein, and neutralizes a coronavirus. In more embodiments, the antibody or antigen binding fragment comprises a V_{L} comprising the amino acid sequence set forth as SEQ ID NO: 5, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 143 and 5, respectively, and specifically binds to a coronavirus spike protein, and neutralizes a coronavirus. The coronavirus can be SARS-CoV-2.

In some embodiments, the disclosed antibodies inhibit viral entry and/or replication.

### p. Monoclonal Antibody B1-182.1 Heavy/B1-182.1 Light_5Mut

In some embodiments, the antibody or antigen binding fragment is based on or derived from the B1-182.1 heavy/B1-182.1 light_5Mut antibody, and specifically binds to a coronavirus spike protein, and neutralizes a coronavirus.

Monoclonal antibody B1-182.1 heavy/B1-182.1 light_5Mut binds an epitope in the receptor binding domain (RBD) of the SARS COV-2 spike protein. As disclosed below, the B1-182.1 heavy/B1-182.1 light_5Mut antibody prevents infection by directly blocking the binding of the virus to the ACE2 viral receptor and it has a competition profile similar to B1-182.1 and A23-58.1. The antibody neutralizes SARS-CoV-2 pseudotyped lentivirus particles. This is 3 to 4-fold more potent than the leading clinical candidate, LY-COV555 and is amongst the most potent reported for antibodies targeting SARS COV-2.

In some embodiments, B1-182.1 heavy/B1-182.1 light_5Mut maintains high potency against the following variants: D614G, N439K/D614G, Y543F/D614G, A222V/D614G, de169-70/D614G and N501/E484K/K417N/D614G and B.1.1.7 (VOC 202012/01) that contains amino acid changes at H69del-V70del-Y144del-N501Y-A570D-D614G-P681H-T716I-S982A-D1118H; and has increased potency against N501Y/D614G. The D614G variant is a dominant variant in circulation. The E484K variants are not neutralized by leading antibodies (e.g. LY-COV555, REGN-10989) or show significant loss in potency (REGN-10933). Y453F variants are not neutralized by REGN-10933. In some embodiments, B1-182.1 heavy/B1-182.1 light_5Mut binds the B.1.1.529 variant.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1 heavy/B1-182.1 light_5Mut antibody, and specifically binds to a coronavirus spike protein, and neutralizes a coronavirus. The coronavirus can be SARS-CoV-2.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 144, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 144, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, 145, and 146, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, 145, and 146, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 144, such as 95%, 96%, 97%, 98% or 99% identical to SEQ ID NO: 144, and the antibody or antigens binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 1, and specifically binds to a coronavirus spike protein, and neutralizes a coronavirus. In more embodiments, the antibody or antigen binding fragment comprises a V_{L} comprising the amino acid sequence set forth as SEQ ID NO: 144, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 1 and 144, respectively, and specifically binds to a coronavirus spike protein, and neutralizes a coronavirus. The coronavirus can be SARS-CoV-2.

In some embodiments, the disclosed antibodies inhibit viral entry and/or replication.

### 1. Additional Antibodies

In some examples, antibodies that bind to an epitope of interest can be identified based on their ability to cross-compete (for example, to competitively inhibit the binding of, in a statistically significant manner) with the antibodies provided herein in binding assays. In other examples, antibodies that bind to an epitope of interest can be identified based on their ability to cross-compete (for example, to competitively inhibit the binding of, in a statistically significant manner) with the one or more of the antibodies provided herein in binding assays.

Human antibodies that bind to the same epitope on the spike of the coronavirus protein, such as the NTD or RBD of the spike protein, to which the disclosed antibodies bind can be produced using any suitable method. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Additional human antibodies that bind to the same epitope can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies and antigen binding fragments that specifically bind to the same epitope can also be isolated by screening combinatorial libraries for antibodies with the desired binding characteristics. For example, by generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Competitive binding assays, similar to those disclosed in the examples section below, can be used to select antibodies with the desired binding characteristics.

### 2. Additional Description of Antibodies and Antigen Binding Fragments

An antibody or antigen binding fragment of the antibodies disclosed herein can be a human antibody or fragment thereof. Chimeric antibodies are also provided. The antibody or antigen binding fragment can include any suitable framework region, such as (but not limited to) a human framework region from another source, or an optimized framework region. Alternatively, a heterologous framework region, such as, but not limited to a mouse or monkey framework region, can be included in the heavy or light chain of the antibodies.

The antibody can be of any isotype. The antibody can be, for example, an IgA, IgM or an IgG antibody, such as IgG₁, IgG₂, IgG₃, or IgG₄. The class of an antibody that specifically binds to a coronavirus spike protein can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved, for example, using a vector or nucleic acid molecule that comprises a C_{L} or C_{H} chain. For example, an antibody that specifically binds the spike protein, that was originally IgG may be class switched to an IgA. Class switching can be used to convert one IgG subclass to another, such as from IgG₁ to IgG₂, IgG₃, or IgG₄.

In some examples, the disclosed antibodies are oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to the spike protein is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### (a) Binding Affinity

In several embodiments, the antibody or antigen binding fragment specifically binds the coronavirus spike protein with an affinity (e.g., measured by K_{D}) of no more than 1.0×10⁻⁸M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹¹ M, or no more than 1.0×10⁻¹ M. K_{D} can be measured, for example, by a radiolabeled antigen binding assay (RIA) performed with the Fab version of an antibody of interest and its antigen. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293(4):865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (NUNC™ Catalog #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57(20):4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 l/well of scintillant (MICROSCINT™-20; PerkinElmer) is added, and the plates are counted on a TOPCOUNT™ gamma counter (PerkinElmer) for ten minutes. Concentrations of each Fab that give less than or equal to 20% of maximal binding are chosen for use in competitive binding assays.

In another assay, K_{D} can be measured using surface plasmon resonance assays using Biolayer interferometry (BLI), see the examples section. In other embodiments, KD can be measured using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 1/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁻⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Affinity can also be measured by high throughput SPR using the Carterra LSA.

### (b) Multispecific Antibodies

In some embodiments, a multi-specific antibody, such as a bi-specific antibody, is provided that comprises an antibody or antigen binding fragment that specifically binds a coronavirus spike protein, as provided herein. Any suitable method can be used to design and produce the multi-specific antibody, such as crosslinking two or more antibodies, antigen binding fragments (such as scFvs) of the same type or of different types. Exemplary methods of making multispecific antibodies include those described in PCT Pub. No. WO2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate).

The multi-specific antibody may have any suitable format that allows for binding to the coronavirus spike protein by the antibody or antigen binding fragment as provided herein. Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Löffler et al., Blood, 95(6):2098-2103, 2000; and BrUhl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the V_{L}-CL (L) or V_{H}-CH1 chains, e.g., to produce a bibody one scFv is fused to the C-term of a Fab chain.

The bispecific tetravalent immunoglobulin known as the dual variable domain immunoglobulin or DVD-immunoglobulin molecule is disclosed in Wu et al., MAbs. 2009; 1:339-47, doi: 10.4161/mabs.1.4.8755, incorporated herein by reference. See also Nat Biotechnol. 2007 November; 25(11):1290-7. doi: 10.1038/nbt1345. Epub 2007 Oct. 14, also incorporated herein by reference. A DVD-immunoglobulin molecule includes two heavy chains and two light chains. Unlike IgG, however, both heavy and light chains of a DVD-immunoglobulin molecule contain an additional variable domain (VD) connected via a linker sequence at the N-termini of the V_{H} and V_{L} of an existing monoclonal antibody (mAb). Thus, when the heavy and the light chains combine, the resulting DVD-immunoglobulin molecule contains four antigen recognition sites, see Jakob et al., Mabs 5: 358-363, 2013, incorporated herein by reference, see FIG. 1 of Jakob et al. for schematic and space-filling diagrams. A DVD-immunoglobulin molecule functions to bind two different antigens on each DFab simultaneously.

The outermost or N-terminal variable domain is termed VD1 and the innermost variable domain is termed VD2; the VD2 is proximal to the C-terminal CH1 or CL. As disclosed in Jakob et al., supra, DVD-immunoglobulin molecules can be manufactured and purified to homogeneity in large quantities, have pharmacological properties similar to those of a conventional IgG₁, and show in vivo efficacy. Any of the disclosed monoclonal antibodies can be included in a DVD-immunoglobulin format.

### (c) Antigen Binding Fragments

Antigen binding fragments are encompassed by the present disclosure, such as Fab, F(ab′)₂, and Fv which include a heavy chain and V_{L} and specifically bind a coronavirus spike protein. These antibody fragments retain the ability to selectively bind with the antigen and are “antigen-binding” fragments. Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the V_{L} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “miniantibody.”

Any suitable method of producing the above-discussed antigen binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.55 Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### (d) Variants

In some embodiments, amino acid sequence variants of the antibodies and bispecific antibodies provided herein are provided. For example, it may be desirable to improve the binding affinity and/or other biological properties of the antibody or bispecific antibody. Amino acid sequence variants of an antibody may be prepared by introducing appropriate modifications into the nucleotide sequence encoding the antibody V_{H} domain and/or V_{L} domain, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 9. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13.

In further embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 21.

In yet other embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 25. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 29.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 33. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 37.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 41. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 45.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 49. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 53.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 57. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 61.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 65. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 69.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 73. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 77.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 81. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 85.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 89. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 93.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 97. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 101.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 105. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 109.

In some embodiments, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody/bispecific antibody, or the light chain of the antibody/bispecific antibody, or the heavy and light chains of the antibody/bispecific antibody, compared to known framework regions, or compared to the framework regions of the antibody, and maintain the specific binding activity for the epitope of the spike protein.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant V_{H} and V_{L} sequences provided above, each CDR either is unaltered, or contains no more than one, two or three amino acid substitutions. In some embodiments of the variant V_{H} and V_{L} sequences provided above, only the framework residues are modified so the CDRs are unchanged.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for the spike protein. In particular examples, the V_{H} amino acid sequence is one of SEQ ID NOs: 1, 9, 17, 25, 33, 41, 49, 57, 65, 73, 81, 89, 97, or 105. In other examples, the V_{L} amino acid sequence is one of SEQ ID NOs: 5, 13, 21, 29, 37, 45, 53, 61, 69, 77, 85, 93, 101, or 109, respectively.

In some embodiments, an antibody or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lee 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several embodiments, the constant region of the antibody or bispecific antibody comprises one or more amino acid substitutions to optimize in vivo half-life of the antibody. The serum half-life of IgG Abs is regulated by the neonatal Fc receptor (FcRn). Thus, in several embodiments, the antibody comprises an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies and antigen binding fragments can be linked to or comprise an Fc polypeptide including any of the substitutions listed above, for example, the Fc polypeptide can include the M428L and N434S substitutions according to the EU index numbering system.

In some embodiments, an antibody or bispecific antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Conjugates

The antibodies, antigen binding fragments, and bispecific antibodies that specifically bind to a coronavirus spike protein, as disclosed herein, can be conjugated to an agent, such as an effector molecule or detectable marker. Both covalent and noncovalent attachment means may be used. Various effector molecules and detectable markers can be used, including (but not limited to) toxins and radioactive agents such as ¹²⁵I, ³²P, ¹⁴C, ³H, and ³⁵S and other labels, target moieties, enzymes and ligands, etc. The choice of a particular effector molecule or detectable marker depends on the particular target molecule or cell, and the desired biological effect.

The procedure for attaching a detectable marker to an antibody, antigen binding fragment, or bispecific antibody. varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody, antigen binding fragment, or bispecific antibody, is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody, antigen binding fragment, or bispecific antibody, and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or bispecific antibody can be determined.

The antibody, antigen binding fragment or bispecific antibody can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody, antigen binding fragment, or bispecific antibody, can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody, antigen binding fragment, or bispecific antibody, may also be conjugated with biotin, and detected through indirect measurement of avidin or streptavidin binding. It should be noted that the avidin itself can be conjugated with an enzyme or a fluorescent label.

The antibody, antigen binding fragment or bispecific antibody, can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody, antigen binding fragment, or bispecific antibody, may also be labeled with a predetermined polypeptide epitope recognized by a secondary reporter (such as leucine zipper pair sequences, binding sites for secondary antibodies, metal binding domains, epitope tags).

The antibody, antigen binding fragment or bispecific antibody, can also be conjugated with a radiolabeled amino acid, for example, for diagnostic purposes. For instance, the radiolabel may be used to detect a coronavirus by radiography, emission spectra, or other diagnostic techniques. Examples of labels for polypeptides include, but are not limited to, the following radioisotopes: ³H, ¹⁴C, ³⁵S, ⁹⁰Y, ^{99m}Tc, ¹¹¹In, ¹²⁵, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of detectable marker moieties per antibody, antigen binding fragment, or bispecific antibody in a conjugate can range, for example, from 1 to 20 moieties per antibody or antigen binding fragment. In some embodiments, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules, such as mRNA) encoding the amino acid sequences of antibodies, antigen binding fragments, bispecific antibodies, and conjugates that specifically bind to a coronavirus spike protein, as disclosed herein, are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several embodiments, nucleic acid molecules can encode the V_{H}, the V_{L}, or both the V_{H} and V_{L} (for example in a bicistronic expression vector) of a disclosed antibody or antigen binding fragment. In some embodiments, the nucleic acid molecules encode an scFv. In several embodiments, the nucleic acid molecules can be expressed in a host cell (such as a mammalian cell) to produce a disclosed antibody or antigen binding fragment. Nucleic acid molecules encoding an scFv are provided.

The genetic code can be used to construct a variety of functionally equivalent nucleic acid sequences, such as nucleic acids which differ in sequence but which encode the same antibody sequence, or encode a conjugate or fusion protein including the V_{L} and/or V_{H} nucleic acid sequence.

In a non-limiting example, an isolated nucleic acid molecule encodes the V_{H} of the A23-58.1, A19-61.1, A19-46.1, A23-105.1, A23-97.1, A19-82.1, A19-1.1, A23-113.1, A20-29.1, A19-30.1, A20-36.1, A20-9.1, A23-80.1 or B1-182.1 antibody. Exemplary nucleic acid sequences are provided herein. In another non-limiting example, the nucleic acid molecule encodes the V_{L} of the A23-58.1, A19-61.1, A19-46.1, A23-105.1, A23-97.1, A19-82.1, A19-1.1, A23-113.1, A20-29.1, A19-30.1, A20-36.1, A20-9.1, A23-80.1 or B1-182.1 monoclonal antibody. In further non-limiting examples, the nucleic acid molecule can encode a bi-specific antibody, such as in DVD-immunoglobulin format. The nucleic acid can also encode an scFv. The nucleic acid molecule can also encode a conjugate.

Nucleic acid molecules encoding the antibodies, antigen binding fragments, bispecific antibodies, and conjugates that specifically bind to a coronavirus spike protein can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L} (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Natl. Acad. Sci. U.S.A., 85(16):5879-5883, 1988; McCafferty et al., Nature, 348:552-554, 1990; Kontermann and DUbel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to a coronavirus spike protein and another antigen. The encoded V_{H} and V_{L} optionally can include a furin cleavage site between the V_{H} and V_{L} domains. Linkers can also be encoded, such as when the nucleic acid molecule encodes a bi-specific antibody in DVD-Ig™ format.

One or more DNA sequences encoding the antibodies, antigen binding fragments, bispecific antibodies, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed antibodies and antigen binding fragments. Methods of stable transfer, meaning that the foreign DNA is continuously maintained in the host may be used. Hybridomas expressing the antibodies of interest are also encompassed by this disclosure.

The expression of nucleic acids encoding the antibodies, antigen binding fragments, and bispecific antibodies (such as DVD-immunoglobulin antibodies) described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lamda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, bispecific antibodies, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, antigen binding fragment, and conjugates need not be 100% pure. Once purified, partially or to homogeneity as desired, if to be used prophylatically, the polypeptides should be substantially free of endotoxin.

Methods for expression of antibodies, antigen binding fragments, bispecific antibodies, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### D. Methods and Compositions

### 1. Inhibiting a Coronavirus Infection

Methods are disclosed herein for the inhibition of a coronavirus infection in a subject, such as a SARS-CoV-2 infection. The methods include administering to the subject an effective amount (that is, an amount effective to inhibit the infection in the subject) of a disclosed antibody, antigen binding fragment, or bispecific antibody, or a nucleic acid encoding such an antibody, antigen binding fragment, or bispecific antibody, to a subject at risk of a coronavirus infection or having the coronavirus infection. The methods can be used pre-exposure or post-exposure. In some embodiments, the antibody or antigen binding fragment can be used in the form of a bi-specific antibody, such as a DVD-Immunoglobulin. The antigen binding fragment can be an scFv.

The infection does not need to be completely eliminated or inhibited for the method to be effective. For example, the method can decrease the infection by a desired amount, for example by at least 10%, at least 20%, at least 50%, at least 60%, at least 70%, at least 80%, at least 90%, at least 95%, at least 98%, or even at least 100% (elimination or prevention of detectable coronavirus infection) as compared to the coronavirus infection in the absence of the treatment. In some embodiments, the subject can also be treated with an effective amount of an additional agent, such as an anti-viral agent.

In some embodiments, administration of an effective amount of a disclosed antibody, antigen binding fragment, bispecific antibody, or nucleic acid molecule, inhibits the establishment of an infection and/or subsequent disease progression in a subject, which can encompass any statistically significant reduction in activity (for example, growth or invasion) or symptoms of the coronavirus infection in the subject.

Methods are disclosed herein for the inhibition of a coronavirus replication in a subject. The methods include administering to the subject an effective amount (that is, an amount effective to inhibit replication in the subject) of a disclosed antibody, antigen binding fragment, bispecific antibody, or a nucleic acid encoding such an antibody, antigen binding fragment, or bispecific antibody, to a subject at risk of a coronavirus infection or having a coronavirus infection. The methods can be used pre-exposure or post-exposure.

Methods are disclosed for treating a coronavirus infection in a subject. Methods are also disclosed for preventing a coronavirus infection in a subject. These methods include administering one or more of the disclosed antibodies, antigen binding fragments, bispecific antibodies, or nucleic acid molecule encoding such molecules, or a composition including such molecules, as disclosed herein.

Antibodies, antigen binding fragments thereof, and bispecific antibodies can be administered by intravenous infusion. Doses of the antibody, antigen binding fragment, or bispecific antibody vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some embodiments, the dose of the antibody, antigen binding fragment or bispecific antibody can be from about 0.5 mg/kg to about 5 mg/kg, such as a dose of about 1 mg/kg, about 2 mg/kg, about 3 mg/kg, about 4 mg/kg or about 5 mg/kg. The antibody, antigen binding fragment, or bispecific antibody is administered according to a dosing schedule determined by a medical practitioner. In some examples, the antibody, antigen binding fragment or bispecific antibody is administered weekly, every two weeks, every three weeks or every four weeks.

In some embodiments, the method of inhibiting the infection in a subject further comprises administration of one or more additional agents to the subject. Additional agents of interest include, but are not limited to, anti-viral agents such as hydroxychloroquine, arbidol, remdesivir, favipiravir, baricitinib, lopinavir/ritonavir, Zinc ions, and interferon beta-lb, or their combinations.

In some embodiments, the method comprises administration of a first antibody that specifically binds to a coronavirus spike protein as disclosed herein and a second antibody that also specifically binds to a coronavirus protein, such as a different epitope of the coronavirus protein In some embodiments, the first antibody is one of A23-58.1, A19-61.1, A19-46.1, A23-105.1, A23-97.1, A19-82.1, A19-1.1, A23-113.1, A20-29.1, A19-30.1, A20-36.1, A20-9.1, A23-80.1 or B1-182.1. In more embodiments, the first antibody is one of A23-58.1, A19-61.1, A19-46.1, A23-105.1, A23-97.1, A19-82.1, A19-1.1, A23-113.1, A20-29.1, A19-30.1, A20-36.1, A20-9.1, A23-80.1, B1-182.1 or B1-182.1_58CDRH3 and the second antibody is another of A23-58.1, A19-61.1, A19-46.1, A23-105.1, A23-97.1, A19-82.1, A19-1.1, A23-113.1, A20-29.1, A19-30.1, A20-36.1, A20-9.1, A23-80.1, B1-182.1, or B1-182.1_58CDRH3. In some embodiment, one antibody binds one epitope of the spike protein, and another antibody binds a different epitope of the spike protein. An effective amount of one, two, three or four, five, or six of A23-58.1, A19-61.1, A19-46.1, A23-105.1, A23-97.1, A19-82.1, A19-1.1, A23-113.1, A20-29.1, A19-30.1, A20-36.1, A20-9.1, A23-80.1, B1-182.1, or B1-182.1_58CDRH3 can be administered to a subject. An effective amount of LY-COV555, or an antibody in this class, can be administered to a subject. In more embodiments, the first antibody is B1-182.1 and the second antibody is A19-46.1. In further embodiments, the first antibody is B1-182.1 and the second antibody is A19-61.1. In more embodiments, the first antibody is B1-182.1_58CDRH3 and the second antibody is A19-46.1. In more embodiments, the first antibody is B1-182.1_58CDRH3 and the second antibody is A19-61.1. In further embodiments, more than two antibodies are administered to the subject. Thus, in some examples, 3, 4, or 5 antibodies are administered to the subject.

In some embodiments, a subject is administered DNA or RNA encoding a disclosed antibody, antigen binding fragment, or bispecific antibody, to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, antigen binding fragments thereof, or bispecific antibody can be placed under the control of a promoter to increase expression. The methods include liposomal delivery of the nucleic acids. Such methods can be applied to the production of an antibody, or antigen binding fragments thereof. In some embodiments, a disclosed antibody or antigen binding fragment is expressed in a subject using the pVRC8400 vector (described in Barouch et al., J. Virol., 79(14), 8828-8834, 2005, which is incorporated by reference herein).

In several embodiments, a subject (such as a human subject at risk of a coronavirus infection or having a coronavirus infection) can be administered an effective amount of an AAV viral vector that comprises one or more nucleic acid molecules encoding a disclosed antibody, antigen binding fragment, or bispecific antibody. The AAV viral vector is designed for expression of the nucleic acid molecules encoding a disclosed antibody, antigen binding fragment, or bispecific antibody, and administration of the effective amount of the AAV viral vector to the subject leads to expression of an effective amount of the antibody, antigen binding fragment, or bispecific antibody in the subject. Non-limiting examples of AAV viral vectors that can be used to express a disclosed antibody, antigen binding fragment, or bispecific antibody in a subject include those provided in Johnson et al., Nat. Med., 15(8):901-906, 2009 and Gardner et al., Nature, 519(7541):87-91, 2015, each of which is incorporated by reference herein in its entirety.

In one embodiment, a nucleic acid encoding a disclosed antibody, antigen binding fragment, or bispecific antibody is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed antibody, antigen binding fragment, or bispecific antibody, conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit a coronavirus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The coronavirus spike protein-specific antibody, antigen binding fragment, or bispecific antibody or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an embodiment, the antibody, antigen binding fragment, bispecific antibody or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, bispecific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

### 2. Compositions

Compositions are provided that include one or more of the coronavirus spike protein-specific antibody, antigen binding fragment, conjugate, or nucleic acid molecule encoding such molecules, that are disclosed herein in a pharmaceutically acceptable carrier. In some embodiments, the composition comprises the A23-58.1, A19-61.1, A19-46.1, A23-105.1, A23-97.1, A19-82.1, A19-1.1, A23-113.1, A20-29.1, A19-30.1, A20-36.1, A20-9.1, A23-80.1, B1-182.1, or B1-182.1_58CDRH3 antibody disclosed herein, or an antigen binding fragment thereof. In some embodiments, the composition comprises two, three, four or more antibodies that specifically bind a coronavirus spike protein. The compositions are useful, for example, for example, for the inhibition or detection of a coronavirus infection, such as a SARS-CoV-2 infection. The compositions are useful, for example, for example, for the inhibition or detection of a coronavirus infection, such as a SARS-CoV-1 infection.

The compositions can be prepared in unit dosage forms, such as in a kit, for administration to a subject. The amount and timing of administration are at the discretion of the administering physician to achieve the desired purposes. The antibody, antigen binding fragment, bispecific antibody, conjugate, or nucleic acid molecule encoding such molecules can be formulated for systemic or local administration. In one example, the, antigen binding fragment, bispecific antibody, conjugate, or nucleic acid molecule encoding such molecules, is formulated for parenteral administration, such as intravenous administration.

In some embodiments, the antibody, antigen binding fragment, bispecific antibody, or conjugate thereof, in the composition is at least 70% (such as at least 75%, at least 80%, at least 85%, at least 90%, at least 95%, at least 96%, at least 97%, at least 98% or at least 99%) pure. In some embodiments, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the antibody, antigen binding fragment, bispecific antibody, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration comprises about 0.01 to about 30 mg/kg of antibody, antigen binding fragment, bispecific antibody, or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some embodiments, the composition can be a liquid formulation including one or more antibodies, antigen binding fragments, or bispecific antibodies, in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Antibodies, an antigen binding fragment thereof, a bispecific antibody, or a nucleic acid encoding such molecules, can be provided in lyophilized form and rehydrated with sterile water before administration, although they are also provided in sterile solutions of known concentration. A solution including the antibody, antigen binding fragment, bispecific antibody, or a nucleic acid encoding such molecules, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 m are generally referred to as nanoparticles, nanospheres, and nanocapsules, respectively. Capillaries have a diameter of approximately 5 m so that only nanoparticles are administered intravenously. Microparticles are typically around 100 m in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

### 3. Methods of Detection and Diagnosis

Methods are also provided for the detection of the presence of a coronavirus spike protein in vitro or in vivo. In one example, the presence of a coronavirus spike protein is detected in a biological sample from a subject and can be used to identify a subject with an infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an antibody, antigen binding fragment, or bispecific antibody, that specifically binds to a coronavirus spike protein, or conjugate thereof (e.g., a conjugate including a detectable marker) under conditions sufficient to form an immune complex, and detecting the immune complex (e.g., by detecting a detectable marker conjugated to the antibody or antigen binding fragment).

In one embodiment, the antibody, antigen binding fragment or bispecific antibody is directly labeled with a detectable marker. In another embodiment, the antibody (or antigen binding fragment or bispecific antibody) that binds the coronavirus spike protein (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody, antigen binding fragment, bispecific antibody or secondary antibody are known and described above, and include various enzymes, prosthetic groups, fluorescent materials, luminescent materials, magnetic agents and radioactive materials.

In some embodiments, the disclosed antibodies, antigen binding fragments thereof, or bispecific antibodies are used to test vaccines. For example, to test if a vaccine composition including a coronavirus spike protein or fragment thereof assumes a conformation including the epitope of a disclosed antibody. Thus, provided herein is a method for testing a vaccine, wherein the method comprises contacting a sample containing the vaccine, such as a coronavirus spike protein immunogen, with a disclosed antibody, antigen binding fragment, or bispecific antibody, under conditions sufficient for formation of an immune complex, and detecting the immune complex, to detect the vaccine including the epitope of interest in the sample. In one example, the detection of the immune complex in the sample indicates that vaccine component, such as the immunogen assumes a conformation capable of binding the antibody or antigen binding fragment.

The method can also include the use of an assay that distinguishes between SARS-CoV-2 as some isolated mAbs only bind to SARS-CoV-2 but not the SARS-CoV-1. In some embodiments, a comparison is made between the binding of a sample to an antibody that binds SARS-CoV-1, and the binding of a sample to an antibody that binds only the SARS-CoV-2. Thus, the disclosed methods can be used to distinguish SARS-CoV-1 and SARS-CoV-2 in a sample.

### EXAMPLES

Monoclonal antibodies A23-58.1 (A23-58.1), A19-61.1 (A19-61.1), A19-46.1 (A19-46.1), A23-105.1 (A23-105.1), A23-97.1 (A23-97.1), A19-82.1 (A19-82.1), A19-1.1 (A19-1.1), A23-113.1 (A23-113.1), A20-29.1 (A20-29.1), A19-30.1 (A19-30.1), A20-36.1 (A20-36.1), A20-9.1 (A20-9.1), A23-80.1 (A23-80.1) and B1-182.1 (B1-182.1) were isolated from single memory B cells from peripheral mononuclear blood cells of a survivor of SARS CoV-2 infection that were sorted for SARS CoV-2 Spike protein binding.

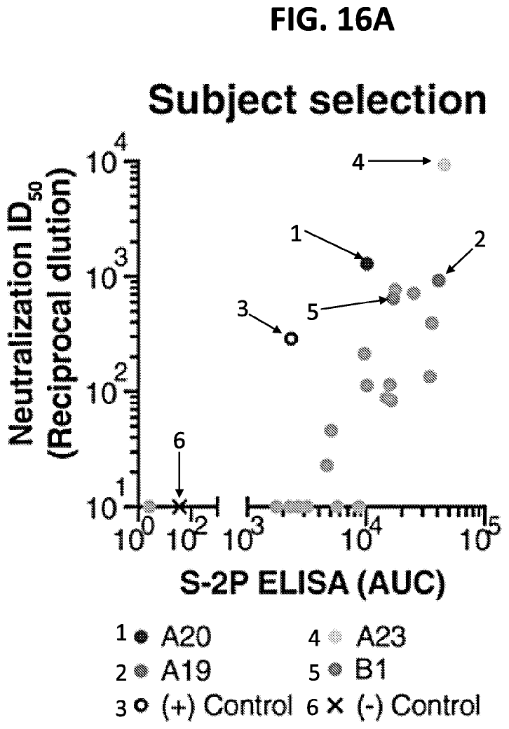

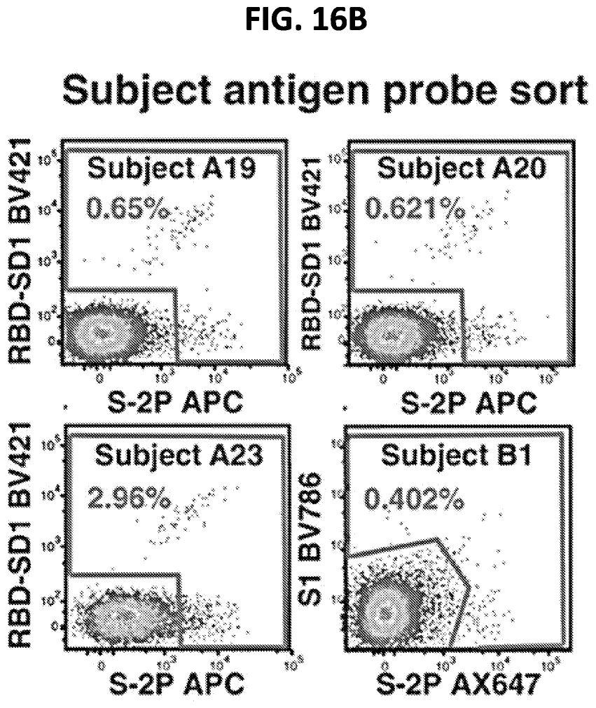

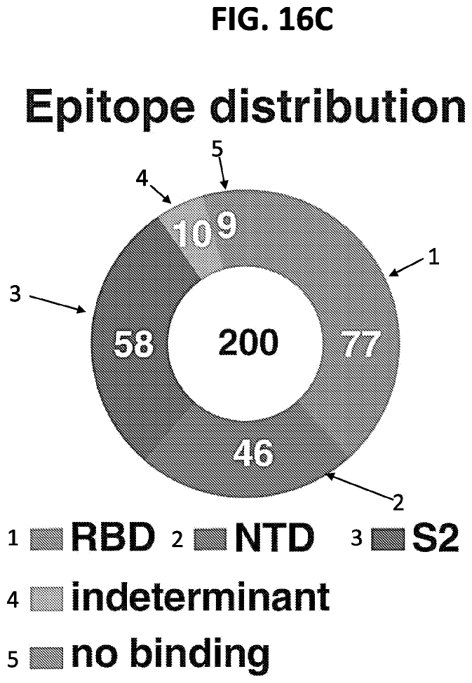

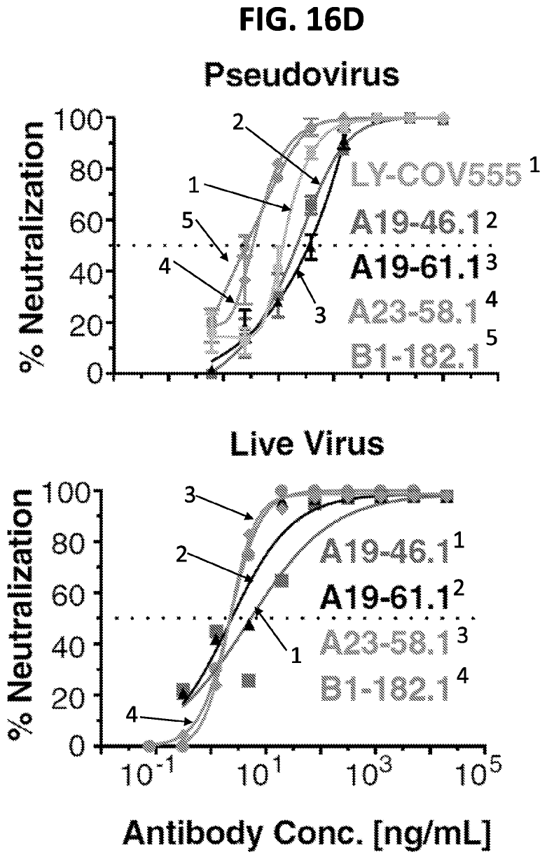

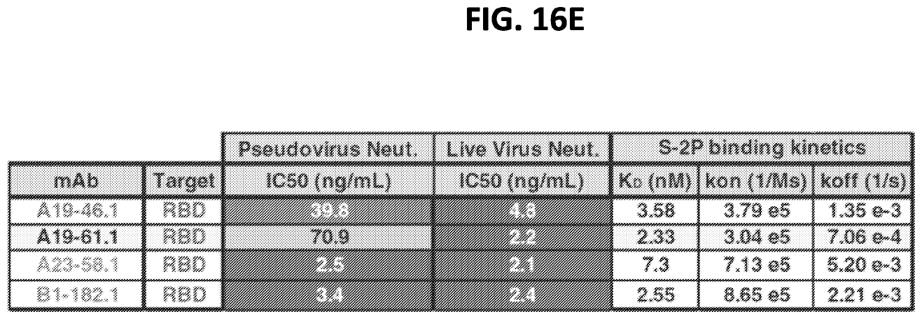

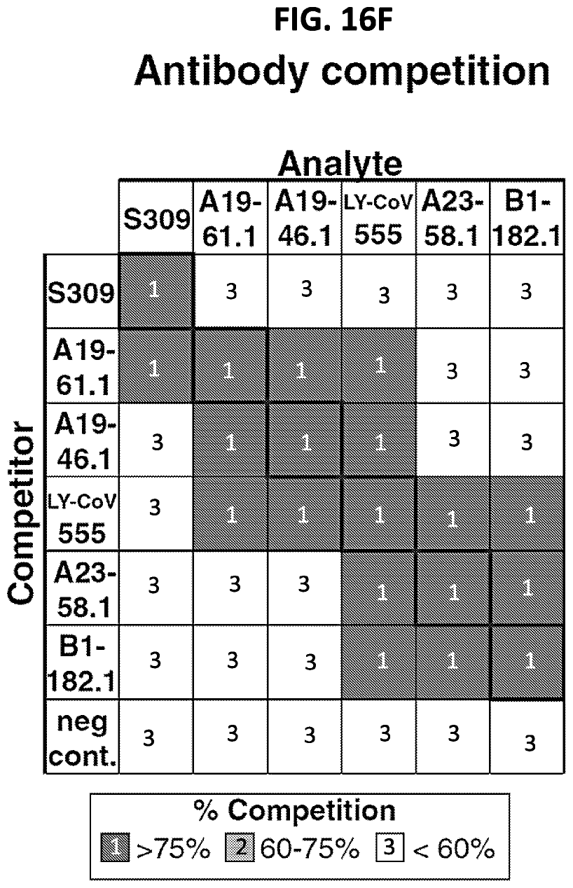

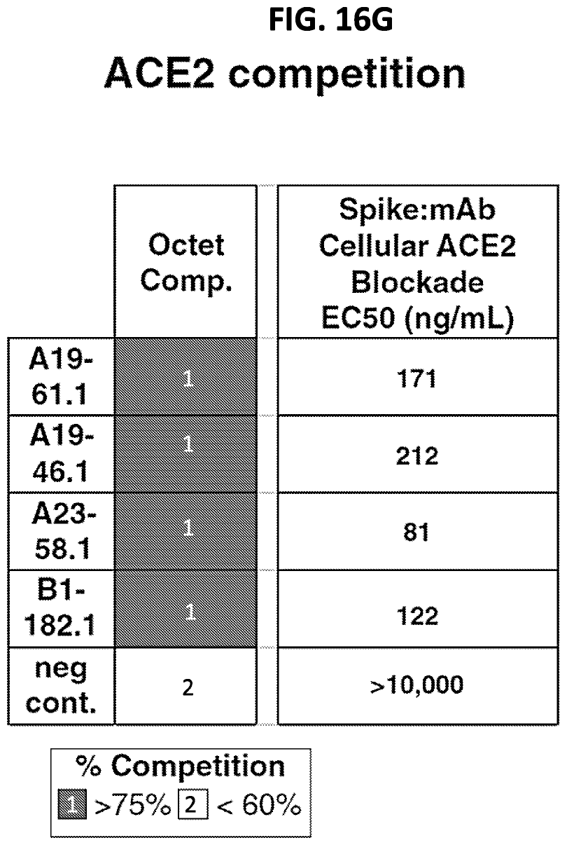

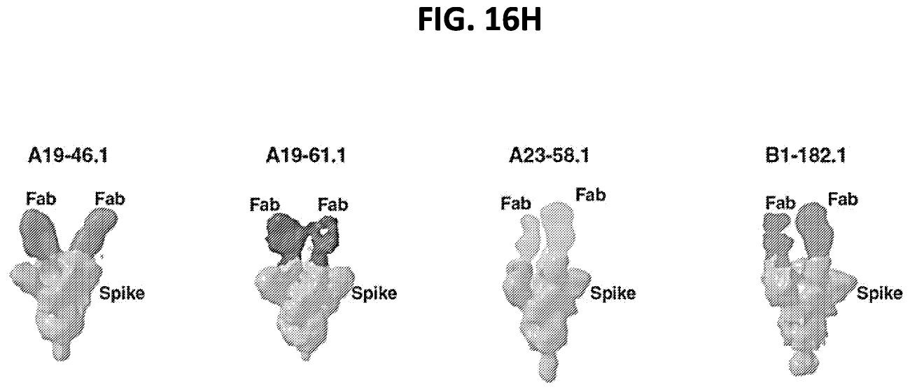

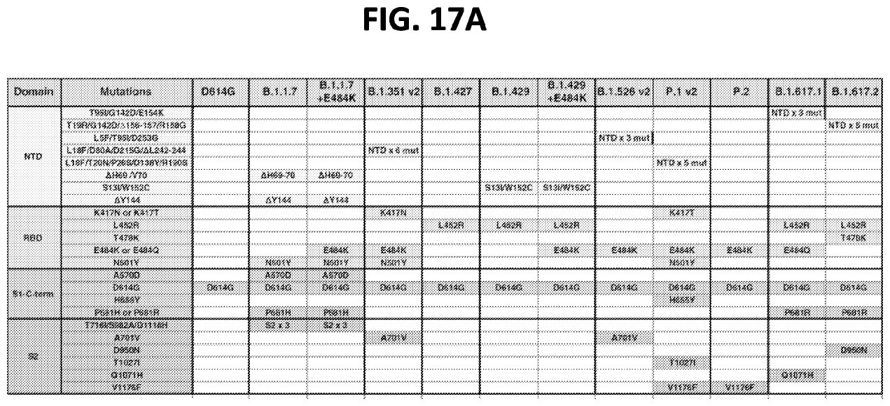

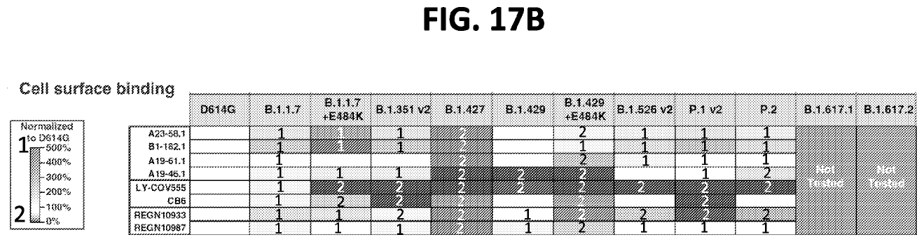

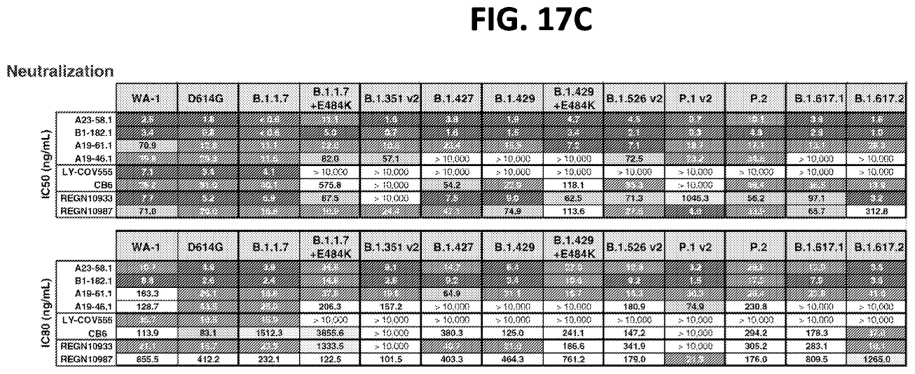

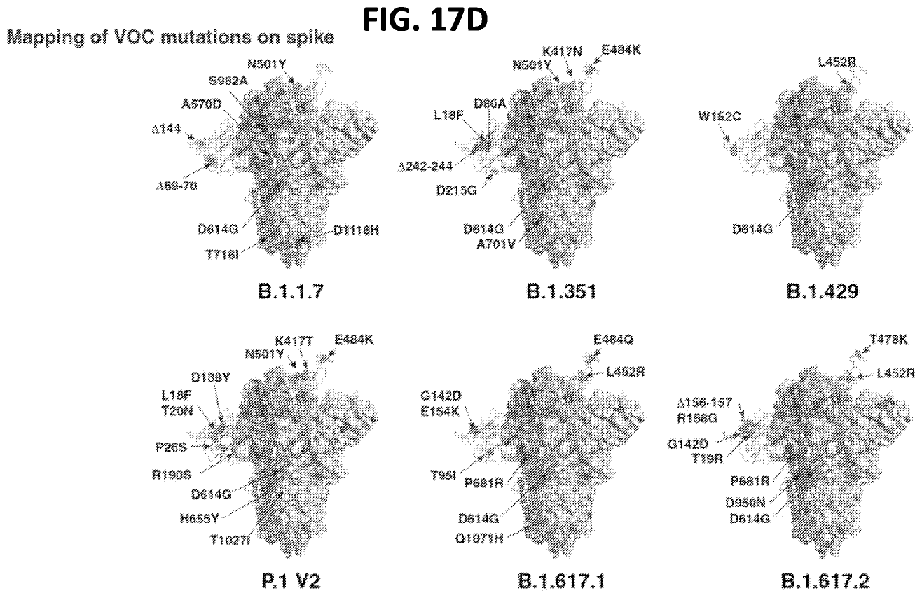

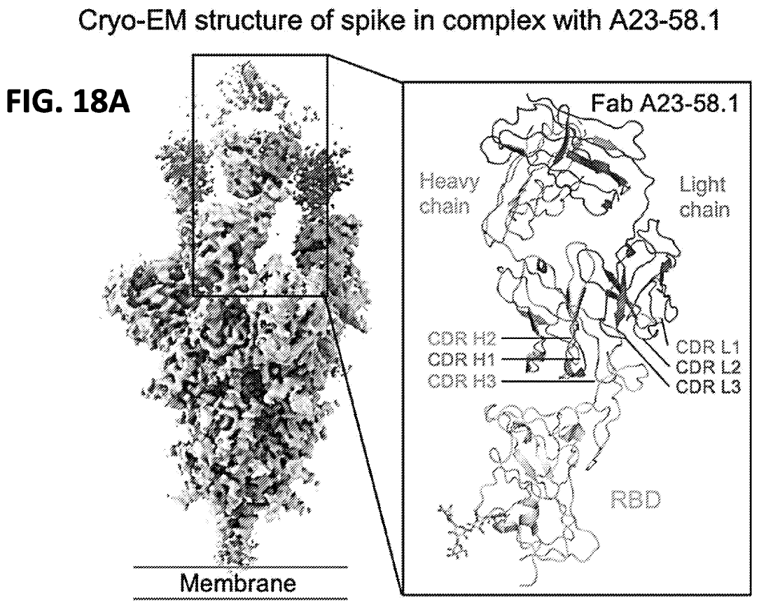

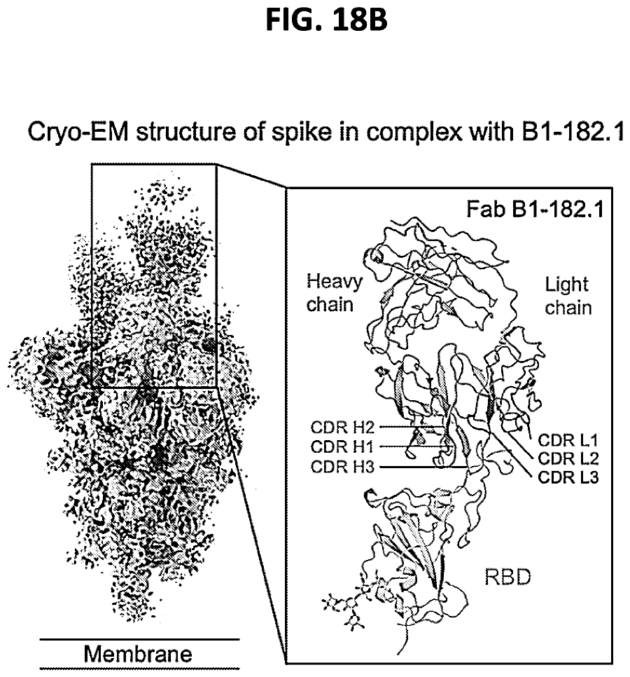

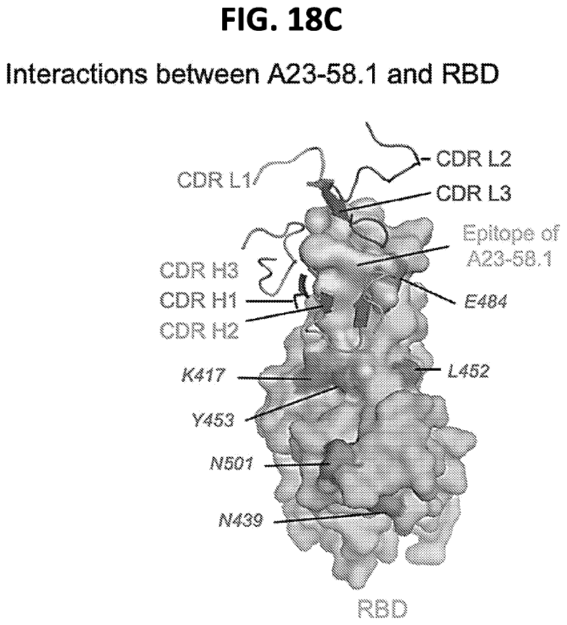

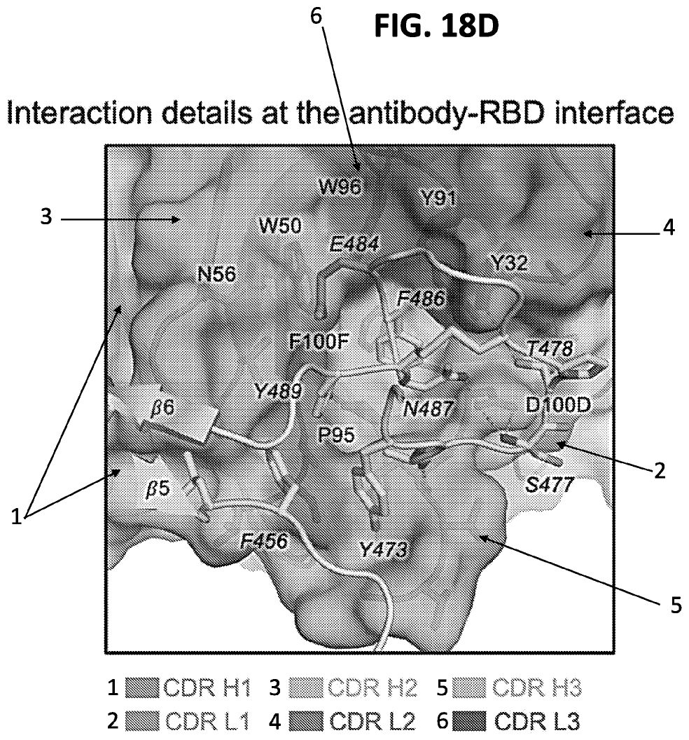

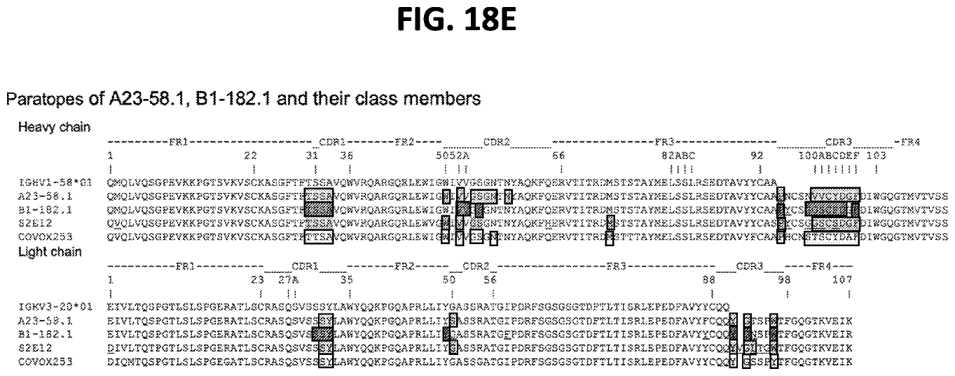

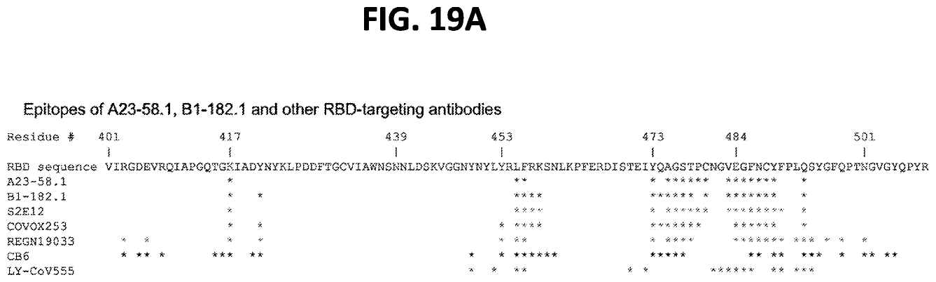

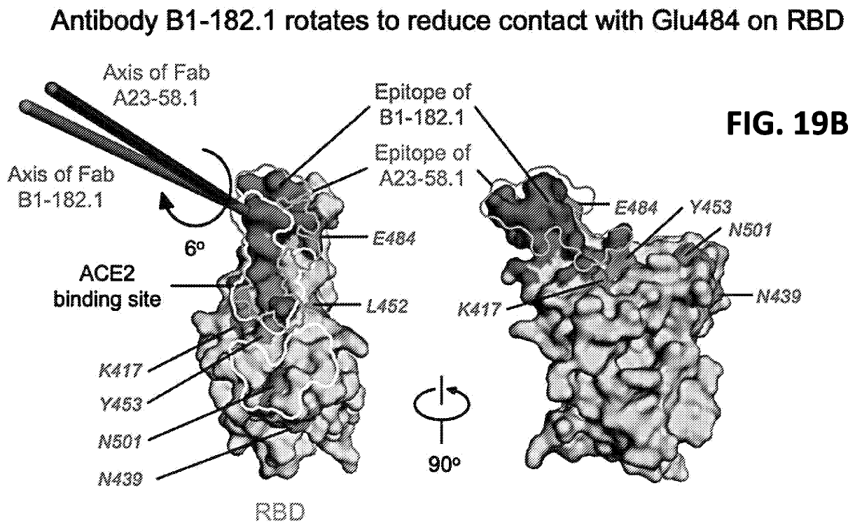

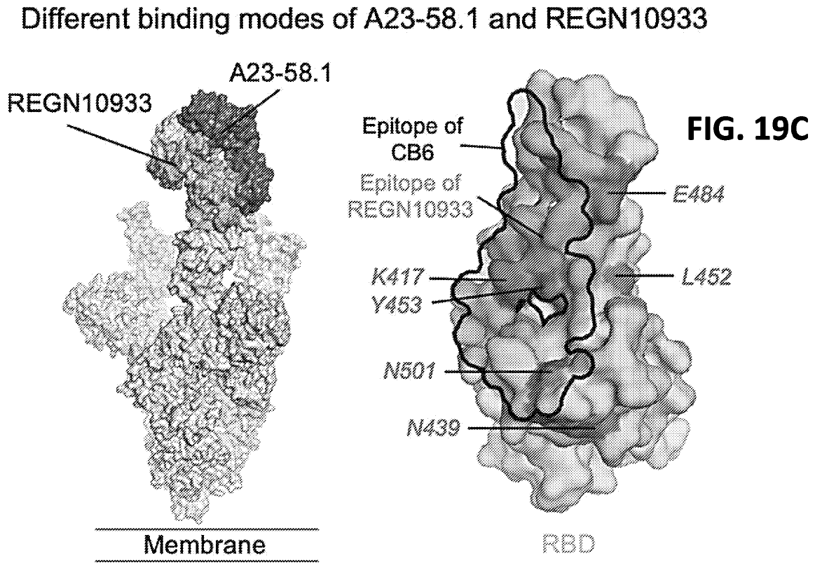

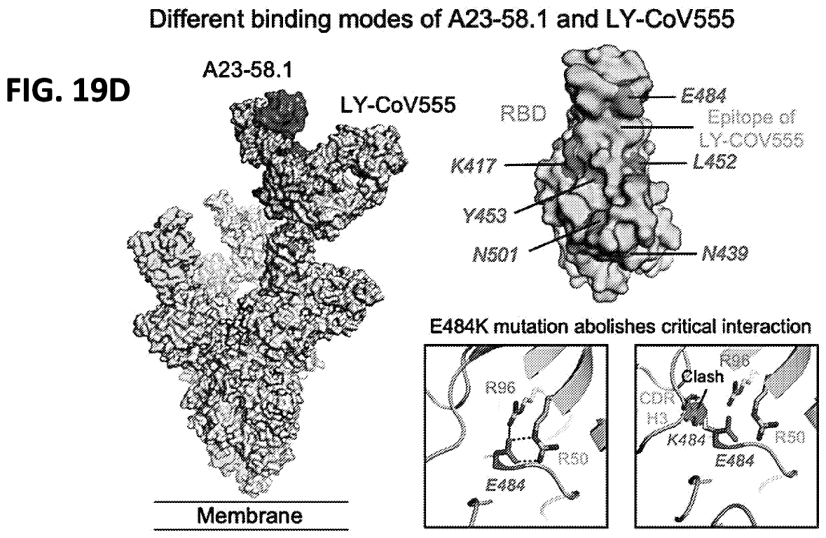

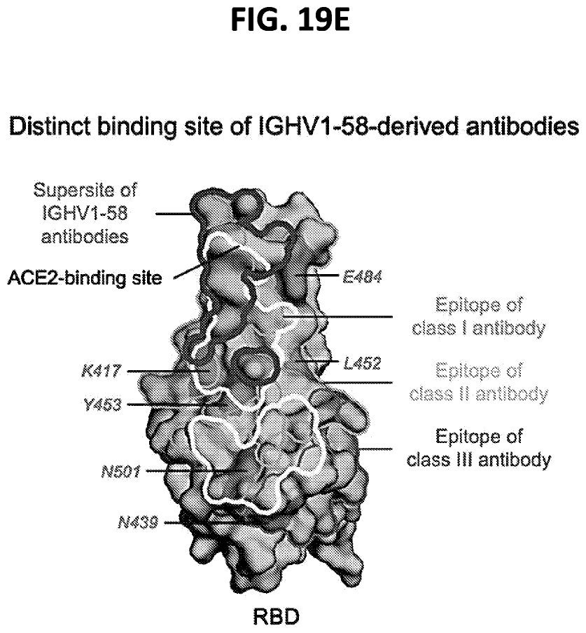

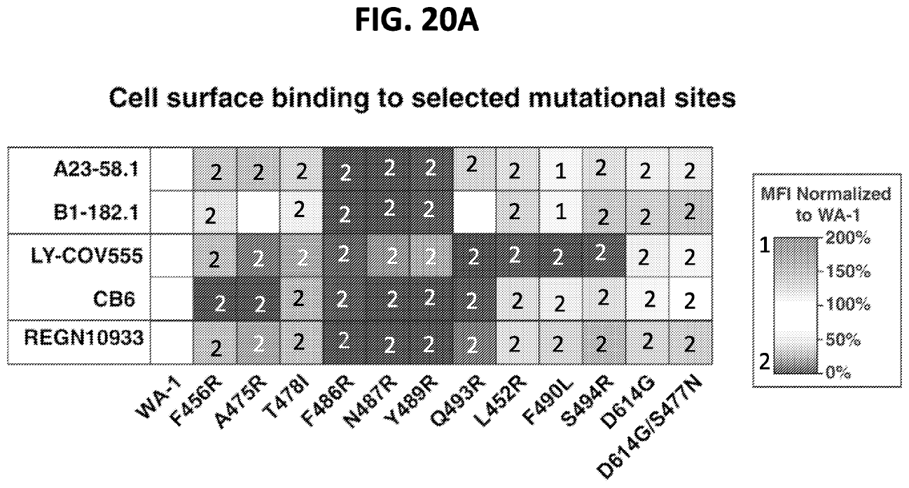

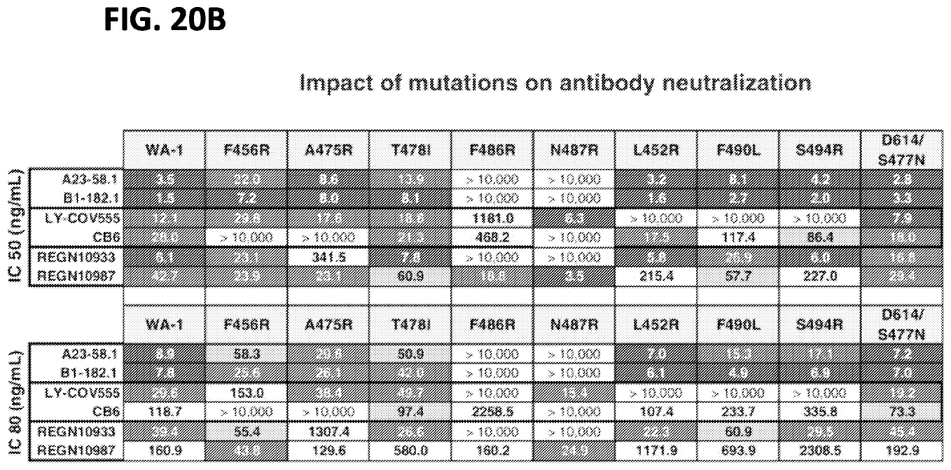

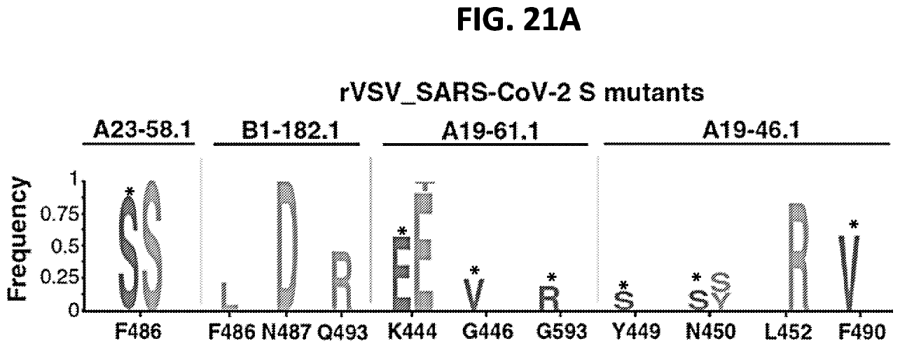

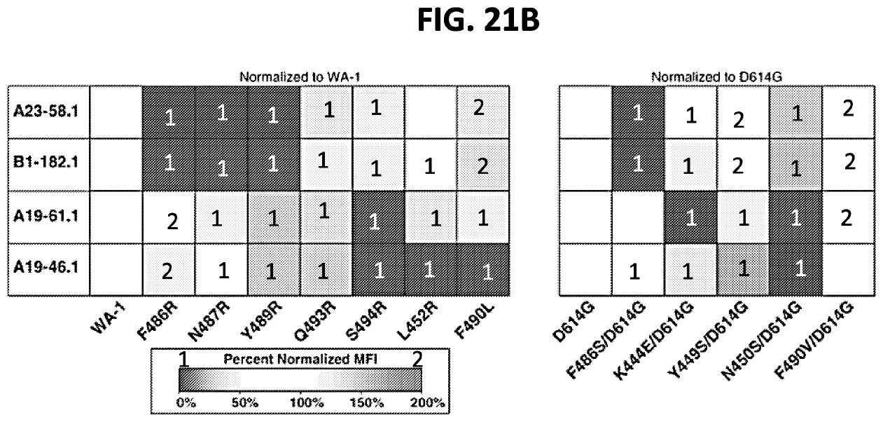

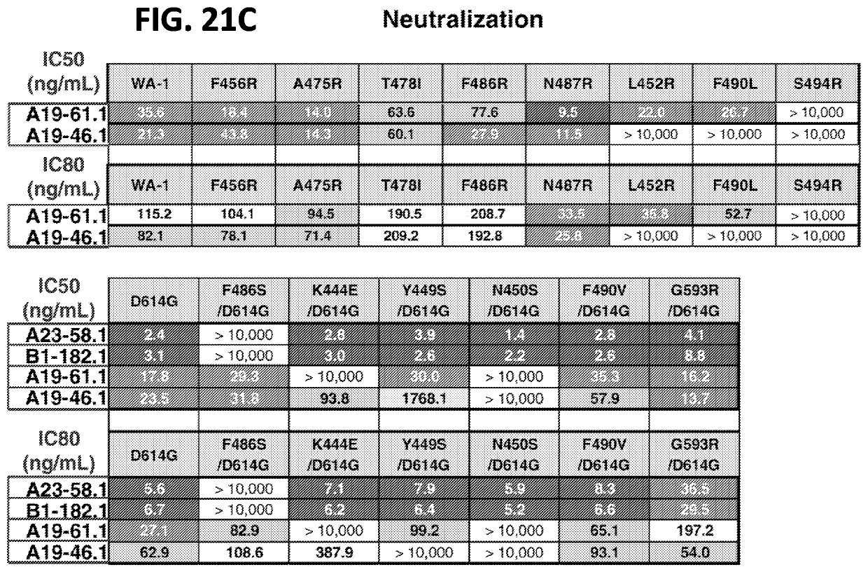

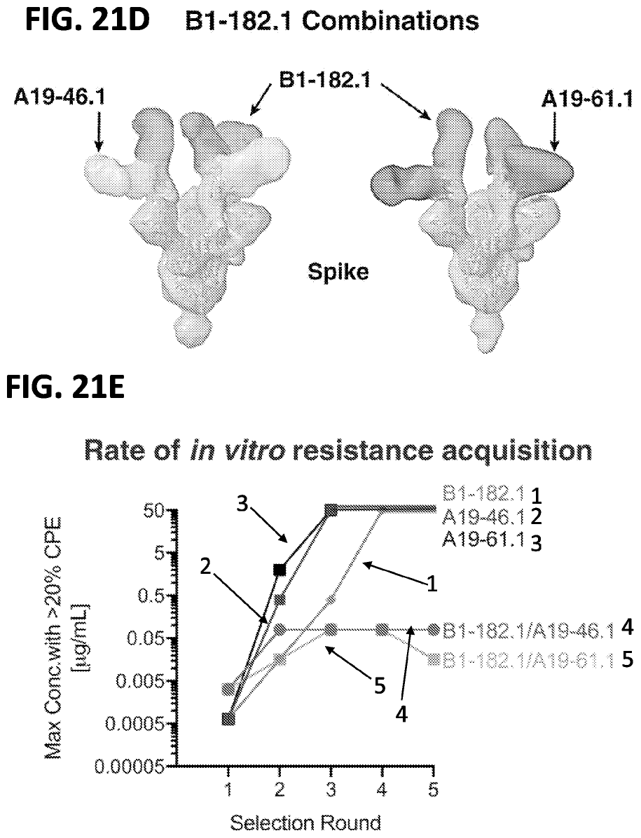

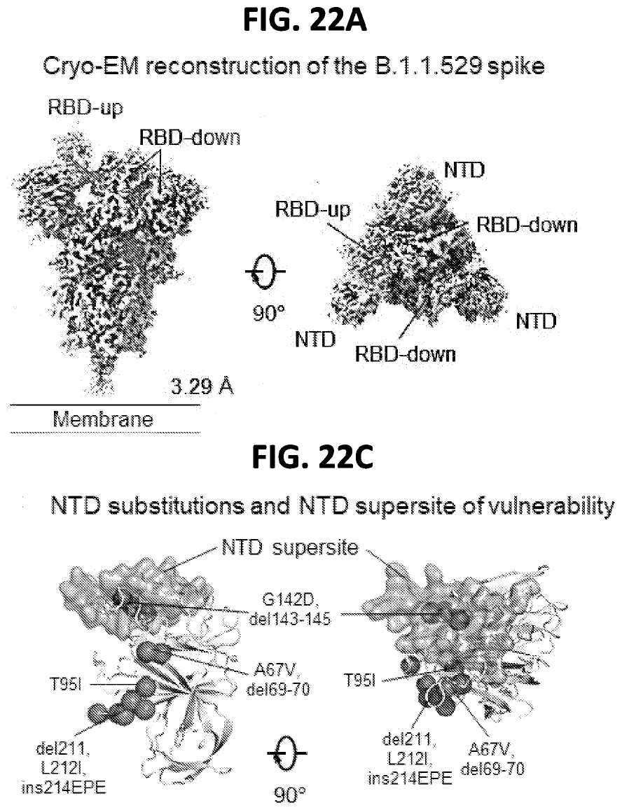

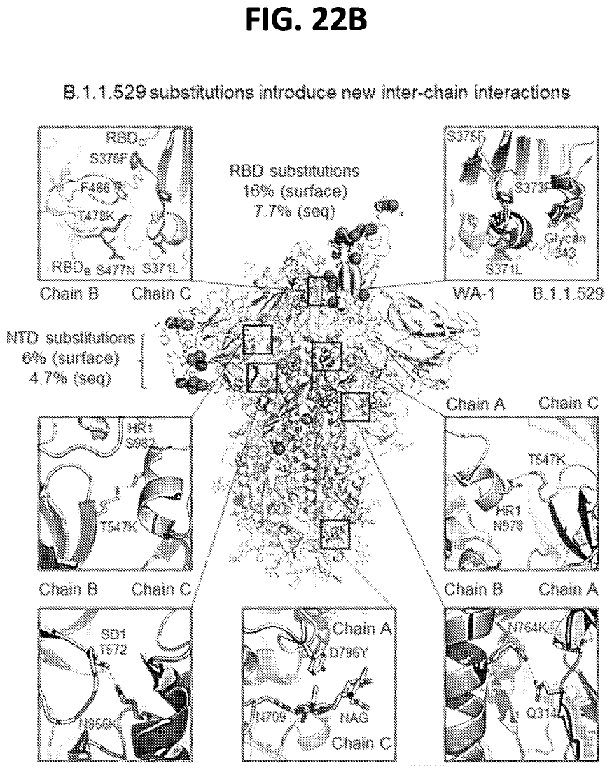

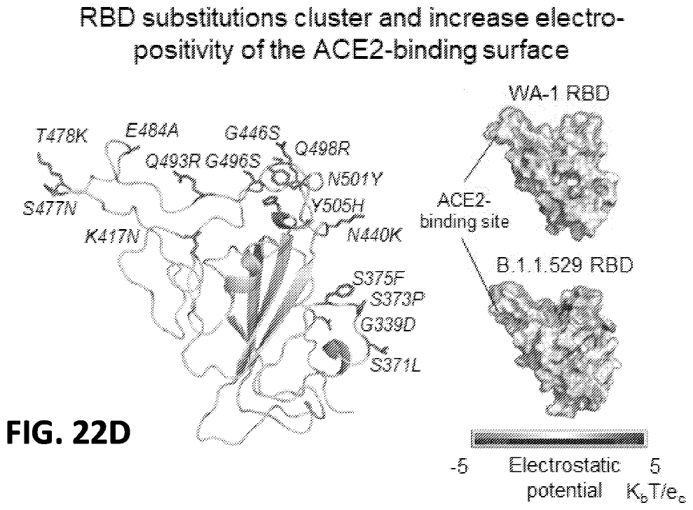

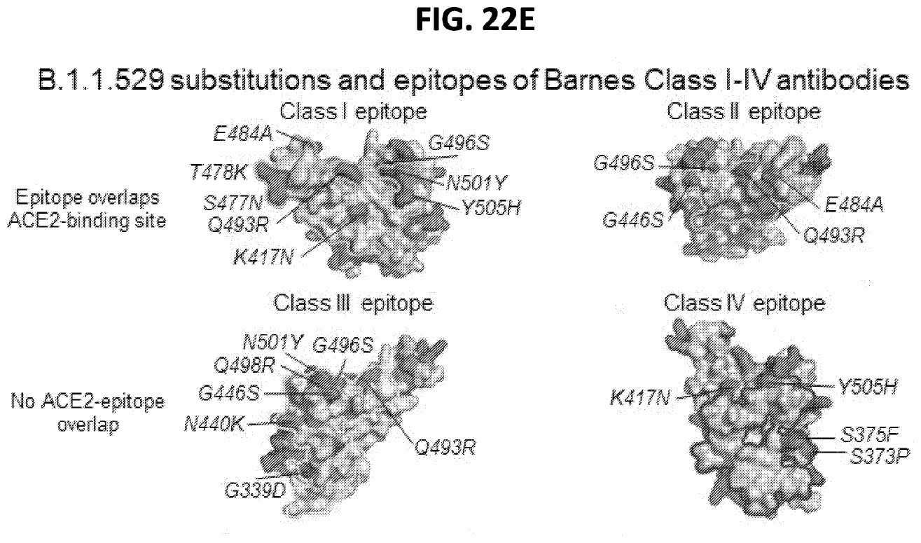

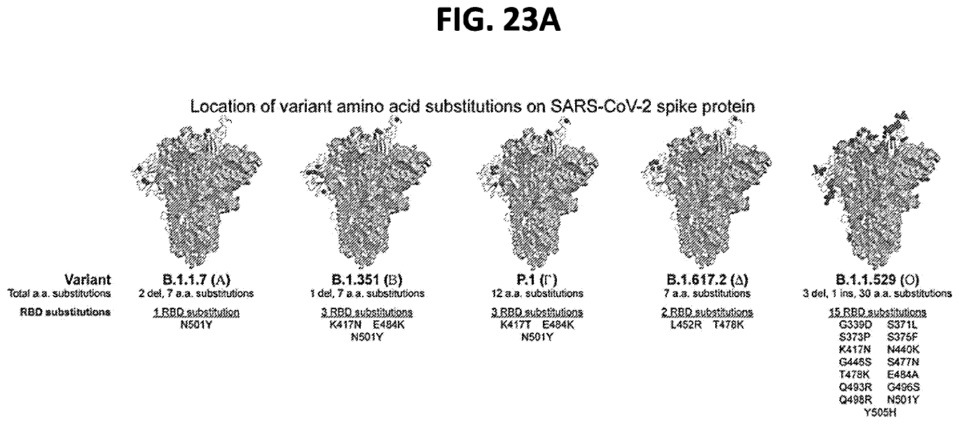

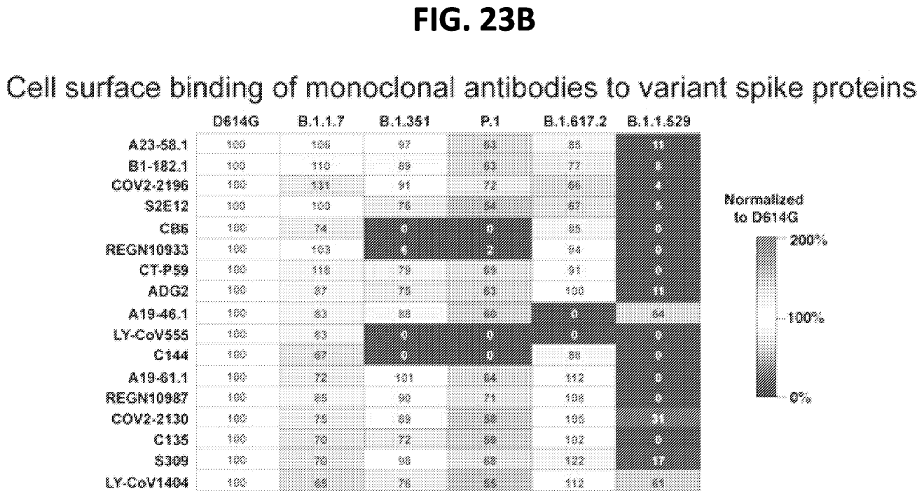

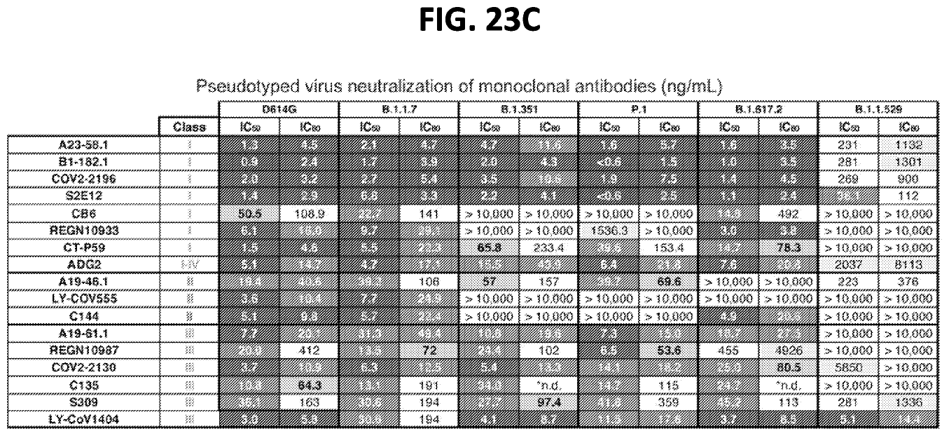

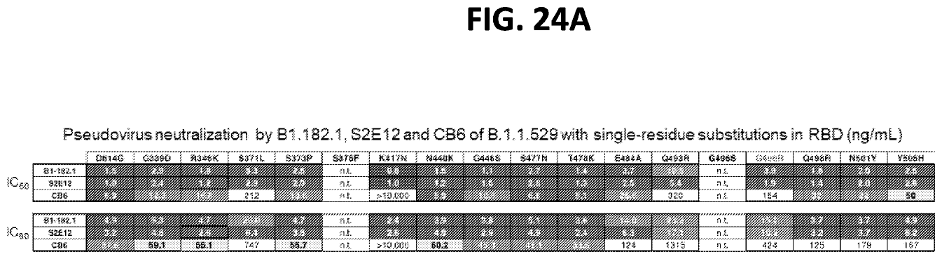

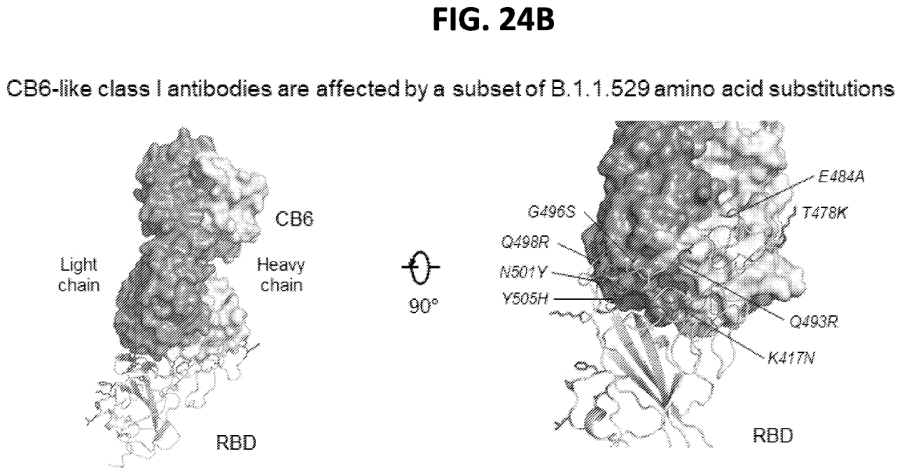

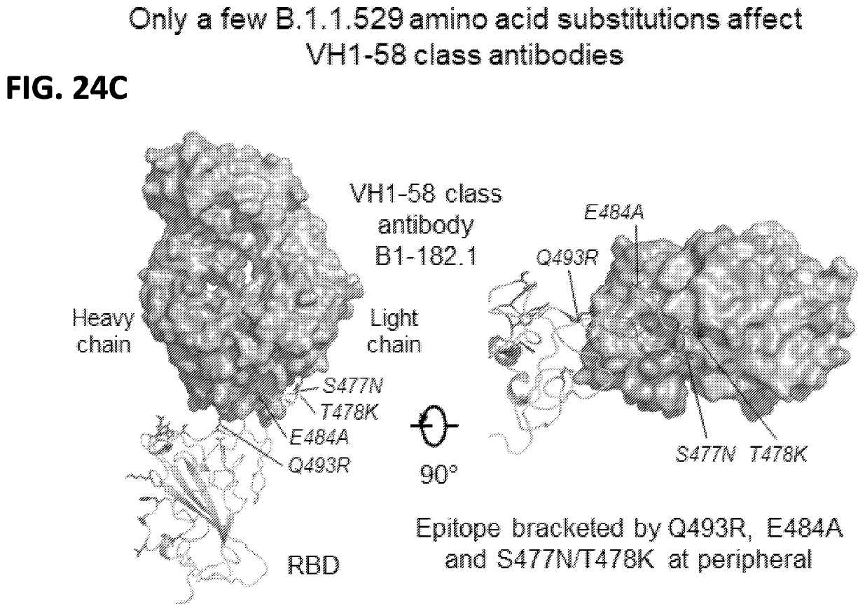

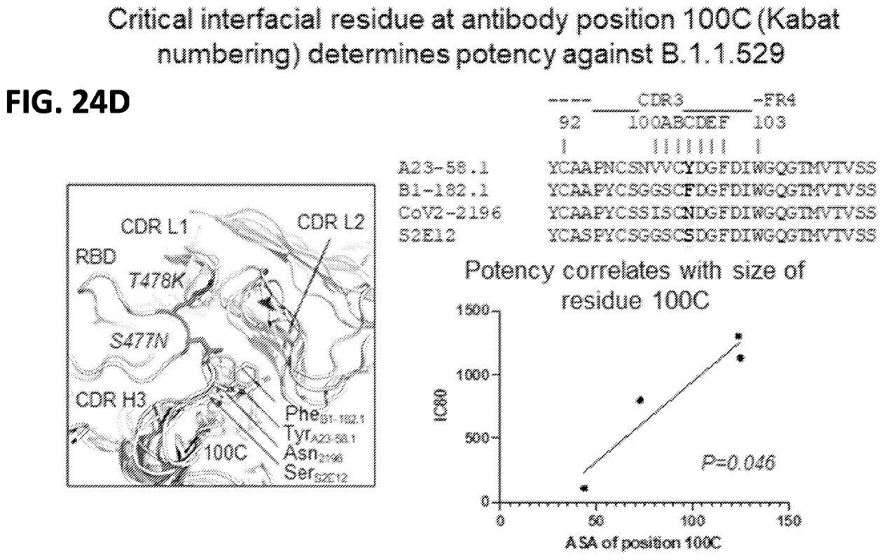

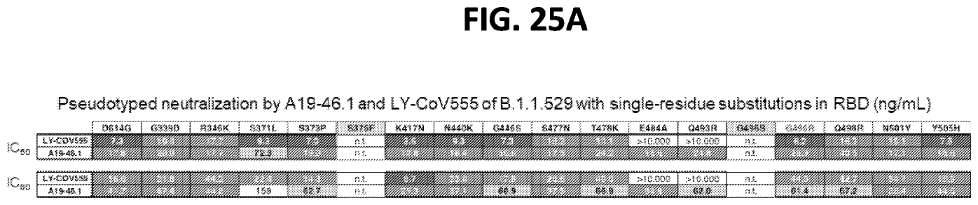

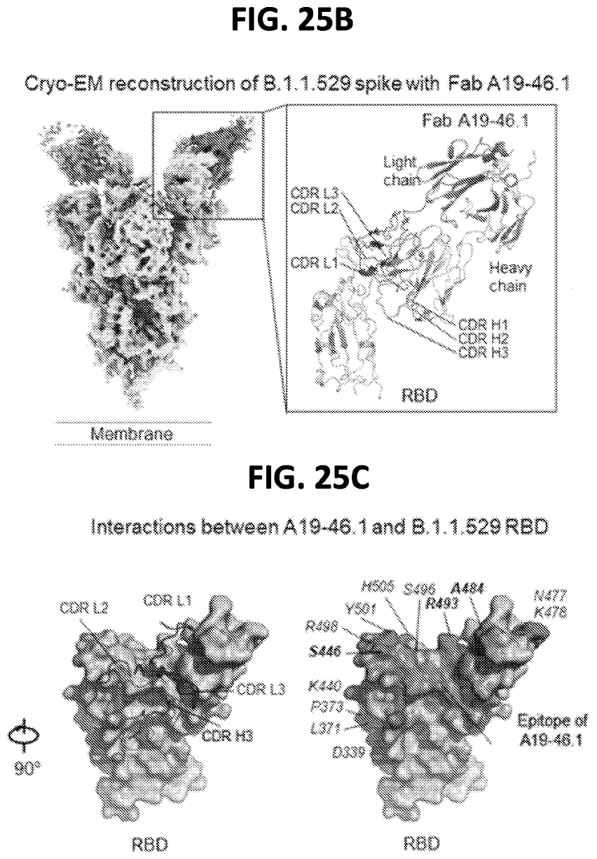

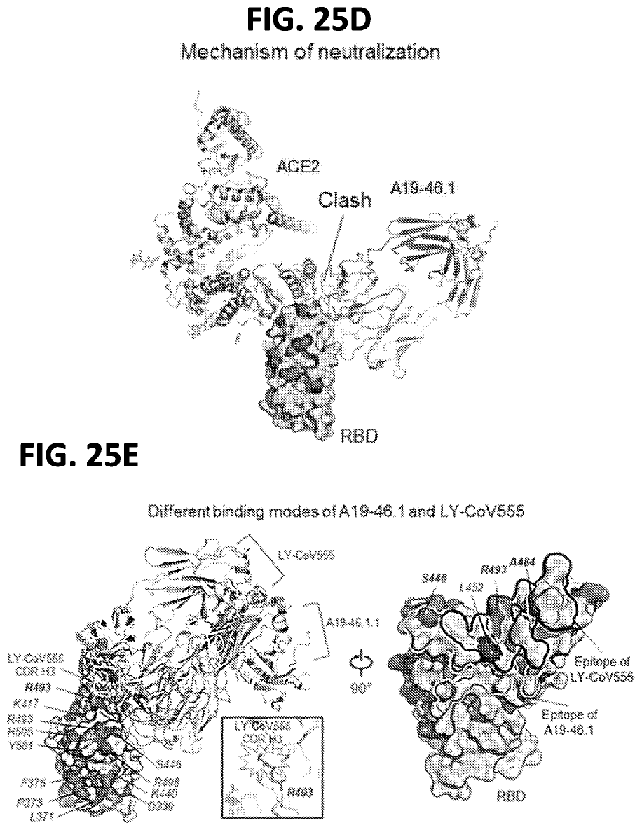

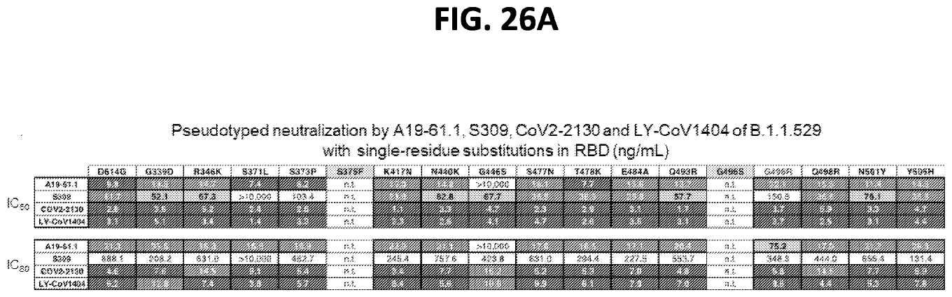

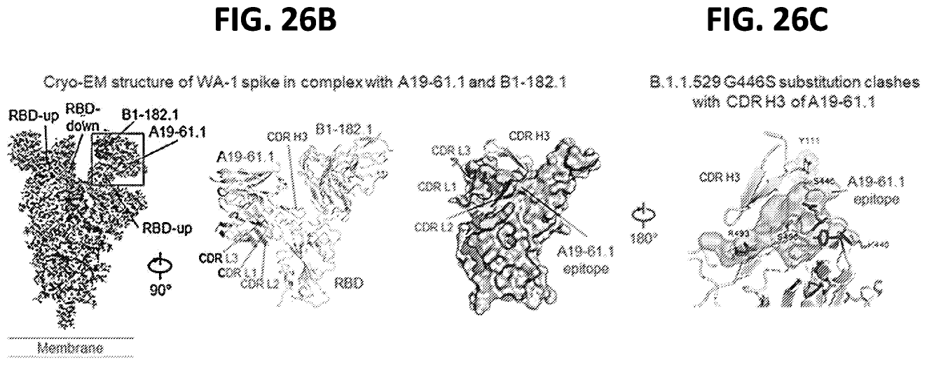

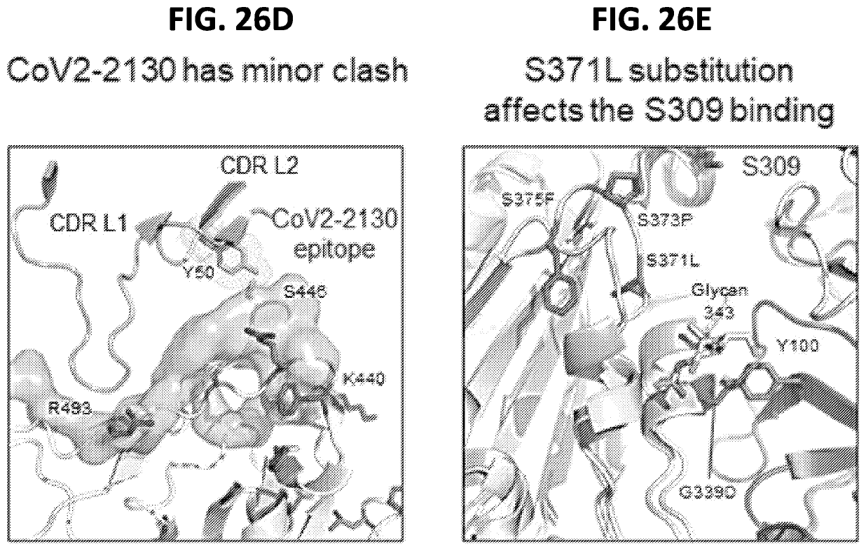

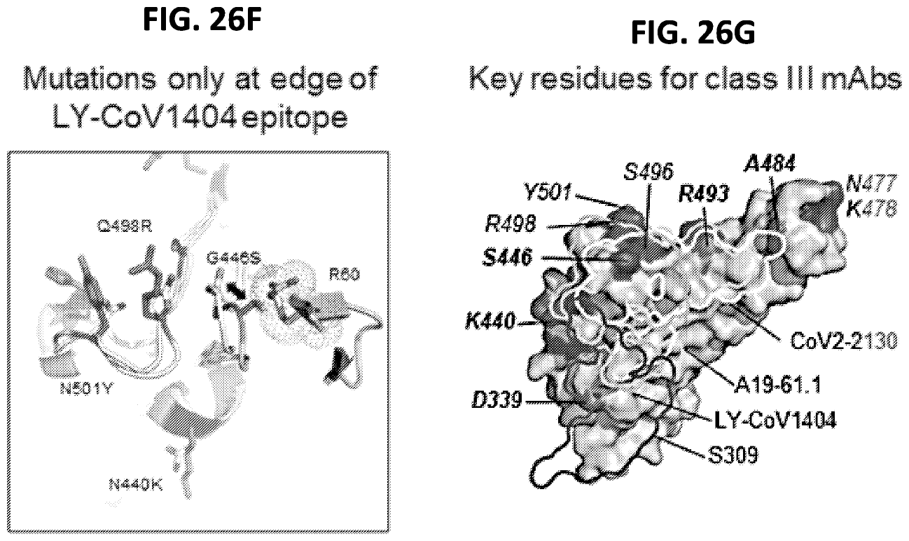

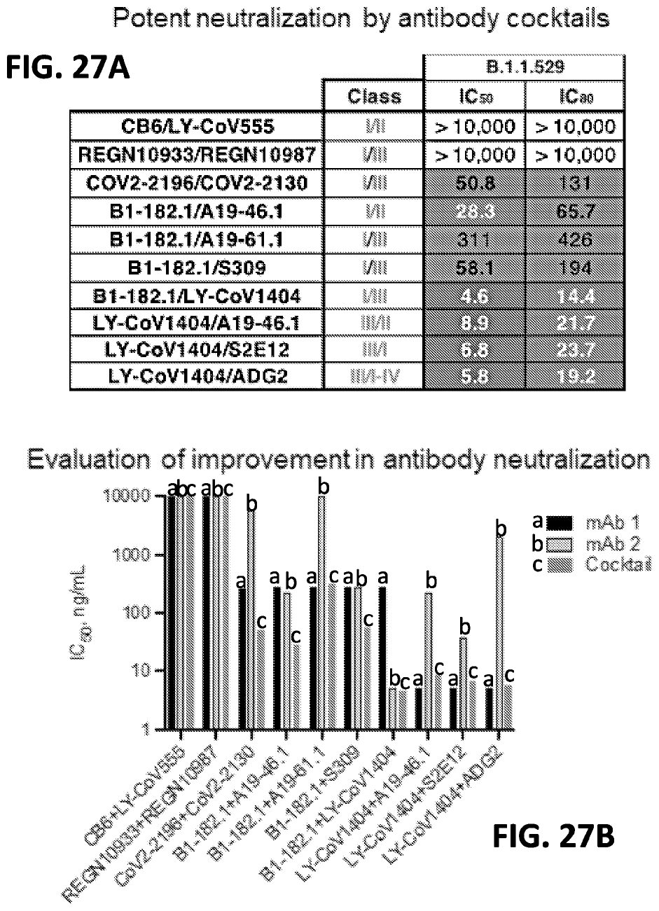

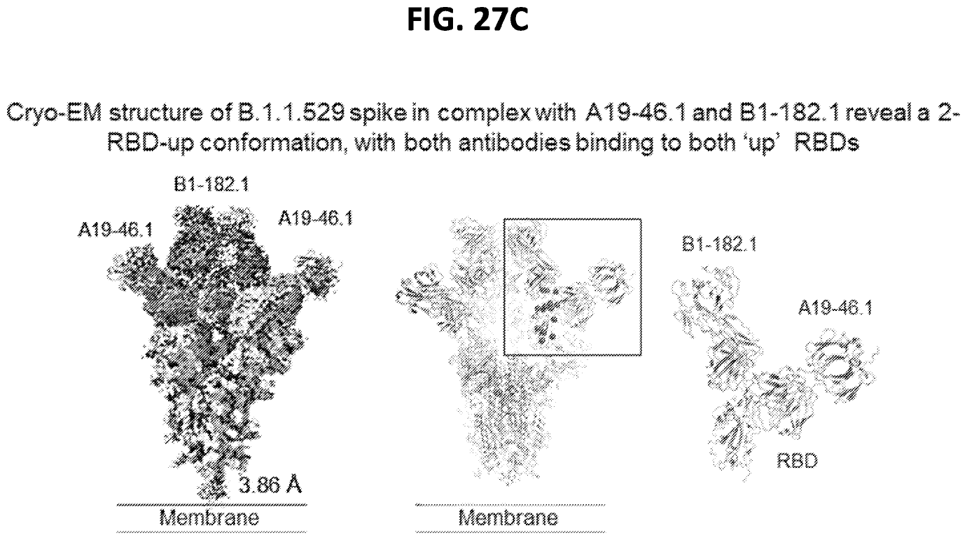

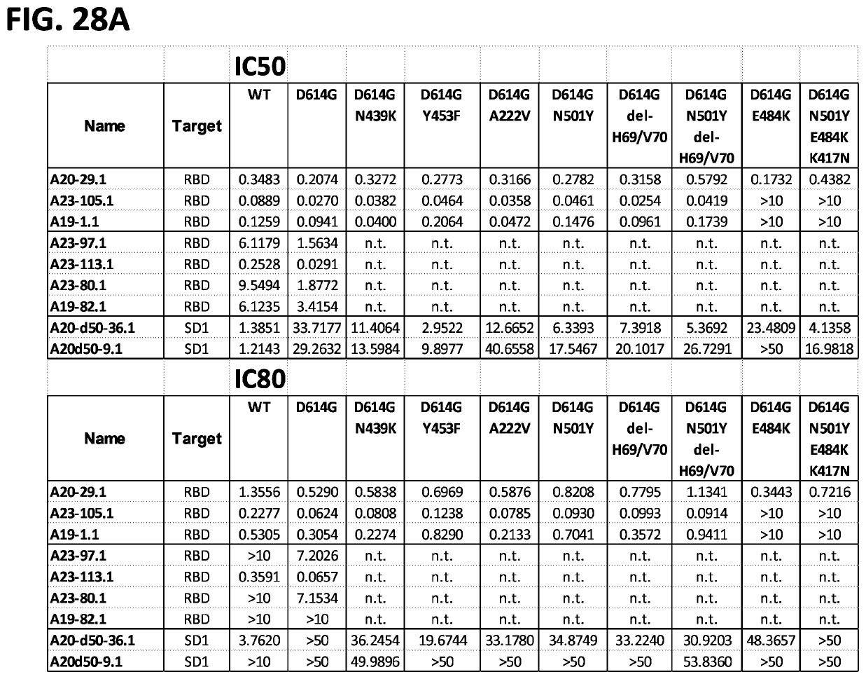

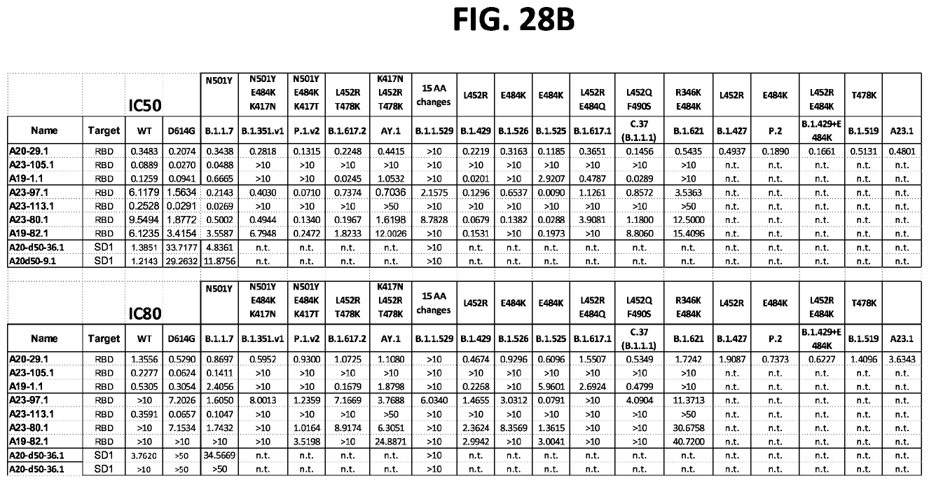

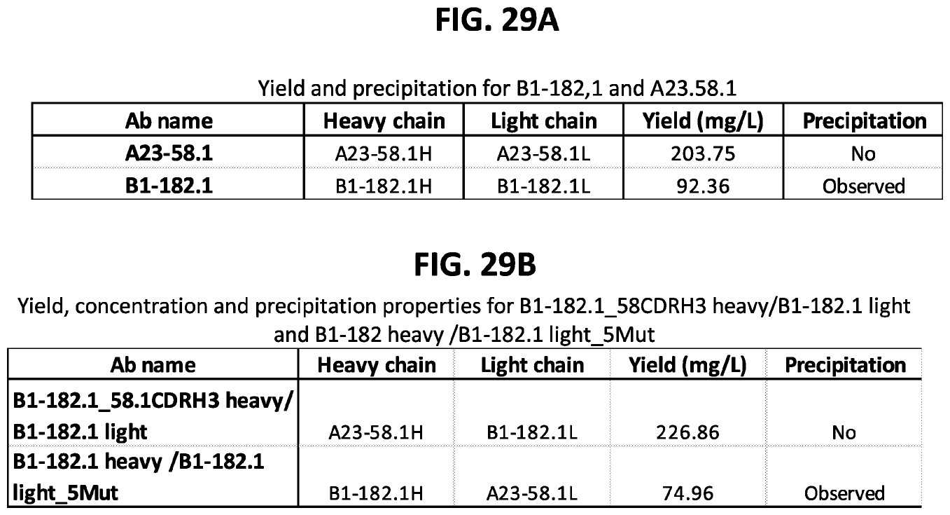

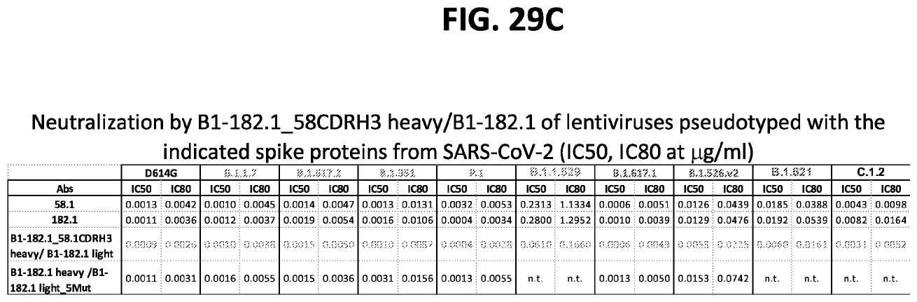

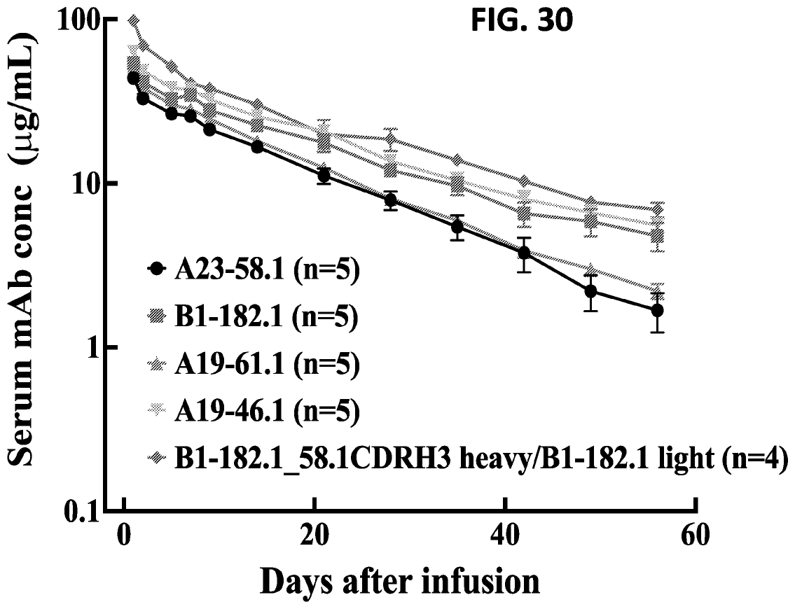

To obtain these antibodies, blood was obtained from twenty-two convalescent subjects, who had experienced mild to moderate symptoms after WA-1-infection, between 25 and 55 days after symptom onset. Four subjects, A19, A20, A23 and B1, had both high neutralizing and binding activity against the WA-1 variant (FIG. 16A) and were selected for antibody isolation efforts. CD19+/CD20+/IgM−/IgA+ or IgG+B cells were sorted for binding to a stabilized version of S (S-2P), the full S1 subunit, or the receptor binding domain plus the subdomain-1 region of S1 (RBD-SD1) (FIG. 16B). In total, we sorted 889 B cells, recovered 709 (80%) paired heavy and light chain antibody sequences and selected 200 antibodies for expression. An MSD binding assay was used to measure binding of these 200 antibodies to stabilized spike, the full S1 subunit, RBD, or NTD. There was a broad response across all spike domains with 77 binding RBD, 46 binding NTD, 58 inferred to bind the S2 subunit based on binding to S, but not to S1, and 19 binding an indeterminant epitope or failing to recognize spike in an MSD binding assay (FIG. 16C).

SMRTseq was used to identify single B-cell receptor sequences. The B cell receptor sequence variable heavy and light chain sequences were synthesized and cloned into human vectors, expressed and the binding, structural and functional capacities. The antibodies are potent neutralizing antibodies and target unique epitopes in the spike glycoprotein of SARS CoV-2.

### Example 1

### In Vitro and In Vivo Assessment of Antibodies Against SARS CoV2

Functional Properties: To determine mAb in vitro functionality, including reactivity with the coronavirus surface spike protein (S). The mAbs were tested for binding to S2P, HexaPro, S1, RBD and/or NTD by ELISA, MSD and biolayer interferometry (BLI).

Epitope Mapping: Global mapping to determine mAb binding properties and epitopes on S2P, HexaPro, S1, RBD and/or NTD was performed by evaluation by ELISA, MSD and using BLI competition with other mAbs and ACE2 which have known epitopes.

Affinity Measurements: Affinity of mAbs to S2P for mAbs was determined using BLI.

Neutralization: Neutralization of virus infection by mAbs was determined using pseudotyped lentivirus particles bearing coronavirus spike protein. Infection caused by the viruses is determined by measuring the expression of a luciferase reporter gene that is encoded by the virus genome.

Structural evaluation: 2D and 3D reconstruction from single particle negative stain electron microscopy class averages and/or CyroEM is used to determine the mode of recognition and molecular interaction that are required for the antibodies to function and bind to target antigen proteins.

### Example 2

### Selection of Coronavirus-Specific Monoclonal Antibodies by Single Cell Sorting and SMRTseq

The monoclonal antibodies whose variable domains are discussed below were isolated using single cell sorting of SARS CoV2 specific memory B cells. Briefly, peripheral blood mononuclear cells were stained with a flow cytometry panel to identify the memory B cell population. Cells were co-stained with fluorescently labeled SARS CoV2 antigen. The probes included RBD, NTD, S1 and S2P. Each probe was labeled in a unique fluorescent color to allow for the phenotyping memory B cells by their capacity to bind each probe and the relative potency of binding to each probe. Memory B cells that were shown to bind to the RBD or S2P probe were sorted as individual cells into wells of 96-well plates. These single cells were subsequently subjected to immunoglobulin heavy and light chain sequencing using the SMRTseq method.

### Example 3

### A23-58.1 (A23-58.1)

Identification of Coronavirus antibodies using FACS probe sorting and SMRTseq sequencing: A23-58.1 is a monoclonal antibody (mAb) whose variable domains were isolated through the pairing of heavy and light chain immunoglobulin genes from a single memory B cell obtained from a SARS CoV2 survivor 48 days following symptom onset. The nucleotide and amino acid sequences for the heavy and light chains of the expressed version of A23-58.1 are below. Variable heavy and light chain immunoglobulin sequence encoding the antigen binding region of A23-58.1 were synthesized and cloned into immunoglobulin expression vectors containing human constant regions for the IgG1 heavy chain and kappa light chain. These vectors were used to express antibody that was purified using standard methods. Table 1:

TABLE. 1
A23-58.1gene family, and nucleotide and amino acid sequences for heavy and
light chains. CDR1, CDR2 and CDR3 are bolded and underlined in the amino acid
sequences

| | V-gene Family | D-gene Family | J-gene Family | Isotype |
|---|---|---|---|---|
| Heavy Chain | IGHV1-58*01 | Not called | IGHJ6*02 | IGHG1*04 |

CAAATGCAGCTGGTGCAGTCTGGGCCTGAGGTGAAGAAGCCTGGGACCTCAGTGAAGGTC
TCCTGCAAGGCTTCTGGATTCACCTTTACTAGCTCTGCTGTGCAGTGGGTGCGACAGGCTC
GTGGACAACGCCTTGAGTGGATAGGATGGATCGTCGTTGGCAGTGGTAACACAAACTACG
CACAGAAGTTCCAGGAAAGAGTCACCATTACCAGGGACATGTCCACAAGCACAGCCTACA
TGGAGCTGAGCAGCCTGAGATCCGAGGACACGGCCGTGTATTACTGTGCGGCACCGAATT
GTAGTAATGTTGTATGCTATGATGGTTTTGATATCTGGGGCCAAGGGACAATGGTCACCGT
CTCTTCAG (SEQ ID NO: 113)
QMQLVQSGPEVKKPGTSVKVSCKASGFTFTSSAVQWVRQARGQRLEWIGWIVVGSGNTNY
AQKFQERVTITRDMSTSTAYMELSSLRSEDTAVYYCAAPNCSNVVCYDGFDIWGQGTMVTV
SS (SEQ ID NO: 25)

| | | |
|---|---|---|
| Light Chain | IGKV3-20*01 | IGKJ1*01 |

GAAATTGTGTTGACGCAGTCTCCAGGCACCCTGTCTTTGTCTCCAGGGGAAAGAGCCACCC
TCTCCTGCAGGGCCAGTCAGAGTGTTAGCAGCAGCTACTTAGCCTGGTACCAGCAGAAAC
CTGGCCAGGCTCCCAGGCTCCTCATCTATAGTGCATCCAGCAGGGCCACTGGCATCCCAG
ACAGGTTCAGTGGCAGTGGGTCTGGGACAGACTTCACTCTCACCATCAGCAGACTGGAGC
CTGAAGATTTTGCAGTGTATTTCTGTCAGCAGTATGGTACCTCACCGTGGACGTTCGGCCA
AGGGACCAAGGTGGAAATCAAAC (SEQ ID NO: 114)
EIVLTQSPGTLSLSPGERATLSCRASQSVSSSYLAWYQQKPGQAPRLLIYSASSRATGIPDRESG
SGSGTDFTLTISRLEPEDFAVYFCQQYGTSPWTFGQGTKVEIK (SEQ ID NO: 29)

### Binding to SARS-CoV-2 Spike Protein

Purified monoclonal antibody A23-58.1 was evaluated for its capacity to bind to four SARS CoV2 Spike derived protein antigens (S2P, S1, RBD and NTD) and the S2P SARS CoV1 spike protein antigen). Binding was determined using ELISA immunoassay and showed that A23-58.1 is able to bind SARS CoV2 S2P, S1 and RBD but not SARS CoV2 NTD or SARS CoV1 S2P (FIG. 1). With the exception of D614G/N439K, which had slightly decreased binding, A23-58.1 maintains or has increased binding to all of the variants tested including D614G, D614G/Y453F,D614G/501Y, D614G/de169-70, D614G/de169-70/N501Y, B.1.1.7, D614G/K471N, D614G/E484K, D614G/K417N/E484K/N501Y and B.1.351 variants, see FIG. 1.

### Epitope Mapping

By assessing how A23-58.1 competes with previously published antibodies and antibodies discovered in this invention report, gross epitope can be determined. Competition classification was determined using Biolayer interferometry (BLI). Briefly, biosensors were loaded with purified SARS CoV2 S2P. The competitor mAb (the mAb determining the class or gross epitope) is then allowed to bind to the antigen and the degree of binding is recorded. Then the analyte mAb is then allowed to bind and the degree of binding is recorded. Percent Inhibition of the binding of the analyte is calculated as follows:

$$\% Inhibition = 100 ( 1 - \frac{signal of analyte binding in the presense of competitor}{signal of analyte binding in the absense of competitor} ) ¶$$

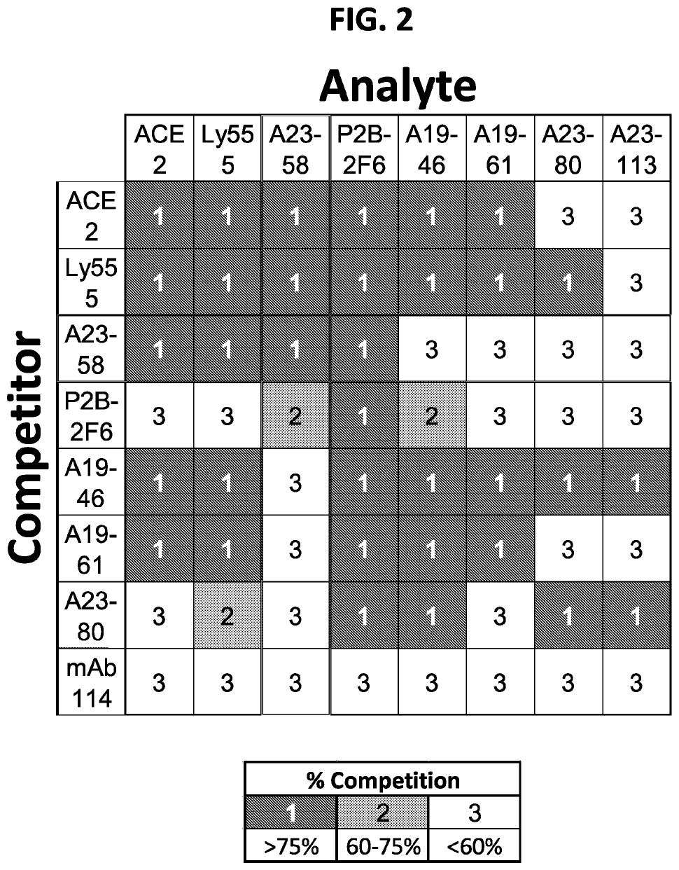

A23-58.1 competition profile is shown in FIG. 2. It shows that while A23-58.1 competes similarly to LY-COV555, it does not compete with A19-46.1, A19-61.1 and A23-80.1 that is competed with LY-COV555. Similarly, A19-46.1, A19-61.1 and A23-80.1 each block binding of LY-COV555 but do not block the binding of A23-58.1. Taken together, this indicates that A23-58.1 has a distinct mode of binding and epitope within the RBD. FIG. 2.

### Kinetics of Binding to the SARS CoV2 S2P Protein

Fab protein generated from A23-58.1 was evaluated for binding to SARS CoV2 S2P using BLI. A23-58.1 Fab shows binding to SARS CoV2 S2P protein with an affinity constant (KD) of 7.3 nM, kₒₙ of 7.13×10⁵ per second and k_{off} of 5.2×10⁻³ per Molar·second.

### Neutralization

A23-58.1 was tested for neutralization activity in a pseudotyped virus entry assay (FIG. 1) A23-58.1 was found to have a neutralization IC₅₀ (0.0025 μg/mL) and IC₈₀ (0.0107 μg/mL) of SARS CoV2 pseudotyped lentivirus particles. This is 3 to 4-fold more potent than the leading clinical candidate LY-COV555 (IC₅₀: 0.0071, IC₈₀: 0.0357 μg/mL). SARS COV-2 Nanoluc live virus neutralization IC₅₀ (0.0021 μg/mL) and IC₈₀ (0.0045 μg/mL) is amongst the most potent reported for antibodies targeting SARS COV-2, see FIG. 1.

Further testing against naturally occurring SARS COV-2 spike variants in pseudotyped neutralization assays showed that A23-58.1 maintains high potency (IC₅₀: <0.0006-0.0058 μg/mL; IC₈₀: 0.0039-0.0183 μg/mL) against the following variants D614G, N439K/D614G, Y453F/D614G, A222V/D614G, N501Y/D614G, de169-70/D614G, N501Y/de169-70/D614G, N501Y/E484K/K417N/D614G and B.1.1.7 (VOC 202012/01) that contains amino acid changes at H69del-V70del-Y144del-N501Y-A570D-D614G-P681H-T716I-S982A-DI 118H. There is slightly reduced but still high potency against E484K/D614G (IC₅₀: 0.0102, IC₈₀: 0.0251 μg/mL) and B.1.351 (IC₅₀: 0.0130, IC₈₀: 0.1196 μg/mL). This is in contrast to LY-CoV555 and REGN-10989 which loses neutralizing capacity (>10 μg/mL) against the E484K/D614G, N501Y/E484K/K417N/D614G and B.1.351 variants. REGN-10933 loses neutralizing capacity (>10 μg/mL) against Y453F/E614G, N501Y/E484K/K417N/D614G and B.1.351. CB6 loses neutralizing activity (>10 μg/mL) against the N501Y/E484K/K417N/D614G and B.1.351 variants. See FIG. 1.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay used for epitope mapping. Briefly, biosensors were loaded with purified SARS CoV2 S2P. The competitor mAb (the mAb being evaluated to determine if it blocks ACE 2 binding) is then allowed to bind to the antigen and the degree of binding is recorded. The soluble ACE2 protein is then allowed to bind and the degree of binding is recorded. Percent Inhibition of the binding of the analyte was calculated as follows:

$$\% Inhibition = 100 ( 1 - \frac{signal of ACE 2 binding in the presense of competitor}{signal of ACE 2 binding in the absense of competitor} )$$

A23-58.1 ACE2 competition profile is shown in FIG. 2 and indicates that A23-58.1 prevents ACE2 from binding to S2P.

### Electron Microscopy Studies

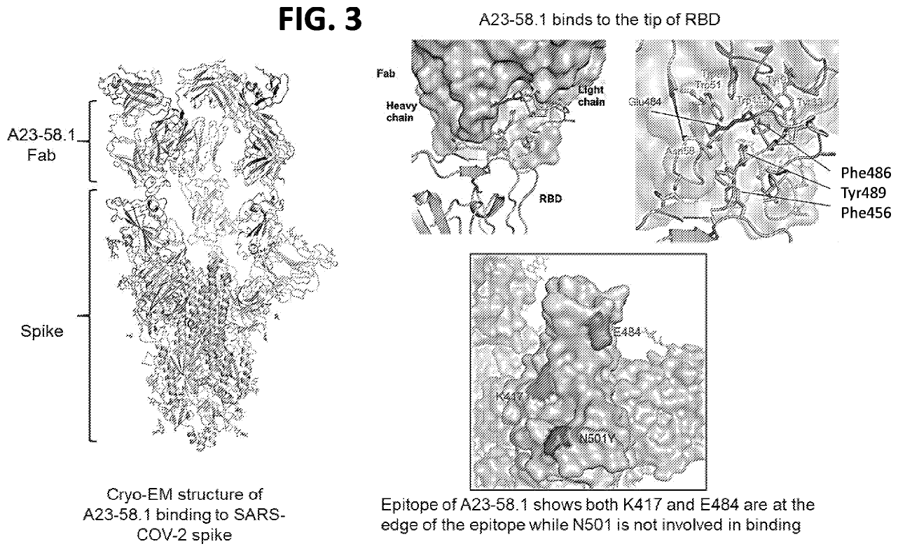

Fab protein was generated from A23-58.1 IgG by enzyme digestion. Excess Fab was incubated in the presence of stabilized 6-proline (S-6P)) spike protein ectodomain (described in Hseih et al, “Structure-based design of prefusion-stabilized SARS-CoV-2 Spikes,” Science, 369(6510):1501-1505, 2020, incorporated by reference herein. The complexed material was then analyzed by cryogenic electron microscopy single particle analysis (FIG. 3). This data indicates that A23-58.1 binds to the spike at 3 Fabs per S-6P trimer with RBD domains in the up position, see FIG. 3.

### Example 4

### A19-61.1 (A19-61.1)

### Identification of Coronavirus Antibodies Using FACS Probe Sorting and SMRTseq Sequencing

A19-61.1 is a monoclonal antibody whose variable domains were isolated through the pairing of heavy and light chain immunoglobulin genes from a single memory B cell obtained from a SARS CoV2 survivor 41 days following symptom onset. Flow cytometry data suggested that the antibody. The nucleotide and amino acid sequences for the heavy and light chains of the expressed version of A19-61.1 can be found in Table 2. Variable heavy and light chain immunoglobulin sequence from Table 2 encoding the antigen binding region of A19-61.1 were synthesized and cloned into immunoglobulin expression vectors containing human constant regions for the IgG1 heavy chain and kappa light chain. These vectors were used to express antibody that was purified using standard methods.

TABLE 2
A19-61.1 gene family, and nucleotide and amino acid sequences for heavy and light
chains. CDR1, CDR2 and CDR3 are bolded and underlined in the amino acid
sequences

| | V-gene Family | D-gene Family | J-gene Family | Isotype |
|---|---|---|---|---|
| Heavy | IGHV3-30-3*01, | IGHD6-19*01 | IGHJ6*02 | IGHGP*01, IGHG3*04, |
| Chain | IGHV3-30*17 | | | IGHG2*06 |

CAGGTGCAGCTGGTGGAGTCTGGGGGAGGCGTGGTCCAGCCTGGGAGGTCCCTGAGACTC
TCCTGTGCAGCCTCTGGATTCACCTTCAGTAGCTATGCTTTCCACTGGGTCCGCCAAGCTC
CGGGCAAGGGGCTGGAGTGGGTGGCAGTTATATCATATGATGGAAGCAATCAATACTACG
CAGACTCCGTGAAGGGCCGATTCACCATCTCCAGAGACAATTCCAAGAACACGCTGTATC
TGCAAATGAACAGCCTGAGAGCTGACGACACGGCTGTGTATTACTGTGCGAGAGATCTGG
CTATAGCAGTGGCTGGTACGTGGCACTATTATAACGGTATGGACGTCTGGGGCCAAGGGA
CCACGGTCACCGTCTCCTCAG (SEQ ID NO: 115)
QVQLVESGGGVVQPGRSLRLSCAASGFTFSSYAFHWVRQAPGKGLEWVAVISYDGSNQYYA
DSVKGRFTISRDNSKNTLYLQMNSLRADDTAVYYCARDLAIAVAGTWHYYNGMDVWGQG
TTVTVSS (SEQ ID NO: 9)

| | | |
|---|---|---|
| Light | IGKV1D-12*02, | IGKJ5*01 |
| Chain | IGKV1-12*02 | |

GACATCCAGATGACCCAGTCTCCATCTTCCGTGTCTGCATCAGTAGGAGACAGAGTCATCA
TCACTTGTCGGGCGAGTCAGGGTATTTCCAGCTGGTTAGCCTGGTATCAGCAGAAACCAG
GGAAAGCCCCTAAGGTCCTGATCTATGATGCATCCAGTTTGCAAAGTGGGGTCCCATCAA
GGTTCAGCGGCAGTGGATATGGGACAGATTTCACTCTCACCATCAGCAGCCTGCAGCCTG
AAGATTCTGCAACTTACTATTGTCAACAGGCTAAAAGTTTTCCGATCACCTTCGGCCAAGG
GACACGACTGGAGATTAAAC (SEQ ID NO: 116)
DIQMTQSPSSVSASVGDRVIITCRASQGISSWLAWYQQKPGKAPKVLIYDASSLQSGVPSRFSG
SGYGTDFTLTISSLQPEDSATYYCQQAKSFPITFGQGTRLEIK (SEQ ID NO: 13)

### Binding to SARS-CoV-2 Spike Protein

Purified monoclonal antibody A19-61.1 was evaluated for its capacity to bind to four SARS CoV2 Spike derived protein antigens (S2P, S1, RBD and NTD) and the S2P SARS CoV1 spike protein antigen (FIG. 1). Binding was determined using ELISA immunoassay and showed that A19-61.1 bound SARS CoV2 S2P, S1 and RBD but not SARS CoV2 NTD or SARS CoV1 S2P. With the exception of D614G/N439K, which had slightly decreased binding, A19-61.1 maintains or has slightly increased binding to all of the variants tested including D614G, D614G/Y453F,D614G/501Y, D614G/de169-70, D614G/de169-70/N501Y, B.1.1.7, D614G/K471N, D614G/E484K, D614G/K417N/E484K/N501Y and B.1.351 variants, see FIG. 1.

### Epitope Mapping

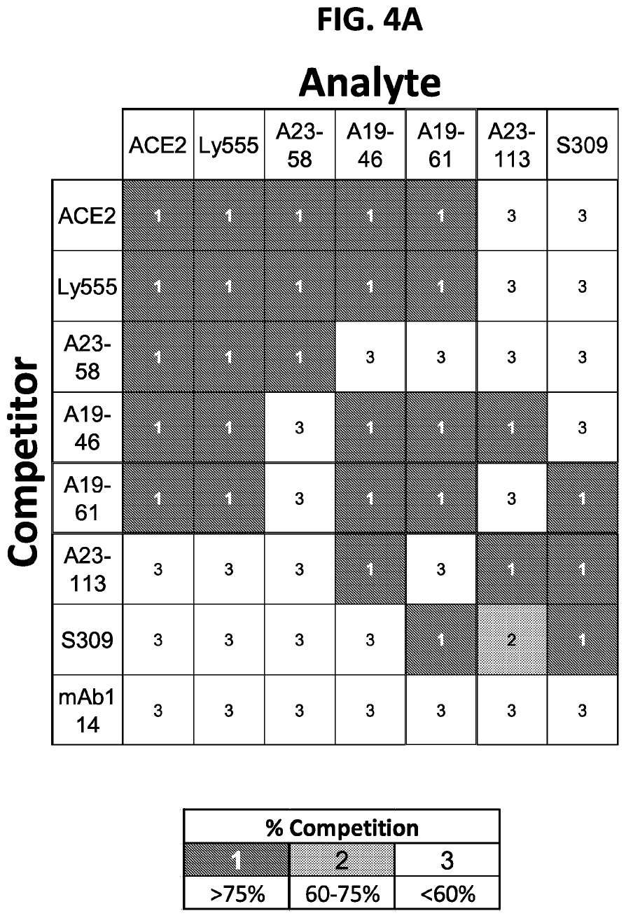

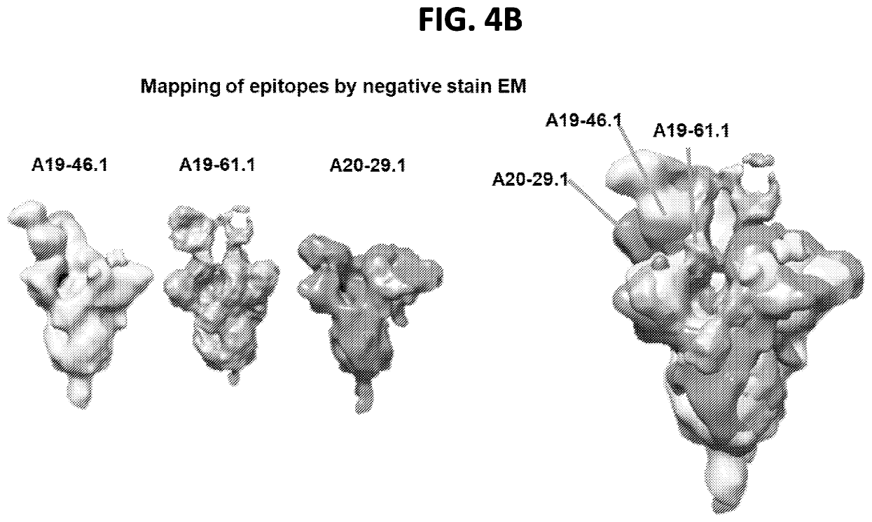

By assessing how A19-61.1 competes with previously published antibodies and the antibodies disclosed herein, gross epitope can be determined. Competition classification was determined using Biolayer interferometry (BLI) assay described above. A19-61.1 competition profile is shown in FIG. 4. A19-61.1 binding blocks 5309 and LY-COV555 binding and both 5309 and LY-COV555 block A19-61.1 binding. However, the rest of the competition profile is different from these two antibodies. For example, there was differential competition with A23-113.1 and A19-46.1 and A23-58.1 (see below). Negative EM indicated this antibody binds to RBD on the spike in the RBD down position with an epitope close to the 3-fold axis of the spike (FIG. 4B). Taken together, this indicates that A19-61.1 has a distinct mode of binding and epitope within the RBD.

### Kinetics of Binding to the SARS CoV2 S2P Protein

Fab protein generated from A19-61.1 was evaluated for binding to SARS CoV2 S2P using BLI. A19-61.1. The Fab showed binding to SARS CoV2 S2P protein with an affinity constant (KD) of 2.33 nM, kₒₙ of 3.04×10⁵ per second and k_{off} of 7.06×10⁻⁴ per Molar·second.

### Neutralization

A19-61.1 was tested for neutralization activity in a pseudotyped virus entry assay (See FIG. 2). A19-61.1 was found to have a neutralization IC₅₀ (0.0709 μg/mL) and IC₈₀ (0.1633 μg/mL) of SARS-CoV-2 pseudotyped lentivirus particles. Further testing against naturally occurring SARS COV-2 spike variants in pseudotyped neutralization assays showed that A19-61.1 maintains high potency (IC₅₀: 0.0020-0.0237 μg/mL; IC₈₀: 0.0131-0.0418 μg/mL) against the following variants D614G, N439K/D614G, Y453F/D614G, A222V/D614G, N501Y/D614G, de169-70/D614G, N501Y/de169-70/D614G, E484K/D614G and N501Y/E484K/K417N/D614G, B.1.1.7 (VOC 202012/01) that contains amino acid changes at H69del-V70del-Y144del-N501Y-A570D-D614G-P681H-T716I-S982A-D1118H and the B.1.351 variant. This is in contrast to LY-CoV555 and REGN-10989 which loses neutralizing capacity (>10 μg/mL) against the E484K/D614G, N501Y/E484K/K417N/D614G and B.1.351 variants. REGN-10933 loses neutralizing capacity (>10 μg/mL) against Y453F/E614G, N501Y/E484K/K417N/D614G and B.1.351. CB6 loses neutralizing activity (>10 μg/mL) against the N501Y/E484K/K417N/D614G and B.1.351 variants. See FIG. 1. SARS COV-2 Nanoluc live virus neutralization IC₅₀ (0.0022 μg/mL) and IC₈₀ (0.0134 μg/mL) is amongst the most potent reported for antibodies targeting SARS COV-2.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A19-61.1 ACE2 competition profile is shown in FIG. 4 and indicates that A19-61.1 prevents ACE2 from binding to S2P. FIG. 4

### Example 5

### A19-46.1 (A19-46.1)

### Identification of Coronavirus Antibodies Using FACS Probe Sorting and SMRTseq Sequencing

A19-46.1 is a mAb whose variable domains were isolated through the pairing of heavy and light chain immunoglobulin genes from a single memory B cell obtained from a SARS CoV2 survivor 41 days following symptom onset. The nucleotide and amino acid sequences for the heavy and light chains of the expressed version of A19-46.1 can be found in Table 3. Variable heavy and light chain immunoglobulin sequence encoding the antigen binding region of A19-46.1 were synthesized and cloned into immunoglobulin expression vectors containing human constant regions for the IgG1 heavy chain and lambda light chain. These vectors were used to express antibody that was purified using standard methods.

TABLE 3
SARS2.A789-d41-46.1 gene family, and nucleotide and amino acid sequences for
heavy and light chains. CDR1, CDR2 and CDR3 are bolded and underlined in
the amino acid sequences

| | V-gene Family | D-gene Family | J-gene Family | Isotype |
|---|---|---|---|---|
| Heavy | IGHV3-30*03, | Not assigned | IGHJ6*02 | IGHG |
| Chain | IGHV3-30-5*01 | | | |

CAGGTGCAGCTGGTGGAGTCTGGGGGAGGCGTGGTCCAGCCTGGGAGGTCCCTGAGACTC
TCCTGTGCAGCCTCTGGATTCACCCTCAGTAGCTATGGCATGCACTGGGTCCGCCAGGCTC
CAGGCAAGGGGCTGGAGTGGGTGGCAGTTATATCATATGATGGAAGTAATAAATACTATG
TAGACTCCGTGAAGGGCCGATTCACCATCTCCAGAGACAATTCCAAGAACACGCTGTATC
TGCAAATGAACAGCCTGAGAGCTGAGGACACGGCTGTGTATTACTGTGCGAGGGGGTGGG
CTTATTGGGAGCTACTCCCTGACTACTACTACGGTATGGACGTCTGGGGCCAAGGGACCA
CGGTCACCGTCTCCTCAG (SEQ ID NO: 117)
QVQLVESGGGVVQPGRSLRLSCAASGFTLSSYGMHWVRQAPGKGLEWVAVISYDGSNKYY
VDSVKGRFTISRDNSKNTLYLQMNSLRAEDTAVYYCARGWAYWELLPDYYYGMDVWGQG
TTVTVSS (SEQ ID NO: 17)

| | | |
|---|---|---|
| Light Chain | IGLV8-61*01 | IGLJ3*01, IGLJ2*01 |

CAGACTGTGGTGACACAGGAGCCATCGTTCTCAGTGTCCCCTGGAGGGACAGTCACACTC
ACTTGTGGCTTGAGCTCTGGCTCAGTCTCTACTGCTTACTTCCCCAGCTGGTACCAGCAGA
CCCCAGGCCAGGCTCCACGCACGCTCATCTACGGTACAAACACTCGCTCTTCTGGGGTCCC
CGATCGCTTCTCTGGCTCCATCCTTGGGAACAAAGCTGCCCTCACCATCACGGGGGCCCAG
GCAGACGATGAATCTGATTATTACTGTGTGCTGTATATGGGTAGAGGCATTGTGGTATTCG
GCGGAGGGACCAAGCTGACCGTCCTAG (SEQ ID NO: 118)
QTVVTQEPSFSVSPGGTVTLTCGLSSGSVSTAYFPSWYQQTPGQAPRTLIYGTNTRSSGVPDRF
SGSILGNKAALTITGAQADDESDYYCVLYMGRGIVVFGGGTKLTVL (SEQ ID NO: 21)

### Binding to SARS-CoV-2 Spike Protein

Purified monoclonal antibody A19-46.1 was evaluated for its capacity to bind to four SARS CoV2 Spike derived protein antigens (S2P, S1, RBD and NTD) and the S2P SARS CoV1 spike protein antigen (FIG. 1). Binding was determined using ELISA immunoassay and showed that A19-46.1 is able to bind strongly to SARS CoV2 S2P, S1 and RBD but not SARS CoV1 S2P. It also bound weakly to SARS CoV2 NTD, see FIG. 1. With the exception of D614G/N439K and D614G/E484K, which have slightly decreased binding, A19-46.1 maintains or has slightly increased binding to all of the variants tested including D614G, D614G/Y453F,D614G/501Y, D614G/de169-70, D614G/de169-70/N501Y, B.1.1.7, D614G/K471N, D614G/K417N/E484K/N501Y and B.1.351 variants, see FIG. 1.

### Epitope Mapping

By assessing how A19-46.1 competes with previously published antibodies and antibodies discovered in this invention report, gross epitope can be determined. Competition classification was determined using Biolayer interferometry (BLI) assay described above.

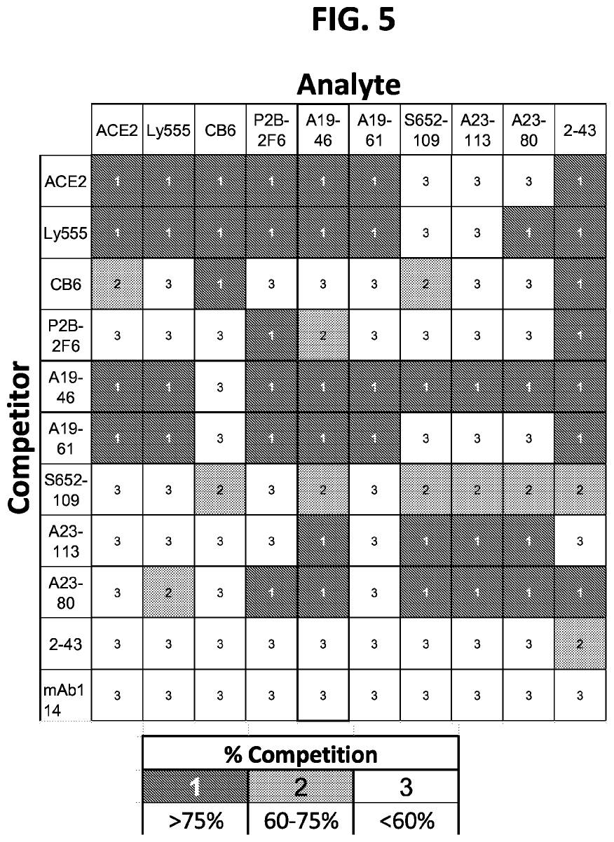

A19-46.1 competition profile is shown in FIG. 5.

Similarly, A19-46.1, A19-61.1 and A23-80.1 each block binding of LY-COV555 but do not block the binding of A23-58.1.

Negative EM indicated this antibody binds to RBD on the spike in the RBD down position, but with an angle that is different to that of A19-61.1 (FIG. 4B). Taken together, this indicates that A19-46.1 has a distinct mode of binding and epitope within the RBD.

### Kinetics of Binding to the SARS CoV2 S2P Protein

Fab protein generated from A19-46.1 was evaluated for binding to SARS CoV2 S2P using BLI. A23-58.1 Fab shows binding to SARS CoV2 S2P protein with an affinity constant (KD) of 3.58 nM, kₒₙ of 3.79×10⁵ per second and k_{off} of 1.35×10⁻³ per Molar·second.

### Neutralization

A19-46.1 was tested for neutralization activity in a pseudotyped virus entry assay (FIG. 1). A19-46.1 was found to have neutralization IC50 (0.0398 μg/mL) and IC80 (0.1287 μg/mL) of SARS-CoV-2 pseudotyped lentivirus particles. Further testing against naturally occurring SARS COV-2 spike variants in pseudotyped neutralization assays showed that A19-46.1 maintains high potency (IC₅₀: 0.0149-0.1265 μg/mL; IC₈₀: 0.0435-0.9036 μg/mL) against the following variants D614G, N439K/D614G, Y453F/D614G, A222V/D614G, N501Y/D614G, de169-70/D614G, N501Y/de169-70/D614G, E484K/D614G and N501Y/E484K/K417N/D614G and B.1.1.7 (VOC 202012/01) that contains amino acid changes at H69del-V70del-Y144del-N501Y-A570D-D614G-P681H-T716I-S982A-D1118H. This is in contrast to LY-CoV555 and REGN-10989 which loses neutralizing capacity (>10 μg/mL) against the E484K/D614G, N501Y/E484K/K417N/D614G and B.1.351 variants. REGN-10933 loses neutralizing capacity (>10 μg/mL) against Y453F/E614G, N501Y/E484K/K417N/D614G and B.1.351. CB6 loses neutralizing activity (>10 μg/mL) against the N501Y/E484K/K417N/D614G and B.1.351 variants. See FIG. 1. SARS COV-2 Nanoluc live virus neutralization IC₅₀ (0.0048 μg/mL) and IC₈₀ (0.0535 μg/mL) is amongst the most potent reported for antibodies targeting SARS COV-2.

### ACE2 Receptor Blocking

SARS CoV-1 and SARS CoV-2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. An A19-46.1 ACE2 competition profile is shown in FIG. 5 and indicates that A19-46.1 prevents ACE2 from binding to S2P.

### Example 6

### 4.5 A23-105.1 (A23-105.1)

### Identification of Coronavirus Antibodies Using FACS Probe Sorting and SMRTseq Sequencing

A23-105.1 is a mAb whose variable domains were isolated through the pairing of heavy and light chain immunoglobulin genes from a single memory B cell obtained from a SARS CoV2 survivor 48 days following symptom onset. The nucleotide and amino acid sequences for the heavy and light chains of the expressed version of A23-105.1 can be found in Table 4. Variable heavy and light chain immunoglobulin sequence from Table 4 encoding the antigen binding region of A23-105.1 were synthesized and cloned into immunoglobulin expression vectors containing human constant regions for the IgG1 heavy chain and kappa light chain. These vectors were used to express antibody that was purified using standard methods.

TABLE 4
A23-105.1 gene family, and nucleotide and amino acid sequences for heavy and
light chains. CDR1, CDR2 and CDR3 are bold and underlined in the amino acid
sequences

| | V-gene Family | D-gene Family | J-gene Family | Isotype |
|---|---|---|---|---|
| Heavy Chain | IGHV3-30-3*01, | Not called | IGHV3-30-3*01, | IGHGP*01, |
| | IGHV3-30*04 | | IGHV3-30*04 | IGHG3*04, |
| | | | | IGHG2*06 |

CAGGTGCAGCTGGTGGAGTCTGGGGGAGGCGTGGTCCAGCCTGGGAGGTCCCTGAGACTC
TCCTGTGCAGCCTCTGGATTCACCTTCAGTAACTATGCTATCCACTGGGTCCGCCAGGCTC
CAGGCAAGGGGCTGGAGTGGGTGGCAGTTATATCATATGATGGAAGCAATAAATACTACG
CAGACTCCGTGAAGGGCCGATTCACCATCTCCAGAGACAATTCCAAGAACACGCTGTATC
TGGAAATGAACAGCCTGAGAGCTGAGGATATGGCTGTGTATTACTGTGCGAGAGTCGGTC
CGTATCAGTATGATAGTAGTGCTGCTTTTGATATCTGGGGCCAAGGGACAATGGTCACCGT
CTCTTCAG (SEQ ID NO: 119)
QVQLVESGGGVVQPGRSLRLSCAASGFTFSNYAIHWVRQAPGKGLEWVAVISYDGSNKYYA
DSVKGRFTISRDNSKNTLYLEMNSLRAEDMAVYYCARVGPYQYDSSAAFDIWGQGTMVTVS
S (SEQ ID NO: 41)

| | | |
|---|---|---|
| Light Chain | IGKV1-5*01 | IGKJ1*01 |

GACATCCAGATGACCCAGTCTCCTTCCACCCTGTCTGCATCTGTAGGAGACAGAGTCACCA
TCACTTGCCGGGCCAGTCAGAGTATTAGTAGCTGGTTGGCCTGGTATCAGCAGAAACCAG
GGCAAGCCCCTAAGCTCCTGATCTATGATGCCTCCAGTTTGGAAAGTGGGGTCCCATCAA
GGTTCAGCGGCAGTGGATCTGGGACAGAATTCACTCTCACCATCAGCAGCCTGCAGCCTG
ATGATTTTGCAACTTATTACTGCCAACAGTATAATAGTTATTCTCGAACGTTCGGCCAAGG
GACCAAGGTGGAAATCAAAC (SEQ ID NO: 120)
DIQMTQSPSTLSASVGDRVTITCRASQSISSWLAWYQQKPGQAPKLLIYDASSLESGVPSRFSG
SGSGTEFTLTISSLQPDDFATYYCQQYNSYSRTFGQGTKVEIK (SEQ ID NO: 45)

### Binding to SARS-CoV-2 Spike Protein

Purified monoclonal antibody A23-105.1 was evaluated for its capacity to bind to four SARS CoV-2 Spike derived protein antigens (S2P, S1, RBD and NTD) and the S2P SARS CoV-1 spike protein antigen (FIG. 1). Binding was determined using ELISA immunoassay and showed that A23-105.1 is able to bind SARS CoV2 S2P, S1 and RBD but not SARS CoV2 NTD or SARS CoV1 S2P. This is consistent with the flow cytometry probe staining and indicates that the epitope is present in the RBD domain.

### Epitope Mapping

By assessing how A23-105.1 competes with previously published antibodies and antibodies discovered in this invention report, gross epitope can be determined. Competition classification was determined using Biolayer interferometry (BLI) assay described above.

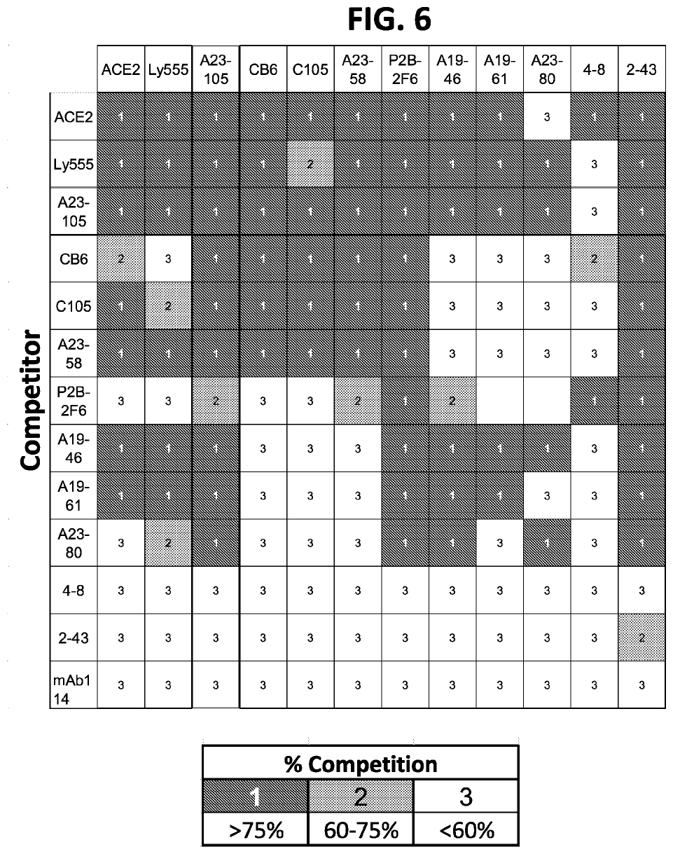

A23-105.1 competition profile is shown in FIG. 6. While A23-105.1 competes similarly to LY-COV555, it does not compete with A19-46.1, A19-61.1 and A23-80.1. Taken together, this indicates that A23-105.1 has a distinct mode of binding and epitope within the RBD.

### Kinetics of Binding to the SARS CoV2 S2P Protein

Fab protein generated from A23-105.1 was evaluated for binding to SARS CoV2 S2P using BLI. A23-105.1 Fab shows binding to SARS CoV2 S2P protein with an affinity constant (K_{D}) of 2.59 nM, kₒₙ of 1.68×10⁵ per second and k_{off} of 4.36×10⁻⁴ per Molar·second.

### Neutralization

A23-105.1 was tested for neutralization activity in a pseudotyped virus entry assay (FIG. 1). A23-105.1 was found to have a neutralization IC₅₀ (0.0889 μg/mL) and IC₈₀ (0.2277 μg/mL) of SARS CoV2 pseudotyped lentivirus particles (FIG. 1). SARS COV-2 Nanoluc live virus neutralization by A23-105.1 shows an IC₅₀ (0.0186 μg/mL) and IC₈₀ (0.06 μg/mL) showing it is highly potent mAb.

### ACE2 Receptor Blocking

SARS CoV-1 and SARS CoV-2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A23-105.1 ACE2 competition profile is shown in FIG. 6 and indicates that A23-105.1 does prevent ACE2 from binding to S2P.

### Example 7

### 4.6 A19-1.1 (A19-1.1)

### Identification of Coronavirus Antibodies Using FACS Probe Sorting and SMRTseq Sequencing

A19-1.1 is a mAb whose variable domains were isolated through the pairing of heavy and light chain immunoglobulin genes from a single memory B cell obtained from a SARS CoV2 survivor 41 days following symptom onset. The nucleotide and amino acid sequences for the heavy and light chains of the expressed version of A19-1.1 can be found in Table 5. Variable heavy and light chain immunoglobulin sequence from Table 5 encoding the antigen binding region of A19-1.1 were synthesized and cloned into immunoglobulin expression vectors containing human constant regions for the IgG1 heavy chain and lambda light chain. These vectors were used to express antibody that was purified using standard methods.

TABLE 5
A19-1.1 gene family, and nucleotide and amino acid sequences for heavy and light
chains. CDR1, CDR2 and CDR3 are bolded and underlined in the amino acid
sequences

| | V-gene Family | D-gene Family | J-gene Family | Isotype |
|---|---|---|---|---|
| Heavy Chain | IGHV3-30-3*01 | Not assigned | IGHJ5*02 | IGHA1*01 |

CAGGTGCAGTTGGTAGAGTCTGGGGGAGGCGTGGTCCAGCCTGGGAGGTCCCTGAGACTC
TCCTGTGCAGCCTCTGGATTCACCTTCACTAATTATGCAATGCACTGGGTCCGCCAGGCTC
CAGGCAAGGGGCTGGAGTGGGTGGCAGTTATATCAAATGATGGAAGCGATAAATACTAC
GCGGACTCCGTGAAGGGCCGATTCACCATCTCCAGAGACAATTCCAAGAGCACGGTTTAT
TTGCAAATGAGCAGCCTGAGACCTGAGGACACGGCTGTGTATTTCTGTGCGAGAGATCCC
CCCCAGGTTCACTGGTCCCTCGACTACTGGGGCCAGGGAACCCTGGTCACCGTCTCCTCAG
(SEQ ID NO: 121)
QVQLVESGGGVVQPGRSLRLSCAASGFTFTNYAMHWVRQAPGKGLEWVAVISNDGSDKYY
ADSVKGRFTISRDNSKSTVYLQMSSLRPEDTAVYFCARDPPQVHWSLDYWGQGTLVTVSS
(SEQ ID NO: 49)

| | | |
|---|---|---|
| Light Chain | IGLV2-8*01 | IGLJ1*01 |

CAGTCTGCCCTGACTCAGCCTCCCTCCGCGTCCGGGTCTCCTGGACAGTCAGTCACCATCT
CCTGCACTGGAACCAGCAGTGACGTTGGTGATTATAACTATGTCTCCTGGTACCAACACCA
CCCAGGCAAAGCCCCCAAACTCATAATTTATGACGTCAGTAAGCGGCCCTCAGGGGTCCC
TGATCGCTTCTCTGGCTCCAAGTCTGGCGACACGGCCTCCCTGACCGTCTCTGGGCTCCAG
GCTGAGGATGAGGCTGATTATTACTGCAGCTCATATGCAGGCAACAACAATGCCGTCTTC
GGAACTGGGACCAAGGTCACCGTCCTAG (SEQ ID NO: 122)
QSALTQPPSASGSPGQSVTISCTGTSSDVGDYNYVSWYQHHPGKAPKLIIYDVSKRPSGVPDRF
SGSKSGDTASLTVSGLQAEDEADYYCSSYAGNNNAVFGTGTKVTVL (SEQ ID NO: 53)

### Binding to SARS-CoV-2 Spike Protein

Purified monoclonal antibody A19-1.1 was evaluated for its capacity to bind to four SARS CoV2 Spike derived protein antigens (S2P, S1, RBD and NTD) and the S2P SARS CoV1 spike protein antigen (FIG. 1). Binding was determined using ELISA immunoassay and showed that A19-1.1 is able to bind SARS CoV2 S2P, S1 and RBD but not SARS CoV-2 NTD or SARS CoV-1 S2P. This is consistent with the flow cytometry probe staining and indicates that the epitope is present in the S1 domain.

### Epitope Mapping

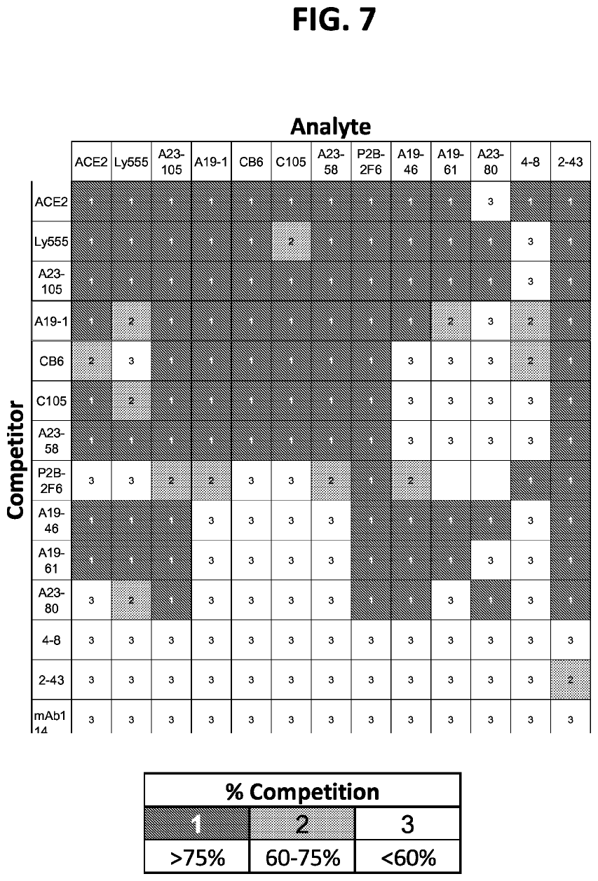

By assessing how A19-1.1 competes with previously published antibodies and antibodies discovered in this invention report, gross epitope can be determined. Competition classification was determined using Biolayer interferometry (BLI) assay described above. The A19-1.1 competition profile is shown in FIG. 7. Taken together, this indicates that A19-1.1 has a distinct mode of binding and epitope within the RBD, but competes with LY-COV555.

### Neutralization

A19-1.1 was tested for neutralization activity in a pseudotyped virus entry assay (FIG. 1). A19-1.1 was found to have a neutralization IC₅₀ (0.1259 μg/mL) and IC₈₀ (0.5305 μg/mL) of SARS CoV2 pseudotyped lentivirus particles.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A19-1.1 ACE2 competition profile is shown in FIG. 7 and indicates that A19-1.1 prevents ACE2 from binding to S2P.

### Example 8

### 4.7 A20-29.1 (A20-29.1)

### Identification of Coronavirus Antibodies Using FACS Probe Sorting and SMRTseq Sequencing

A20-29.1 is a mAb whose variable domains were isolated through the pairing of heavy and light chain immunoglobulin genes from a single memory B cell obtained from a SARS CoV2 survivor 50 days following symptom onset. Flow cytometry data suggested that the antibody. The nucleotide and amino acid sequences for the heavy and light chains of the expressed version of A20-29.1 can be found in Table 6. Variable heavy and light chain immunoglobulin sequence from Table 6 encoding the antigen binding region of A20-29.1 were synthesized and cloned into immunoglobulin expression vectors containing human constant regions for the IgG1 heavy chain and kappa light chain. These vectors were used to express antibody that was purified using standard methods.

TABLE 6
A20-29.1 gene family, and nucleotide and amino acid sequences for heavy and
light chains. CDR1, CDR2 and CDR3 are bolded and underlined in red in the
amino acid sequences

| | V-gene Family | D-gene Family | J-gene Family | Isotype |
|---|---|---|---|---|
| Heavy Chain | IGHV3-9*01 | IGHD3-10*01 | IGHJ4*02 | IGHGP*01, |
| | | | | IGHG3*04, |
| | | | | IGHG2*06 |

GAAGTGCAGCTGGTGGAGTCTGGGGGAGGCTTGGTACAGCCTGGCAGGTCCCTGAGACTC
TCCTGTGCAGCCTCTGGATTCACGTTTGATGATTATGCCATGCACTGGGTCCGGCAAACTC
CAGGGAAGGGCCTGGAGTGGGTCTCAGGTATTAGTTGGAATAGTGGTGACATAGACTATG
CGGACTCTGTGAAGGGCCGATTCACCATCTCCAGAGACAACGCCAAGAACTCCCTGTATC
TGCAAATGAACAGTCTGAGAACTGAGGACACGGCCTTGTATTACTGTACAAAAGGGTGGT
TCGGGGAGTTCTTCGGGGCCGGGTCGATATGTGACTACTGGGGCCAGGGAACCCTGGTCA
CCGTCTCCTCAG (SEQ ID NO: 123)
EVQLVESGGGLVQPGRSLRLSCAASGFTFDDYAMHWVRQTPGKGLEWVSGISWNSGDIDYA
DSVKGRFTISRDNAKNSLYLQMNSLRTEDTALYYCTKGWFGEFFGAGSICDYWGQGTLVTV
SS (SEQ ID NO: 33)

| | | |
|---|---|---|
| Light Chain | IGKV3-15*01, | IGKJ1*01 |
| | IGKV3D-15*01 | |

GAAATAGTGATGACGCAGTCTCCAGCCACCCTGTCTGTGTCTCCAGGGGAAAGAGCCACC
CTCTCCTGCAGGGCCAGTCAGAGTGTTAGCAACAACTTAGCCTGGTACCAGCAGAAACCT
GGCCAGGCTCCCAGGCTCCTCATCTATGGTGCATCCACCAGGGCCACTGGTATCCCAGCCA
GGTTCAGTGGCAGTGGGTCTGGGACAGAGTTCACTCTCACCATCAGCAGCCTGCAGTCTG
AAGATTTTGCAGTTTATTACTGTCAGCAGTATAATAACTGGCCGTTGTTCGGCCAAGGGAC
CAAGGTGGAAATCAAAC (SEQ ID NO: 124)
EIVMTQSPATLSVSPGERATLSCRASQSVSNNLAWYQQKPGQAPRLLIYGASTRATGIPARFSG
SGSGTEFTLTISSLQSEDFAVYYCQQYNNWPLFGQGTKVEIK (SEQ ID NO: 37)

### Binding to SARS-CoV-2 Spike Protein

Purified monoclonal antibody A20-29.1 was evaluated for its capacity to bind to four SARS CoV-2 Spike derived protein antigens (S2P, S1, RBD and NTD) and the S2P SARS CoV1 spike protein antigen (FIG. 1). Binding was determined using ELISA immunoassay and showed that A20-29.1 is able to bind SARS CoV2 S2P, S1 and RBD but not SARS CoV2 NTD. This is consistent with the flow cytometry probe staining and indicates that the epitope is present in the RBD domain. Additionally, A20-29.1 binds to SARS CoV-1 S2P, indicating cross coronavirus binding activity

### Epitope Mapping

By assessing how A20-29.1 competes with previously published antibodies and antibodies discovered in this invention report, gross epitope can be determined. Competition classification was determined using Biolayer interferometry (BLI) assay described above.

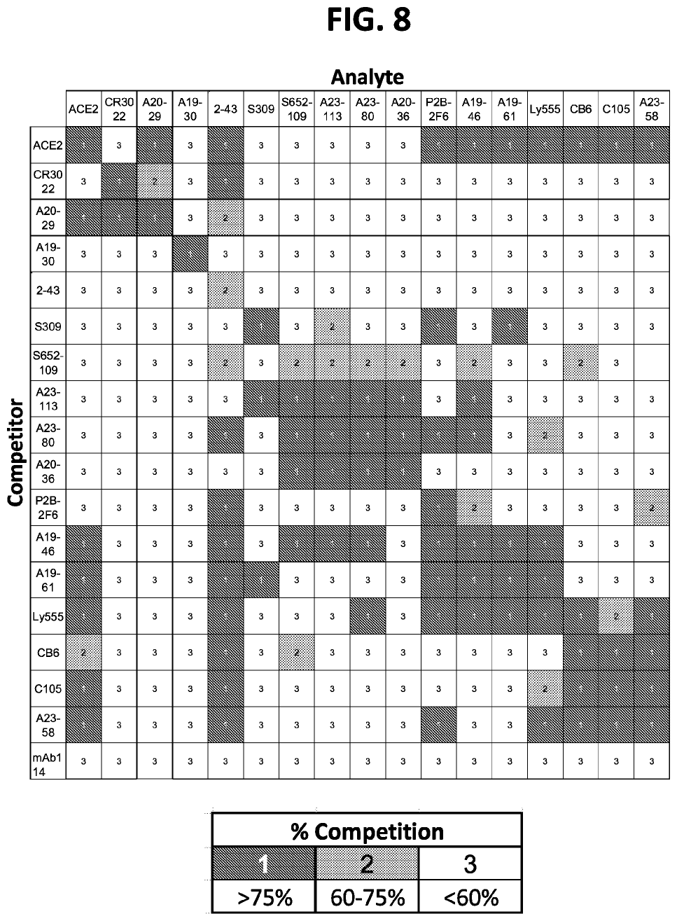

A20-29.1 competition profile is shown in FIG. 8. Negative stain EM of A20-29.1, A19-46.1 and A19-61.1 confirmed these antibodies binds to RBD of the spike with RBD in the down position (FIG. 4B). A20-29.1 has a distinct angle of approach to the RBD domain compared to A19-46.1 and A19-61.1. Epitope of A20-29.1 is located at the base of RBD and those of A19-46.1 and A19-61.1 are located on RBD regions closer to the 3-fold axis of the spike. Taken together, this indicates that A20-29.1 has a distinct mode of binding and epitope within the RBD.

### Kinetics of Binding to the SARS CoV2 S2P Protein

Fab protein generated from A20-29.1 was evaluated for binding to SARS CoV2 S2P using. A20-29.1 Fab shows binding to SARS CoV2 S2P protein with an affinity constant (K_{D}) of 0.263 nM, kₒₙ of 4.8×10⁵ per second and k_{off} of 1.26×10⁻⁴ per Molar·second.

### Neutralization

A20-29.1 was tested for neutralization activity in a pseudotyped virus entry assay (FIG. 1). A20-29.1 was found to have a neutralization IC₅₀ (0.3483 μg/mL) and IC₈₀ (1.3556 μg/mL) of SARS CoV2 pseudotyped lentivirus particles. Further testing against naturally occurring SARS COV-2 spike variants in pseudotyped neutralization assays showed that A20-29.1 maintains similar potency (IC₅₀: 0.1732-0.5792 μg/mL; IC₈₀: 0.3443-1.1341 μg/mL) against the following variants D614G, N439K/D614G, Y453F/D614G, A222V/D614G, N501Y/D614G, de169-70/D614G, N501Y/de169-70/D614G, E484K/D614G and N501Y/E484K/K417N/D614G. This is in contrast to LY-CoV555 and REGN-10989 which loses neutralizing capacity (>10 μg/mL) against the E484K/D614G and N501Y/E484K/K417N/D614G variants, and REGN-10933 which loses neutralizing capacity (>10 μg/mL) against Y453F/E614G variants and LY-CoV555, CB6, REGN-10989, REGN-10933 which lose neutralizing activity against the N501Y/E484K/K417N/D614G variant.

### ACE2 Receptor Blocking

SARS CoV-1 and SARS CoV-2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A20-29.1 ACE2 competition profile is shown in FIG. 8 and indicates that A20-29.1 does not prevent ACE2 from binding to S2P. Coupled with lack of competition with antibodies in the LY-COV555 class, it indicates that A20-29.1 is a highly neutralizing antibody that an be used as a non-competing partner for antibodies within the LY-COV555 competition class.

### Example 9

### 4.8 A19-30.1 (A19-30.1)

### Identification of Coronavirus Antibodies Using FACS Probe Sorting and SMRTseq Sequencing

A19-30.1 is a mAb whose variable domains were isolated through the pairing of heavy and light chain immunoglobulin genes from a single memory B cell obtained from a SARS CoV2 survivor 41 days following symptom onset. Flow cytometry data suggested that the antibody. The nucleotide and amino acid sequences for the heavy and light chains of the expressed version of A19-30.1 can be found in Table 7. Variable heavy and light chain immunoglobulin sequence from Table 7 encoding the antigen binding region of A19-30.1 were synthesized and cloned into immunoglobulin expression vectors containing human constant regions for the IgG1 heavy chain and lambda light chain. These vectors were used to express antibody that was purified using standard methods.

TABLE 7
A19-30.1 gene family, and nucleotide and amino acid sequences for heavy and light
chains. CDR1, CDR2 and CDR3 are bolded and underlined in red in the amino acid
sequences

| | V-gene Family | D-gene Family | J-gene Family | Isotype |
|---|---|---|---|---|
| Heavy | IGHV3-30-5*01, | Not | IGHJ4*02 | IGHGP*01, IGHG3*04, |
| Chain | IGHV3-30*18 | called | | IGHG2*06 |

CAGGTGCAGCTGGTGGAGTCTGGGGGAGGCGTGGTCCAGCCTGGGAGGTCCCTGAGACTC
TCCTGTGCAGCCTCTGGATTCACCTTCAGTAACTATGGCATGCACTGGGTCCGCCAGGCTC
CAGGCAAGGGGCTGGAGTGGCTGGCAGTTATATCATATGATGGAAGTAATAAATACTATG
CGGACTCCGTGAAGGGCCGATTCACCATCTCCAGAGACAATTCCAAGAACACGCTGTTTC
TGCAAATGAACAGCCTGAGAGCTGAGGACACGGCTGTGTATTACTGTGCGAAAGAGTCGC
AATTCGGGGAGTTATTCGAAGCCTTAGACTACTGGGGCCAGGGAACCCTGGTCACCGTCT
CCTCAG (SEQ ID NO: 125)
QVQLVESGGGVVQPGRSLRLSCAASGFTFSNYGMHWVRQAPGKGLEWLAVISYDGSNKYY
ADSVKGRFTISRDNSKNTLFLQMNSLRAEDTAVYYCAKESQFGELFEALDYWGQGTLVTVS
S (SEQ ID NO: 57)

| | | |
|---|---|---|
| Light | IGLV3-10*01 | IGLJ3*02 |
| Chain | | |

TCCTATGAGCTGACACAGCCACCCTCGGTGTCAGTGTCCCCAGGACAAACGGCCAGGATC
ACCTGCTCTGGAGATGCATTGCCAAGAAAATATGCTTTTTGGTACCAGCAGAAGTCAGGC
CAGGCCCCTGTGCTGGTCATCTCTGAGGACAGCAAACGACCCTCCGGGATCCCTGAGAGA
TTCTCTGGCTCCAGCTCAGGGACAATGGCCACCTTGACTATCAGTGGGGCCCAGGTGGAG
GATGAAGCTGACTACTACTGTTACTCAACAGACAGCAGTGGTAATCATAGGGTGTTCGGC
GGAGGGACCAAACTGACCGTCCTAG (SEQ ID NO: 126)
SYELTQPPSVSVSPGQTARITCSGDALPRKYAFWYQQKSGQAPVLVISEDSKRPSGIPERFSGSS
SGTMATLTISGAQVEDEADYYCYSTDSSGNHRVFGGGTKLTVL (SEQ ID NO: 61)

### Binding to SARS-CoV-2 Spike Protein

Purified monoclonal antibody A19-30.1 was evaluated for its capacity to bind to four SARS CoV2 Spike derived protein antigens (S2P, S1, RBD and NTD) and the S2P SARS CoV1 spike protein antigen (FIG. 1). Binding was determined using ELISA immunoassay and showed that A19-30.1 is able to bind SARS CoV2 S2P, S1 and RBD but not SARS CoV2 NTD or SARS CoV1 S2P. This indicates that the epitope is present in the RBD domain.

### Epitope Mapping

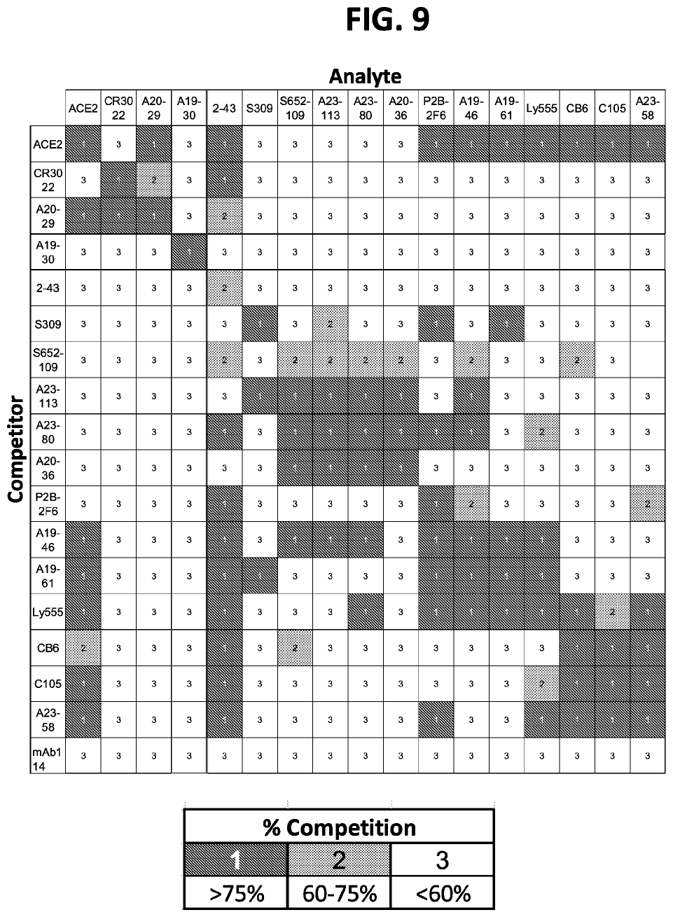

By assessing how A19-30.1 competes with previously published antibodies and antibodies discovered in this invention report, gross epitope can be determined. Competition classification was determined using Biolayer interferometry (BLI) assay described above. A19-30.1 competition profile is shown in FIG. 9. Taken together, this indicates that A19-30.1 has a distinct mode of binding and epitope within the RBD.

### Kinetics of Binding to the SARS CoV2 S2P Protein

Fab protein generated from A19-30.1 was evaluated for binding to SARS CoV-2 S2P using BLI. A19-30.1Fab shows binding to SARS CoV2 S2P protein with an affinity constant (K_{D}) of 0.916 nM, kₒₙ of 7.62×10⁻⁴ per second and k_{off} of 6.95×10⁻⁵ per Molar·second.

### Neutralization

A19-30.1 was tested for neutralization activity in a pseudotyped virus entry assay (FIG. 1). A19-30.1 was found to be non-neutralizing against of SARS CoV2 pseudotyped lentivirus particles. This suggests that it act by antibody Fc-dependent mechanisms to kill infected cells or directly lyse virus particle, such as antibody-dependent cellular cytotoxicity, antibody-dependent phagocytosis and/or antibody-dependent complement-mediated killing.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A19-30.1 ACE2 competition profile is shown in FIG. 9, and indicates that A19-30.1 does not prevent ACE2 from binding to S2P. Coupled with lack of competition with antibodies in the LY-COV555 class, this indicates that A19-30.1 is a potently neutralizing antibody that can be used as a non-competing partner for antibodies within the LY-COV555 competition class.

### Example 10

### 4.9 A20-36.1 (A20-36.1)

### Identification of Coronavirus Antibodies Using FACS Probe Sorting and SMRTseq Sequencing

A20-36.1 is a mAb whose variable domains were isolated through the pairing of heavy and light chain immunoglobulin genes from a single memory B cell obtained from a SARS CoV-2 survivor 50 days following symptom onset. The nucleotide and amino acid sequences for the heavy and light chains of the expressed version of A20-36.1 can be found in Table 8. Variable heavy and light chain immunoglobulin sequence from Table 8 encoding the antigen binding region of A20-36.1 were synthesized and cloned into immunoglobulin expression vectors containing human constant regions for the IgG1 heavy chain and lambda light chain. These vectors were used to express antibody that was purified using standard methods.

TABLE 8
A20-36.1 gene family, and nucleotide and amino acid sequences for heavy
and light chains. CDR1, CDR2 and CDR3 are bolded and underlined in the
amino acid sequences

| | V-gene Family | D-gene Family | J-gene Family | Isotype |
|---|---|---|---|---|
| Heavy | IGHV3-33*01 | IGHD3-9*01 | IGHJ4*02 | IGHG1*04 |
| Chain | | | | |

CAGGTGCAGCTGGTGGAGTCTGGGGGAGGCGTGGTCCAGCCTGGGAGGT
CCCTGAGACTCTCCTGTGCAGCGTCTGGATTCATCTTCAGTAGCTATGGC
GTGCACTGGGTCCGCCAGGCTCCAGGCAAGGGGCTGGAGTGGGTGGCAG
TTATATGGCATGATGAAAGTAATAAAGACTATGCAGACTCCGTGAAGGG
CCGATTCACCATCTCCAGAGACAATTCCAAGAACACGGTGTATCTGCAAA
TGAACAGCCTGAGAGCCGAGGACACGGCTATGTATTATTGTGCGAGAGA
TGGTTACGATTTTTTGACTGGGGCTTACGAGCTTGACTACTGGGGCCAGG
GAACCCTGGTCACCGTCTCCTCAG (SEQ ID NO: 127)
QVQLVESGGGVVQPGRSLRLSCAASGFIFSSYGVHWVRQAPGKGLEWVAV
IWHDESNKDYADSVKGRFTISRDNSKNTVYLQMNSLRAEDTAMYYCARD
GYDFLTGAYELDYWGQGTLVTVSS (SEQ ID NO: 65)

| | | |
|---|---|---|
| Light | IGLV3-25*02 | IGLJ3*02 |
| Chain | | |

TCCTATGAGCTGACACAGCCACCCTCGGTGTCAGTGTCCCCAGGACAGAC
GGCCAGGATCACCTGCTCTGGAGATGCATTGCCAAAGCAATATGCTTATT
GGTACCAGCAGAAGCCAGGCCAGGCCCCTGTGGTGGTGATATATAAAGA
CAGTGAGAGGCCCTCAGGGATCCCTGAGCGATTCTCTGGCTCCAGCTCAG
GGACAACAGTCACGTTGACCATCAGTGGAGTCCAGGCAGAAGATGAGGC
TGACTATTACTGTCAATCAGCAGACAGCAGTGGCACTTGGGTGTTCGGCG
GAGGGACCAAACTGACCGTCCTAG (SEQ ID NO: 128)
SYELTQPPSVSVSPGQTARITCSGDALPKQYAYWYQQKPGQAPVVVIYKDS
ERPSGIPERFSGSSSGTTVTLTISGVQAEDEADYYCQSADSSGTWVFGGGTK
LTVL (SEQ ID NO: 69)

### Binding to SARS-CoV-2 Spike Protein

Purified monoclonal antibody A20-36.1 was evaluated for its capacity to bind to four SARS CoV2 Spike derived protein antigens (S2P, S1, RBD and NTD) and the S2P SARS CoV1 spike protein antigen (FIG. 1). Binding was determined using ELISA immunoassay and showed that A20-36.1 is able to bind SARS CoV-2 S2P and S1 but not SARS CoV-2 RBD or NTD. Follow-up mapping ELISA looking at SD1 and SD2 domains for S1 suggested that the antibody is targeting a region in SD2 of S1. This data is consistent with the flow cytometry probe staining. Furthermore, the antibody was shown to bind to the SARS CoV-1 S2P.

### Epitope Mapping

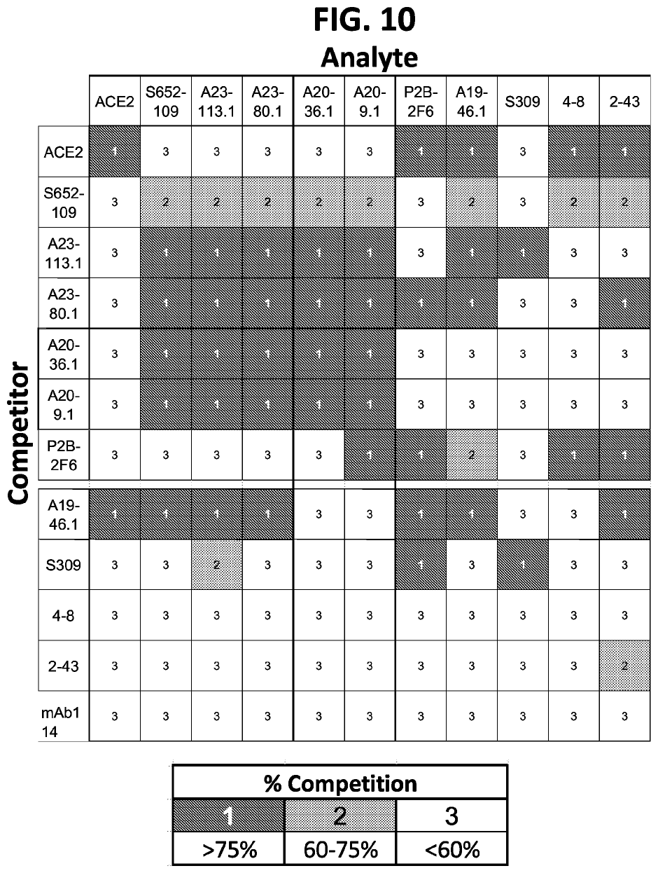

By assessing how A20-36.1 competes with previously published antibodies and antibodies disclosed herein, gross epitope can be determined. Competition classification was determined using Biolayer interferometry (BLI) assay described above. A20-36.1 competition profile is shown in FIG. 10. Taken together, this indicates that A20-36.1 has a distinct mode of binding and epitope within the SD2 region of S1.

### Kinetics of Binding to the SARS CoV2 S2P Protein

Fab protein generated from A20-36.1 was evaluated for binding to SARS CoV2 S2P using BLI. A23-58.1 Fab shows binding to SARS CoV2 S2P protein with an affinity constant (K_{D}) of 1.82 nM, kₒₙ of 5.99×105 per second and k_{off} of 1.09×10⁻³ per Molar·second.

### Neutralization

A20-36.1 was tested for neutralization activity in a pseudotyped virus entry assay (See Table 4.9.2-1). A20-36.1 was found to have a neutralization IC₅₀ (1.3851 μg/mL) and IC₈₀ (3.7620 μg/mL) of SARS CoV2 pseudotyped lentivirus particles.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A20-36.1 ACE2 competition profile is shown in FIG. 10, and indicates that A20-36.1 does not prevent ACE2 from binding to S2P. Coupled with lack of competition with antibodies in the LY-COV555 class, it indicates that A20-36.1 is a neutralizing antibody can be used as a non-competing partner for antibodies within the LY-COV555 competition class.

### Example 11

### 4.10 A23-97.1 (A23-97.1)

### Identification of Coronavirus Antibodies Using FACS Probe Sorting and SMRTseq Sequencing

A23-97.1 is a mAb whose variable domains were isolated through the pairing of heavy and light chain immunoglobulin genes from a single memory B cell obtained from a SARS CoV2 survivor 48 days following symptom onset. Flow cytometry data suggested that the antibody. The nucleotide and amino acid sequences for the heavy and light chains of the expressed version of A23-97.1 can be found in Table 9. Variable heavy and light chain immunoglobulin sequence from Table 9 encoding the antigen binding region of A23-97.1 were synthesized and cloned into immunoglobulin expression vectors containing human constant regions for the IgG1 heavy chain and lambda light chain. These vectors were used to express antibody that was purified using standard methods.

TABLE 9
A23-97.1 gene family, and nucleotide and amino acid sequences for heavy and
light chains. CDR1, CDR2 and CDR3 are bolded and underlined in the amino acid
sequences

| | V-gene Family | D-gene Family | J-gene Family | Isotype |
|---|---|---|---|---|
| Heavy | IGHV3-30-5*02, | IGHD3-22*01 | IGHJ4*02 | IGHG |
| Chain | IGHV3-30*02 | | | |

CAGGTGCAGCTGGTGGAGTCTGGGGGAGGCGTGGTCCAGCCTGGGGGGTCCCTGAGACTC
TCCTGTGCAGCGTCTGGATTCACCTTCAGTAGCTTTGGCATGCACTGGGTCCGCCAGGCTC
CAGGCAAGGGGCTGGAGTGGGTGGCATTTATACGGTATGATGGAAGTAATAAATACTATG
CAGACTCCGTGAAGGGCCGATTCACCATCTCCAGGGACAATTCCAAGAACACGCTGTATC
TGCAAATGAACAGCCTGAGAGCTGAGGACACGGCTGTGTATTATTGTGCGAAGACAGAAC
TATATTACTATGATAGTAGTGGCCCATTGGGGTGGGGCCAGGGAACCCTGGTCACCGTCTC
CTCAG (SEQ ID NO: 129)
QVQLVESGGGVVQPGGSLRLSCAASGFTFSSFGMHWVRQAPGKGLEWVAFIRYDGSNKYY
ADSVKGRFTISRDNSKNTLYLQMNSLRAEDTAVYYCAKTELYYYDSSGPLGWGQGTLVTVS
S (SEQ ID NO: 73)

| | | |
|---|---|---|
| Light Chain | IGKV1-5*01 | IGKJ1*01 |

GACATCCAGATGACCCAGTCTCCTTCCACCCTGTCTGCATCTGTAGGAGACAGAGTCACCA
TCACTTGCCGGGCCAGTCAGAGTATTACTAGCTGGTTGGCCTGGTATCAGCAGAAACCAG
GGAAAGCCCCTAAGCTCCTGATCTATGATGCCTCCAGTTTGGAAAGTGGTGTCCCATCAAG
GTTCAGCGGCAGTGGATCTGGGACAGAATTCACTCTCACCATCAGCAGCCTGCAGCCTGA
TGATTTTGCAACTTATTACTGCCAACAGTATAATAGTTATCCGTGGACGTTCGGCCAAGGG
ACCAAGGTGGAAATCAAAC (SEQ ID NO: 130)
DIQMTQSPSTLSASVGDRVTITCRASQSITSWLAWYQQKPGKAPKLLIYDASSLESGVPSRFSG
SGSGTEFTLTISSLQPDDFATYYCQQYNSYPWTFGQGTKVEIK (SEQ ID NO: 77)

### Binding to SARS-CoV-2 Spike Protein

Purified monoclonal antibody A23-97.1 was evaluated for its capacity to bind to four SARS CoV-2 Spike derived protein antigens (S2P, S1, RBD and NTD) and the S2P SARS CoV1 spike protein antigen (FIG. 1). Binding was determined using ELISA immunoassay and showed that A23-97.1 is able to bind SARS CoV2 S2P, S1 and RBD but not SARS CoV2 NTD. This is consistent with the flow cytometry probe staining and indicates that the epitope is present in the RBD domain. In addition, it was shown to bind to SARS CoV1 S2P.

### Epitope Mapping

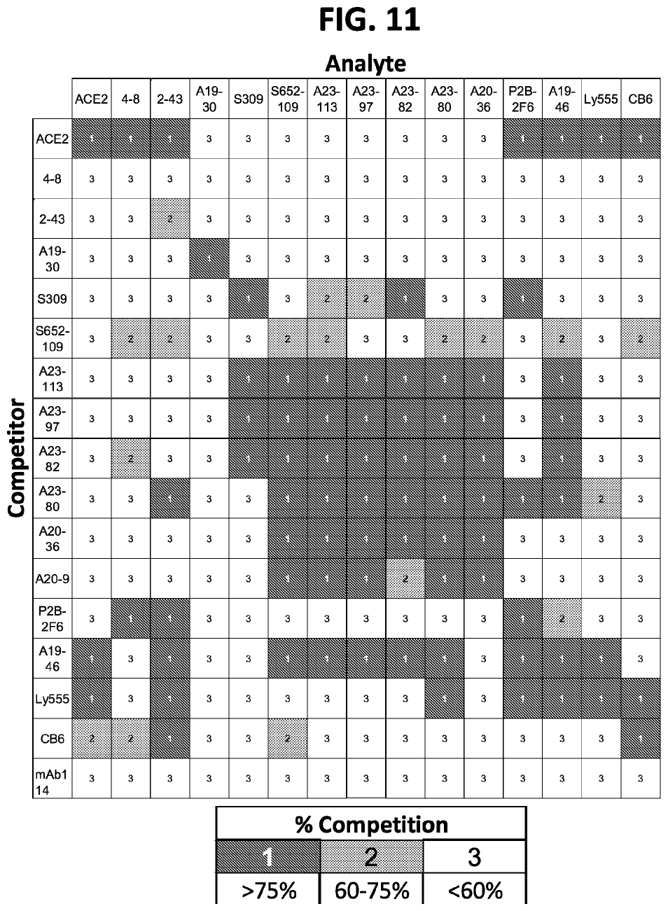

By assessing how A23-97.1 competes with previously published antibodies and antibodies discovered in this invention report, gross epitope can be determined. Competition classification was determined using Biolayer interferometry (BLI) assay described above. A23-97.1 competition profile is shown in FIG. 11. Taken together, this indicates that A23-97.1 has a distinct mode of binding and epitope within the RBD that is similar to other antibodies in this report but unique from published antibodies.

### Kinetics of Binding to the SARS CoV2 S2P Protein

Fab protein generated from A23-97.1 was evaluated for binding to SARS CoV2 S2P using BLI. A23-97.1 Fab shows binding to SARS CoV2 S2P protein with an affinity constant (KD) of 0.263 nM, kₒₙ of 7.10×10⁵ per second and k_{off} of 1.87×10⁻⁴ per Molar·second.

### Neutralization

A23-97.1 was tested for neutralization activity in a pseudotyped virus entry assay (FIG. 1). A23-97.1 was found to have a neutralization IC₅₀ (0.4842 μg/mL) and IC₈₀ (5.2782 μg/mL) of SARS CoV2 pseudotyped lentivirus particles.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A23-97.1 ACE2 competition profile is shown in FIG. 11 and indicates that A23-97.1 does NOT prevent ACE2 from binding to S2P. Coupled with lack of competition with antibodies in the LY-COV555 class, it indicates that A23-97.1 is a neutralizing antibody that can be used as a non-competing partner for antibodies within the LY-COV555 competition class.

### Example 12

### 4.11 A23-113.1 (A23-113.1)

### Identification of Coronavirus Antibodies Using FACS Probe Sorting and SMRTseq Sequencing

A23-113.1 is a mAb whose variable domains were isolated through the pairing of heavy and light chain immunoglobulin genes from a single memory B cell obtained from a SARS CoV-2 survivor 48 days following symptom onset. The nucleotide and amino acid sequences for the heavy and light chains of the expressed version of A23-113.1 can be found in Table 10. Variable heavy and light chain immunoglobulin sequence from Table 10 encoding the antigen binding region of A23-113.1 were synthesized and cloned into immunoglobulin expression vectors containing human constant regions for the IgG1 heavy chain and kappa light chain. These vectors were used to express antibody that was purified using standard methods.

TABLE 10
A23-113.1 gene family, and nucleotide and amino acid sequences for heavy and
light chains. CDR1, CDR2 and CDR3 are bold and underlined in the amino acid
sequences

| | V-gene Family | D-gene Family | J-gene Family | Isotype |
|---|---|---|---|---|
| Heavy Chain | IGHV3-30-5*01, | Not called | IGHJ4*02 | IGHGP*01, |
| | IGHV3-30*18 | | | IGHG3*04, |
| | | | | IGHG2*06 |

CAGGTGCACCTGGAGGAGTCTGGGGGAGCCGTGGTCCAGCCTGGGAGGTCCCTGAGACTC
TCCTGTGCAGCCTCTGGATTCACCTTCAGTAGCTATGGCATGCACTGGGTCCGCCAGGCCC
CAGGCAAGGGGCTGGAGTGGGTGGCAGTTATATCACATGATGGAAGTTATAAGTACTATG
CAGACTCCGTGAAGGGCCGATTCACCATCTCCAGAGACAATTCCAAGGACACGCTCTATC
TGCAAACGAACAGCCTGAGAGCTGAGGACACGGCTATGTATTACTGTGCGAAAAGCTATG
GTTATTGGATGGCCTACTTTGACTACTGGGGCCAGGGAACCCTGGTCACCGTCTCCTCAG
(SEQ ID NO: 131)
QVHLEESGGAVVQPGRSLRLSCAASGFTFSSYGMHWVRQAPGKGLEWVAVISHDGSYKYY
ADSVKGRFTISRDNSKDTLYLQTNSLRAEDTAMYYCAKSYGYWMAYFDYWGQGTLVTVSS
(SEQ ID NO: 81)

| | | |
|---|---|---|
| Light Chain | IGKV1-27*01 | IGKJ4*01 |

GACATCCAGATGACCCAGTCTCCATCCTCCCTGTCTGCATCTGTAGGAGACAGAGTCACCA
TCACTTGCCGGGCGAGTCAGGACATTAGCAATTATTTAGCCTGGTATCAGCAGAAACCAG
GGAAAGTTCCTAAGCTCCTGATCTATGCTGCATCCACTTTGCAATCAGGGGTCCCATCTCG
GTTCAGTGGCAGTGGATCTGGGACAGATTTCACTCTCACCATCAGCAGCCTGCAGCCTGA
AGATGTTGCAACTTATTACTGTCAAAAGTATAACAGTCCCTGGCACACTTTCGGCGGAGG
GACCAAGGTGGAGATCAAAC (SEQ ID NO: 132)
DIQMTQSPSSLSASVGDRVTITCRASQDISNYLAWYQQKPGKVPKLLIYAASTLQSGVPSRFSG
SGSGTDFTLTISSLQPEDVATYYCQKYNSPWHTFGGGTKVEIK (SEQ ID NO: 85)

### Binding to SARS-CoV-2 Spike Protein

Purified monoclonal antibody A23-113.1 was evaluated for its capacity to bind to four SARS CoV-2 Spike derived protein antigens (S2P, S1, RBD and NTD) and the S2P SARS CoV1 spike protein antigen (FIG. 1). Binding was determined using ELISA immunoassay and showed that A23-113.1 is able to bind SARS CoV2 S2P, S1 and RBD but not SARS CoV2 NTD. This is consistent with the flow cytometry probe staining and indicates that the epitope is present in the RBD domain. In addition, it was shown to bind to SARS CoV1 S2P.

### Epitope Mapping

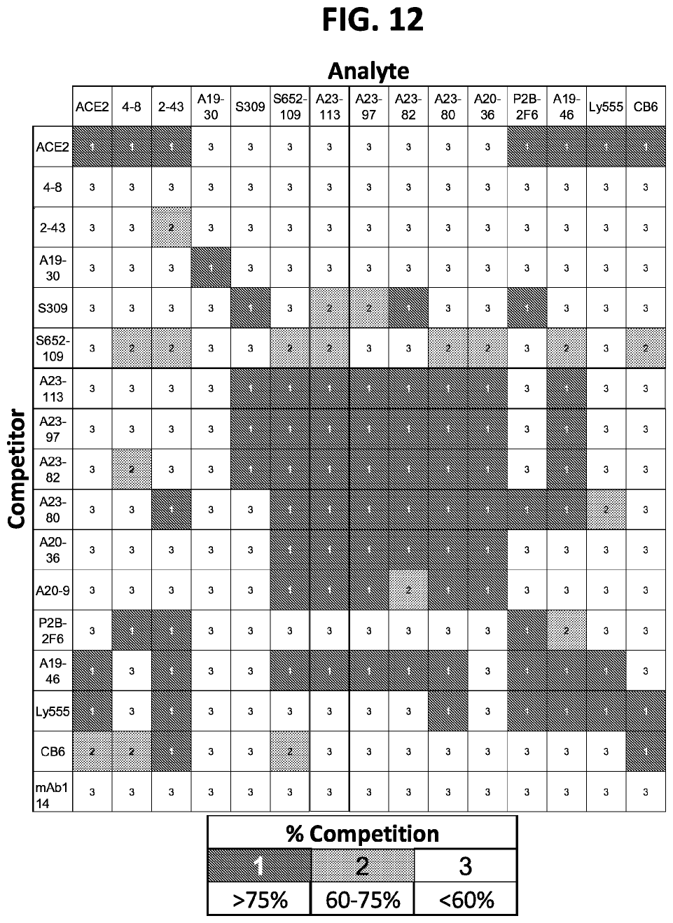

By assessing how A23-113.1 competes with previously published antibodies and antibodies disclosed herein, gross epitope can be determined. Competition classification was determined using Biolayer interferometry (BLI) assay described above. A23-113.1 competition profile is shown in FIG. 12. Taken together, this indicates that A23-113.1 has a distinct mode of binding and epitope within the RBD that is similar to other antibodies disclosed herein but unique from other antibodies.

### Kinetics of Binding to the SARS CoV2 S2P Protein

Fab protein generated from A23-113.1 was evaluated for binding to SARS CoV-2 S2P using BLI. A23-113.1. Fab shows binding to SARS CoV2 S2P protein with an affinity constant (K_{D}) of 1.61 nM, kₒₙ of 8.676×10⁵ per second and k_{off} of 1.567×10⁻³ per Molar·second.

### Neutralization

A23-113.1 was tested for neutralization activity in a pseudotyped virus entry assay (FIG. 1). A23-113.1 was found to have a neutralization IC₅₀ (0.3927 μg/mL) and IC₈₀ (7.8096 μg/mL) of SARS CoV2 pseudotyped lentivirus particles.

### ACE2 Receptor Blocking

SARS CoV-1 and SARS CoV-2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A23-113.1 ACE2 competition profile is shown in FIG. 12 and indicates that A23-113.1 does NOT prevents ACE2 from binding to S2P.

### Example 13

### 4.12 A23-80.1 (A23-80.1)

### Identification of Coronavirus Antibodies Using FACS Probe Sorting and SMRTseq Sequencing

A23-80.1 is a mAb whose variable domains were isolated through the pairing of heavy and light chain immunoglobulin genes from a single memory B cell obtained from a SARS CoV-2 survivor 48 days following symptom onset. The nucleotide and amino acid sequences for the heavy and light chains of the expressed version of A23-80.1 can be found in Table 11. Variable heavy and light chain immunoglobulin sequence from Table 11 encoding the antigen binding region of A23-80.1 were synthesized and cloned into immunoglobulin expression vectors containing human constant regions for the IgG1 heavy chain and kappa light chain. These vectors were used to express antibody that was purified using standard methods.

TABLE 11
A23-80.1 gene family, and nucleotide and amino acid sequences for heavy and
light chains. CDR1, CDR2 and CDR3 are bold and underlined in the amino acid
sequences

| | V-gene Family | D-gene Family | J-gene Family | Isotype |
|---|---|---|---|---|
| Heavy Chain | IGHV1-18*01 | IGHD2-15*01 | IGHJ4*02 | IGHGP*01, |
| | | | | IGHG3*04, |
| | | | | IGHG2*06 |

CAGGTTCAGCTGGTGCAGTCTGGAGCTGAGGTGAAGAAGCCTGGGGCCTCAGTGAAGGTC
TCCTGCAAGGCTTCCGGTTACACCTTTACCAGCAATGGAGTCACCTGGGTGCGACAGGCCC
CTGGACAAGGGCTTGAGTGGATGGGATGGATCAGCACTTACAATGGAGACACAAACTATG
CACAGAAGCTCCAGGGCAGAGTCTCCATGACCACAGACACATCCACGCGCACAGTTTACA
TGGAGCTGAGGAGCCTGAGATCTGACGACACGGCCGTCTATTACTGTGCGAGGGTGGGGG
ATGCATATTGTAGTGGTGGTAGCTGCTATCACTTTGACTACTGGGGCCAGGGAACCCTGGT
CACCGTCTCCTCAG (SEQ ID NO: 133)
QVQLVQSGAEVKKPGASVKVSCKASGYTFTSNGVTWVRQAPGQGLEWMGWISTYNGDTN
YAQKLQGRVSMTTDTSTRTVYMELRSLRSDDTAVYYCARVGDAYCSGGSCYHFDYWGQG
TLVTVSS (SEQ ID NO: 89)

| | | |
|---|---|---|
| Light Chain | IGKV3-15*01, | IGKJ3*01 |
| | IGKV3D-15*01 | |

GAAATAGTGATGACGCAGTCTCCAGCCACCCTGTCTGTGTCTCCAGGGGAAAGAGCCACC
CTCTCCTGCAGGGCCAGTCAGAGTGTTAGCACCAACTTAGCCTGGTACCAGCAGAAGCCT
GGCCAGGCTCCCAGGCTCCTCATCTATGGTGCATCCACCAGGGCCACTGGTATCCCAGCCA
GGTTCAGTGGCAGTGGGTCTGGGACAGAGTTCACTCTCACCATCAGCAGCCTGCAGTCTG
AAGATTTTGCACTTTATTACTGTCAGCAGTATGATAACTGGCCTCCGGAATTCACTTTCGG
CCCTGGGACCAAAGTGGATATCAAAC (SEQ ID NO: 134)
EIVMTQSPATLSVSPGERATLSCRASQSVSTNLAWYQQKPGQAPRLLIYGASTRATGIPARFSG
SGSGTEFTLTISSLQSEDFALYYCQQYDNWPPEFTFGPGTKVDIK (SEQ ID NO: 93)

### Binding to SARS-CoV-2 Spike Protein

Purified monoclonal antibody A23-80.1 was evaluated for its capacity to bind to four SARS CoV-2 Spike derived protein antigens (S2P, S1, RBD and NTD) and the S2P SARS CoV-1 spike protein antigen (FIG. 1). Binding was determined using ELISA immunoassay and showed that A23-80.1 is able to bind SARS CoV-2 S2P, S1 and RBD but not SARS CoV-2 NTD or SARS CoV-1 S2P. This is consistent with the flow cytometry probe staining and indicates that the epitope is present in the RBD domain.

### Epitope Mapping

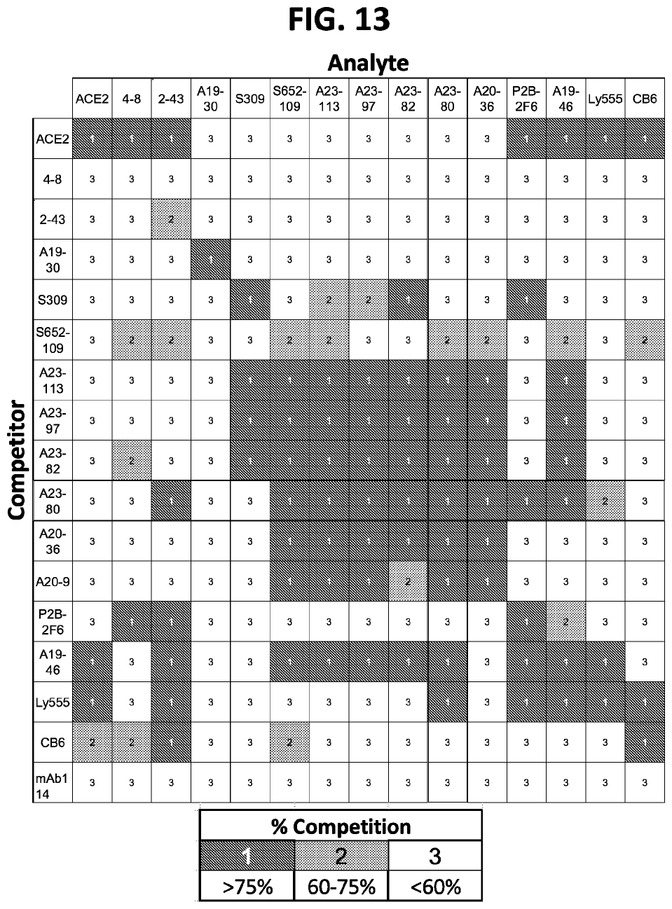

By assessing how A23-80.1 competes with previously published antibodies and antibodies disclosed herein, gross epitope can be determined. Competition classification was determined using Biolayer interferometry (BLI) assay described above. A23-80.1 competition profile is shown in FIG. 13. Taken together, the data indicates that A23-80.1 has a distinct mode of binding and epitope within the RBD.

### Kinetics of Binding to the SARS CoV2 S2P Protein

Fab protein generated from A23-80.1 was evaluated for binding to SARS CoV2 S2P using BLI. A23-80.1 Fab shows binding to SARS CoV2 S2P protein with an affinity constant (K_{D}) of 0.443 nM, kₒₙ of 3.32×10⁵ per second and k_{off} of 1=0.47×10⁻⁴ per Molar·second.

### Neutralization

A23-80.1 was tested for neutralization activity in a pseudotyped virus entry assay (FIG. 1). A23-80.1 was found to have a neutralization IC₅₀ of 0.3211 μg/mL of SARS CoV2 pseudotyped lentivirus particles.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A23-80.1 ACE2 competition profile is shown in FIG. 13 and indicates that A23-80.1 does NOT prevents ACE2 from binding to S2P.

### Example 14

### 4.13 A19-82.1 (A19-82.1)

### Identification of Coronavirus Antibodies Using FACS Probe Sorting and SMRTseq Sequencing

A19-82.1 is a mAb whose variable domains were isolated through the pairing of heavy and light chain immunoglobulin genes from a single memory B cell obtained from a SARS CoV2 survivor 41 days following symptom onset. Flow cytometry data suggested that the antibody. The nucleotide and amino acid sequences for the heavy and light chains of the expressed version of A19-82.1 can be found in Table 12. Variable heavy and light chain immunoglobulin sequence from Table 12 encoding the antigen binding region of A19-82.1 were synthesized and cloned into immunoglobulin expression vectors containing human constant regions for the IgG1 heavy chain and lambda light chain. These vectors were used to express antibody that was purified using standard methods.

TABLE 12
Table 4.13.1-1 A19-82.1 gene family, and nucleotide and amino acid sequences
for heavy and light chains. CDR1, CDR2 and CDR3 are bold and underlined in the
amino acid sequences

| | V-gene Family | D-gene Family | J-gene Family | Isotype |
|---|---|---|---|---|
| Heavy Chain | IGHV3-30-5*01, | Not called | IGHJ4*02 | IGHGP*01, |
| | IGHV3-30*18 | | | IGHG3*04, |
| | | | | IGHG2*06 |

CAGGTGCAGCTGGTGGAGTCTGGGGGAGGCGTGGTCCAGCCTGGGAGGTCCCTGAGACTC
TCCTGTGTAGTCTCTGGACTCATTTTCAGTACCTATGACATGCACTGGGTCCGCCAGGCTC
CAGGCAAGGGGCTGGAGTGGGTGGCAGTTATATCATATGATGGAAGTTACAAACACTATG
CAGACTCCGTGAAGGGCCGATTCACCATCTCCAGAGACAGTTCCAAGAACACGCTGTATC
TGCAAATGAACAGCCTGAGACCTGAAGACACGGCTGTCTATTACTGTGCGAAAGGGGAGG
GAGTAGTGGCTGGTACGGGGAAGTTTGACTACTGGGGCCAGGGAACCCTGGTCACCGTCT
CCTCAG (SEQ ID NO: 135)
QVQLVESGGGVVQPGRSLRLSCVVSGLIFSTYDMHWVRQAPGKGLEWVAVISYDGSYKHY
ADSVKGRFTISRDSSKNTLYLQMNSLRPEDTAVYYCAKGEGVVAGTGKFDYWGQGTLVTV
SS (SEQ ID NO: 97)

| | | |
|---|---|---|
| Light Chain | IGLV3-21*02 | IGLJ3*02 |

TCCTATGTGCTGACTCAGCCACCCTCGGTGTCAGTGGCCCCAGGACAGACGGCCAGGATT
ACCTGTGGGGGAAACAACATTGGAAGTAAAAGTGTGCACTGGTACCAGCAGAAGCCAGG
CCAGGCCCCTGTGCTGGTCGTCTATGATGATAGTGACCGGCCCTCAGGGATCCCTGAGCG
ATTCTCTGGCTCCAACTCTGGGAACACGGCCACCCTGACCATCAGCAGGGTCGAGGCCGG
GGATGAGGCCGACTATTACTGTCAGGTGTGGGATGGTAGTGGTGATCCTTGGGTGTTCGG
CGGAGGGACCAAGCTGACCGTCCTAG (SEQ ID NO: 136)
SYVLTQPPSVSVAPGQTARITCGGNNIGSKSVHWYQQKPGQAPVLVVYDDSDRPSGIPERFSG
SNSGNTATLTISRVEAGDEADYYCQVWDGSGDPWVFGGGTKLTVL (SEQ ID NO: 101)

### Binding to SARS-CoV-2 Spike Protein

Purified monoclonal antibody A19-82.1 was evaluated for its capacity to bind to four SARS CoV2 Spike derived protein antigens (S2P, S1, RBD and NTD) and the S2P SARS CoV1 spike protein antigen (FIG. 1). Binding was determined using ELISA immunoassay and showed that A19-82.1 is able to bind SARS CoV2 S2P, S1 and RBD but not SARS CoV2 NTD. This is consistent with the flow cytometry probe staining and indicates that the epitope is present in the RBD domain. In addition, it was shown to bind to SARS CoV1 S2P.

### Epitope Mapping

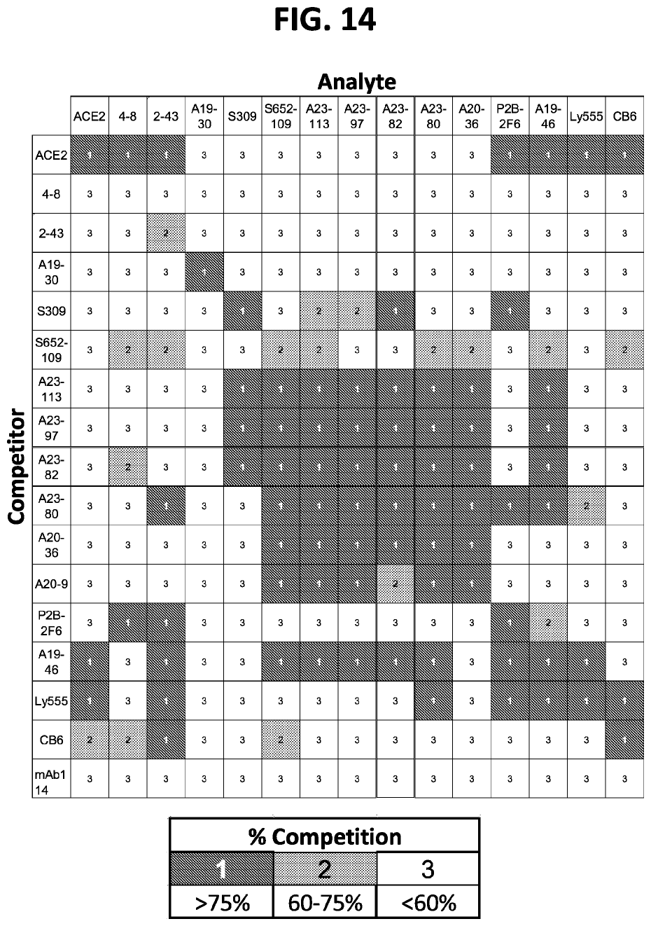

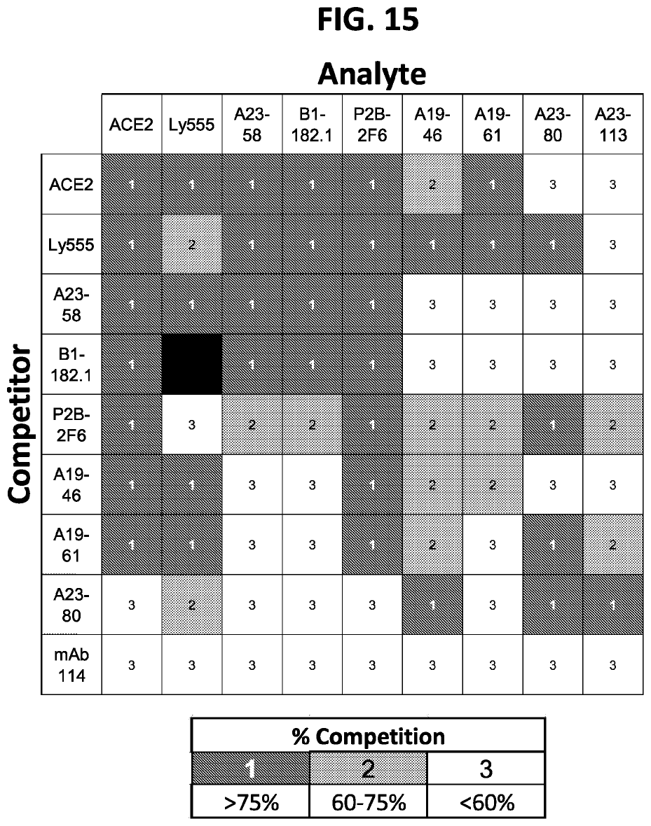

By assessing how A19-82.1 competes with previously published antibodies and antibodies disclosed herein, gross epitope can be determined. Competition classification was determined using Biolayer interferometry (BLI) assay described above. A19-82.1 competition profile is shown in FIG. 14. Taken together, the data indicate that A19-82.1 has a distinct mode of binding and epitope within the RBD.

### Kinetics of Binding to the SARS CoV2 S2P Protein

Fab protein generated from A19-82.1 was evaluated for binding to SARS CoV2 S2P using BLI. A19-82.1 Fab shows binding to SARS CoV2 S2P protein with an affinity constant (K_{D}) of 5.21 nM, kₒₙ of 5.41×10⁵ per second and k_{off} of 2.82×10⁻³ per Molar·second.

### Neutralization

A19-82.1 was tested for neutralization activity in a pseudotyped virus entry assay (FIG. 1). A19-82.1 was found to have a neutralization IC₅₀ (0.7203 μg/mL) and IC₈₀ (5.9260 μg/mL) of SARS CoV2 pseudotyped lentivirus particles.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A19-82.1 ACE2 competition profile is shown in FIG. 14 and indicates that A19-82.1 does NOT prevent ACE2 from binding to S2P. Coupled with lack of competition with antibodies in the LY-COV555 class, it indicates that A19-82.1 is a neutralizing antibody that can be used as a non-competing partner for antibodies within the LY-COV555 competition class.

### Example 15

### 4.14 A20-9.1 (A20-9.1)

### Identification of Coronavirus Antibodies Using FACS Probe Sorting and SMRTseq Sequencing

A20-9.1 is a mAb whose variable domains were isolated through the pairing of heavy and light chain immunoglobulin genes from a single memory B cell obtained from a SARS CoV2 survivor 50 days following symptom onset. Flow cytometry data suggested that the antibody. The nucleotide and amino acid sequences for the heavy and light chains of the expressed version of A20-9.1 can be found in Table 13. Variable heavy and light chain immunoglobulin sequence from Table 13 encoding the antigen binding region of A20-9.1 were synthesized and cloned into immunoglobulin expression vectors containing human constant regions for the IgG1 heavy chain and lambda light chain. These vectors were used to express antibody that was purified using standard methods.

TABLE 13
A20-9.1 gene family, and nucleotide and amino acid sequences for heavy and
light chains. CDR1, CDR2 and CDR3 are bolded and underlined in the amino
acid sequences

| | V-gene Family | D-gene Family | J-gene Family | Isotype |
|---|---|---|---|---|
| Heavy Chain | IGHV3-30-5*01, | Not called | IGHJ5*02 | IGHGP*01, |
| | IGHV3-30*18 | | | IGHG3*04, |
| | | | | IGHG2*06 |

CAGGTGCAGCTGGTGGAGTCTGGGGGAGGCGTGGTCCAGCCTGGGAGGTCCCTGAGACTC
TCCTGTGCAGCCTCTGGATTCACCTTCAGTAGCTATGGCATGCACTGGGTCCGCCAGGCTC
CAGGCAAGGGGCTGGAGTGGGTGGCATTTATATCATATGATGGAAGTAATAAATACTATG
CAGACTCCGTGAAGGGCCGATTCACCATCTCCAGAGACAATTCCAAGAACACTCTGTATC
TGCAAATGAACAGCCTGCGAGCTGAGGACACGGCTGTGTATTACTGTGCGAAAGATTATT
GGTCAGTAGCAGCTGGTACTAGCTGGTTCGACCCCTGGGGCCAGGGAACCCTGGTCAGCG
TCTCCTCAG (SEQ ID NO: 137)
QVQLVESGGGVVQPGRSLRLSCAASGFTFSSYGMHWVRQAPGKGLEWVAFISYDGSNKYY
ADSVKGRFTISRDNSKNTLYLQMNSLRAEDTAVYYCAKDYWSVAAGTSWFDPWGQGTLVS
VSS (SEQ ID NO: 105)

| | | |
|---|---|---|
| Light Chain | IGLV3-25*02 | IGLJ3*02 |

TCCTATGAGCTGACACAGCCACCCTCGGTGTCAGTGTCCCCAGGACAGACGGCCAGGATC
ACCTGCTCTGGAGATGCATTGCCAAAGCAATATGCTTATTGGTACCAGCAGAAGCCAGGC
CAGGCCCCTGTGGTGGTGATATATAAAGACAGTGAGAGGCCCTCAGGGATCCCTGAGCGA
TTCTCTGGCTCCAGCTCAGGGACAACAGTCACGTTGACCATCAGTGGAGTCCAGGCAGAA
GACGAGGCTGACTATTACTGTCAATCAGCAGACAGCAGTGGTACTTGGGTGTTCGGCGGA
GGGACCAAGCTGACCGTCCTAG (SEQ ID NO: 138)
SYELTQPPSVSVSPGQTARITCSGDALPKQYAYWYQQKPGQAPVVVIYKDSERPSGIPERFSGS
SSGTTVTLTISGVQAEDEADYYCQSADSSGTWVFGGGTKLTVL (SEQ ID NO: 109)

### Binding to SARS-CoV-2 Spike Protein

Purified monoclonal antibody A20-9.1 was evaluated for its capacity to bind to four SARS CoV-2 Spike derived protein antigens (S2P, S1, RBD and NTD) and the S2P SARS CoV-1 spike protein antigen (FIG. 1). Binding was determined using ELISA immunoassay and showed that A20-9.1 is able to bind SARS CoV2 S2P and S1 but not SARS CoV2 RBD or NTD. Follow-up mapping ELISA looking at SD1 and SD2 domains for S1 suggested that the antibody is targeting a region in SD2 of S1. This data is consistent with the flow cytometry probe staining. Furthermore, the antibody was unable to bind to the SARS CoV1 S2P.

### Epitope Mapping

By assessing how A20-9.1 competes with previously published antibodies and antibodies disclosed herein, gross epitope can be determined. Competition classification was determined using Biolayer interferometry (BLI) assay described above. A20-9.1 competition profile is shown in FIG. 10. Taken together, this indicates that A20-9.1 has a distinct mode of binding and epitope within the RBD.

### Kinetics of Binding to the SARS CoV2 S2P Protein

Fab protein generated from A20-9.1 was evaluated for binding to SARS CoV2 S2P using BLI. A20-9.1 Fab shows binding to SARS CoV2 S2P protein with an affinity constant (K_{D}) of 18.9 nM, kₒₙ of 5.55×10⁵ per second and k_{off} of 1.05×10⁻² per Molar·second.

### Neutralization

A20-9.1 was tested for neutralization activity in a pseudotyped virus entry assay (FIG. 1). A20-9.1 was found to have a neutralization IC₅₀ of 1.2143 μg/mL for SARS CoV2 pseudotyped lentivirus particles.

### ACE2 Receptor Blocking

SARS CoV1 and SARS CoV2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. A20-9.1 ACE2 competition profile is shown in FIG. 10 and indicates that A20-9.1 does NOT prevent ACE2 from binding to S2P. Coupled with lack of competition with antibodies in the LY-COV555 class, this indicates that A20-9.1 is a neutralizing antibody that can be used as a non-competing partner for antibodies within the LY-COV555 competition class.

### Example 16

### 4.15 B1-182.1 (B1-182.1)

### Identification of Coronavirus Antibodies Using FACS Probe Sorting and SMRTseq Sequencing

B1-182.1 is a mAb whose variable domains were isolated through the pairing of heavy and light chain immunoglobulin genes from a single memory B cell obtained from a SARS CoV2 survivor 48 days following symptom onset. The nucleotide and amino acid sequences for the heavy and light chains of the expressed version of B1-182.1 can be found in Table 14. Variable heavy and light chain immunoglobulin sequence from Table 14 encoding the antigen binding region of B1-182.1 were synthesized and cloned into immunoglobulin expression vectors containing human constant regions for the IgG1 heavy chain and kappa light chain. These vectors were used to express antibody that was purified using standard methods.

TABLE 14
B1-182.1 gene family, and nucleotide and amino acid sequences for heavy and
light chains. CDR1, CDR2 and CDR3 are Bolded and underlined in the amino acid
sequences

| | V-gene Family | D-gene Family | J-gene Family | Isotype |
|---|---|---|---|---|
| Heavy Chain | IGHV1-58*01 | IGHD2-15*01 | IGHJ3*02 | IGHG3*04, IGHG2*06 |

CAAATGCAGCTGGTGCAGTCTGGGCCTGAGGTGAAGAAGCCTGGGACCTCAGTGAAGGTC
TCCTGCAAGGCTTCTGGATTCACCTTTACTAGCTCTGCTGTGCAGTGGGTGCGACAGGCTC
GTGGACAACGCCTTGAGTGGATAGGATGGATCGTCGTTGGCAGTGGTAACACAAACTACG
CACAGAAGTTCCAGGAAAGAGTCACCATTACCAGGGACATGTCCACAAGCACAGCCTATA
TGGAGCTGAGCAGCCTGAGATCCGAGGACACGGCCGTGTATTACTGTGCGGCCCCTTACT
GTAGTGGTGGTAGCTGCTTTGATGGTTTTGATATCTGGGGCCAAGGGACAATGGTCACCGT
CTCTTCAG (SEQ ID NO: 139)
QMQLVQSGPEVKKPGTSVKVSCKASGFTFTSSAVQWVRQARGQRLEWIGWIVVGSGNTNY
AQKFQERVTITRDMSTSTAYMELSSLRSEDTAVYYCAAPYCSGGSCFDGFDIWGQGTMVTV
SS (SEQ ID NO: 1)

| | | |
|---|---|---|
| Light Chain | IGKV3-20*01 | IGKJ1*01 |

GAAATTGTGTTGACGCAGTCTCCAGGCACCCTGTCTTTGTCTCCAGGGGAAAGAGCCACCC
TCTCCTGCAGGGCCAGTCAGAGTGTTAGCAGCAGCTACTTAGCCTGGTACCAGCAGAAAC
CTGGCCAGGCTCCCAGGCTCCTCATCTATGGTGCATCCAGCAGGGCCACTGGCTTCCCAGA
CAGGTTCAGTGGCAGTGGGTCTGGGACAGACTTCACTCTCACCATCAGCAGACTGGAGCC
TGAAGATTTTGCAGTGTATTACTGTCAGCAGTATGGTAACTCACCCTGGACGTTCGGCCAA
GGGACCAAGGTGGAAATCAGAC (SEQ ID NO: 140)
EIVLTQSPGTLSLSPGERATLSCRASQSVSSSYLAWYQQKPGQAPRLLIYGASSRATGFPDRFS
GSGSGTDFTLTISRLEPEDFAVYYCQQYGNSPWTFGQGTKVEIR (SEQ ID NO: 5)

### Binding to SARS-CoV-2 Spike Protein

Purified monoclonal antibody B1-182.1 was evaluated for its capacity to bind to four SARS CoV-2 Spike derived protein antigens (S2P, S1, RBD and NTD) and the S2P SARS CoV-1 spike protein antigen. Binding was determined using ELISA immunoassay and showed that B001-182.1 is able to bind SARS CoV-2 S2P, S1 and RBD but not SARS CoV-2 NTD or SARS CoV-1 S2P. With the exception of D614G/N439K, which had slightly decreased binding, B1-182.1 maintains or has increased binding to all of the variants tested including D614G, D614G/Y453F,D614G/501Y, D614G/de169-70, D614G/de169-70/N501Y, B.1.1.7, D614G/K471N, D614G/E484K, D614G/K417N/E484K/N501Y and B.1.351 variants, see FIG. 1.

### Epitope Mapping

By assessing how B1-182.1 competes with previously published antibodies and antibodies disclosed herein, gross epitope can be determined. Competition classification was determined using Biolayer interferometry (BLI) found that B1-182.1 has the same competition profile as A23-58.1, described above, and is in a unique competition group.

### Neutralization

B1-182.1 was tested for neutralization activity in a pseudotyped virus entry assay (See FIG. 1). B1-182.1 was found to have a neutralization IC₅₀ (0.0034 μg/mL) and IC₈₀ (0.0088 μg/mL) of SARS CoV2 pseudotyped lentivirus particles. This is 2 to 3-fold more potent than LY-COV555 (IC50: 0.0071, IC80: 0.0357 μg/mL). SARS COV-2 Nanoluc live virus neutralization IC₅₀ (0.0022 μg/mL) and IC₈₀ (0.0054 μg/mL) is amongst the most potent reported for antibodies targeting SARS COV-2.

Further testing against naturally occurring SARS COV-2 spike variants in pseudotyped neutralization assays showed that B1-182.1 maintains high potency (IC₅₀: <0.0006-0.0045 μg/mL; IC₈₀: <0.0006-0.0115 μg/mL) against the following variants D614G, N439K/D614G, Y453F/D614G, A222V/D614G, N501Y/D614G, de169-70/D614G, N501Y/de169-70/D614G, E484K/D614G, N501Y/E484K/K417N/D614G and B.1.1.7 (VOC 202012/01) that contains amino acid changes at H69del-V70del-Y144del-N501Y-A570D-D614G-P681H-T716I-S982A-D1118H. This is in contrast to LY-CoV555 and REGN-10989 which loses neutralizing capacity (>10 μg/mL) against the E484K/D614G, N501Y/E484K/K417N/D614G and B.1.351 variants. REGN-10933 loses neutralizing capacity (>10 μg/mL) against Y453F/E614G, N501Y/E484K/K417N/D614G and B.1.351. CB6 loses neutralizing activity (>10 μg/mL) against the N501Y/E484K/K417N/D614G and B.1.351 variants, see FIG. 1.

### ACE2 Receptor Blocking

SARS CoV-1 and SARS CoV-2 spike proteins target cells expressing by binding to ACE2 via the receptor binding domain on the spike protein. Given the important requirement for ACE2 binding in the virus life cycle, this vulnerability is exploited by several classes of antibodies that neutralize infection. Inhibition of ACE2 binding that is due to antibody binding of the spike glycoprotein can be performed using a modified version of the BLI competition assay described above. B1-182.1 ACE2 competition profile indicates that B1-182.1 prevents ACE2 from binding to S2P.

### Example 16

### Further Characterization

Abbreviated names were assigned to the antibodies, as shown in the table below, and further evaluation was conducted. Wang et al., Science, 373, eabh1766, Aug. 13, 2021, pages 1-14 and the supplemental materials are incorporated by reference herein.

Pseudovirus neutralization assays using the WA-1 spike showed that 4 RBD targeting antibodies, A19-46.1, A19-61.1, A23-58.1 and B1-182.1 were especially potent (IC₅₀ 2.5-70.9 ng/mL) (FIGS. 16D-16E). WA-1 live virus neutralization (Hou et al., Cell. 182, 429-446.e14 (2020)) revealed similar high potent neutralization by all four antibodies (IC₅₀ 2.1-4.8 ng/mL) (FIGS. 16D-16E). All four antibody Fabs exhibited nanomolar affinity for SARS-CoV-2 S-2P (i.e., 2.3-7.3 nM), consistent with their potent neutralization (FIG. 16E).

Antibodies targeting the RBD can be categorized into four general classes (i.e., Class I-IV) based on competition with the ACE2 target cell receptor protein for binding to S and recognition of the up or -down state of the three RBDs in S (Barnes et al., Nature. 588, 682-687 (2020)). LY-CoV555 is a therapeutic antibody that binds RBD in both the up and down states, blocks ACE2 binding and is categorized as Class II. However, despite potent activity against WA-1, VOCs have been reported to contain mutations that confer resistance to LY-CoV555 (Wang et al., Nature. 593, 130-135 (2021); Jones et al., Sci. Transl. Med. (2021), doi:10.1126/scitranslmed.abf1906; Chen et al., N. Engl. J. Med. 384, 229-237 (2021)) and similarly binding antibodies. It was therefore examined whether the epitopes targeted by the four high-potency antibodies were distinct from LY-CoV555. A surface plasmon resonance-based (SPR) competition binding assay was used to compare the binding profile of these antibodies to LY-CoV555. While LY-CoV555 competed with A19-46.1, A19-61.1, A23-58.1 and B1-182.1 (and vice versa), their overall competition profiles were not the same. A23-58.1 and B1-182.1 exhibit similar binding profiles and A19-61.1 and A19-46.1 likewise display a shared competition binding profile in the SPR assay. However, the latter two antibodies can be distinguished from each other due to A19-61.1 competition with the class III antibody S309 (Pinto et al., Nature. 583, 290-295 (2020)) (FIG. 16F) which binds an epitope in RBD that is accessible in the up or down position but does not compete with ACE2 binding (Barnes et al., Nature. 588, 682-687 (2020)).

To determine if the antibodies block ACE2 binding, biolayer interferometry ACE2-competition and cell surface binding assays were used to show that all four antibodies prevent the binding of ACE2 to spike (FIG. 16G). This suggested that A19-46.1, A23-58.1 and B1-182.1 neutralized infection by directly blocking the interaction of RBD with ACE2 and would be classified as either Class I (ACE2 blocking, binding RBD up only) or II (ACE2 blocking, binding RBD up or down) RBD antibodies (Barnes et al., supra). A19-61.1 competition with 5309 and ACE2 binding suggests that it binds at least partly outside of the ACE2 binding motif but may sterically block ACE2 binding similar to the Class III antibody REGN10987. To refine the classification of these antibodies, negative stain 3D reconstruction was performed, and it was found that A19-46.1 and A19-61.1 bound near one another with all RBDs in the down position (FIG. 16H), consistent with them being Class II and Class III antibodies, respectively. Similarly, A23-58.1 and B1-182.1 bound to overlapping regions when RBDs are in the up position, suggesting that they are Class I antibodies.

### Example 17

### Antibody Binding & Neutralization Against Circulating Variants

Because each donor subject was infected with a variant close to the ancestral WA-1, antibody activity was evaluated against recently emerged variants like D614G, which has become the dominant variant across the world (Korber et al., Cell. 182, 812-827.e19 (2020)). Similar to LY-CoV555, neutralization potency was increased against D614G compared to WA-1, with the IC₅₀ and IC₈₀ of each experimental antibody 1.4 to 6.3-fold lower than that seen for the WA-1 (IC₅₀ of 0.8-20.3 ng/ml and IC₈₀ of 2.6-43.5 ng/ml) (FIGS. 17A, and 17C).

Next, antibody binding was assessed to D614G and 9 additional cell surface expressed spike variants that have appeared subsequent to WA-1 and that are not considered variants of concern or interest (i.e., B.1.1.7.14, B.1.258.24, Y453F/D614G, Ap.1, B.1.388, DH69-70/N501Y/D614G, K417N/D614G, B.1.1.345, B.1.77.31) (6-9, 22). Experimental antibodies were compared to four antibodies that are in clinical use (LY-CoV555, REGN10933, REGN10987 and CB6, aka LY-CoV016). All control and experimental antibodies showed a minor reduction in binding (<2-fold) to B.1.258.24 (N439K/D614G). Despite this, their neutralization capacities were not significantly impacted, with the exception of REGN10987 (2.00 mg/mL) as reported previously (Thomson et al., Cell. 184, 1171-1187.e20 (2021)). While none of the experimental antibodies showed large reductions in binding, LY-CoV555, CB6 (Shi et al., Nature. 584, 120-124 (2020)) and REGN10933 (Hansen et al., Science. 369, 1010-1014 (2020)) each showed significant (>10-fold) binding deficits to one or more variants (i.e., Y453F/D614G, K417N/D614G, B.1.1.345 or B.1.177.31) in these cell-based binding assays.

The capacity was evaluated of each antibody to neutralize lentiviral particles pseudotyped with the same 10 variant spike proteins. Consistent with published data, REGN10933 did not neutralize Y453F/D614G or B.1.177.31 (K417N/E484K/N501Y/D614G) (12, 14, 26); CB6 did not neutralize B.1.177.31; and LY-CoV555 and REGN109333 showed significant potency reductions (28-fold to knockout) for neutralization of viruses containing E484K (Wibmer et al., Nat. Med. 27, 622-625 (2021); Wang et al., Nature. 593, 130-135 (2021)). Relative to WA-1, the A23-58.1 IC₅₀ neutralization was 3-fold lower for DH69-70/N501Y/D614G (0.9 ng/mL), 5-fold lower for Ap.1 (<0.6 ng/mL) and, while A23-58.1 maintained high potency, neutralization against B.1.1.345 was increased 4-fold (10.2 ng/mL). Neutralization by B1-182.1 maintained high-potency (IC₅₀<3.2 ng/mL) for all variants and showed more than 4-fold improved potency for 6 of the 10 variants tested (IC₅₀<0.8 ng/mL). For A19-61.1 variant neutralization was 3 to 6-fold more potent than WA-1 (WA-1 IC₅₀ 70.9 ng/mL; variants IC₅₀ 11.1-23.7 ng/mL). Finally, neutralization by A19-46.1 was similar to WA-1 for all variants except B.1.1.345 and B.1.177.31, which were still highly potent despite having IC₅₀ values that were 2 to 3-fold less active (B.1.1.345: 95.0 ng/ml; B.1.177.311: 61.8 ng/ml; WA-1: 39.8 ng/mL). Together, these data show the capacity of these newly identified antibodies to maintain high neutralization potency against a diverse panel of 10 variant spike proteins.

### Example 18

### Antibody Binding & Neutralization of Variants of Interest and Concern

Neutralization was analyzed for 13 circulating variants of interest/concern, some of which have high-transmissibility, including B.1.1.7, B.1.351, B.1.427, B.1.429, B.1.526, P.1, P.2, B.1.617.1 and B.1.617.2 (Rambaout et al., available on the internet, virological.org/t/preliminary-genomic-characterisation-of-an-emergent-sars-cov-2-lineage-in-the-uk-defined-by-a-novel-set-of-spike-mutations/5631 Tegally et al. Nature. 592, 438-443 (2021); Hou et al., Science. 370, 1464-1468 (2020)) (FIGS. 17A-17D). Consistent with published data, it was found that: LY-CoV555, CB6, REGN10933 and REGN10987 maintained high potency against B.1.1.7 (IC₅₀ 0.1-40.1 ng/mL) and LY-CoV555 and CB6 were unable to neutralize B.1.351 v.1, B.1.351 v2, P.1 v1 or P.1.v2 variants (IC₅₀>10,000 ng/mL) (FIGS. 17A-17D) (Wibmer et al., Nat. Med. 27, 622-625 (2021); Wang et al., Nature. 593, 130-135 (2021); Baum et al., Science. 369, 1014-1018 (2020)); LY-CoV555 was unable to neutralize B.1.526 v2, B.1.617.1 and B.1.617.2; CB6 showed 5 to 27-fold worse activity against B.1.1.7+E484K and B.1.429+E484K but remained active against B.1.617.1 and B.1.617.2; REGN10933 showed 9 to 200-fold reduction in neutralization against variants with mutations at E484 (i.e., B.1.1.7+E484K, B.1.429+E484K, B.1.526 v2,P.1 v1/v2 and B.1.617.1) and maintained activity against B.1.617.2 which does not contain a mutation at E484 (FIG. 17A-17D); REGN10987 maintained or had slightly increased potency against each of the VOC/VOIs except B.1.617.2 which showed a 4-fold reduction in activity (FIGS. 17A-17D). In comparison, A23-58.1, B1-182.1, A19-46.1 and A19-61.1 maintained similar or improved potency (IC₅₀<0.6-11.5 ng/mL) against B.1.1.7 and B.1.1.7+E484K relative to WA-1 (FIGS>17A-17D). The potency of A19-46.1 was within 2.5-fold or lower relative to WA-1 for all variants (IC₅₀ 11.5-101.4 ng/mL vs. WA-1 39.8 ng/mL) except those containing L452R (IC₅₀>10,000 ng/mL) (i.e., B.1.427, B.1.429, B.1.429+E484K, B.1.617.1 and B.1.617.2) (FIGS. 17A-17D). Further analyses showed that A23-58.1, B1-182.1 and A19-61.1 maintained high potency against all VOC/VOIs (IC₅₀<0.6-28.3 ng/mL), including the recently identified B.1.617.1 and B.1.617.2 (FIGS. 17A-17D). These results indicate that despite being isolated from subjects infected with early ancestral SARS-CoV-2 viruses, each of these antibodies have highly potent reactivity against VOCs.

### Example 19

### Structural and Functional Analysis of VH1-58 Antibodies

The two most potent antibodies, A23-58.1 and B1-182.1, shared highly similar gene family usage in their heavy and light chains, despite being from different donors. Both use IGHV1-58 heavy chains and IGKV3-20/IGKJ1 light chains and a similarly low levels of SHM (<0.7%). This antibody gene family combination has been identified in other COVID-19 convalescent subjects and has been proposed as a public clonotype (Tortorici et al., Science. 370, 950-957 (2020); Robbiani et al., Nature. 584, 437-442 (2020); Zost et al., Nature. 584, 443-449 (2020); Dejnirattisai et al., Cell. 184, 2183-2200.e22 (2021)). To gain structural insights on the interaction between this class of antibodies and the SARS-CoV-2 spike, cryo-EM reconstructions were obtained for structures of the Fab A23-58.1 bound to a stabilized WA-1 S at 3.39 Å resolution and of the Fab B1-182.1 bound to a stabilized WA-1 S at 3.15 Å resolution (Figure FIG. 18A, FIG. 18B). This revealed that the antibody bound to spike with all RBDs in the up position, confirming the negative stain results (FIG. 17H). However, the cryo-EM reconstruction densities of the interface between RBD and Fab were poor due to conformational variation.

To resolve the antibody-antigen interface, local refinement was performed, and the local resolution was improved to 3.89 Å for A23-58.1 and to 3.71 Å for B1-182.1. Since both A23-58.1 and B1-182.1 recognized the RBD in very similar way, the RBD-A23-58.1 structure was used for detailed analysis. Antibody A23-58.1 binds to an epitope on the RBD that faces the 3-fold axis of the spike and is accessible only in the RBD-up conformation (FIG. 18A). The interaction buried a total of 619 Å² surface area from the antibody and 624 Å² from the spike. The A23-58.1 paratope constituted all six complementarity-determining regions (CDR) with heavy chain and light chain contributing 74% and 26% of the binding surface area, respectively (FIGS. 18C and 18E). The 14-residue-long CDR H3, which is 48% of the heavy chain paratope, kinks at Pro95 and Phe100F (Kabat numbering scheme for antibody residues) to form a foot-like loop that is stabilized by an intra-loop disulfide bond between Cys97 and Cys100B at the arch. A glycan was observed at the CDR H3 Asn96. The CDRs formed an interfacial crater with a depth of ˜10 Å and a diameter of ˜20 Å at the opening. Paratope residues inside the crater were primarily aromatic or hydrophobic. CDR H3 Pro95 and Phe100F lined the bottom, and CDR H1 Ala33, CDR H2 Trp50 and Va152, and CDR H3 Val100A lined the heavy chain side of the crater (FIGS. 18D and 18E). On the light chain side, CDR L1 Tyr32 and CDR L3 residues Tyr91 and Trp96 provided 80% of the light chain binding surface (FIGS. 18D and 18E). In contrast, paratope residues at the rim of the crater are mainly hydrophilic, for example, Asp100D formed hydrogen bonds with Ser477 and Asn487 of the RBD (FIG. 18D).

The A23-58.1 epitope comprised residues between b5 and b6 at the tip of RBD (FIG. 18D, FIG. 19A). With the protruding Phe486 dipping into the crater formed by the CDRs, these residues formed a hook-like motif that is stabilized by an intra-loop disulfide bond between Cys480 and Cys488. Aromatic residues, including Phe456, Tyr473, Phe486 and Tyr489, provided 48% (299 Å²) of the epitope (FIG. 18D). Lys417 and Glu484, which are located at the outer edge of the epitope, contributed only 3.7% of the binding surface (FIG. 18C). Overall, the cryo-EM analysis provides a structural basis for the potent neutralization of the E484K/Q mutant by A23-58.1.

The binding modes and sequences of A23-58.1 and B1-182.1 are very similar to those of previously reported IGHV1-58/IGKV3-20-derived antibodies, such as S2E12 (Tortorici et al., Science. 370, 950-957 (2020)), COVOX 253 (Dejnirattisai et al., Cell. 184, 2183-2200.e22 (2021)) and CoV2-2196 (Dong et al., bioRxiv Prepr. Serv. Biol. (2021), doi:10.1101/2021.01.27.428529), confirming that they are members of the same structural class (FIG. 18E). To understand why B1-182.1 is highly effective at neutralizing the emerging VOCs, its binding mode was compared with A23-58.1. Analysis indicated that B1-182.1 rotated about 6 degrees along the long axis of Fab from that of A23-58.1 (FIG. 19B). This rotation on one hand increased B1-182.1 CDR L1 contacts on invariant regions of RBD to strengthen binding (FIG. 19B) and on the other hand critically reduced contact on Glu484 to 6 Å² and main-chain only comparing to ˜40 Å² main- and side-chain contacts for A58.1 and S2E12 (FIG. 19B). Overall, the subtle changes in antibody mode of recognition to regions on RBD harboring variant mutations provided structural basis on the effectiveness of B1-182.1 and A23-58.1 on neutralization of VOCs.

To understand how A23-58.1 and B1-182.1 overcome mutations that cause reduced antibody potency against virus variants, the antibody-RBD complex structures of CB6 (PDB ID 7C01) (Shi et al., Nature. 584, 120-124 (2020)), REGN10933 (PDB ID 6XDG) (Hansen et al., Science. 369, 1010-1014 (2020); Baum et al., Science. 370, 950-957 (2020)) and LY-CoV555 (PDB ID 7KMG) (Jones et al., Sci. Transl. Med. (2021), doi:10.1126/scitranslmed.abf1906) were superimposed with the A23-58.1 structure over the RDB region. Both REGN10933 and CB6 bind to the same side of the RBD as A23-58.1 (FIG. 19C). However, the binding surfaces of REGN10933 and CB6 were shifted towards the saddle of the open RBD and encompassed residues Lys417, Tyr453, Glu484 and Asn501 (FIG. 19C); mutations K417N and Y453F thus would abolish key interactions and lead to the loss of neutralization for both REGN10933 and CB6 (FIGS. 17A-17D). In contrast, LY-CoV555 approached the RBD from a different angle with its epitope encompassing Glu484 and Lys452 (FIG. 19D). Structural examination indicates that E484K/Q abolishes key interactions with CDR H2 Arg50 and CDR L3 Arg96 of LY-CoV555. In addition, both E484K/Q (FIG. 19D) and L452R mutations cause clashes with heavy chain of LY-CoV555. When compared with epitopes of class I, II and III antibodies (Dejnirattisai et al., supra), the supersite defined by common contacts of the IGHV1-58-derived antibodies (A23-58.1, B1-182.1, S2E12 and COVOX253) had minimal interactions with residues at the mutational hotspots (FIG. 19E). These structural data suggest that the binding modes of A23-58.1 and B1-182.1 enabled their high effectiveness against the new SARS-CoV-2 VOCs.

Based on the structural analysis the relative contribution of predicted contact residues on binding and neutralization (FIG. 19A) was investigated. Cell surface expressed spike binding to A23-58.1 and B1-182.1 was knocked out by F486R, N487R, and Y489R (FIG. 20A), resulting in a lack of neutralization for viruses pseudotyped with spikes containing these mutations (FIG. 20B). In contrast, binding and neutralization of A19-46.1 and A19-61.1 were minimally impacted by these changes (FIGS. 21B, and 21C). CB6, LY-CoV555 and REGN10933 binding and neutralization were also impacted by the three mutations, consistent with the structural analysis that these residues are shared contact(s) with A23-58.1 and B1-182.1. Taken together, the shared binding and neutralization defects suggest that the hook-like motif and CDR crater are critical for the binding of antibodies within the VH1-58 public class.

### Example 20

### Generation and Testing of Escape Mutations

To explore critical contact residues and mechanisms of escape that might be generated during the course of infection, antibody selection pressure was applied to replication competent vesicular stomatitis virus (rcVSV) expressing the WA-1 SARS-CoV-2 spike (rcVSV-SARS2) (Deterle et al., Cell Host Microbe. 28, 486-496.e6 (2020)) to identify spike mutations that confer in vitro resistance against A23-58.1, B1-182.1, A19-46.1 or A19-61.1. rcVSV-SARS2 was incubated with increasing concentrations of antibody, and cultures from the highest concentration of antibody with >20% cytopathic effect (CPE), relative to no infection control, were carried forward into a second round of selection to drive resistance (Baum et al., Science. 369, 1014-1018 (2020)). A shift to higher antibody concentrations required for neutralization indicates the presence of resistant viruses. To gain insight into spike mutations driving resistance, Illumina-based shotgun sequencing was performed. Variants present at a frequency of greater than 5% and increasing from round 1 to round 2 were considered to be positively selected resistant viruses. For A19-46.1, escape mutations were generated at four sites: Y449S (freq. 15%), N450S (freq. 16%), N450Y (freq. 14%), L452R (freq. 83%) and F490V (freq. 58%) (FIG. 21A). The most dominant, L452R, is consistent with the previous finding that B.1.427, B.1.429, B.1.617.1 and B.1.617.2 were resistant to A19-46.1 (FIGS. 17A-17D). Interestingly, while F490V did not knockout neutralization, F490L did, suggesting that F490V may require additional mutations to escape to occur (FIGS. 21A-21C). Since Y449, N450 and L452 are immediately adjacent to S494, it was tested whether S494R would also disrupt binding and neutralization (FIGS. 21A-21C) and found that this mutation mediates neutralization escape. Each of the identified residue locations were confirmed by binding and/or neutralization and would be expected to be accessible when RBD is in the up or down position, and several are shared by Class II RBD antibodies (Barnes et al., Nature. 588, 682-687 (2020); Rappazzo et al., Science. 371, 823-829 (2021)) and REGN10933 (Hansen et al., Science. 369, 1010-1014 (2020); Barnes et al., Cell. 182, 828-842.e16 (2020)).

Three residues were positively selected in the presence of A19-61.1: K444E/T (freq. 7-93%), G446V (freq. 24%) and G593R (freq. 19%) (FIG. 21A). There was no overlap with those selected by A19-46.1. G593R is located outside the RBD domain, did not impact neutralization and may therefore represent a false positive. The highest frequency change was K444E represented 57-93% of the sequences in replicate experiments (FIG. 21A). This residue is critical for the binding of Class III RBD antibodies such as REGN10987 (Barnes et al., Nature. 588, 682-687 (2020); Hansen et al., Science. 369, 1010-1014 (2020); Baum et al., Science. 369, 1014-1018 (2020); Barnes et al., Cell. 182, 828-842.e16 (2020)). Due to the proximity of S494 to K444 and G446, S494R was tested for escape potential and shown to mediate escape from A19-61.1 neutralization. These results are consistent with A19-61.1 targeting a distinct epitope from REGN10987 and other Class III RBD antibodies.

For A23-58.1, a single F486S mutation (freq. 91-98%) was positively selected. Similarly, B1-182.1 escape was mediated by F486L (21%), N487D (100%) and Q493R (45%). Q493R, had minimal impact on binding and was not found to impact neutralization (FIG. 21B, C). However, F486, N487 and Y489 were all in agreement with previous structural analysis (FIG. 18D, FIG. 20A-21B, FIG. 21A-21E). F486 is located at the tip of RBD hook and contacts the binding interface in the antibody crater where aromatic side chains dominantly form the hook and crater interface (FIG. 18D). Therefore, the loss in activity may occur through replacement of a hydrophobic aromatic residue (phenylalanine) with a small polar side chain (serine) (FIG. 18D).

### Example 21

### Potential Escape Risk and Mitigation

To probe the relevance of in vitro derived resistance variants to potential clinical resistance, the relative frequency of variants containing escape mutations present in the GISAID sequence database was investigated using the COVID-19 Viral Genome Analysis Pipeline (available on the internet, cov.lanl.gov) (Korber et al., Cell. 182, 812-827.e19 (2020)) in which, as of May 7, 2021, there were 1,062,910 entries. Of the residues noted to mediate escape or resistance to A19-46.1 (i.e., Y449S, N450S/Y, L452R, F490L/V and S494R), only F490L (0.02%) and L452R (2.27%) were present at greater than 0.01%. For the A19-61.1 escape mutations (i.e., K444E, G446V, S494R), only G446V has been noted in the database >0.01% (0.03%). Finally, for A23-58.1 and B1-182.1 ancestral WA-1 residues F486, N487 and Y489 were present in >99.96% of sequences and only F486L was noted in the database at >0.01% (0.03%). While the relative lack of A19-61.1, A23-58.1 and B1-182.1 escape mutations in circulating viruses could reflect either under-sampling or the absence of selection pressure, it may also suggest that the in vitro derived mutations may exact a fitness cost on the virus.

Viral genome sequencing has suggested that in addition to spread via transmission, convergent selection of de novo mutations may be occurring (Rambaut eet al., available on the internet, virological.org/t/preliminary-genomic-characterisation-of-an-emergent-sars-cov-2-lineage-in-the-uk-defined-by-a-novel-set-of-spike-mutations/563Gary, Virological. virological.org (2021), (available at on the internet, virological.org/t/mutations-arising-in-sars-cov-2-spike-on-sustained-human-to-human-transmission-and-human-to-animal-passage/578); Tegally et al., Nature. 592, 438-443 (2021); Faria et al., (2021), availble on the internet, virological.org/t/genomic-characterisation-of-an-emergent-sars-cov-2-lineage-in-manaus-preliminary-findings/586; Naveca et al., 2021, availble on the internet, virological.org/t/phylogenetic-relationship-of-sars-cov-2-sequences-from-amazonas-with-emerging-brazilian-variants-harboring-mutations-e484k-and-n501y-in-the-spike-protein/585; Korber et al., Cell. 182, 812-827.e19 (2020); Rappazzo et al., Science. 371, 823-829 (2021)). Therefore, effective therapeutic antibody approaches might require new antibodies or combinations of antibodies to mitigate the impact of mutations. Based on their complementary modes of spike recognition and breadth of neutralizing activity, combination of B1-182.1 with either A19-46.1 or A19-61.1 may decrease the rate of in vitro resistance acquisition compared to each antibody alone. Consistent with the competition data (FIG. 16F), negative stain EM 3D reconstructions show that the Fabs in both combinations were able to simultaneously engage spike with the RBDs in the up position (FIG. 21D). Binding was observed for up to 3 Fabs of B1-182.1 and 3 Fabs of A19-46.1 or A19-61.1 per spike in the observed particles (FIG. 21D), indicating that the epitopes of A19-46.1 and A19-61.1 on the spike are accessible in both RBD up and down positions (FIG. 1H and FIG. 21D). The absence of observed RBD-down classes suggests the possibility that the combination induces a preferred mode of RBD-up engagement (i.e., RBD up vs. RBD down) due the requirement of B1-182.1 or A23-58.1 for RBD-up binding.

Next, the capacity was evaluated of individual antibodies or combinations to prevent the appearance of rcVSV SARS-CoV-2-induced cytopathic effect (CPE) through multiple rounds of passaging in the presence of increasing concentrations of antibodies. In each round, the well with the highest concentration of antibody with at least 20% CPE was carried forward into the next round. It was found that wells with A19-61.1 or A785.46.1 single antibody treatment reached the 20% CPE threshold in their 50 mg/mL well after 3 rounds of selection (FIG. 21E). Similarly, B1-182.1 single antibody treatment reached >20% CPE in the 50 mg/mL wells after 4 rounds (FIG. 21E). Conversely, for both dual treatments (i.e., B1-182.1/A19-46.1 or B1-182.1/A19-61.1) the 20% CPE threshold was reached at a concentration of only 0.08 mg/mL and did not progress to higher concentrations despite 5 rounds of passaging (FIG. 21E). Thus, combinations can lower the risk that a natural variant will lead to the complete loss of neutralizing activity and suggests a path forward for these antibodies as combination therapies.

The results show that highly potent neutralizing antibodies with activity against VOCs was present in at least 3 of 4 convalescent subjects who had been infected with ancestral variants of SARS-CoV-2 (see, for example, FIGS. 16A-16H). Furthermore, the structural analyses, the relative paucity of potential escape variants in the GSAID genome database, the identification of public clonotypes (Tortorici et al., Science. 370, 950-957 (2020); Robbiani et al., Nature. 584, 437-442 (2020)) and the fact that each subject had mild to moderate illness all suggest that these antibodies were generated in subjects who rapidly controlled their infection and were not likely to have been generated due to the generation of a E484 escape mutation during the course of illness. Taken together, these data establish the rationale for a vaccine boosting regimen that may be used to selectively induce immune responses that increase the breadth and potency of antibodies targeting specific RBD regions of the spike glycoprotein (e.g., VH1-58 supersite). Since both variant sequence analysis and in vitro time to escape experiments suggest that combinations of these antibodies may have a lower risk for loss of neutralizing activity, these antibodies represent a potential means to achieve both breadth against current VOCs and to mitigate risk against those that may develop in the future.

### Example 22

### Additional Materials and Methods

Isolation of PBMCs from SARS CoV-2 subjects: Human convalescent sera samples were obtained 25 to 55 days following symptom onset from adults with previous mild to moderate SARS-CoV-2 infection. Whole blood was collected in vacutainer tubes, which were inverted gently to remix cells prior to standard FICOLL®-Hypaque density gradient centrifugation (Pharmacia; Uppsala, Sweden) to isolate PBMCs. PBMCs were frozen in heat-inactivated fetal calf serum containing 10% dimethylsulfoxide in a FORMA® CRYOMED® cell freezer (Marietta, OH). Cells were stored at ˜<140° C.

Expression and Purification of Protein: For expression of soluble SARS CoV-2 S-2P protein, manufacturer's instructions were followed. Briefly, plasmid was transfected using EXPIFECTAMINE® into EXP1293® cells (Life Technology, #A14635, A14527) and the cultures enhanced 16-24 hours post-transfection. Following 4-5 days incubations at 120 rpm, 37° C., 9% CO2, supernatant was harvested, clarified via centrifugation, and buffer exchanged into 1×PBS. Protein of interests were then isolated by affinity chromatography using Streptactin resin (Life science) followed by size exclusion chromatography on a SUPEROSE® 6 increase 10/300 column (GE healthcare).

Expression and purification of biotinylated S-2P, NTD, RBD-SD1 and Hexapro used in binding assays were produced by an in-column biotinylation method as previously described (Zhou et al., Cell Rep. 33, 108322 (2020)). Using full-length SARS-Cov2 S and human ACE2 cDNA ORF clone vector (Sino Biological, Inc) as the template to generate S1 or ACE2 dimer proteins. The S1 PCR fragment (1˜681aa) was digested with Xbal and BamHI and cloned into the VRC8400 with HRV3C-his (6×) or Avi-HRV3C-his (6×) tag on the C-terminal. The ACE2 PCR fragment (1-740aa) was digested with Xbal and BamHI and cloned into the VRC8400 with Avi-HRV3C-single chain-human Fc-his (6×) tag on the C-terminal. All constructs were confirmed by sequencing. Proteins were expressed in Expi293 cells by transfection with expression vectors encoding corresponding genes. The transfected cells were cultured in shaker incubator at 120 rpm, 37° C., 9% CO2 for 4-5 days. Culture supernatants were harvested and filtered, and proteins were purified through a Hispur Ni-NTA resin (Thermo Scientific, #88221) and following a HILOAD® 16/600 SUPERDEX® 200 column (GE healthcare, Piscataway NJ) according to manufacturer's instructions. The protein purity was confirmed by SDS-PAGE.

Probe conjugation: SARS CoV-2 Spike trimer (S-2P) and subdomains (NTD, RBD-SD1, S1) were produced by transient transfection of 293 Freestyle cells as previously described (Wrapp et al., Science. 367, 1260-1263 (2020)). Avi-tagged S1 was biotinylated using the BirA biotin-protein ligase reaction kit (Avidity, #BirA500) according to the manufacturer's instructions. The S-2P, RBD-SD1, and NTD proteins were produced by an in-column biotinylation method as previously described (Zhou et al., Cell Rep. 33, 108322 (2020)). Successful biotinylation was confirmed using Bio-Layer Interferometry, by testing the ability of biotinylated protein to bind to streptavidin sensors. Retention of antigenicity was confirmed by testing biotinylated proteins against a panel of cross-reactive SARS-CoV and SARS CoV-2 human monoclonal antibodies. Biotinylated probes were conjugated using either allophycocyanin (APC)-, Ax647, BV421-, BV786, BV711-, or BV570-labeled streptavidin. Reactions were prepared at a 4:1 molecular ratio of biotinylated protein to streptavidin, with every monomer labeled. Labeled streptavidin was added in % increments and in the dark at 4° C. (rotating) for 20 minutes in between each addition. Optimal titers were determined using splenocytes from immunized mice and validated with SARS CoV-2 convalescent human PBMC.

Isolation of and sequencing of antibodies by single B cell sorting: Cryopreserved human PBMCs from four COVID-19 convalescent donors were thawed and stained with Live/DEAD Fixable Aqua Dead Cell Stain kit (Cat #134957, ThermoFisher). After washing, cells were stained with a cocktail of anti-human antibodies, including CD3 (cat #317332, Biolegend), CD8 (cat #301048, Biolegend), CD56 (cat #318340, Biolegend), CD14 (cat #301842, Biolegend), CD19 (Cat #IM2708U, Becknan Coulter), CD20 (cat #302314, Biolegend), IgG (Cat #555786, BD Biosciences), IgA (Cat #130-114-001, Miltenyi), IgM (Cat #561285, BD Biosciences) and subsequently stained with fluorescently labeled SARS-CoV-2 S-2P (APC or Ax647), S1 (BV786 or BV571), RBD-SD1 (BV421) and NTD (BV711 or BV421) probes. Antigen-specific memory B cells (CD3-CD19+CD20+IgG+ or IgGA+ and S-2P+ and/or RBD+ for the donors Subjects A19, A20 and A23, S-2P+ and/or NTD+ for the donor Subject B1) were sorted using a FACSYMPHONY® S6 (BD Sciences) into Buffer TCL (Qiagen) with 1% 2-mercaptoethanol (ThermoFisher Scientific). Nucleic acids were purified using RNAClean magnetic beads (Beckman Coulter) followed by reverse transcription using oligo-dT linked to a custom adapter sequence and template switching using SMARTSCRIBE® RT (Takara). PCR amplification was carried out using SeqAmp DNA Polymerase (Takara). A portion of the amplified cDNA was enriched for B cell receptor sequences using forward primers complementary to the template switch oligo and reverse primers against the IgA (GAGGCTCAGCGGGAAGACCTTGGGGCTGGTCGG, SEQ ID NO: 142) IgG, IgK, and IgX (38) constant regions. Enriched products were made into Illumina-ready sequencing libraries using the NEXTERA® XT DNA Library Kit with Unique Dual Indexes (ILLUMINA®). The ILLUMINA®-ready libraries were sequenced by paired end 150 cycle MiSeq reads. The resulting reads were demultiplexed using an in-house script and V(D)J sequences were assembled using BALDR in unfiltered mode. Poor or incomplete assemblies or those with low read support were removed, and the filtered contigs were re-annotated with SONAR v4.2 in single cell mode. A subset of the final antibodies was manually selected for synthesis based on multiple considerations, including gene usage, somatic hypermutation levels, CDRH3 length, convergent rearrangements, and specificity implied by flow cytometry.

Synthesis, cloning and expression of monoclonal antibodies: Sequences were selected for synthesis to sample expanded clonal lineages within our dataset and convergent rearrangements both among donors in our cohort and compared to the public literature. In addition, a variety of sequences were synthesized that were designed to be representative of the whole dataset along several dimensions, including apparent epitope based on flow data; V gene usage; somatic hypermutation levels; CDRH3 length; and isotype. Variable heavy chain sequences were human codon optimized, synthesized and cloned into a VRC8400 (CMV/R expression vector)-based IgG1 vector containing an HRV3C protease site (McLellan et al., Nature. 480, 336-43 (2011)) as previously described (Moyo-Gwete et al., N. Engl. J. Med. 2 (2021), doi:10.1056/NEJMc2104192). Similarly, variable lambda and kappa light chain sequences were human codon optimized, synthesized and cloned into CMV/R-based lambda or kappa chain expression vectors, as appropriate (Genscript). Previously published antibody vectors for LY-COV555 (Barnes et al., Nature. 588, 682-687 (2020) and mAb114 (Misasi et al., Science. 351, 1343-6 (2016)) were used. The antibodies: REGN10933 was produced from published sequences (25) and kindly provided by Devin Sok from Scripps. For antibodies where vectors were unavailable (e.g., S309, CB6), published amino acids sequences were used for synthesis and cloning into corresponding pVRC8400 vectors (42,43). For antibody expression, equal amounts of heavy and light chain plasmid DNA were transfected into Expi293 cells (Life Technology) by using Expi293 transfection reagent (Life Technology). The transfected cells were cultured in shaker incubator at 120 rpm, 37° C., 9% CO2 for 4-5 days. Culture supernatants were harvested and filtered, mAbs were purified over Protein A (GE Health Science) columns. Each antibody was eluted with IgG elution buffer (Pierce) and immediately neutralized with one tenth volume of 1M Tris-HCL pH 8.0. The antibodies were then buffer exchanged as least twice in PBS by dialysis.

ELISA method description: Testing is performed using the automated ELISA method as detailed in VRC-VIP SOP 5500 Automated ELISA on Integrated Automation System. Quantification of IgG concentrations in serum/plasma are performed with a Beckman BIOMEK® based automation platform. The SARS-CoV-2 S-2P (VRC-SARS-CoV-2 S-2P (15-1208)-3C-His8-Strep2×2) and RBD (Ragon-SARS-CoV-2 S-RBD (319-529)-His8-SBP) Antigen are coated onto IMMULON® 4HBX flat bottom plates overnight for 16 hours at 4° C. at a concentration of 2 mg/mL and 4 mg/mL, respectively. Proteins were produced and provided. Antigen concentrations were defined during assay development and antigen lot titration. Plates are washed and blocked (3% milk TPBS) for 1 hour at room temperature. Duplicate serial 4-fold dilutions covering the range of 1:100-1:1638400 (8-dilution series) of the test sample (diluted in 1% milk in TPBS) are incubated at room temperature for 2 hours followed by Horseradish Peroxidase-labeled goat anti-human antibody detection (1 hour at room temperature) (Thermo Fisher Catalogue #A1881), and TMB substrate (15 minutes at room temperature; DAKO Catalogue #S1599) addition. Color development is stopped by addition of sulfuric acid and plates are read within 30 minutes at 450 nm and 650 nm via the Molecular Devices Paradigm plate reader. Each plate harbors a negative control (assay diluent), positive control (SARS-CoV-2 S2-specific monoclonal antibody 5-652-112 spiked in NHS and/or pool of COVID-19 convalescent sera) and batches of 5 specimen run in duplicates. All controls are trended over time.

Endpoint Titer dilution from raw OD data is interpolated using the plate background OD+10 STDEV by asymmetric sigmoidal 5-pl curve fit of the test sample. In the rare event, the asymmetric sigmoidal 5-pl curve failed to interpolate the endpoint titer, a sigmoidal 4-pl curve is used for the analysis. Area under the curve (AUC) is calculated with baseline anchored by the plate background OD+10 STDEV. Data analysis is performed using Microsoft Excel and GraphPad Prism Version 8.0.

Assignment of major binding determinant using MSD binding assay: MSD 384-well streptavidin-coated plates (MSD, cat #L21SA) were blocked with MSD 5% Blocker A solution (MSD, cat #R93AA), using 35 ul per well. These plates were then incubated for 30 to 60 minutes at room temperature. Plates were washed with 1× Phosphate Buffered Saline+0.05% TWEEN® 20 (PBST) on a BIOTEK® 405TS automated microplate washer. Five SARS CoV-2 capture antigens were used. Capture antigens consisted of VRC-produced S1, S-2P, S6P (HEXAPRO®), RBD, and NTD. All antigens were AVI-tag biotinylated using BirA (Avidity, cat #BirA500) AVI-tag specific biotinylation following manufacturer's instructions except S1. For S1, an Invitrogen FLUOREPORTER™ Mini-Biotin-XX Protein Labeling Kit (Thermo Fisher, cat #F6347) was utilized to achieve random biotinylation. Antigen coating solutions were prepared for S1, S-2P, S6P, RBD, and NTD at optimized concentrations of 0.5, 0.25, 1, 0.5, and 0.25 μg/mL, respectively. These solutions were then added to MSD 384-well plates, using 10 μL per well. Each full antigen set is intended to test one plate of experimental SARS CoV-2 monoclonal antibodies (mAbs) at one dilution. Once capture antigen solutions were added, plates were incubated for 1 hour at room temperature on a HEIDOLPH® Titramax 1000 (HEIDOLPH®, part #544-12200-00) vibrational plate shaker at 1000 rpm. During this time, experimental SARS CoV-2 mAb dilution plates were prepared. Using this initial plate, 3 dilution plates were created at dilution factors of 1:100, 1:1000, and 1:10000. Dilutions were performed in 1% assay diluent (MSD 5% Blocker A solution diluted 1:5 in PBST). Positive control mAbs S652-109 (SARS Cov-2 RDB specific) and S652-112 (SARS CoV-2 S1, S-2P, S6P, and NTD specific) and negative control mAb VRCO1 (anti-HIV) were added to all dilution plates at a uniform concentration of 0.05 μg/mL. Once mAb dilution plates were prepared, MSD 384-well plates were washed as above. The content of each 96-well dilution plate was added to the MSD 384-well plates, using 10 μL per well. MSD 384-well plates were then incubated for 1 hour at room temperature on vibrational plate shaker at 1000 rpm. MSD 384-well plates were washed as above, and MSD Sulfo-Tag labeled goat anti-human secondary detection antibody (MSD, cat #R32AJ) solution was added to plates at a concentration of 0.5 μg/mL, using 10 μL per well. Plates were again incubated for 1 hour at room temperature on vibrational plate shaker at 1000 rpm. MSD 1× Read Buffer T (MSD, cat #R92TC) was added to MSD 384-well plates, using 35 μL per well. MSD 384-well plates were then read using MSD Sector S 600 imager. Gross binding epitope of S-2P or Hexapro positive antibodies was assigned into the following groups: RBD (i.e., RBD+ or RBD+/S1+ AND NTD−), NTD (i.e., NTD+ or NTD+/S1+ AND RBD−), S2 (i.e., S1−, RBD− AND NTD−) or indeterminant (i.e., mixed positive). Antibodies lacking binding to any of the antigens were assigned to the “no binding” group.

Full-length S constructs: cDNAs encoding full-length S from SARS CoV-2 (GENBANK® ID: QHD43416.1, as available on Jan. 1, 2022, incorporated herein by reference) were synthesized, cloned into the mammalian expression vector VRC8400 (Barouch et al., J. Virol. 79, 8828-8834 (2005); Cantanzaro et al., Vaccine. 25, 4085-92 (2007)) and confirmed by sequencing. S containing D614G amino acid change was generated using the wild-type (wt) S sequence. Other variants containing single or multiple aa changes in the S gene from the S wt or D614G were made by mutagenesis using QUICKCHANGE® lightning Multi Site-Directed Mutagenesis Kit (cat #210515, Agilent). The S variants, N439K, Y453F, A222V, E484K, K417N, S477N, N501Y, delH69/V70, N501Y-delH69/V70, N501Y-E484K-K417N, B.1.1.7 (H69del-V70del-Y144del-N501Y-A570D-P681H-T716I-S982A-D1118H), B.1.351.v1 (L18F-D80A-D215G-(L242-244)del-R246I-K417N-E484K-N501Y-A701V), B.1.351.v2 (L18F-D80A-D215G-(L242-244)del-K417N-E484K-N501Y-A701V), B.1.427 (L452R-D614G), B.1.429 (S13I-W152C-L452R-D614G), B.1.526.v2 (L5F-T951-D253G-E484K-D614G-A701V), P.1.v1 (L18F-T20N-P26S-D138Y-R190S-K417T-E484K-N501Y-D614G-H655Y-T1027I), P.1.v2 (L18F-T20N-P26S-D138Y-R190S-K417T-E484K-N501Y-D614G-H655Y-T1027I-V7116F), P.2 (E484K-D614G-V7116F), B.1.617.1 (T95I-G412D-E154K-L452R-E484Q-D614G-P681R-Q1071H), B.1.617.2 (T19R-G142D-de1156-157-R158G-L452R-T478K-D614G-P681R-D950N) and antibody escape mutations, F486S, K444E, Y449S, N450S and F490V were generated based on S D614G while the antibody contact residue mutations, F456R, A475R, T478I, F486R, Y489R, N487R, L452R, F490L, Q493R, S494R on S wt. These full-length S plasmids were used for pseudovirus production and for cell surface binding assays.

Pseudovirus neutralization assay: S-containing lentiviral pseudovirions were produced by co-transfection of packaging plasmid pCMVdR8.2, transducing plasmid pHR′ CMV-Luc, a TMPRSS2 plasmid and S plasmids from SARS CoV-2 variants into 293T (ATCC) cells using FUGENE® 6 transfection reagent (Promega, Madison, WI) (44-45). 293T-ACE2 cells were plated into 96-well white/black Isoplates (PerkinElmer, Waltham, MA) at 5,000 cells per well the day before infection of SARS CoV-2 pseudovirus. Serial dilutions of mAbs were mixed with titrated pseudovirus, incubated for 45 minutes at 37° C. and added to 293T-ACE2 cells in triplicate. Following 2 hours (h) of incubation, wells were replenished with 150 ml of fresh media. Cells were lysed 72 h later, and luciferase activity was measured with Microbeta (Perkin Elmer). Percent neutralization and neutralization IC₅₀s, IC₈₀ s were calculated using GraphPad Prism 8.0.2. Serum neutralization assays were performed as above excepting all human sera had an input starting serial dilution of 1:20 and neutralization was quantified as the inhibition dilution 50% (ID50) of virus entry. Alternative method pseudovirus neutralization assay in Figure S3 utilized a 1^{st} generation lentivirus system and was performed as in Wibmer et al (Nat. Med. 27, 622-625 (2021)).

Cell surface binding: HEK293T cells were transiently transfected with plasmids encoding full length SARS CoV-2 spike variants using LIPOFECTAMINE® 3000 (L3000-001, ThermoFisher) following the manufacturer's protocol. After 40 hours, the cells were harvested and incubated with monoclonal antibodies (1 μg/ml) for 30 minutes. After incubation with the antibodies, the cells were washed and incubated with an allophycocyanin conjugated anti-human IgG (709-136-149, Jackson Immunoresearch Laboratories) for another 30 minutes. The cells were then washed and fixed with 1% paraformaldehyde (15712-5, Electron Microscopy Sciences). The samples were then acquired in a BD LSRFORTESSA™ X-50 flow cytometer (BD biosciences) and analyzed using FLOWJO® (BD biosciences). Mean fluorescent intensity (MFI) for antibody binding to S wt or D614G was set up as 100%. The MFI of the antibody binding to each variant was normalized to S wt or D614G.

Competitive mAb binding assay using surface plasmon resonance: Monoclonal antibody (mAb) competition assays were performed on a BIACORE® 8K+(CYTIVA®) surface plasmon resonance spectrometer. Anti-histidine IgG₁ antibody was immobilized on Series S Sensor Chip CM5 (CYTIVA®) using a His capture kit (CYTIVA®), per the manufacturer's instructions. 1×PBS-P+(CYTIVA®) was used for running buffer and diluent, unless noted. 8× His-tagged SARS-CoV-2 Spike protein containing 2 proline stabilization mutations, K986P and V987P, (S-2P) (4) was captured on the active sensor surface. “Competitor” mAb or a negative control mAb114 (37) were first injected over both active and reference surfaces, followed by “analyte” mAb. Between cycles, sensor surfaces were regenerated with 10 mM glycine, pH 1.5 (CYTIVA®).

For data analysis, sensorgrams were aligned to Y (Response Units, RUs)=0, beginning at the beginning of each mAb binding phase in BIACORE® 8K Insights Evaluation Software (CYTIVA®). Reference-subtracted, relative “analyte binding late” report points (in RUs) were used to determine percent competition for each mAb. Maximum analyte binding for each mAb was first defined by change in RUs during analyte binding phase when negative control mAb was used as competitor mAb. Percent competition (% C) was calculated using the following formula: % C=100*[1−((analyte mAb binding RUs when S-2P-specific mAb is used as competitor)/(maximum analyte binding RUs when negative control mAb is used as competitor))].

Competitive ACE2 binding assay using biolayer interferometry: Antibody cross-competition was determined based on biolayer interferometry using a FORTÉBIO® Octet HTX instrument. His1K biosensors (FORTEBIO®) were equilibrated for >600 s in Blocking Buffer (1% BSA (Sigma)+0.01% TWEEN®-20 (Sigma)+0.01% Sodium Azide (Sigma)+PBS (Gibco), pH7.4) prior to loading with his tagged S-2P protein (10 μg/mL in Blocking Buffer) for 1200s. Following loading, sensors were incubated for 420s in Blocking Buffer prior to incubation with competitor mAbs (30 mg/mL in Blocking Buffer) or ACE2 (266 nM in Blocking Buffer) for 1200s. Sensors were then incubated in Blocking buffer for 30s prior to incubation with ACE2 (266 nM in Blocking Buffer) for 1200s. Percent competition (PC) of ACE2 mAbs binding to competitor-bound S-2P was determined using the equation: PC=100−[(ACE2 binding in the presence competitor mAb)/(ACE2 binding in the absence of competitor mAb)]×100. All the assays were performed in duplicate and with agitation set to 1,000 rpm at 30° C.

Inhibition of S protein binding to cell surface ACE2: Serial dilutions of mAb IgG and Fab were mixed with pre-titrated biotinylated S trimer (S-2P), incubated for 30 minutes at RT and added to BHK21 cells stably expressing hACE2 on cell surface. Following 30 minutes of incubation on ice, the cells were washed and incubated with an BV421 conjugated Streptavidin (cat #563259, BD Biosciences) for another 30 minutes. The cells were then washed and fixed with 1% paraformaldehyde (15712-5, Electron Microscopy Sciences). The samples were then acquired in a BD LSRFORTESSA® X-50 flow cytometer (BD biosciences) and analyzed using FLOWJO® (BD biosciences). Mean fluorescent intensity (MFI) for S protein binding to cell surface was set up as 100%. Percent inhibition of S protein binding to cell surface ACE2 by mAb IgG and EC50s were calculated using GraphPad Prism 8.0.2.

Live virus neutralization assay: Full-length SARS CoV-2 virus based on the Seattle Washington strain was designed to express nanoluciferase (nLuc) and was recovered via reverse genetics and described previously (Hou et al., Cell. 182, 429-446.e14 (2020)). Virus titers were measured in Vero E6 USAMRIID cells, as defined by plaque forming units (PFU) per ml, in a 6-well plate format in quadruplicate biological replicates for accuracy. For the 96-well neutralization assay, Vero E6 USAMRID cells were plated at 20,000 cells per well the day prior in clear bottom black walled plates. Cells were inspected to ensure confluency on the day of assay. Serially diluted mAbs were mixed in equal volume with diluted virus. Antibody-virus and virus only mixtures were then incubated at 37° C. with 5% CO₂ for one hour. Following incubation, serially diluted mAbs and virus only controls were added in duplicate to the cells at 75 PFU at 37° C. with 5% CO₂. After 24 hours, cells were lysed, and luciferase activity was measured via NANO-GLO® Luciferase Assay System (Promega) according to the manufacturer specifications. Luminescence was measured by a SPECTRAMAX® M3 plate reader (Molecular Devices, San Jose, CA). Virus neutralization titers were defined as the sample dilution at which a 50% reduction in RLU was observed relative to the average of the virus control wells. Live virus neutralization assays described above were performed with approved standard operating procedures for SARS CoV-2 in a biosafety level 3 (BSL-3) facility.

Production of Fab fragments from monoclonal antibodies: To generate mAb-Fab, IgG was incubated with HRV3C protease (EMD Millipore) at a ratio of 100 units per 10 mg IgG with HRV 3C Protease Cleavage Buffer (150 mM NaCl, 50 mM Tris-HCl, pH 7.5) at 4° C. overnight. Fab was purified by collecting flowthrough from Protein A column (GE Health Science), and Fab purity was confirmed by SDS-PAGE.

Determination of binding kinetics of Fab: A FORTEBIO® OCTET® HTX instrument was used to measure binding kinetics of the Fab of A23-58.1, B1-182.1, A19-46.1 and A19-61.1 to SARS CoV-2 S-2P protein. SA biosensors (FORTEBIO®) were equilibrated for >600 s in Blocking Buffer (1% BSA (Sigma) +0.01% Tween-20 (Sigma)+0.01% Sodium Azide (Sigma)+PBS (Gibco), pH7.4) prior to loading with biotinylated S-2P protein (1.5 mg/mL in Blocking Buffer) for 600s. Following loading, sensors were incubated for 420s in Blocking Buffer prior to binding assessment of the Fabs. Association of Fabs was measured for 300 s and dissociation was measured for up to 3,600 s in Blocking Buffer. All the assays were performed with agitation set to 1,000 rpm at 30° C. Data analysis and curve fitting were carried out using OCTET® analysis software, version 11-12. Experimental data were fitted using a 1:1 binding model. Global analyses of the complete data sets assuming binding was reversible (full dissociation) were carried out using nonlinear least-squares fitting allowing a single set of binding parameters to be obtained simultaneously for all concentrations used in each experiment.

Negative-stain electron microscopy: Protein samples were diluted to a concentration of approximately 0.02 mg/ml with 10 mM HEPES, pH 7.4, supplemented with 150 mM NaCl. A 4.8-pl drop of the diluted sample was placed on a freshly glow-discharged carbon-coated copper grid for 15 seconds (s). The drop was then removed with filter paper, and the grid was washed with three drops of the same buffer. Protein molecules adsorbed to the carbon were negatively stained by applying consecutively three drops of 0.75% uranyl formate, and the grid was allowed to air-dry. Datasets were collected using a Thermo Scientific Talos F200C transmission electron microscope operated at 200 kV and equipped with a Ceta camera. The nominal magnification was 57,000×, corresponding to a pixel size of 2.53 Å, and the defocus was set at ˜1.2 μm. Data was collected automatically using EPU. Single particle analysis was performed using CRYOSPARC™ (Punjani et al., Nat. Methods. 14, 290-296 (2017)).

Cryo-EM specimen preparation and data collection: The stabilized SARS CoV-2 spike HexaPro (Hseih et al., Science. 369, 1501-1505 (2020) was mixed with Fab A23-58.1 or B1-182.1 at a molar ratio of 1.2 Fab per protomer in PBS. The final spike protein concentration was 0.5 mg/ml. n-Dodecyl $-D-maltoside (DDM) detergent was added shortly before vitrification to a concentration of 0.005%. Quantifoil R 2/2 gold grids were subjected to glow discharging in a PELCO EASIGLOW™ device (air pressure: 0.39 mBar, current: 20 mA, duration: 30 s) immediately before specimen preparation. Cryo-EM grids were prepared using an FEI VITROBOT® Mark IV plunger with the following settings: chamber temperature of 4° C., chamber humidity of 95%, blotting force of −5, blotting time of 3 s, and drop volume of 2.7 μl. Datasets were collected on a Thermo Scientific Titan Krios G3 electron microscope equipped with a GATAN® QUANTUM GIF® energy filter (slit width: 20 eV) and a GATAN® K3 direct electron detector. Four movies per hole were recorded in the counting mode using Latitude software. The dose rate was 14.65 e-/s/pixel.

Cryo-EM data processing and modelfitting: Data process workflow, including Motion correction, CTF estimation, particle picking and extraction, 2D classification, ab initio reconstruction, homogeneous refinement, heterogeneous refinement, non-uniform refinement, local refinement and local resolution estimation, were carried out with C1 symmetry in CRYOSPARC® 2.15 (Punjani et al., Nat. Methods. 14, 290-296 (2017)). For local refinement to resolve the RBD-antibody interface, a mask for the entire spike-antibody complex without the RBD-antibody region was used to extract the particles and a mask encompassing the RBD-antibody region was used for refinement. The overall resolution was 3.39 Å and 3.15 Å for the map of A23-58.1- and B1-182.1-bound spike, 3.89 Å and 3.71 Å for the map of RBD:antibody interface after local refinement, respectively. The coordinates for the SARS-CoV-2 spike with three ACE2 molecules bound at pH 7.4 (PDB ID: 7KMS) were used as initial models for fitting the cryo-EM map. Iterative manual model building and real space refinement were carried out in Coot (Emsley et al., Acta Crystallogr. Sect. D Biol. Crystallogr. 60, 2126-2132 (2004)) and in PHENIX® (Afonine et al., Acta Crystallogr. Sect. D Biol. Crystallogr. 68, 3-367 (2012)), respectively. Molprobity (Davis et al., Nucleic Acids Res. 32, 615-619 (2004)) was used to validate geometry and check structure quality at each iteration step. UCSF Chimera and ChimeraX were used for map fitting and manipulation (Pettersen et al., J. Comput. Chem. 25, 1605-1612 (2004)).

Selection of rcVSV SARS CoV-2 virus escape variants using monoclonal antibodies: A replication competent vesicular stomatitis virus (rcVSV) with its native glycoprotein replaced by the Wuhan-1 spike protein (rcVSV SARS CoV-2) that contains a 21 amino acid deletion at the C-terminal region (Dieterle et al., Cell Host Microbe. 28, 486-496.e6 (2020)). Passage 7 virus was passaged twice on Vero cells to obtain a polyclonal stock. A single plaque from this₉ₜh passage was double plaque purified and expanded on Vero cells to create monoclonal virus population. The reference genome for this stock was sequence using Illumina-based sequencing as described below.

To select for virus escape variants, an equal volume of clonal population of rcVSV SARS CoV-2 was mixed with serial dilutions of antibodies (5-fold) in DMEM supplemented with 10% FCS and Glutamine to give an MOI of 0.1-0.001 at the desired final antibody concentration (range 5.le-6 to 50 mg/ml and 0 mg/ml). Virus:antibody mixtures were incubated at room temperature for 1 hour. After incubation, 300 μl of virus:antibody mixtures were added to 1×10⁵ Vero E6 cells in 12 well plates for 1 hour at 37° C., 5% CO₂. The plates were rotated every 15 minutes to prevent drying. After absorption, 700 μl of additional antibodies mixture was added to each well at their respective concentration. Cells were incubated for 72 hrs at 37° C., 5% CO₂. Virus replication was monitored using cytopathic effect and supernatant was collected from the wells with cytopathic effect. Harvested supernatant was clarified by centrifugation at 3750 rpm for 10 minutes. For the subsequent rounds of selection, clarified supernatant from the well with the highest concentration of antibody that has CPE >20% supernatant was diluted prior to being mixed with equal volume of antibodies as in the initial round of selection. Infection, monitoring and collection of supernatants was performed as in the initial round.

Shotgun sequencing of rcVSV SARS CoV2 supernatants: Total RNA was extracted from clarified supernatants using QIAMP® viral RNA mini extraction kit (Qiagen) following the manufacturer's recommended protocol. Purified RNA was fragmented using NEBNEXT® Ultra II RNA Library Prep reagents, then reverse transcribed using random hexamers, and double-stranded cDNA was synthesized (New England BioLabs) as previously described (Ssemwanga et al., J. Infect. Dis. 217, 1530-1534 (2018)). Double-stranded cDNA was purified using magnetic beads (MAGBIO GENOMICS, INC.®) and barcoded ILLUMINA®-ready libraries were subsequently prepared (New England BioLabs). The libraries were sequenced as paired-end 2×150 base pair NextSeq 2000 reads.

Spike SNP variant calls of rcVSV antibody induced revertants: Raw sequencing reads were demultiplexed and trimmed to remove adaptor sequences and low quality bases. They were then aligned against the reference viral genome with Bowtie (v2.4.2). Single nucleotide polymorphisms (SNPs) were called using HaplotypeCaller from the Genome Analysis Tool Kit (GATK, v4.1.9.0). The HaplotypeCaller parameter, “—sample-ploidy”, was set to 100 in order to identify SNPs with a prevalence of at least 1%. SNPs for all samples were then aggregated, interrogated and translated using custom scripts. A SNP and correlated amino acid translation for the spike protein was considered positive if it was present at a frequency of greater than 0.1 (10%) and showed an increasing frequency from round 1 to round 2 of the antibody selections.

Multiplex SAR2 variant binding assay: Multiplexed Plates (96 well) precoated with SARS Cov2 spike (WA-1), SARS Cov2 RBD (WA-1), SARS Cov2 spike (B.1.351), SARS Cov2 spike (B.1.1.7), SARS Cov2 spike (P.1), SARS Cov2 RBD (B.1.351), SARS Cov2 RBD (B.1.1.7), SARS Cov2 RBD (P.1) and BSA are supplied by the manufacturer. On the day of the assay, the plate is blocked for 60 minutes with MSD Blocker A (5% BSA). The blocking solution is washed off and test samples are applied to the wells at 4 dilution (1:100, 1:500, 1:2500 and 1:10,000) unless otherwise specified and allowed to incubate with shaking for two hours. Plates are washed and Sulfo-tag labeled anti IgG antibody is applied to the wells and allowed to associate with complexed coated antigen-sample antibody within the assay wells. Plates were washed to remove unbound detection antibody. A read solution containing ECL substrate was applied to the wells, and the plate is entered into the MSD Sector instrument. A current was applied to the plate and areas of well surface where sample antibody has complexed with coated antigen and labeled reporter emitted light in the presence of the ECL substrate. The MSD Sector instrument quantitated the amount of light emitted and reported this ECL unit response as a result for each sample and standard of the plate. Magnitude of ECL response was directly proportional to the extent of binding antibody in the test article. All calculations were performed within Excel and the GraphPad Prism software, version 7.0. Readouts were provided as Area Under Curve (AUC).

### Example 23

### Cryo-EM Structure of B.1.1.529 (Omicron) Spike

To provide insight into the impact of B.1.1.529 mutations on spike, produced the two proline-stabilized (S2P) (Wrapp et al., Science. 367, 1260-1263 (2020)) B.1.1.529 spike were expressed and produced, and single particle cryo-EM data was collected to obtain a structure of the trimeric ectodomain at 3.29 Å resolution (FIG. 22A), see also the table below.

TABLE
Cryo-EM Data Collection, Refinement and Validation Statistics
for SARS COV-2 Spike and Antibody Complexes.

| | | | | | |
|---|---|---|---|---|---|
| | | SARS- | | | |
| | SARS- | CoV-2 | SARS- | | |
| | CoV-2 | Omicron | CoV-2 | SARS- | |
| | Omicron | spike | Omicron | CoV-2 | B1- |
| | spike | in complex | spike | spike in | 182.1:A19- |
| SARS- | in | with A19- | with A19- | complex | 61.1:RBD |
| CoV-2 | complex | 46.1 after | 46.1 | with B1- | complex |
| Omicron | with A19- | local | and B1- | 182.1 and | after local |
| spike | 46.1 | refinement | 182.1 | A19-61.1 | refinement |
| (EMD- | (EMD- | (EMD- | (EMD- | (EMD- | (EMD- |
| 25792) | 25807) | 25806) | 25808) | 25794) | 25797) |
| (PDB | (PDB | (PDB | (PDB | (PDB | (PDB |
| 7TB4) | 7TCA) | 7TC9) | 7TCC) | 7TB8) | 7TBF) |

Data collection
and processing

| Magnification | 105,000 | 105,000 | | 105,000 | 105,000 | |
|---|---|---|---|---|---|---|
| Voltage (kV) | 300 | 300 | | 300 | 300 | |
| Electron exposure | 40.0 | 40.0 | | 40.0 | 40.0 | |
| (e⁻/Å²) | | | | | | |
| Defocus range (μm) | −1.0 to −2.5 μm | −1.0 to −2.5 μm | | −1.0 to −2.5 μm | −1.0 to −2.5 μm | |
| Pixel size (Å) | 0.855 | 0.855 | | 0.855 | 0.873 | |
| Symmetry imposed | C1 | C1 | | C1 | C1 | |
| Final particle | 266434 | 79077 | | 46244 | 358526 | |
| images (no.) | | | | | | |
| Map resolution (Å) | 3.29 | 3.85 | 5.08 | 3.86 | 2.83 | 3.10 |
| FSC threshold | 0.5 | 0.5 | | 0.5 | 0.5 | 0.5 |
| Refinement | | | | | | |
| Initial model used | 7MM0 | 7TB4 | 7TB4 | 7TB4 | 7MM0 | 7TB8 |
| (PDB code) | | | | | | |
| Model resolution | 3.15 | 3.29 | 3.29 | 3.29 | 3.15 | 2.83 |
| (Å) | | | | | | |
| FSC threshold | 0.5 | 0.5 | 0.5 | 0.5 | 0.5 | 0.5 |
| Map sharpening B | −130.7 | −94.1 | −282 | −70.5 | −61.8 | −46.5 |
| factor (Å²) | | | | | | |
| Model composition | | | | | | |
| Non-hydrogen | 26899 | 33706 | 4298 | 37713 | 33230 | 6617 |
| atoms | | | | | | |
| Protein residues | 3329 | 4244 | 642 | 4749 | 4189 | 857 |
| Ligands | 59 | 56 | 1 | 57 | 43 | 2 |
| B factors | | | | | | |
| (Å²)(mean) | | | | | | |
| Protein | 97.2 | 214.4 | 178.0 | 74.3 | 76.0 | 51.1 |
| Ligand | 97.4 | 151.5 | 154.5 | 170.39 | 60.0 | 52.0 |
| R.m.s. deviations | | | | | | |
| Bond lengths (Å) | 0.005 | 0.002 | 0.003 | 0.006 | 0.003 | 0.007 |
| Bond angles (°) | 0.655 | 0.587 | 0.735 | 0.742 | 0.620 | 0.966 |
| Validation | | | | | | |
| MolProbity score | 1.95 | 1.97 | 2.45 | 2.35 | 1.86 | 2.11 |
| Clash score | 7.9 | 9.7 | 24.4 | 18.8 | 7.3 | 8.3 |
| Poor rotamers | 0.1 | 0.03 | 0 | 0.02 | 0 | 0 |
| (%) | | | | | | |
| Ramachandran plot | | | | | | |
| Favored (%) | 91.2 | 92.6 | 89.3 | 88.9 | 92.8 | 85.0 |
| Allowed | 8.5 | 7.1 | 10.1 | 10.8 | 6.9 | 13.9 |
| Disallowed | 0.3 | 0.3 | 0.6 | 0.3 | 0.3 | 1.2 |

Like other D614G containing variants, the most prevalent spike conformation comprised the single-receptor-binding domain (RBD)-up conformation (Yurkovetskiy et al., Cell. 183, 739-751.e8 (2020)). B.1.1.529 mutations present in the spike gene resulted in 3 deletions of 2, 3 and 1 amino acids, a single insertion of 3 amino acids and 30 amino acid substitutions in the spike ectodomain. As expected from the ˜3% variation in sequence, the B.1.1.529 spike structure was extremely similar to the WA-1 spike structure with an overall Ca-backbone RMSD of 1.8 Å (0.5 Å for the S2 region); however, minor conformational changes were observed in a few places. For example, the RBD S371L/S373P/S375F substitutions changed the conformation of their residing loop, with Phe375 in the RBD-up protomer formed Phe-Phe interaction with Phe486 in the neighboring RBD-down protomer (FIG. 22B), helping to stabilize the single-RBD-up conformation. Amino acid changes were denser in the N-terminal domain (NTD) and RBD, where a majority of neutralization occurs, though RMSDs remained low (0.6 Å and 1.2 Å for NTD and RBD, respectively). Notably, about half the B.1.1.529 alterations in sequence outside NTD and RBD involved new interactions, both hydrophobic, such as Tyr796 with glycan on Asn709, and electrostatic, such as Lys547 and Lys856 interacting with residues in HR1 SD1 on neighboring protomers (FIG. 22B, see also the table below).

TABLE
B.1.1.529 mutation introduced hydrogen bonds and salt bridges.

| | | | |
|---|---|---|---|
| ## | Protomer 1 | Dist. [Å] | Protomer 2 |

Hydrogen bonds

| | | | |
|---|---|---|---|
| 1 | C:SER 982[OG | 2.51 | B:LYS 547[NZ] |
| 2 | C:LYS 856[NZ] | 2.60 | B:THR 572[OG1] |
| 3 | B:LYS 764[NZ] | 2.78 | A:GLN 314[OE1] |
| 4 | B:LYS 856[NZ] | 2.99 | A:THR 572[OG1] |
| 6 | C:LYS 547[NZ] | 3.89 | A:ASN 978[OD1] |

Salt bridges

| | | | |
|---|---|---|---|
| 1 | B:LYS 856[NZ] | 3.94 | A:ASP 568[OD2] |
| 2 | C:ASP 571[OD2] | 3.42 | A:LYS 856[NZ] |

These heightened interprotomer interactions suggested a need to maintain trimer stability. Differential scanning calorimetry indicated the B.1.1.529 spike to have folding energy similar to the original WA-1 strain.

NTD changes altered ˜6% of the solvent accessible surface on this domain, and several were located directly on or proximal to the NTD-supersite of vulnerability (Cerutti et al., Cell Host Microbe. 29, 1-15 (2021)), where prior variants had mutations that substantially reduced neutralization by NTD antibodies. Other NTD changes were proximal to a pocket, proposed to be the site of bilirubin binding (Ross et al., Sci. Adv. 7, 1-15 (2021)), which also binds antibody (Cerutti et al., Cell Rep. 37, 109928 (2021)) (FIG. 22C).

RBD alterations changed ˜16% of the solvent accessible surface on this domain and were constrained to the outward facing ridge of the domain (FIG. 22D), covering much of the surface of the trimeric spike apex. Several amino acid changes involved basic substitutions, resulting in a substantial increase in RBD electro-positivity (FIG. 22D). Overall, RBD changes were located proximal to binding surfaces for the ACE2 receptor (Lan et al., Nature. 581, 215-220 (2020)) (FIG. 22D) as well as to recognition sites for potently neutralizing antibodies (FIG. 22E) (Barnes et al., Nature. 588, 682-687 (2020); Robbiani et al., Nature. 584, 437-442 (2020); Wang et al., Science (80). 373, 0-15 (2021)).

### Example 24

### FUNCTIONAL Assessment of Variant Binding to ACE2

When pathogens infect a new species, sustained transmission leads to adaptations that optimize replication, immune-avoidance and transmission. One hypothesis for the efficient species adaptation and transmission of SARS-CoV-2 in humans is that the virus spikes are evolving to optimize binding to the host receptor protein, ACE2. As a first test of this hypothesis, a flow cytometric assay was used to evaluate binding of human ACE2 to cells expressing variant spike proteins. The binding of soluble dimeric ACE2 to B.1.1.7 (Rambaut et al, 2020, available on the internet, virological.org/t/preliminary-genomic-characterisation-of-an-emergent-sars-cov-2-lineage-in-the-uk-defined-by-a-novel-set-of-spike-mutations/563), B.1.351 (Beta) (Tegally et al., Nature. 592, 438-443 (2021), P.1 (Gamma) (Faria et al., 2021, available on the internet, virological.org/t/genomic-characterisation-of-an-emergent-sars-cov-2-lineage-in-manaus-preliminary-findings/586; Naveca et al., 2021, available on the internet at virological.org/t/phylogenetic-relationship-of-sars-cov-2-sequences-from-amazonas-with-emerging-brazilian-variants-harboring-mutations-e484k-and-n501y-in-the-spike-protein/585) or B.1.617.2 (Delta) (WHO, COVID-19 weekly epidemiological update. World Heal. Organ., 1-23 (2021)) spikes was evaluated and compared to the ancestral D614G spike. The earliest variants, B.1.1.7, B.1.351 and P.1, first recognized in December 2020 and January 2021 contain an RBD mutation at N501Y, which has been proposed to increase binding to ACE2 (Starr, et al., Cell. 182, 1295-1310.e20 (2020)). Cell surface ACE2 binding to B.1.1.7 was 124% of D614G, while B.1.351, and P.1 were 69% and 76%, respectively, demonstrating that these variants do not have a substantial increase in ACE2 affinity. Next, binding was evaluated to the B.1.1.529 spike protein and observed ACE2 binding to be 200% that of D614G variant.

Since cell-surface binding may involve other factors, such as might be impacted by the increased electro-positivity of the RBD noted above, and formal kinetic measurements are challenging to obtain using cell-based assays, ACE2 binding affinity was further investigated using surface plasmon resonance measurements of soluble dimeric human ACE2 to S2P spike trimers generated from the ancestral WA-1 and 6 subsequent variants: D614G, B.1.351, P.1, B.1.617.2, B.1.1.7 and B.1.1.529. It was observed that both WA-1 and D614G, which have identical RBD sequences, have similar affinities (K_{D}=1.1 nM and 0.73 nM, respectively). It was noted that the affinity for B.1.617.2, which contains two RBD mutations, was ˜3-fold worse (KD=2.4 nM) than the D614G S2P trimer. B.1.1.7, B.1.351, P.1 and B.1.1.529, which each contain N501Y substitutions, were evaluated and affinities of 0.59 nM, 1.7 nM, 0.85 nM and 3.8 nM, respectively, were found. Collectively, the cell surface and S2P binding results show minimal improved affinity in some, but not all spikes, and suggests that, while SARS-CoV-2 maintains nanomolar affinity to ACE2, spike variant evolution appears to be driven primarily by immune pressure.

### Example 25

### Variant Binding and Neutralization by Individual Monoclonal Antibodies

To define of the impact SARS CoV-2 variant amino acid changes on the binding and neutralization of monoclonal antibodies, 17 highly potent antibodies targeting the spike RBD (Barnes et al., Nature. 588, 682-687 (2020); Robbiani et al., Nature. 584, 437-442 (2020); Ryu et al. Biochem. Biophys. Res. Commun. 578, 91-96 (2021); Kim et al., Nat. Commun. 12, 1-10 (2021).; Rappazzo et al., Science. 371, 823-829 (2021); Wetendorf et al., LY-CoV1404 potently neutralizes SARS-CoV-2 variants (2021); Tortorici et al., Science. 370, 950-957 (2020); Jones et al., Sci. Transl. Med. (2021), doi:10.1126/scitranslmed.abf1906; Shi et al., Nature. 584, 120-124 (2020); Hansen et al., Science. 369, 1010-1014 (2020); Zoost et al., Nature. 584, 443-449 (2020); Pinto et al., Nature. 583, 290-295 (2020); Piccoli et al., Cell. 183, 1024-1042.e21 (2020); Dejnirattisai et al., bioRxiv, in press, doi:10.1101/2021.12.03.471045; Greaney et al., Nat. Commun. 12 (2021), doi:10.1038/s41467-021-24435-8); including 13 antibodies currently under clinical investigation or approved for use under expanded use authorization (EUA) by the United States Food and Drug Administration were expressed and purified. All antibodies bound and neutralized B.1.1.7 comparable to the ancestral D614G and consistent with the single 501Y substitution being outside each antibody's binding epitope (FIG. 23A-23C). The addition of two more RBD substitutions, K417N and E484K (FIG. 23A) in the B.1.351 and P1 variants eliminated binding by two Class I antibodies, CB6 and REGN10933 and two Class II antibodies, LY-CoV555 and C144 (FIG. 23B). Neutralization of B.1.351 and P1 by CB6, LY-CoV555 and C144 was completely abolished, while REGN10933 was eliminated for B.1.351 and reduced >250-fold for P.1. In addition, while binding of CT-P59 to B.1.351 and P.1 variants was minimally changed (69-79%), neutralization was decreased 26-43-fold (IC₅₀ 65.8 and 39.6 ng/mL) (FIG. 23B, 23C). The remaining antibodies showed minimal binding changes and a <3.6-fold difference in neutralization IC₅₀ (FIG. 23B, 23C). An evaluation of the antibodies in the panel against B.1.617.2 revealed minimal changes in binding for all antibodies except A19-46.1 and LY-CoV555, which were 0% of D614G (FIG. 23B). Neutralization assays using B.1.617.2 pseudovirus showed that the REGN10987 IC₅₀ was 22.7-fold lower than D614G and neutralization values for A19-46.1 and LY-CoV555 were each >10,000 ng/mL (FIG. 23C). These data are consistent with previous results that showed both A19-46.1 and LY-CoV555 were sensitive to the L452R mutations present in B.1.617.2 (Wang et al., Science (80) 373, 0-15 (2021)).

For B.1.1.529, it was noted that all but three antibodies showed binding less than 31% of D614G. It is interesting to note that COV2-2196, S2E12, B1-182.1 and A23-58.1 utilize the same VH1-58 gene in their heavy chain and target a similar region on the RBD (i.e., VH1-58 supersite), but show differential binding to the B.1.1.529 (i.e., 4%, 5%, 8% and 11%, respectively) and B.1.617.2 (i.e., 66%, 67%, 77% and 85%, respectively) (FIG. 23B). Even though the absolute differences in binding are minimal, the shared trend may be reflective of how the RBD tip mutation at T478K mutation is accommodated by each of these antibodies. Finally, LY-CoV1404 revealed 61% binding to B.1.1.529 spike. Taken together, cell surface binding suggests that while both A19-46.1 and LY-CoV1404 are likely to retain potent neutralizing activity against B.1.1.529, the remaining antibodies in our panel might show decreased neutralizing activity.

Using the same panel of monoclonal antibodies, each antibody's capacity to neutralize the B.1.1.529 variant was further assayed. While VH1-58 supersite antibodies (Class I) show high neutralization activity against other variants, antibodies targeting the supersite were 40 to 126-fold worse (IC₅₀ 38-269 ng/ml) against B.1.1.529 viruses than D614G (IC₅₀ 0.9-2.0 ng/ml) (FIG. 23C). In addition, two other antibodies, CB6 (Class I) and ADG2 (Class I/IV) were shown to be severely impacted (IC₅₀>10,000 ng/mL CB6 and 2037 ng/mL ADG2 to B.1.1.529 vs 31 and 50.5 ng/mL to D614G, respectively) (FIG. 23C). Class II antibodies (i.e., LY-CoV555, C144, A19-46.1) were next analyzed and it was found that amongst these, neutralization by LY-CoV555 and C144 was completely abolished (IC₅₀>10,000 ng/mL B.1.1.529 vs 3.6 and 5.1 ng/mL D614G, respectively). In contrast, it was found that the A19-46.1 IC₅₀ neutralization was 223 ng/mL for B.1.1.529 vs 19.4 ng/mL for D614G (FIG. 23C) and was <6 fold of the previously reported IC₅₀ for WA-1 (39.8 ng/mL) (Wang et al., Science (80) 373, 0-15 (2021)). The Class III antibodies (i.e., A19-61.1, REGN10987, COV2-2130, C135, LY-CoV1404) were analyzed and it was noted that neutralization activity of A19-61.1, REGN10987 and C135 was completely abolished (IC₅₀>10,000 ng/mL B.1.1.529 vs 19.4, 20.0, 10.8 ng/mL, respectively on D614G), CoV2-2130 decreased 1581-fold (IC₅₀ 5850 ng/mL B.1.1.529 vs 3.7 ng/mL D614G) and that of S309 decreased by -8-fold (IC₅₀ 281 ng/mL B.1.1.529 vs 36.1 ng/mL D614G) (FIG. 23C). Strikingly, in contrast to all of the other antibodies, it was found that the neutralization of LY-CoV1404 against B.1.1.529 was unchanged relative to D614G (IC₅₀ 5.1 ng/mL for B.1.1.529 vs 3 ng/mL for D614G) (FIG. 2C). Taken together, these data demonstrate that the mutations present in B.1.1.529 mediate resistance to antibodies.

### Example 26

### Structural and Functional Basis of Class I Antibody Neutralization, Escape and Retained Potency

It was sought to determine the functional basis of B.1.1.529 neutralization and escape for Class I antibodies and to understand how potent neutralization might be retained. Class I antibodies, CB6, B1-182.1 and S2E12, were analyzed with differential B.1.1.529 neutralization (FIG. 23C). CB6 was first analyzed using virus particles containing single amino acid substitutions representing 13 of 15 single amino acid changes on the RBD of B.1.1.529 (i.e., all but S375F and G496S) or G496R; while several minimally changed their neutralization IC₅₀, only Y505H, S371L, Q493R and K417N decreased neutralization >5-fold, with IC₅₀ of 50, 212, 320, and >10,000 ng/mL, respectively (FIG. 24A). This suggests that B.1.1.529 evades CB6-like antibodies through multiple mutations. Docking of the RBD-bound CB6 onto the B.1.1.529 structure revealed several B.1.1.529 residues may potentially clash with CB6. Especially, K417N, Q493R and Y505H were positioned to cause severe steric hindrance to the CB6 paratope, consistent with the neutralization data (FIG. 24B). Two VH1-58 supersite antibodies, B1-182.1 and S2E12, which have highly similar amino acid sequences but show ˜6-fold difference in B.1.1.529 neutralizing, were analyzed. These two antibodies remained highly potent (<10.6 ng/mL IC₅₀) for all virus particles with single RBD mutations, with the largest change for Q493R, which caused a 7 and 5.4-fold decrease of neutralization for B1-182.1 and S2E12, respectively. These small differences in neutralization from single mutations suggest that multiple mutations of B.1.1.529 are working in concert to mediate escape from VH1-58 supersite antibodies. Docking of the RBD-bound B1-182.1 onto the B.1.1.529 structure indicated that the epitopes of these VH1-58-derived antibodies were confined by Q493R, S477N, T478K and E484A (FIG. 24C). With R493 pressing on one side of the antibody like a thumb, N477/K478 squeezed onto the other side of the antibody at the heavy-light chain interface like index and middle fingers (FIG. 24C). Analysis of the docked RBD-antibody complex showed that N477/K478 positioned at the junction formed by CDR H3, CDR L1 and L2 with slight clashes to a region centered at CDR H3 residue 100C (Kabat numbering) (FIG. 24D). Sequence alignment of CDR H3 of VH1-58-derived antibodies indicated that residue 100C varied in sidechain sizes, from serine in S2E12 to tyrosine in A23-58.1. Analysis showed that size of 100C reversely correlated with neutralization potency IC₈₀ (p=0.046) (FIG. 24D, FIG. 23C), suggesting VH1-58 antibodies could alleviate escape imposed by the B.1.1.529 mutations through reduced side chain size at position 100C to minimize clashes from N477/K478.

### Example 27

### Structural and Functional Basis of Class II Antibody Neutralization, Escape and Retained Potency

The functional basis of B.1.1.529 neutralization and escape was determined for two Class II antibodies, LY-CoV555 (Jones et al., Sci. Transl. Med. (2021), doi:10.1126/scitranslmed.abf1906) and A19-46.1 (Wang et al., supra), which have B.1.1.529 IC₅₀ of >10,000 and 223 ng/mL, respectively (FIG. 23C). By assessing the impact of each of the single amino acid changes in RBD from B.1.1.529, it was found that for LY-CoV555, either E484A or Q493R resulted in complete loss of LY-CoV555 neutralization (IC₅₀>10,000 ng/mL) (FIG. 25A), while the same mutations did not affect A19-46.1. For A19-46.1, no individual mutation reduce neutralization to the level noted in B.1.1.529; S371L had the highest effect, reducing the IC₅₀ to 72.3 ng/mL relative to 223 ng/mL for B.1.1.529. One potential explanation for this is that the Phe-Phe interaction, between 375 and 486, occurs in the context of three B1.1.529 alterations, S371L/S373P/S375F (FIG. 22B).

To understand the structural basis of A19-46.1 neutralization of B.1.1.529, we obtained cryo-EM structure of the B.1.1.529 spike in complex with Fab A19-46.1 at 3.86 Å resolution (FIG. 25B). The structure revealed that two Fabs bound to the RBD in the “up” conformation in each spike with the third RBD in down position. Focused local refinement of the antibody-RBD region resolved the antibody-RBD interface (FIG. 25B, right). Consistent with previous mapping and negative stain EM data, A19-46.1 binds to a region on RBD generally targeted by the Class II antibodies with an angle approximately 45 degrees towards the viral membrane. Binding involves all light chain CDRs and only CDR H3 of the heavy chain and buried a total of 805 Å² interface area from the antibody (FIG. 25C, left). With the light chain latching to the outer rim of the RBD and providing about 70% of the binding surface, A19-46.1 uses its 17-residue-long CDR H3 to form parallel strand interactions with RBD residues 345-350 (FIG. 25B, right) like a sway brace. Docking RBD-bound ACE2 to the A19-46.1-RBD complex indicated that the bound antibody sterically clashes with ACE2 (FIG. 25D), providing the structural basis for its neutralization of B.1.1.529.

The 686 Å2 epitope of A19-46.1 is located within an RBD region that lacks amino acid changes found in B.1.1.529. Of the 15 amino acid changes on RBD, three of residues, S446, A484 and R493, positioned at the edge of epitope with their side chains contributing 8% of the binding surface. LY-CoV555, which targets the same region as a class II antibody, completely lost activity against B.1.1529. To gain structural insights on the viral escape of LY-CoV555, the LY-CoV555-RBD complex was superimposed onto the B.1.1.529 RBD. Even though LY-CoV555 approached the RBD with similar orientation to that of A19-46.1 (FIG. 25E), its epitope shifted up to the ridge of the RBD and embraced B.1.1.529 alterations A484 and R493 within the boundary (FIG. 25E). Inspection of the superimposed structures indicated B.1.1.529 alteration R493 caused steric clash with the CDR H3 of LY-CoV555, explaining the escape of B.1.1.529 from LY-CoV555 neutralization. Overall, the location of the epitope and the angle of approach allowed A19-46.1 to effectively neutralize B.1.1.529.

### Example 28

### Structural and Functional Basis of Class III Antibody Neutralization, Escape and Retained Potency

To evaluate the functional basis of B.1.1.529 neutralization and escape for Class III antibodies and to understand how potent neutralization might be retained, a panel of Class III antibodies with differential potency, including A19-61.1, COV2-2130, S309 and LY-CoV1404 (FIG. 26A) was investigated. Assessment of the impact of each of the 15 mutations in RBD revealed the G446S amino acid change results in a complete loss in activity for A19-61.1; consistent with the complete loss of function of this antibody against B.1.1.529. For S309, S373P and G496R were observed to result in small changes to neutralization. Surprisingly, while 5309 retains moderate neutralizing activity against B.1.1.529, it was found found the S371L amino acid change to abolish 5309 neutralization. This suggests that combinations of S371L with other B.1.1.529 mutations can result in structural changes in spike that allows 5309 to partially overcome the S371L change. The evaluation of COV2-2130 did not identify significant differences in neutralization, suggesting a role for an untested mutation or combinations of amino acid changes for the decrease in neutralization potency observed against the full virus. Finally, consistent with the overall high potency of LY-CoV1404 against all tested VOCs, an amino acid change was not identified that impacted its function.

To understand the structural basis of Class III antibody neutralization and viral escape, the cryo-EM structure of WA-1 S2P was determined in complex with Fab A19-61.1 (and Fab B1-182.1 to aid EM resolution of local refinement) at 2.83 Å resolution (FIG. 26B). The structure revealed that two RBDs were in the up-conformation with both antibodies bound, and the third RBD was in the down-position with only A19-61.1 bound, indicating A19-61.1 could recognize RBD in both up and down conformation (FIG. 26B). Local refinement of the RBD-Fab A19-61.1 region showed that A19-61.1 targeted the Class III epitope with interactions provided by the 18-residue-long CDR H3 from the heavy chain, and all CDRs from the light chain (FIG. 26B). Docking the A19-61.1 structure to the B.1.1.529 spike structure indicated B.1.1.529 mutations S446, R493 and S496 might interfere with A19-61.1. Analysis of the side chain interaction identified Y111 in CDR H3 posed severe clash with S446 in RBD that could not be resolved by loop flexibility (FIG. 26C), explaining the loss of A19-61.1 neutralization against G446S-containing SARS-CoV-2 variants.

Neutralization assays indicated that COV2-2130, S309 and LY-CoV1404 retained neutralization potency against B.1.1.529. Docking of CoV2-2130 indicated that CoV2-2130 targeted a very similar epitope to that of A19-61.1 with interactions mainly mediated by its CDR L1 and L2 and avoiding close contact with R493 and S496. However, the OH group at the tip of Y50 in CDR L2 posed a minor clash with S446 in RBD, explaining the structural basis for the partial conservation of neutralization by CoV2-2130 (FIG. 26D). Antibody S309 showed higher potency against B.1.1.529 than CoV2-2130. Docked complex of S309 and RBD showed the G339D mutation is located inside the epitope and clashes with CDR H3 Y100, however, the void space between S309 and RBD might accommodate an alternate tyrosine rotamer. The S371L/S373P/S375F mutations changed the conformation of their residing loop and may push the glycan on N343 towards S309 to reduce binding (FIG. 26E). LY-CoV1404 was not affected by B.1.1.529 mutations. Docking of the LY-CoV1404 onto the B.1.1.529 RBD identified four amino acid substitutions located at the edge of its epitope. Three of the residues, K440, R498 and Y501, only make limited side chain interactions with LY-CoV1404. The 4^{th} residue, G446S, appeared to cause a potential clash with CDR H2 R60. However, comparison of both LY-CoV1404-bound and non-bound RBD indicated the loop where S446 resided had conformational flexibility to allow LY-CoV1404 binding (FIG. 26F). Overall, the epitopes to Class III antibodies were mainly located on mutation-free RDB surfaces with edges contacting a few B.1.1.529 alterations (FIG. 26G). LY-CoV1404 retained high potency by accommodating all four B.1.1.529 alteration at edge of its epitope by exploiting loop mobility or by minimizing side chain interactions.

### Example 29

### Synergistic Neutralization by the Combination of B1-182-1 and A19-46.1

The combination of B1-182.1 and either A19-46.1 or A19-61.1 mitigated mutational escape in an in vitro virus escape assay; suggesting the possibility of synergistic neutralization. It was therefore hypothesized that there are combinations of antibodies which lead to an increase in B1.1.529 neutralization beyond that of the two antibodies alone. As a test of this hypothesis, the neutralization of B.1.1.529 pseudotyped viruses was determined by clinically utilized cocktails or various combinations of B1-182.1, A19-46.1, A19-61.1, LY-CoV1404, ADG2 and 5309. Of the 10 combinations evaluated only COV2-2196/COV2-2130, B1-182.1/A19-46.1 and B1-182.1/5309 neutralized B.1.1.529 with an appreciably improved potency (i.e., IC₅₀ of 50.8, 28.3 and 58.1 ng/mL) over the individual component antibodies (FIGS. 27A, 27B). Each of these utilized a V_{H}-158 supersite antibody and showed a 5 to 115-fold improvement over the component antibodies (FIG. 27B), suggesting an effect that is more than an additive for the specific combination against B.1.1.529.

To understand the structural basis of the improved neutralization by the cocktail of B1-182.1 and A19-46.1 the cryo-EM structure of the B.1.1.529 S2P spike in complex with Fabs of B1-182.1 and A19-46.1 was determined at 3.86 Å resolution (FIG. 27C). The prevalent 3D reconstruction revealed that the spike recognized by the combination of these two antibodies was the 3-RBD-up conformation with both Fabs bound to each RBD (Fabs on one of the RBDs were lower in occupancy). The spike had a 1.6 Å RMSD relative to the 3-RBD-up WA-1 structure (PDB ID: 7KMS). Overall, the structure showed that both Class I and II antibodies were capable of simultaneously recognizing the same RBD, and the combination increased the overall stoichiometry compared to two Fabs per trimer observed in the S2P-A19-46.1 structure described above. Of all the antibodies tested, all VH1-58-derived antibodies retained reasonable level of neutralization against B.1.1.529 while members of other antibody classes suffered complete loss of activity. VH1-58 antibodies have minimal numbers of impacting B.1.1.529 alterations in their epitopes and can evolve means to alleviate the impact. Without being bound by theory, it is possible that the binding of the first antibody induced the spike into RBD-up-conformation and facilitated binding of the second RBD-up-conformation preferring antibody, thereby synergistically increasing the neutralization potency of the cocktail compared to the individual antibodies.

SARS-CoV2 variants of concern provide a window into the co-evolution of key host-pathogen interactions between the viral spike, human ACE2 receptor and the human immune system. The RBD is a major target for neutralizing antibodies in both survivors and vaccinees. Since 15 of the 37 mutations in the B.1.1.529 variant spike reside within the RBD, there is a great need to understand the mechanisms by which RBD variations evolve, what constraints exist on the evolution and whether there are approaches that can be taken to exploit this understanding to develop and maintain effective antibody therapeutics and vaccines.

A series of functional and structural studies were used to define the mechanisms by which B.1.1.529 is either neutralized by or mediates escape from host immunity. To functionally frame our analyses, the Barnes classification, which categorizes antibodies based on their binding to the ACE2 binding site and the position of RBD, was used. The findings for Class I VH1-58 supersite showed that B.1.1.529 requires a series of mutation that are not individually deleterious to bracket the antibody and reduce its potency. The data suggests that VH1-58 antibodies can alleviate the deleterious impact of this pinching effect by reducing the size of CDR H3 residue 100C to avoid clashes from B.1.1.529 mutations.

For the Class II antibody A19-46.1, the angle of approach and a long-CDRH3 combine allow it to target the mutation-free face on RBD and minimize contacting the mutations on the ridge of B.1.1.529 RBD. It was observed that A19-46.1 binding requires the RBD-up conformation, and that the S371L substitution, which is located away from the A19-46.1 epitope and near the RBD hinge, partially reduces the neutralization of A19-46.1. Comparing the effect of S371L on neutralization by A19-46.1 and LY-CoV555 (FIG. 25A), which recognizes both RBD-up and -down conformation, suggested that L371 (and potentially P373/F375) is critical for controlling the RBD-up or -down conformation in B.1.1.529. This concept is supported by the finding that combination with a Class I antibody (such as B1-182.1) synergistically enhances A19-46.1 neutralization (FIG. 27A).

For Class III antibodies, only one prototype antibody showed complete loss of B.1.1.529 neutralization. Using structural and functional approaches it was determined that viral escape was mediated by the G446S amino acid change. This result indicates that potent Class III antibodies might be induced through structure-based vaccine designs that mask residue 446 in RBD. Additionally, the existence of G446S sensitive and resistant antibodies with significant epitope overlap suggest the use of spikes with G446S substitution can be utilized to evaluate the quality of Class III immune response in serum-based epitope mapping assays (Ko et al., PLOS Pathog. 17, e1009431 (2021)1 Corbett et al., Science (80-). 374, 1343-1353 (2021)).

The disclosed analyses evidence that S309 and COV2-2196 neutralized to similar degrees. Unlike other antibodies, the highly potent LY-CoV1404 does not lose neutralization potency against B.1.1.529. Combinations of antibodies were identified that show more than additive increases in neutralization against B.1.1.529, including COV2-2196/COV2-2130, B1-182.1/A19-46.1 and B1-182.1/S309. Each pair contains a VH1-58 supersite antibody that binds RBD in the up position. Without being bound by theory, pairing antibodies that neutralize better in the up-RBD conformation with these VH1-58 antibodies may provide a mechanism for better neutralization by the former. The S371L/S373P/S375F alterations in the RBD-up protomer form interprotomer interactions to RBD in the RBD-down protomer and stabilize the B.1.1.529 spike into a single-RBD-up conformation. RBD-up-preferring antibody like the VH1-58-derived B1-182.1, which is not affected by S371L substitution, can effectively break up the interaction to induce the 3-RBD-up conformation and therefore, enhance binding of other antibodies (such as A19-46.1) that require the RBD up-conformation. The identification of SARS-CoV-2 monoclonal antibodies that cooperatively function is supports the concept of using combinations to both enhance potency and mitigate the risk of escape.

FIG. 28 shows additional neutralization data.

### Example 30

### Additional Antibodies

Antibodies B1-182.1 and A23-58.1 were potent antibodies with broad neutralizing activity against SARS-CoV-2 variants. B1-182.1 and A23-58.1 have highly similar sequences but slight differences in neutralization potency, with B1-182.1 generally having better neutralizing activity compared to A23-58.1. However, following transient transfection of both antibodies, it was noted that A23-58.1 has a higher yield (i.e., total amount) and higher capacity of concentration (FIG. 29A). In addition, during antibody purification, B1-182.1 precipitates in large quantities (FIG. 29A), while A23-58.1 does not precipitate. In order to improve yield of B1.182.1, increase the ability of B1.182.1 to be concentrated and to mitigate the B1-182.1 precipitation issues, the features of A23-58.1 were to design antibodies that were variations of B1-182.1. Antibodies that were B1-182.1 variations were identified that maintain or improve the potency over the parental B1-182.1 antibody against SARS-CoV-2 variants.

To determine whether the changes should focus on the heavy chain, light chain or both, two variant antibodies were designed: (1) B1-182.1 with the CDRH3 region of B1-182.1 replaced with the CDRH3 region from A23-58.1 (B1-182.1_58.1CDRH3 heavy/B1-182.1 light); (2) B1-182.1 heavy with B1-182.1 light chain with G50S, F58I, Y87F, N93T and R107K (Kabat numbering) changes (B1-182 heavy/B1-182.1 light_5Mut). These sequences are shown below:

| | |
|---|---|
| Name: | B1-182.1_58.1CDRH3 heavy/B1-182.1 light |

Heavy Chain
QMQLVQSGPEVKKPGTSVKVSCKASGFTFTSSAVQWVRQARGQRLEWIGWIVVGSGNTNYAQK
FQERVTITRDMSTSTAYMELSSLRSEDTAVYYCAAPNCSNVVCYDGFDIWGQGTMVTVSS (SEQ
ID NO: 143)
Light Chain
EIVLTQSPGTLSLSPGERATLSCRASQSVSSSYLAWYQQKPGQAPRLLIYGASSRATGFPDRESGS
GSGTDFTLTISRLEPEDFAVYYCQQYGNSPWTFGQGTKVEIR (SEQ ID NO: 5)

| | |
|---|---|
| Variant name: | B1-182.1 heavy /B1-182.1 light_5Mut |

Heavy Chain
QMQLVQSGPEVKKPGTSVKVSCKASGFTFTSSAVQWVRQARGQRLEWIGWIVVGSGNTNYAQK
FQERVTITRDMSTSTAYMELSSLRSEDTAVYYCAAPYCSGGSCFDGFDIWGQGTMVTVSS (SEQ
ID NO: 1)
Light Chain
EIVLTQSPGTLSLSPGERATLSCRASQSVSSSYLAWYQQKPGQAPRLLIYSASSRATGIPDRESGSG
SGTDFTLTISRLEPEDFAVYFCQQYGTSPWTFGQGTKVEIK (SEQ ID NO: 144)

It was found that B1-182.1_58 CDRH3 heavy/B1-182.1 light had an improved yield, ability to concentrate and did not precipitate, whereas B1-182 heavy/B1-182.1 light_5Mut did not have these features (FIG. 29B). Furthermore, the capacity was tested for B1-182.1_58 CDRH3 heavy/B1-182.1 light to neutralize a selected set of SARS-CoV-2 variants and it was shown that B1-182.1_58CDRH3 heavy/B1-182.1 light has similar neutralization potency and breadth to B1-182.1 (FIG. 29C).

### Example 31

### In Vivo Pharmacokinetic Properties

The SARS-CoV-2 monoclonal antibodies, A23-58.1, B1-182.1, A19-61.1, A19-46.1 and B1-182.1_58.1CDRH3 heavy/B1-182.1 light were assessed for their in vivo pharmacokinetic properties in human FcRn transgenic mice (See Avery et al., MAbs 8(6): 1064-78, 2016, doi: 10.1080/19420862.2016.1193660). Each antibody was infused at a dose of 5 mg/kg to 4-5 animals and serum samples were collected at days 0, 1, 2, 5, 7, 9, 14, 21 and 28, and weekly up to week 8 after injection. Serum mAb levels were quantified using ELISA plates coated with the SARS-CoV-2 S2P protein. FIG. 30 shows the sera levels for each antibody group, with levels maintained above 1 μg/mL up to day 56 post infusion in most animals, with slightly higher levels observed for B1-182.1_58.1CDRH3 heavy/B1-182.1 light, A19-46.1 and B1-182.1 compared to A23-58.1 and A19-61.1 at day 56.

The in vivo half-life for each antibody was calculated using a non-compartment model in the WinNonLin software package and are presented in the table below. The average half-life was calculated to be 13.1, 16.3, 13.5, 17.2 and 14.1 days for A23-58.1, B1-182.1, A19-61.1, A19-46.1 and B1-182.1_58.1CDRH3 heavy/B1-182.1 light antibodies, respectively. There was no significant difference in the half-lives between the different antibodies suggesting that they all have comparable in vivo pharmacokinetic profile in this mouse model.

### In Vivo Half-Lives for SARS-CoV-2 Antibodies in Human FcRn Transgenic Mice

| | Half-life |
|---|---|
| Antibody | Day (SEM) |
| A23-58.1 | 13.1 (1.0) |
| B1-182.1 | 16.3 (1.1) |
| A19-61.1 | 13.5 (0.7) |
| A19-46.1 | 17.2 (1.0) |
| B1-182.1_58.1CDRH3 | 14.1 (1.3) |
| heavy/B1-182.1 light | |

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. An isolated monoclonal antibody or antigen binding fragment thereof, comprising:
a) a heavy chain variable (V_{H}) region and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as SEQ ID NOs: 1 and 5, respectively;
b) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 9 and 13, respectively;
c) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 17 and 21, respectively;
d) a V_{H} and a V_{L} comprising a HCDR1, a HCDR2, and a HCDR3, and a 1LCDR1, a LCDR2, and a LCDR3 of the V_{H} and V_{L} set forth as SEQ ID NOs: 25 and 29, respectively;
e) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 33 and 37, respectively;
f) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 41 and 45, respectively;
g) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 49 and 53, respectively;
h) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 57 and 61, respectively;
i) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 65 and 69, respectively;
j) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 73 and 77, respectively,
k) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 81 and 85;
l) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 89 and 93;
m) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 97 and 101;
n) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 105 and 109; or
o) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 143 and 5, respectively,
and wherein the monoclonal antibody specifically binds to a coronavirus spike protein, and neutralizes SARS-CoV-2.

2. The isolated monoclonal or antigen binding fragment of claim 1, wherein
a) the HCDR1, the HCDR2, the HCDR3, the LCDR1, the LCDR2, and the LCDR3 comprise the amino acids sequences set forth as SEQ ID NOs: 2, 3, 4, 6, 7, and 8, respectively;
b) the HCDR1, the HCDR2, the HCDR3, the LCDR1, the LCDR2, and the LCDR3 comprise the amino acids sequences set forth as SEQ ID NOs: 10, 11, 12, 14, 15, and 16, respectively;
c) the HCDR1, the HCDR2, the HCDR3, the LCDR1, the LCDR2, and the LCDR3 comprise the amino acids sequences set forth as SEQ ID NOs: 18, 19, 20, 22, 23, and 24 respectively;
d) the HCDR1, the HCDR2, the HCDR3, the LCDR1, the LCDR2, and the LCDR3 comprise the amino acids sequences set forth as SEQ ID NOs: 26, 27, 28, 30, 31, and 32 respectively;
e) the HCDR1, the HCDR2, the HCDR3, the LCDR1, the LCDR2, and the LCDR3 comprise the amino acids sequences set forth as SEQ ID NOs: 34, 35, 36, 38, 39, and 40 respectively;
f) the HCDR1, the HCDR2, the HCDR3, the LCDR1, the LCDR2, and the LCDR3 comprise the amino acids sequences set forth as SEQ ID NOs: 42, 43, 44, 46, 47, and 48, respectively;
g) the HCDR1, the HCDR2, the HCDR3, the LCDR1, the LCDR2, and the LCDR3 comprise the amino acids sequences set forth as SEQ ID NOs: 50, 51, 52, 54, 55, and 56, respectively;
h) the HCDR1, the HCDR2, the HCDR3, the LCDR1, the LCDR2, and the LCDR3 comprise the amino acids sequences set forth as SEQ ID NOs: 58, 59, 60, 62, 63, and 64, respectively;
i) the HCDR1, the HCDR2, the HCDR3, the LCDR1, the LCDR2, and the LCDR3 comprise the amino acids sequences set forth as SEQ ID NOs: 66, 67, 68, 70, 71, and 72, respectively;
j) the HCDR1, the HCDR2, the HCDR3, the LCDR1, the LCDR2, and the LCDR3 comprise the amino acids sequences set forth as SEQ ID NOs: 74, 75, 76, 78, 79, and 80, respectively;
k) the HCDR1, the HCDR2, the HCDR3, the LCDR1, the LCDR2, and the LCDR3 comprise the amino acids sequences set forth as SEQ ID NOs: 82, 83, 84, 86, 87, and 88, respectively;
l) the HCDR1, the HCDR2, the HCDR3, the LCDR1, the LCDR2, and the LCDR3 comprise the amino acids sequences set forth as SEQ ID NOs: 90, 91, 92, 94, 95, and 96, respectively;
m) the HCDR1, the HCDR2, the HCDR3, the LCDR1, the LCDR2, and the LCDR3 comprise the amino acids sequences set forth as SEQ ID NOs: 98, 99, 100, 102, 103, and 104, respectively;
n) the HCDR1, the HCDR2, the HCDR3, the LCDR1, the LCDR2, and the LCDR3 comprise the amino acids sequences set forth as SEQ ID NOs: 106, 107, 108, 110, 111, and 112, respectively; or
o) the HCDR1, the HCDR2, the HCDR3, the LCDR1, the LCDR2, and the LCDR3 comprise the amino acids sequences set forth as SEQ ID NOs: 2, 3, 58, 6, 7, 8, respectively.

3. The isolated monoclonal or antigen binding fragment of claim 2, wherein
a) the V_{H} and the V_{L} comprise the amino acid sequences at least 90% identical to the amino acid sequences set forth as SEQ ID NOs: 1 and 5, respectively;
b) the V_{H} and the V_{L} comprise the amino acid sequences at least 90% identical to the amino acid sequences set forth as SEQ ID NOs: 9 and 13, respectively;
c) the V_{H} and the V_{L} comprise the amino acid sequences at least 90% identical to the amino acid sequences set forth as SEQ ID NOs: 17 and 21, respectively;
d) the V_{H} and the V_{L} comprise the amino acid sequences at least 90% identical to the amino acid sequences set forth as SEQ ID NOs: 25 and 29, respectively;
e) the V_{H} and the V_{L} comprise the amino acid sequences at least 90% identical to the amino acid sequences set forth as SEQ ID NOs: 33 and 37, respectively;
f) the V_{H} and the V_{L} comprise the amino acid sequences at least 90% identical to the amino acid sequences set forth as SEQ ID NOs: 41 and 45, respectively;
g) the V_{H} and the V_{L} comprise the amino acid sequences at least 90% identical to the amino acid sequences set forth as SEQ ID NOs: 49 and 53, respectively;
h) the V_{H} and the V_{L} comprise the amino acid sequences at least 90% identical to the amino acid sequences set forth as SEQ ID NOs: 57 and 61, respectively;
i) the V_{H} and the V_{L} comprise the amino acid sequences at least 90% identical to the amino acid sequences set forth as SEQ ID NOs: 65 and 69, respectively;
j) the V_{H} and the V_{L} comprise the amino acid sequences at least 90% identical to the amino acid sequences set forth as SEQ ID NOs: 73 and 77, respectively;
k) the V_{H} and the V_{L} comprise the amino acid sequences at least 90% identical to the amino acid sequences set forth as SEQ ID NOs: 81 and 85, respectively;
l) the V_{H} and the V_{L} comprise the amino acid sequences at least 90% identical to the amino acid sequences set forth as SEQ ID NOs: 89 and 93, respectively;
m) the V_{H} and the V_{L} comprise the amino acid sequences at least 90% identical to the amino acid sequences set forth as SEQ ID NOs: 97 and 101, respectively;
n) the V_{H} and the V_{L} comprise the amino acid sequences at least 90% identical to the amino acid sequences set forth as SEQ ID NOs: 105 and 109, respectively; or
o) the V_{H} and the V_{L} comprise the amino acid sequences at least 90% identical to the amino acid sequences set forth as SEQ ID NOs: 143 and 5, respectively.

4. The isolated monoclonal antibody or antigen binding fragment of claim 1, comprising a human framework region.

5. The isolated monoclonal antibody or antigen binding fragment of any one of claim 1, wherein:
a) the V_{H} and the V_{L} comprise the amino acid sequences set forth as SEQ ID NOs: 1 and 5, respectively;
b) the V_{H} and the V_{L} comprise the amino acid sequences set forth as SEQ ID NOs: 9 and 13, respectively;
c) the V_{H} and the V_{L} comprise the amino acid sequences set forth as SEQ ID NOs: 17 and 21, respectively;
d) the V_{H} and the V_{L} comprise the amino acid sequences set forth as SEQ ID NOs: 25 and 29, respectively;
e) the V_{H} and the V_{L} comprise the amino acid sequences set forth as SEQ ID NOs: 33 and 37, respectively;
f) the V_{H} and the V_{L} comprise the amino acid sequences set forth as SEQ ID NOs: 41 and 45, respectively;
g) the V_{H} and the V_{L} comprise the amino acid sequences set forth as SEQ ID NOs: 49 and 53, respectively;
h) the V_{H} and the V_{L} comprise the amino acid sequences set forth as SEQ ID NOs: 57 and 61, respectively;
i) the V_{H} and the V_{L} comprise the amino acid sequences set forth as SEQ ID NOs: 65 and 69, respectively;
j) the V_{H} and the V_{L} comprise the amino acid sequences set forth as SEQ ID NOs: 73 and 77, respectively;
k) the V_{H} and the V_{L} comprise the amino acid sequences set forth as SEQ ID NOs: 81 and 85, respectively;
l) the V_{H} and the V_{L} comprise the amino acid sequences set forth as SEQ ID NOs: 89 and 93, respectively;
m) the V_{H} and the V_{L} comprise the amino acid sequences set forth as SEQ ID NOs: 97 and 101, respectively;
n) the V_{H} and the V_{L} comprise the amino acid sequences set forth as SEQ ID NOs: 105 and 109, respectively; or
o) the V_{H} and the V_{L} comprise the amino acid sequences set forth as SEQ ID NOs: 143 and 5, respectively.

6. The isolated monoclonal antibody of claim 1, wherein the antibody comprises a human constant domain.

7. The isolated monoclonal antibody of claim 1, wherein the antibody is a human antibody.

8. The isolated monoclonal antibody of claim 1, wherein the antibody is an IgA.

9. The isolated monoclonal antibody of claim 1, comprising a recombinant constant domain comprising a modification that increases the half-life of the antibody.

10. The isolated monoclonal antibody of claim 9, wherein the modification increases binding to the neonatal Fc receptor.

11. The isolated monoclonal antibody or antigen binding fragment of claim 1, wherein the antibody specifically binds an N-terminal domain of the coronavirus spike protein.

12. The isolated monoclonal antibody or antigen binding fragment of claim 1, wherein the antibody specifically binds a receptor binding domain (RBD) of the coronavirus spike protein.

13. The isolated monoclonal antibody or antigen binding fragment of claim 1, wherein the antibody neutralizes SARS-CoV-1.

14. The antigen binding fragment of claim 1.

15. The antigen binding fragment of claim 14, wherein the antigen binding fragment is a Fv, Fab, F(ab′)₂, scFV or a scFV₂ fragment.

16. The isolated monoclonal antibody or antigen binding fragment of claim 1, conjugated to a detectable marker.

17. A bispecific antibody comprising the monoclonal antibody or antigen binding fragment of claim 1.

18. The bispecific antibody of claim 17, wherein the bispecific antibody is a dual variable domain immunoglobulin.

19. An isolated nucleic acid molecule encoding the isolated monoclonal antibody or antigen binding fragment of claim 1, a V_{H} or V_{L} of the isolated monoclonal antibody of claim 1, a dual variable domain immunoglobulin comprising the antigen binding fragment.

20. The isolated nucleic acid molecule of claim 19, wherein the nucleic acid molecule is a cDNA sequence encoding the V_{H} or V_{L}.

21. The nucleic acid molecule of claim 1, operably linked to a promoter.

22. A vector comprising the nucleic acid molecule of claim 21.

23. A host cell comprising the nucleic acid molecule of claim 19, or a vector comprising the nucleic acid molecule.

24. A pharmaceutical, comprising an effective amount of the monoclonal antibody or the antigen binding fragment of claim 1, a bispecific antibody comprising the monoclonal antibody or antigen binding fragment, a nucleic acid molecule encoding the monoclonal antibody, antigen binding fragment or bispecific antibody, or a vector comprising the nucleic acid molecule; and
a pharmaceutically acceptable carrier.

25. A method of producing an antibody or antigen binding fragment that specifically binds to a SARS-CoV-2 spike protein, comprising:
expressing one or more nucleic acid molecules encoding the monoclonal antibody or antigen binding fragment of claim 1 in a host cell; and
purifying the monoclonal antibody or antigen binding fragment.

26. A method of detecting the presence of a coronavirus in a biological sample from a subject, comprising:
contacting the biological sample with an effective amount of the antibody or antigen binding fragment of claim 1 under conditions sufficient to form an immune complex; and
detecting the presence of the immune complex in the biological sample, wherein the presence of the immune complex in the biological sample indicates the presence of the coronavirus in the sample.

27. The method of claim 26, wherein detecting the detecting the presence of the immune complex in the biological sample indicates that the subject has a SARS-CoV-2 infection.

28. A method of inhibiting a coronavirus infection in a subject, comprising administering an effective amount of the pharmaceutical composition of claim 24 to the subject, wherein the subject has or is at risk of a coronavirus infection.

29. The method of claim 28, wherein the coronavirus is SARS-CoV-2.

30-31. (canceled)

32. The method of claim 29 wherein the SARS-CoV-2 is the B.1.1.529 variant.


SEQUENCE LISTING (156 sequences)

SEQ ID NO: 1 - protein, 123 aa, homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSS

SEQ ID NO: 2 - protein, 8 aa, homo sapiens
GFTFTSSA

SEQ ID NO: 3 - protein, 8 aa, homo sapiens
IVVGSGNT

SEQ ID NO: 4 - protein, 18 aa, homo sapiens
CAAPYCSGGSCFDGFDIW

SEQ ID NO: 5 - protein, 108 aa, homo sapiens
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIR

SEQ ID NO: 6 - protein, 7 aa, homo sapiens
QSVSSSY

SEQ ID NO: 7 - protein, 3 aa, homo sapiens
GAS

SEQ ID NO: 8 - protein, 11 aa, homo sapiens [sequence omitted]

SEQ ID NO: 9 - protein, 127 aa, homo sapiens
QVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSS

SEQ ID NO: 10 - protein, 8 aa, homo sapiens
GFTFSSYA

SEQ ID NO: 11 - protein, 8 aa, homo sapiens
ISYDGSNQ

SEQ ID NO: 12 - protein, 22 aa, homo sapiens
CARDLAIAVAGTWHYYNGMDVW

SEQ ID NO: 13 - protein, 107 aa, homo sapiens
DIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIK

SEQ ID NO: 14 - protein, 6 aa, homo sapiens
QGISSW

SEQ ID NO: 15 - protein, 3 aa, homo sapiens
DAS

SEQ ID NO: 16 - protein, 11 aa, homo sapiens [sequence omitted]

SEQ ID NO: 17 - protein, 126 aa, homo sapiens
QVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSS

SEQ ID NO: 18 - protein, 8 aa, homo sapiens
GFTLSSYG

SEQ ID NO: 19 - protein, 8 aa, homo sapiens
ISYDGSNK

SEQ ID NO: 20 - protein, 21 aa, homo sapiens
CARGWAYWELLPDYYYGMDVW

SEQ ID NO: 21 - protein, 110 aa, homo sapiens
QTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVL

SEQ ID NO: 22 - protein, 9 aa, homo sapiens
SGSVSTAYF

SEQ ID NO: 23 - protein, 3 aa, homo sapiens
GTN

SEQ ID NO: 24 - protein, 12 aa, homo sapiens
CVLYMGRGIVVF

SEQ ID NO: 25 - protein, 123 aa, homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSS

SEQ ID NO: 26 - protein, 8 aa, homo sapiens
GFTFTSSA

SEQ ID NO: 27 - protein, 8 aa, homo sapiens
IVVGSGNT

SEQ ID NO: 28 - protein, 18 aa, homo sapiens
CAAPNCSNVVCYDGFDIW

SEQ ID NO: 29 - protein, 108 aa, homo sapiens
EIVLTQSPGTLSLSPGERATLSCRASQSVSSSYLAWYQQKPGQAPRLLIYSASSRATGIPDRFSGSGSGTDFTLTISRLEPEDFAVYFCQQYGTSPWTFGQGTKVEIK

SEQ ID NO: 30 - protein, 7 aa, homo sapiens
QSVSSSY

SEQ ID NO: 31 - protein, 3 aa, homo sapiens
SAS

SEQ ID NO: 32 - protein, 11 aa, homo sapiens [sequence omitted]

SEQ ID NO: 33 - protein, 124 aa, homo sapiens
EVQLVESGGGLVQPGRSLRLSCAASGFTFDDYAMHWVRQTPGKGLEWVSGISWNSGDIDYADSVKGRFTISRDNAKNSLYLQMNSLRTEDTALYYCTKGWFGEFFGAGSICDYWGQGTLVTVSS

SEQ ID NO: 34 - protein, 8 aa, homo sapiens
GFTFDDYA

SEQ ID NO: 35 - protein, 8 aa, homo sapiens
ISWNSGDI

SEQ ID NO: 36 - protein, 19 aa, homo sapiens
CTKGWFGEFFGAGSICDYW

SEQ ID NO: 37 - protein, 106 aa, homo sapiens
EIVMTQSPATLSVSPGERATLSCRASQSVSNNLAWYQQKPGQAPRLLIYGASTRATGIPARFSGSGSGTEFTLTISSLQSEDFAVYYCQQYNNWPLFGQGTKVEIK

SEQ ID NO: 38 - protein, 6 aa, homo sapiens
QSVSNN

SEQ ID NO: 39 - protein, 3 aa, homo sapiens
GAS

SEQ ID NO: 40 - protein, 10 aa, homo sapiens [sequence omitted]

SEQ ID NO: 41 - protein, 123 aa, homo sapiens
QVQLVESGGGVVQPGRSLRLSCAASGFTFSNYAIHWVRQAPGKGLEWVAVISYDGSNKYYADSVKGRFTISRDNSKNTLYLEMNSLRAEDMAVYYCARVGPYQYDSSAAFDIWGQGTMVTVSS

SEQ ID NO: 42 - protein, 8 aa, homo sapiens
GFTFSNYA

SEQ ID NO: 43 - protein, 8 aa, homo sapiens
ISYDGSNK

SEQ ID NO: 44 - protein, 18 aa, homo sapiens
CARVGPYQYDSSAAFDIW

SEQ ID NO: 45 - protein, 107 aa, homo sapiens
DIQMTQSPSTLSASVGDRVTITCRASQSISSWLAWYQQKPGQAPKLLIYDASSLESGVPSRFSGSGSGTEFTLTISSLQPDDFATYYCQQYNSYSRTFGQGTKVEIK

SEQ ID NO: 46 - protein, 6 aa, homo sapiens
QSISSW

SEQ ID NO: 47 - protein, 3 aa, homo sapiens
DAS

SEQ ID NO: 48 - protein, 11 aa, homo sapiens [sequence omitted]

SEQ ID NO: 49 - protein, 120 aa, homo sapiens
QVQLVESGGGVVQPGRSLRLSCAASGFTFTNYAMHWVRQAPGKGLEWVAVISNDGSDKYYADSVKGRFTISRDNSKSTVYLQMSSLRPEDTAVYFCARDPPQVHWSLDYWGQGTLVTVSS

SEQ ID NO: 50 - protein, 8 aa, homo sapiens
GFTFTNYA

SEQ ID NO: 51 - protein, 8 aa, homo sapiens
ISNDGSDK

SEQ ID NO: 52 - protein, 15 aa, homo sapiens
CARDPPQVHWSLDYW

SEQ ID NO: 53 - protein, 110 aa, homo sapiens
QSALTQPPSASGSPGQSVTISCTGTSSDVGDYNYVSWYQHHPGKAPKLIIYDVSKRPSGVPDRFSGSKSGDTASLTVSGLQAEDEADYYCSSYAGNNNAVFGTGTKVTVL

SEQ ID NO: 54 - protein, 9 aa, homo sapiens
SSDVGDYNY

SEQ ID NO: 55 - protein, 3 aa, homo sapiens
DVS

SEQ ID NO: 56 - protein, 12 aa, homo sapiens
CSSYAGNNNAVF

SEQ ID NO: 57 - protein, 122 aa, homo sapiens
QVQLVESGGGVVQPGRSLRLSCAASGFTFSNYGMHWVRQAPGKGLEWLAVISYDGSNKYYADSVKGRFTISRDNSKNTLFLQMNSLRAEDTAVYYCAKESQFGELFEALDYWGQGTLVTVSS

SEQ ID NO: 58 - protein, 8 aa, homo sapiens
GFTFSNYG

SEQ ID NO: 59 - protein, 8 aa, homo sapiens
ISYDGSNK

SEQ ID NO: 60 - protein, 17 aa, homo sapiens
CAKESQFGELFEALDYW

SEQ ID NO: 61 - protein, 108 aa, homo sapiens
SYELTQPPSVSVSPGQTARITCSGDALPRKYAFWYQQKSGQAPVLVISEDSKRPSGIPERFSGSSSGTMATLTISGAQVEDEADYYCYSTDSSGNHRVFGGGTKLTVL

SEQ ID NO: 62 - protein, 6 aa, homo sapiens
ALPRKY

SEQ ID NO: 63 - protein, 3 aa, homo sapiens
EDS

SEQ ID NO: 64 - protein, 13 aa, homo sapiens [sequence omitted]

SEQ ID NO: 65 - protein, 123 aa, homo sapiens
QVQLVESGGGVVQPGRSLRLSCAASGFIFSSYGVHWVRQAPGKGLEWVAVIWHDESNKDYADSVKGRFTISRDNSKNTVYLQMNSLRAEDTAMYYCARDGYDFLTGAYELDYWGQGTLVTVSS

SEQ ID NO: 66 - protein, 8 aa, homo sapiens
GFIFSSYG

SEQ ID NO: 67 - protein, 8 aa, homo sapiens
IWHDESNK

SEQ ID NO: 68 - protein, 18 aa, homo sapiens
CARDGYDFLTGAYELDYW

SEQ ID NO: 69 - protein, 107 aa, homo sapiens
SYELTQPPSVSVSPGQTARITCSGDALPKQYAYWYQQKPGQAPVVVIYKDSERPSGIPERFSGSSSGTTVTLTISGVQAEDEADYYCQSADSSGTWVFGGGTKLTVL

SEQ ID NO: 70 - protein, 6 aa, homo sapiens
ALPKQY

SEQ ID NO: 71 - protein, 3 aa, homo sapiens
KDS

SEQ ID NO: 72 - protein, 12 aa, homo sapiens [sequence omitted]

SEQ ID NO: 73 - protein, 122 aa, homo sapiens
QVQLVESGGGVVQPGGSLRLSCAASGFTFSSFGMHWVRQAPGKGLEWVAFIRYDGSNKYYADSVKGRFTISRDNSKNTLYLQMNSLRAEDTAVYYCAKTELYYYDSSGPLGWGQGTLVTVSS

SEQ ID NO: 74 - protein, 8 aa, homo sapiens
GFTFSSFG

SEQ ID NO: 75 - protein, 8 aa, homo sapiens
IRYDGSNK

SEQ ID NO: 76 - protein, 17 aa, homo sapiens
CAKTELYYYDSSGPLGW

SEQ ID NO: 77 - protein, 107 aa, homo sapiens
DIQMTQSPSTLSASVGDRVTITCRASQSITSWLAWYQQKPGKAPKLLIYDASSLESGVPSRFSGSGSGTEFTLTISSLQPDDFATYYCQQYNSYPWTFGQGTKVEIK

SEQ ID NO: 78 - protein, 6 aa, homo sapiens
QSITSW

SEQ ID NO: 79 - protein, 3 aa, homo sapiens
DAS

SEQ ID NO: 80 - protein, 11 aa, homo sapiens
CQQYNSYPWTF

SEQ ID NO: 81 - protein, 120 aa, homo sapiens
QVHLEESGGAVVQPGRSLRLSCAASGFTFSSYGMHWVRQAPGKGLEWVAVISHDGSYKYYADSVKGRFTISRDNSKDTLYLQTNSLRAEDTAMYYCAKSYGYWMAYFDYWGQGTLVTVSS

SEQ ID NO: 82 - protein, 8 aa, homo sapiens
GFTFSSYG

SEQ ID NO: 83 - protein, 8 aa, homo sapiens
ISHDGSYK

SEQ ID NO: 84 - protein, 15 aa, homo sapiens
CAKSYGYWMAYFDYW

SEQ ID NO: 85 - protein, 107 aa, homo sapiens
DIQMTQSPSSLSASVGDRVTITCRASQDISNYLAWYQQKPGKVPKLLIYAASTLQSGVPSRFSGSGSGTDFTLTISSLQPEDVATYYCQKYNSPWHTFGGGTKVEIK

SEQ ID NO: 86 - protein, 6 aa, homo sapiens
QDISNY

SEQ ID NO: 87 - protein, 3 aa, homo sapiens
AAS

SEQ ID NO: 88 - protein, 11 aa, homo sapiens
CQKYNSPWHTF

SEQ ID NO: 89 - protein, 125 aa, homo sapiens
QVQLVQSGAEVKKPGASVKVSCKASGYTFTSNGVTWVRQAPGQGLEWMGWISTYNGDTNYAQKLQGRVSMTTDTSTRTVYMELRSLRSDDTAVYYCARVGDAYCSGGSCYHFDYWGQGTLVTVSS

SEQ ID NO: 90 - protein, 8 aa, homo sapiens
GYTFTSNG

SEQ ID NO: 91 - protein, 8 aa, homo sapiens
ISTYNGDT

SEQ ID NO: 92 - protein, 20 aa, homo sapiens
CARVGDAYCSGGSCYHFDYW

SEQ ID NO: 93 - protein, 109 aa, homo sapiens
EIVMTQSPATLSVSPGERATLSCRASQSVSTNLAWYQQKPGQAPRLLIYGASTRATGIPARFSGSGSGTEFTLTISSLQSEDFALYYCQQYDNWPPEFTFGPGTKVDIK

SEQ ID NO: 94 - protein, 6 aa, homo sapiens
QSVSTN

SEQ ID NO: 95 - protein, 3 aa, homo sapiens
GAS

SEQ ID NO: 96 - protein, 13 aa, homo sapiens
CQQYDNWPPEFTF

SEQ ID NO: 97 - protein, 122 aa, homo sapiens
QVQLVESGGGVVQPGRSLRLSCVVSGLIFSTYDMHWVRQAPGKGLEWVAVISYDGSYKHYADSVKGRFTISRDSSKNTLYLQMNSLRPEDTAVYYCAKGEGVVAGTGKFDYWGQGTLVTVSS

SEQ ID NO: 98 - protein, 8 aa, homo sapiens
GLIFSTYD

SEQ ID NO: 99 - protein, 8 aa, homo sapiens
ISYDGSYK

SEQ ID NO: 100 - protein, 17 aa, homo sapiens
CAKGEGVVAGTGKFDYW

SEQ ID NO: 101 - protein, 108 aa, homo sapiens
SYVLTQPPSVSVAPGQTARITCGGNNIGSKSVHWYQQKPGQAPVLVVYDDSDRPSGIPERFSGSNSGNTATLTISRVEAGDEADYYCQVWDGSGDPWVFGGGTKLTVL

SEQ ID NO: 102 - protein, 6 aa, homo sapiens
NIGSKS

SEQ ID NO: 103 - protein, 3 aa, homo sapiens
DDS

SEQ ID NO: 104 - protein, 13 aa, homo sapiens
CQVWDGSGDPWVF

SEQ ID NO: 105 - protein, 123 aa, homo sapiens
QVQLVESGGGVVQPGRSLRLSCAASGFTFSSYGMHWVRQAPGKGLEWVAFISYDGSNKYYADSVKGRFTISRDNSKNTLYLQMNSLRAEDTAVYYCAKDYWSVAAGTSWFDPWGQGTLVSVSS

SEQ ID NO: 106 - protein, 8 aa, homo sapiens
GFTFSSYG

SEQ ID NO: 107 - protein, 8 aa, homo sapiens
ISYDGSNK

SEQ ID NO: 108 - protein, 18 aa, homo sapiens
CAKDYWSVAAGTSWFDPW

SEQ ID NO: 109 - protein, 107 aa, homo sapiens; identical to SEQ ID NO: 69

SEQ ID NO: 110 - protein, 6 aa, homo sapiens
ALPKQY

SEQ ID NO: 111 - protein, 3 aa, homo sapiens
KDS

SEQ ID NO: 112 - protein, 12 aa, homo sapiens [sequence omitted]

SEQ ID NO: 113 - DNA, 370 bp, homo sapiens
caaatgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcaccgaattgtagtaatgttgtatgctatgatggttttgatatctggggccaagggacaatggtcaccgtctcttcag

SEQ ID NO: 114 - DNA, 325 bp, homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatagtgcatccagcagggccactggcatcccagacaggttcagtggcagtgggtctgggacagacttcactctcaccatcagcagactggagcctgaagattttgcagtgtatttctgtcagcagtatggtacctcaccgtggacgttcggccaagggaccaaggtggaaatcaaac

SEQ ID NO: 115 - DNA, 382 bp, homo sapiens
caggtgcagctggtggagtctgggggaggcgtggtccagcctgggaggtccctgagactctcctgtgcagcctctggattcaccttcagtagctatgctttccactgggtccgccaagctccgggcaaggggctggagtgggtggcagttatatcatatgatggaagcaatcaatactacgcagactccgtgaagggccgattcaccatctccagagacaattccaagaacacgctgtatctgcaaatgaacagcctgagagctgacgacacggctgtgtattactgtgcgagagatctggctatagcagtggctggtacgtggcactattataacggtatggacgtctggggccaagggaccacggtcaccgtctcctcag

SEQ ID NO: 116 - DNA, 322 bp, homo sapiens
gacatccagatgacccagtctccatcttccgtgtctgcatcagtaggagacagagtcatcatcacttgtcgggcgagtcagggtatttccagctggttagcctggtatcagcagaaaccagggaaagcccctaaggtcctgatctatgatgcatccagtttgcaaagtggggtcccatcaaggttcagcggcagtggatatgggacagatttcactctcaccatcagcagcctgcagcctgaagattctgcaacttactattgtcaacaggctaaaagttttccgatcaccttcggccaagggacacgactggagattaaac

SEQ ID NO: 117 - DNA, 379 bp, homo sapiens
caggtgcagctggtggagtctgggggaggcgtggtccagcctgggaggtccctgagactctcctgtgcagcctctggattcaccctcagtagctatggcatgcactgggtccgccaggctccaggcaaggggctggagtgggtggcagttatatcatatgatggaagtaataaatactatgtagactccgtgaagggccgattcaccatctccagagacaattccaagaacacgctgtatctgcaaatgaacagcctgagagctgaggacacggctgtgtattactgtgcgagggggtgggcttattgggagctactccctgactactactacggtatggacgtctggggccaagggaccacggtcaccgtctcctcag

SEQ ID NO: 118 - DNA, 331 bp, homo sapiens
cagactgtggtgacacaggagccatcgttctcagtgtcccctggagggacagtcacactcacttgtggcttgagctctggctcagtctctactgcttacttccccagctggtaccagcagaccccaggccaggctccacgcacgctcatctacggtacaaacactcgctcttctggggtccccgatcgcttctctggctccatccttgggaacaaagctgccctcaccatcacgggggcccaggcagacgatgaatctgattattactgtgtgctgtatatgggtagaggcattgtggtattcggcggagggaccaagctgaccgtcctag

SEQ ID NO: 119 - DNA, 370 bp, homo sapiens
caggtgcagctggtggagtctgggggaggcgtggtccagcctgggaggtccctgagactctcctgtgcagcctctggattcaccttcagtaactatgctatccactgggtccgccaggctccaggcaaggggctggagtgggtggcagttatatcatatgatggaagcaataaatactacgcagactccgtgaagggccgattcaccatctccagagacaattccaagaacacgctgtatctggaaatgaacagcctgagagctgaggatatggctgtgtattactgtgcgagagtcggtccgtatcagtatgatagtagtgctgcttttgatatctggggccaagggacaatggtcaccgtctcttcag

SEQ ID NO: 120 - DNA, 322 bp, homo sapiens
gacatccagatgacccagtctccttccaccctgtctgcatctgtaggagacagagtcaccatcacttgccgggccagtcagagtattagtagctggttggcctggtatcagcagaaaccagggcaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttattctcgaacgttcggccaagggaccaaggtggaaatcaaac

SEQ ID NO: 121 - DNA, 361 bp, homo sapiens
caggtgcagttggtagagtctgggggaggcgtggtccagcctgggaggtccctgagactctcctgtgcagcctctggattcaccttcactaattatgcaatgcactgggtccgccaggctccaggcaaggggctggagtgggtggcagttatatcaaatgatggaagcgataaatactacgcggactccgtgaagggccgattcaccatctccagagacaattccaagagcacggtttatttgcaaatgagcagcctgagacctgaggacacggctgtgtatttctgtgcgagagatcccccccaggttcactggtccctcgactactggggccagggaaccctggtcaccgtctcctcag

SEQ ID NO: 122 - DNA, 331 bp, homo sapiens
cagtctgccctgactcagcctccctccgcgtccgggtctcctggacagtcagtcaccatctcctgcactggaaccagcagtgacgttggtgattataactatgtctcctggtaccaacaccacccaggcaaagcccccaaactcataatttatgacgtcagtaagcggccctcaggggtccctgatcgcttctctggctccaagtctggcgacacggcctccctgaccgtctctgggctccaggctgaggatgaggctgattattactgcagctcatatgcaggcaacaacaatgccgtcttcggaactgggaccaaggtcaccgtcctag

SEQ ID NO: 123 - DNA, 373 bp, homo sapiens
gaagtgcagctggtggagtctgggggaggcttggtacagcctggcaggtccctgagactctcctgtgcagcctctggattcacgtttgatgattatgccatgcactgggtccggcaaactccagggaagggcctggagtgggtctcaggtattagttggaatagtggtgacatagactatgcggactctgtgaagggccgattcaccatctccagagacaacgccaagaactccctgtatctgcaaatgaacagtctgagaactgaggacacggccttgtattactgtacaaaagggtggttcggggagttcttcggggccgggtcgatatgtgactactggggccagggaaccctggtcaccgtctcctcag

SEQ ID NO: 124 - DNA, 319 bp, homo sapiens
gaaatagtgatgacgcagtctccagccaccctgtctgtgtctccaggggaaagagccaccctctcctgcagggccagtcagagtgttagcaacaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgttgttcggccaagggaccaaggtggaaatcaaac

SEQ ID NO: 125 - DNA, 367 bp, homo sapiens
caggtgcagctggtggagtctgggggaggcgtggtccagcctgggaggtccctgagactctcctgtgcagcctctggattcaccttcagtaactatggcatgcactgggtccgccaggctccaggcaaggggctggagtggctggcagttatatcatatgatggaagtaataaatactatgcggactccgtgaagggccgattcaccatctccagagacaattccaagaacacgctgtttctgcaaatgaacagcctgagagctgaggacacggctgtgtattactgtgcgaaagagtcgcaattcggggagttattcgaagccttagactactggggccagggaaccctggtcaccgtctcctcag

SEQ ID NO: 126 - DNA, 325 bp, homo sapiens
tcctatgagctgacacagccaccctcggtgtcagtgtccccaggacaaacggccaggatcacctgctctggagatgcattgccaagaaaatatgctttttggtaccagcagaagtcaggccaggcccctgtgctggtcatctctgaggacagcaaacgaccctccgggatccctgagagattctctggctccagctcagggacaatggccaccttgactatcagtggggcccaggtggaggatgaagctgactactactgttactcaacagacagcagtggtaatcatagggtgttcggcggagggaccaaactgaccgtcctag

SEQ ID NO: 127 - DNA, 370 bp, homo sapiens
caggtgcagctggtggagtctgggggaggcgtggtccagcctgggaggtccctgagactctcctgtgcagcgtctggattcatcttcagtagctatggcgtgcactgggtccgccaggctccaggcaaggggctggagtgggtggcagttatatggcatgatgaaagtaataaagactatgcagactccgtgaagggccgattcaccatctccagagacaattccaagaacacggtgtatctgcaaatgaacagcctgagagccgaggacacggctatgtattattgtgcgagagatggttacgattttttgactggggcttacgagcttgactactggggccagggaaccctggtcaccgtctcctcag

SEQ ID NO: 128 - DNA, 322 bp, homo sapiens
tcctatgagctgacacagccaccctcggtgtcagtgtccccaggacagacggccaggatcacctgctctggagatgcattgccaaagcaatatgcttattggtaccagcagaagccaggccaggcccctgtggtggtgatatataaagacagtgagaggccctcagggatccctgagcgattctctggctccagctcagggacaacagtcacgttgaccatcagtggagtccaggcagaagatgaggctgactattactgtcaatcagcagacagcagtggcacttgggtgttcggcggagggaccaaactgaccgtcctag

SEQ ID NO: 129 - DNA, 367 bp, homo sapiens
caggtgcagctggtggagtctgggggaggcgtggtccagcctggggggtccctgagactctcctgtgcagcgtctggattcaccttcagtagctttggcatgcactgggtccgccaggctccaggcaaggggctggagtgggtggcatttatacggtatgatggaagtaataaatactatgcagactccgtgaagggccgattcaccatctccagggacaattccaagaacacgctgtatctgcaaatgaacagcctgagagctgaggacacggctgtgtattattgtgcgaagacagaactatattactatgatagtagtggcccattggggtggggccagggaaccctggtcaccgtctcctcag

SEQ ID NO: 130 - DNA, 322 bp, homo sapiens
gacatccagatgacccagtctccttccaccctgtctgcatctgtaggagacagagtcaccatcacttgccgggccagtcagagtattactagctggttggcctggtatcagcagaaaccagggaaagcccctaagctcctgatctatgatgcctccagtttggaaagtggtgtcccatcaaggttcagcggcagtggatctgggacagaattcactctcaccatcagcagcctgcagcctgatgattttgcaacttattactgccaacagtataatagttatccgtggacgttcggccaagggaccaaggtggaaatcaaac

SEQ ID NO: 131 - DNA, 361 bp, homo sapiens
caggtgcacctggaggagtctgggggagccgtggtccagcctgggaggtccctgagactctcctgtgcagcctctggattcaccttcagtagctatggcatgcactgggtccgccaggccccaggcaaggggctggagtgggtggcagttatatcacatgatggaagttataagtactatgcagactccgtgaagggccgattcaccatctccagagacaattccaaggacacgctctatctgcaaacgaacagcctgagagctgaggacacggctatgtattactgtgcgaaaagctatggttattggatggcctactttgactactggggccagggaaccctggtcaccgtctcctcag

SEQ ID NO: 132 - DNA, 322 bp, homo sapiens
gacatccagatgacccagtctccatcctccctgtctgcatctgtaggagacagagtcaccatcacttgccgggcgagtcaggacattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtccctggcacactttcggcggagggaccaaggtggagatcaaac

SEQ ID NO: 133 - DNA, 376 bp, homo sapiens
caggttcagctggtgcagtctggagctgaggtgaagaagcctggggcctcagtgaaggtctcctgcaaggcttccggttacacctttaccagcaatggagtcacctgggtgcgacaggcccctggacaagggcttgagtggatgggatggatcagcacttacaatggagacacaaactatgcacagaagctccagggcagagtctccatgaccacagacacatccacgcgcacagtttacatggagctgaggagcctgagatctgacgacacggccgtctattactgtgcgagggtgggggatgcatattgtagtggtggtagctgctatcactttgactactggggccagggaaccctggtcaccgtctcctcag

SEQ ID NO: 134 - DNA, 328 bp, homo sapiens
gaaatagtgatgacgcagtctccagccaccctgtctgtgtctccaggggaaagagccaccctctcctgcagggccagtcagagtgttagcaccaacttagcctggtaccagcagaagcctggccaggctcccaggctcctcatctatggtgcatccaccagggccactggtatcccagccaggttcagtggcagtgggtctgggacagagttcactctcaccatcagcagcctgcagtctgaagattttgcactttattactgtcagcagtatgataactggcctccggaattcactttcggccctgggaccaaagtggatatcaaac

SEQ ID NO: 135 - DNA, 367 bp, homo sapiens
caggtgcagctggtggagtctgggggaggcgtggtccagcctgggaggtccctgagactctcctgtgtagtctctggactcattttcagtacctatgacatgcactgggtccgccaggctccaggcaaggggctggagtgggtggcagttatatcatatgatggaagttacaaacactatgcagactccgtgaagggccgattcaccatctccagagacagttccaagaacacgctgtatctgcaaatgaacagcctgagacctgaagacacggctgtctattactgtgcgaaaggggagggagtagtggctggtacggggaagtttgactactggggccagggaaccctggtcaccgtctcctcag

SEQ ID NO: 136 - DNA, 325 bp, homo sapiens
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagtgaccggccctcagggatccctgagcgattctctggctccaactctgggaacacggccaccctgaccatcagcagggtcgaggccggggatgaggccgactattactgtcaggtgtgggatggtagtggtgatccttgggtgttcggcggagggaccaagctgaccgtcctag

SEQ ID NO: 137 - DNA, 370 bp, homo sapiens
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcatttatatcatatgatggaagtaataaatactatgcagactccgtgaagggccgattcaccatctccagagacaattccaagaacactctgtatctgcaaatgaacagcctgcgagctgaggacacggctgtgtattactgtgcgaaagattattggtcagtagcagctggtactagctggttcgacccctggggccagggaaccctggtcagcgtctcctcag

SEQ ID NO: 138 - DNA, 322 bp, homo sapiens
tcctatgagctgacacagccaccctcggtgtcagtgtccccaggacagacggccaggatcacctgctctggagatgcattgccaaagcaatatgcttattggtaccagcagaagccaggccaggcccctgtggtggtgatatataaagacagtgagaggccctcagggatccctgagcgattctctggctccagctcagggacaacagtcacgttgaccatcagtggagtccaggcagaagacgaggctgactattactgtcaatcagcagacagcagtggtacttgggtgttcggcggagggaccaagctgaccgtcctag

SEQ ID NO: 139 - DNA, 370 bp, homo sapiens
caaatgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctatatggagctgagcagcctgagatccgaggacacggccgtgtattactgtgcggccccttactgtagtggtggtagctgctttgatggttttgatatctggggccaagggacaatggtcaccgtctcttcag

SEQ ID NO: 140 - DNA, 325 bp, homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcttcccagacaggttcagtggcagtgggtctgggacagacttcactctcaccatcagcagactggagcctgaagattttgcagtgtattactgtcagcagtatggtaactcaccctggacgttcggccaagggaccaaggtggaaatcagac

SEQ ID NO: 141 - protein, 109 aa, homo sapiens
VIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYR

SEQ ID NO: 142 - DNA, 33 bp, homo sapiens
gaggctcagcgggaagaccttggggctggtcgg

SEQ ID NO: 143 - protein, 122 aa, artificial sequence
  recombinant antibody sequence
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVS

SEQ ID NO: 144 - protein, 108 aa, artificial sequence; identical to SEQ ID NO: 29
  recombinant antibody sequence

SEQ ID NO: 145 - protein, 3 aa, artificial sequence
  recombinant antibody sequence
SAS

SEQ ID NO: 146 - protein, 11 aa, artificial sequence [sequence omitted]

SEQ ID NO: 147 - protein, 98 aa, homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 148 - protein, 123 aa, homo sapiens
QVQLVQSGPEVKKPGTSVRVSCKASGFTFTSSAVQWVRQARGQRLEWVGWIVVGSGNTNYAQKFHERVTITRDMSTSTAYMELSSLRSEDTAVYYCASPYCSGGSCSDGFDIWGQGTMVTVSS

SEQ ID NO: 149 - protein, 123 aa, homo sapiens
QVQLVQSGPEVKKPGTSVKVSCKASGFTFTTSAVQWVRQARGQRLEWIGWIVVGSGNTNYAQKFQERVTITRDMSTTTAYMELSSLRSEDTAVYFCAAPHCNSTSCYDAFDIWGQGTMVTVSS

SEQ ID NO: 150 - protein, 91 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQ

SEQ ID NO: 151 - protein, 109 aa, homo sapiens
DIVLTQTPGTLSLSPGERATLSCRASQSVSSSYLAWYQQKPGQAPRLLIYGASSRATGIPDRFSGSGSGTDFTLTISRLEPEDFAVYYCQQYVGLTGWTFGQGTKVEIK

SEQ ID NO: 152 - protein, 108 aa, homo sapiens
DIQMTQSPGTLSLSPGEGATLSCRASQSVSSSYLAWYQQKPGQAPRLLIYGASSGATGIPDRFSGSGSGTDFTLTISRLEPEDFAVYYCQQYGSSPYTFGQGTKVEIK

SEQ ID NO: 153 - protein, 29 aa, homo sapiens
YCAAPNCSNVVCYDGFDIWGQGTMVTVSS

SEQ ID NO: 154 - protein, 29 aa, homo sapiens
YCAAPYCSGGSCFDGFDIWGQGTMVTVSS

SEQ ID NO: 155 - protein, 29 aa, homo sapiens
YCAAPYCSSISCNDGFDIWGQGTMVTVSS

SEQ ID NO: 156 - protein, 29 aa, homo sapiens
YCASPYCSGGSCSDGFDIWGQGTMVTVSS